(19) United States
(12) Patent Application Publication
(10) Pub. No.: US 2022/0025466 A1
(43) Pub. Date: Jan. 27, 2022

(54) DIFFERENTIAL METHYLATION

(71) Applicant: UCL Business LTD, London (GB)

(72) Inventors: Vitor Hugo De Sousa Teixeira, London (GB); Samuel Janes, London (GB); Christodoulos P. Pipinikas, London (GB); Adam James Pennycuick, London (GB)

(21) Appl. No.: 17/309,348

(22) PCT Filed: Nov. 29, 2019
(86) PCT No.: PCT/GB2019/053386

§ 371 Date: May 20, 2021

(30) Foreign Application Priority Data
Nov. 29, 2018 (GB) 1819452.2

(51) Int. Cl. C12Q 1/6886

(52) U.S. Cl. CPC C12Q 1/6886; C12Q 2600/112; C12Q 2600/154

(57) ABSTRACT

The present invention relates to a method of identifying whether or not an individual has a cancer, a pre-invasive lesion that will progress to a cancer, or a pre-cancerous cell population that will progress to a cancer based on a methylation heterogeneity index (MHI). The present invention also relates to a method of treating and/or preventing a cancer and/or treating a pre-invasive lesion that will progress to a cancer or a pre-cancerous cell population that will progress to a cancer in an individual, the method comprising: identifying a cancer, a pre-invasive lesion that will progress to a cancer, or a pre-cancerous cell population that will progress to a cancer based on an MHI. The present invention also relates to an MHI and uses thereof, for identifying in an individual a cancer, a pre-invasive lesion that will progress to a cancer, or a pre-cancerous cell population that will progress to a cancer.

### FIELD OF THE INVENTION

The present invention relates to a Methylation Heterogeneity Index (MHI) derived from the differential methylation of DNA from an individual.

The present invention also relates to a method of identifying whether or not an individual has a cancer, a pre-invasive lesion that will progress to a cancer, or a pre-cancerous cell population that will progress to a cancer based on an MHI determined for DNA sample taken from the individual.

The present invention also relates to a method of treating and/or preventing a cancer and/or treating a pre-invasive lesion that will progress to a cancer or a pre-cancerous cell population that will progress to a cancer in an individual, the method comprising:

identifying a cancer, a pre-invasive lesion that will progress to a cancer, or a pre-cancerous cell population that will progress to a cancer in the individual by performing a method comprising determining an MHI for a DNA sample taken from the individual; and

administering a cancer therapy to the individual.

The present invention also relates to an MHI as described herein for identifying in an individual a cancer, a pre-invasive lesion that will progress to a cancer, or a pre-cancerous cell population that will progress to a cancer.

The present invention also relates to uses of an MHI as described herein.

### BACKGROUND TO THE INVENTION

Cancer represents a group of diseases involving abnormal cell growth with the potential to invade or spread to other parts of the body. Cancer often develops from pre-cancerous lesions and pre-cancerous cell populations. Lung cancer is the commonest cause of cancer death worldwide with 1.5 million deaths per year [1]. Lung squamous cell carcinoma (LUSC) is the most common subtype in parts of Europe and second in the U.S.A [2]. Before progression to invasive LUSC, there is step-wise evolution of ever more disordered pre-invasive lesions, ranging from mild and moderate dysplasia (low-grade lesions) to severe dysplasia and carcinoma-in-situ (CIS; high-grade lesions) [3]. Thus, lung cancer and LUSC in particular is a suitable model for cancer progression. The accessibility of the proximal airways allows detection and monitoring of these lesions using high-resolution diagnostic approaches such as autofluorescence bronchoscopy (AFB) [4]. This technique enables the acquisition of tissue throughout the natural history of LUSC, providing an excellent model to study early tumorigenesis in human patients.

The molecular alterations that occur in cells before a cancer manifests are largely uncharted. Lung carcinoma-in-situ (CIS) lesions are the pre-invasive precursor to squamous cell carcinoma. While microscopically identical, their future is in equipoise with half progressing to invasive cancer and half regressing or remaining static. The cellular basis of this clinical observation is unknown.

Clinically, the optimal management of pre-invasive airway lesions remains unclear, despite the availability of surgery, radiotherapy and ablative techniques [5]. AFB with biopsy allows assessment of the size, gross morphology and histopathology of pre-invasive lesions but cannot distinguish lesions that will ultimately progress to invasive tumours from those that will spontaneously regress. As such, indiscriminate surgical resection of pre-invasive lesions or external beam radiotherapy represents over-treatment: lesions will spontaneously regress in 30% of cases, patient co-morbidity and poor lung function impart considerable risk, and the presence of field cancerization means independent lung cancers frequently emerge at sites outside resection or therapy margins [6].

Improved assays for the accurate diagnosis and management of pre-cancerous lesions and/or cancer are sought and would be of significant clinical and economic benefit, particularly assays which are non-invasive and allow the discrimination between progressive and regressive lesions, thus avoiding the need for unnecessary medical intervention.

### SUMMARY OF THE INVENTION

Provided is a new understanding of cancer precursor biology, based on a unique collection of high-grade pre-invasive lung lesions which were followed-up under conservative clinical management.

The present disclosure delineates changes in the genomic architecture, genome-wide gene expression and DNA methylation of pre-invasive cancers with known histological evidence of subsequent disease progression or regression. The CIS genome shares many of the hallmarks of advanced, invasive LUSC but marked genomic, transcriptomic and epigenetic differences exist between lesions that are benign and those that will progress to cancer. The disclosure demonstrate the use of these differences in predicting outcome over current clinical practice. This disclosure represents the first whole genome sequencing data of pre-invasive lung lesions and offers the first insight into the molecular map of early lung squamous cancer pathogenesis, foretelling an era in which molecular profiling will enable personally tailored therapeutic decisions for patients with pre-cancerous lesions, for example, pre-invasive lung disease.

Genomic, transcriptomic and epigenomic landscape of CIS have been profiled in a unique patient cohort with longitudinally monitored pre-invasive disease. Predictive modelling identifies which lesions will progress with remarkable accuracy.

Progression-specific methylation changes on a background of widespread heterogeneity, alongside a strong chromosomal instability signature have been identified. Mutations and copy number changes characteristic of cancer and chart their emergence, offering a window into early carcinogenesis have also been identified. This has enabled the provision of a novel MHI, with particular utility in the diagnosis of and monitoring of pre-cancerous lesions and/or cancer. Methods of the invention allow more efficient patient risk stratification for the purposes of providing better treatment and/or to help plan and manage patient care.

Thus, the invention provides a method of identifying whether or not an individual has a cancer, a pre-invasive lesion that will progress to a cancer, or a pre-cancerous cell population that will progress to a cancer, the method comprising:

(a) providing a DNA sample which has been taken from the individual;

(b) performing an assay to determine the methylation status (β) value for at least 100 different differentially methylated positions (DMPs) in the DNA sample;

(b) determining a methylation heterogeneity index (MHI) for the DNA sample, wherein the MHI is defined as the proportion of the assayed DMPs which have a β value which is intermediate between 1 and 0; and

(c) identifying whether or not the individual has a cancer, a pre-invasive lesion that will progress to a cancer, or a pre-cancerous cell population that will progress to a cancer, based on the MHI.

In any of the methods of the present invention described herein, one or more of the at least 100 DMPs may be a CpG.

In any of the methods of the present invention described herein, an intermediate β value may be defined as tₗₒ<β<tₕᵢ. In any of the methods of the present invention described herein, tₗₒ may be about 0.2, about 0.21, about 0.22, about 0.23, about 0.24, about 0.25, about 0.26, about 0.27, about 0.28, about 0.29, or about 0.3, and/or tₕᵢ may be about 0.8, about 0.81, about 0.82, about 0.83, about 0.84, about 0.85, about 0.86, about 0.87, about 0.88, about 0.89, about 0.9, about 0.91, about 0.92, about 0.93, about 0.94, or about 0.95. Preferably, tₗₒ is about 0.26 and tₕᵢ is about 0.88.

In any of the methods of the present invention described herein, the individual may be identified as having a cancer, a pre-invasive lesion that will progress to a cancer, or a pre-cancerous cell population that will progress to a cancer if the MHI determined for the DNA sample is greater than a threshold value. The threshold value may be from about 0.25 to about 0.45, from about 0.28 to about 0.42, from about 0.3 to about 0.4, from about 0.32 to about 0.38, or from about 0.34 to about 0.38.

In any of the methods of the present invention described herein, the individual may be identified as having a cancer, a pre-invasive lesion that will progress to a cancer, or a pre-cancerous cell population that will progress to a cancer if the MHI determined for the DNA sample is greater than from about 0.24 to about 0.45, from about 0.28 to about 0.42, from about 0.3 to about 0.4, from about 0.32 to about 0.38, or from about 0.34 to about 0.38.

In any of the methods of the present invention described herein, in step (b) a β value may be determined for at least 200, at least 300, at least 500, at least 750, at least 1000, at least 1250, at least 1500, at least 2000, at least 5000, at least 10,000, at least 50,000, at least 100,000, at least 150,000, at least 200,000, at least 250,000, at least 300,000, at least 3500,00, at least 400,000, at least 450,000, or at least 500,000 DMPs.

In any of the methods of the present invention described herein, the DMPs may be substantially randomly distributed throughout the genome.

In any of the methods of the present invention described herein, in step (b) at least about 100,000, at least about 200,000, at least about 300,000, at least about 400, 000 at least 500,000, at least about 750,000, at least 1×10⁶, at least 1×10⁷, at least 1×10⁸, or at least 1×10⁹ individual DNA molecules are assayed per DMP.

Any of the methods of the present invention described herein may achieve an ROC AUC of at least about 0.5, at least about 0.51, at least about 0.52, at least about 0.53, at least about 0.54, at least about 0.55, at least about 0.56, at least about 0.57, at least about 0.58, at least about 0.59, at least about 0.6, at least about 0.61, at least about 0.62, at least about 0.63, at least about 0.64, at least about 0.65, at least about 0.66, at least about 0.67, at least about 0.68, at least about 0.69, at least about 0.7, at least about 0.71, at least about 0.72, at least about 0.73, at least about 0.74, at least about 0.75, at least about 0.76, at least about 0.77, at least about 0.78, at least about 0.79, at least about 0.8, at least about 0.81, at least about 0.82, at least about 0.83, at least about 0.84, at least about 0.85, at least about 0.86, at least about 0.87, at least about 0.88, at least about 0.89, at least about 0.9, at least about 0.91, at least about 0.92, at least about 0.93, at least about 0.94, at least about 0.95, at least about 0.96, at least about 0.97, at least about 0.98, or at least about 0.99.

Any of the methods of the present invention described herein may achieve a specificity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%

Any of the methods of the present invention described herein may achieve a sensitivity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%.

The present invention also provides a method of the present invention described herein for identifying whether or not an individual has a cancer, which achieves an ROC AUC of at least about 0.9, at least about 0.91, at least about 0.92, at least about 0.93, at least about 0.94, at least about 0.95, or at least about 0.96, preferably wherein the method achieves an ROC AUC of about 0.95 or about 0.96, optionally wherein the cancer is a lung cancer, preferably wherein the lung cancer is lung squamous cell carcinoma (LUSC).

The present invention also provides a method of the present invention described herein for identifying whether or not an individual has a pre-invasive lesion that will progress to a cancer, which achieves an ROC AUC of at least about 0.66, at least about 0.67, at least about 0.68, at least about 0.69, at least about 0.7, at least about 0.71, at least about 0.72, at least about 0.73, at least about 0.74, or at least about 0.75, preferably wherein the method achieves an ROC AUC of about 0.74 or about 0.75, optionally wherein the pre-invasive lesion is a pre-invasive lung lesion, optionally wherein the pre-invasive lung lesion is a lung carcinoma in situ (CIS).

In any of the methods of the present invention described herein, an intermediate β value may be defined as 0.26<β<0.88. In any of the methods of the present invention described herein, an intermediate β value may be defined as 0.26<β<0.88 and in step (b) a β value may be determined for at least about 1500 DMPs. In any of the methods of the present invention described herein, an intermediate β value may be defined as 0.26<β<0.88 and in step (b) a β value may be determined for about 2000 DMPs.

In any of the methods of the present invention described herein, an intermediate β value may be defined as 0.26<β<0.88 and in step (b) a β value may be determined for at least about 1500 DMPs and the threshold value may be from about 0.25 to about 0.45, from about 0.3 to about 0.4, from about 0.32 to about 0.38, or from about 0.34 to about 0.36.

In any of the methods of the present invention described herein, an intermediate β value may be defined as 0.26<β<0.88 and in step (b) a β value may be determined for about 2000 DMPs and the threshold value may be from about 0.25 to about 0.45. In any of the methods of the present invention described herein, an intermediate β value may be defined as 0.26<β<0.88 and in step (b) a β value may be determined for about 2000 DMPs and the threshold value may be from about 0.3 to about 0.4. In any of the methods of the present invention described herein, an intermediate β value may be defined as 0.26<β<0.88 and in step (b) a β value may be determined for about 2000 DMPs and the threshold value may be from about 0.32 to about 0.38. In any of the methods of the present invention described herein, an intermediate β value may be defined as 0.26<β<0.88 and in step (b) a β value may be determined for about 2000 DMPs and the threshold value may be from about 0.34 to about 0.36.

Step (b) may comprise bisulphite conversion of the DNA. Step (b) may comprise:

(i) performing a sequencing step to determine the sequence of the DNA molecules, preferably wherein before sequencing an amplification step is performed, preferably wherein the amplification step is performed by PCR; and/or

(ii) (a) hybridising the DNA to an array comprising probes capable of discriminating between methylated and non-methylated forms of DNA and applying a detection system to the array to discriminate methylated and non-methylated forms of DNA, optionally wherein before hybridisation an amplification step is performed, preferably wherein the amplification step is performed by PCR; or
(b) performing an amplification step using methylation-specific primers, wherein the methylation status of the DNA is determined by the presence or absence of an amplified product.

In any of the methods of the present invention described herein, the DNA sample may have been taken from a tissue, a bodily fluid and/or a circulating material previously obtained from the individual. The DNA sample may have been taken from a tissue which has been obtained from a biopsy, optionally wherein the tissue, the bodily fluid or the circulating material is suspected of harbouring a cancer, a pre-invasive lesion, or a pre-cancerous cell population.

In any of the methods of the present invention described herein, the assay to determine the methylation status of the DMPs may output a signal for methylated CpGs (M) and a signal for the unmethylated CpGs (U). The β value may be calculated as intensity of M/(intensity of U+intensity of M+100). The signals for M and U are fluorescent signals.

The present invention also provides a method of treating and/or preventing a cancer and/or treating a pre-invasive lesion that will progress to a cancer or a pre-cancerous cell population that will progress to a cancer in an individual, the method comprising:

identifying a cancer, a pre-invasive lesion that will progress to a cancer, or a pre-cancerous cell population that will progress to a cancer in the individual by performing a method of the present invention described herein which is a method for identifying whether or not an individual has a cancer, a pre-invasive lesion that will progress to a cancer, or a pre-cancerous cell population that will progress to a cancer; and

administering a cancer therapy to the individual, optionally wherein the therapy comprises surgical intervention.

The present invention also provides a methylation heterogeneity index (MHI) as defined herein for identifying in an individual a cancer, a pre-invasive lesion that will progress to a cancer, or a pre-cancerous cell population that will progress to a cancer. The MHI may be determined by performing a method of the present invention described herein.

In any of the embodiments of the present invention described herein, the DNA sample may be from an individual:
(a) not suspected of having a pre-invasive lesion, a pre-cancerous cell population, or a cancer;
(b) suspected of having a pre-invasive lesion or a pre-cancerous cell population but not suspected of having a cancer;
(c) having a pre-invasive lesion or a pre-cancerous cell population but not suspected of having a cancer;
(d) having a pre-invasive lesion or a pre-cancerous cell population and suspected of having a cancer;
(e) suspected of having a pre-invasive lesion or a pre-cancerous cell population;
(f) having a pre-invasive lesion or a pre-cancerous cell population;
(g) suspected of having a cancer; or
(h) having a cancer.

In any of the embodiments of the present invention described herein, the pre-invasive lesion is a solid lesion or the cancer is a solid tumour. In any of the embodiments of the present invention described herein:

(a) the pre-invasive lesion or pre-cancerous cell population is present in the central nervous system, the eye, the ear, nose or throat, the skin, the lung, the bone, an endocrine tissue, breast tissue, the digestive system, the reproductive system, the liver, or the kidney;

(b) the cancer is a cancer of the central nervous system, the eye, the ear, nose or throat, the skin, the lung, the bone, an endocrine tissue, breast tissue, the digestive system, the reproductive system, the liver, or the kidney;

(c) the pre-invasive lesion is normal epithelium, tissue hyperplasia, dysplasia, or lung carcinoma in situ (CIS); and/or

(d) the cancer is a lung cancer, optionally wherein the lung cancer is a lung squamous cell carcinoma (LUSC).

### BRIEF DESCRIPTION OF THE FIGURES

FIG. 1. Demographic and clinical characteristics of patients in the whole-genome sequencing, methylation discovery and validation, and gene expression discovery and validation datasets.

FIG. 2. Analysis of pre-invasive lung carcinoma-in-situ (CIS) lesions. (A) Detection of bronchial pre-invasive CIS lesions by autofluorescence bronchoscopy. (B) Histological outcomes of bronchial pre-invasive lesions. (C) Overview of the study protocol. Patients with identified CIS lesions underwent repeat bronchoscopy and re-biopsy every 4 months. Definitive cancer treatment was only performed if pathological evidence of progression to invasive cancer was detected. The ‘index biopsy’ profiled in this study refers to the biopsy immediately preceding progression to invasive cancer or regression to low-grade dysplasia or normal epithelium. (D) Venn diagram of different-omics analyses performed on laser capture microdissection (LCM)-captured CIS lesions. Due to the small size of bronchial biopsies, not all analyses were performed on all samples.

FIG. 3. Genomic aberrations in pre-invasive lung carcinoma-in-situ (CIS) lesions. Circos diagram comparing CIS genomic profiles with The Cancer Genome Atlas (TCGA) LUSC data. The outer histogram (A), shows mutation frequencies of all genes in TCGA data. The inner histogram (D) shows mutation frequencies in the CIS data presented herein. Profiles appear similar and no statistically significant differences were identified between the two datasets. Genes previously identified as potential drivers of lung cancer are labelled. Between the two histograms, average copy number changes are shown for TCGA data (B) and CIS data (C). Copy number gains are shown in red, losses in blue. Although there were some differences between whole-genome and whole-exome sequencing techniques, it was observed that many similar features between the two; for example, gains in 3q and 5p, which are well recognised features of squamous cell lung cancer. In the centre of the circos plot, 39 rings represent the copy number profiles of the 39 samples descried herein, illustrating the individual contribution of each sample to the average values presented (E).

FIG. 4. Altered methylation and gene expression in lung carcinoma-in-situ (CIS) lesions. (A) Hierarchical clustering of 1335 significantly differentially expressed genes in progressive and regressive CIS lesions. Biological and clinical factors including age at diagnosis, gender, smoking history (pack years) and COPD status had no effect on CIS lesion gene expression profile (high expression=purple, e.g., the trend in the top left quadrant of (A), low expression=orange). (B) Hierarchical clustering of the top 1000 significantly differentially methylated positions (DMPs) between progressive and regressive CIS lesions and controls. Biological and clinical factors including age at diagnosis, gender and smoking history (pack years) status had no effect on the methylation profile (hypomethylated DMPs=blue, hypermethylated DMPs=orange, e.g., the trend in the bottom right quadrant of (B)). (C) Principle component analysis of all profiled genes in progressive and regressive CIS lesions showing a clear distinction between progressive and regressive groups (p=0.0017). (D) Principle component analysis of all methylation data in progressive, regressive and control CIS lesions showing a clear distinction between progressive and regressive groups (p=6.8×10-25). P values were calculated using multivariate ANOVA.

FIG. 5. Carcinoma-in-situ (CIS) gene expression and methylation profiles are predictive of progression to cancer. (A) Probability plot based on a 291-gene signature for correct class prediction (discovery set—red circles indicate progressive lesions, e.g., top right; green circles indicate regressive lesions, e.g., bottom left). (B) Challenging the 291-gene signature on a CIS validation set. Area under the curve (AUC) is 1 using Receiver Operating Characteristic (ROC) analysis. (C) Application of the 291-gene signature to TCGA LUSC data. The signature described herein classified TCGA LUSC vs TCGA controls samples with AUC of 0.81 (green circles indicate TCGA controls (left portion of graph), orange circles indicate TCGA LUSC (right portion of graph). (D) Distribution of methylation beta values across the genome in TCGA controls, CIS regressive and progressive and TCGA LUSC samples. Most probes are regulated at 0 or 1 in normal tissue but this regulation is reduced in both regressive and progressive CIS and TCGA LUSC samples. (E) Methylation Heterogeneity Index (MHI), defined as counts of methylation probes with 0.26<β<0.88, for each sample. MHI is higher in regressive and progressive CIS and TCGA LUSC compared with TCGA controls and this can be used as an accurate predictor with AUC=0.96 for TCGA LUSC vs TCGA controls and AUC=0.74 for progressive vs regressive CIS. (F) Histogram of AUC values calculated by performing the same analysis used in (E) 10,000 times, with each run limited to a different random sample of 2,000 probes (AUC mean for TCGA LUSC vs TCGA controls is 0.95 (95% CI 0.92-0.98)). This demonstrates that a random sample of methylation probes is an accurate predictor using this method.

FIG. 6. Chromosomal instability is associated with progression to cancer. (A) Mean expression of CIN-associated genes in CIS samples. Progressive and regressive CIS samples are well differentiated with AUC=0.96. Green circles indicate regressive CIS lesions; red circles indicate progressive CIS. (B) Plot of NEK2 expression across CIS samples demonstrates increasing expression with progression to cancer. Expression of this gene alone classifies progressive vs regressive CIS with AUC=0.93. (C) Pathway analysis of gene expression data between progressive and regressive CIS shows a strong chromosomal instability (CIN) signal. This signal remains strong when cell cycle genes are removed from the CIN70 signature. (D) Pathway analysis of methylation data demonstrating several cancer-related pathways up-regulated in progressive CIS compared with regressive CIS.

FIG. 7. Experimental workflow. Flow diagram illustrating which profiling techniques were applied to which samples. Biopsies taken from index CIS lesions were stored as fresh frozen (FF) and formalin-fixed paraffin embedded (FFPE). DNA was extracted from FF biopsies. The first 54 samples studied that had sufficient extracted DNA passing quality control (QC) underwent first methylation profiling, then whole-genome sequencing (WGS) when sufficient remaining DNA was available. Due to the low DNA quantity extracted from some biopsies, the methylation dataset (n=54) was larger than the WGS data set (n=29), therefore the subsequent 10 samples underwent WGS directly without methylation profiling. RNA was extracted from FFPE samples and underwent gene expression profiling when RNA passed QC. To ensure validity of our conclusions across orthogonal platforms we used Illumina microarrays to profile a discovery set of 33 samples, then subsequently used Affymetrix microarrays to profile an independent validation set of 18 further samples.

FIG. 8. Mutational signatures of carcinoma-in-situ (CIS) lesions. (A-D) The contribution of each of five pre-selected mutational signatures to each lesion is shown. These five mutational signatures, associated with CpG deamination (1), APOBEC (2 and 13), tobacco (4) and unknown aetiology (5), were selected based on an initial run using all 30 mutational signatures, which showed that these were present in the data and in signature extractions from lung squamous cell cancer (LUSC) datasets. The number of substitutions attributed to each signature is shown (A-B) as well as the proportion of mutations attributed to each mutational signature (C-D). Samples from the same patient share the same identifier except for the final letter; for example, PD21883a and PD21883d are two samples from the same patient. (e) Comparison of the mutational signatures of CIS lesions to those found in lung squamous cell cancer (LUSC). LUSC data were downloaded from TCGA and mutations called with our algorithms. All mutations from all samples from each cancer type were pooled for this analysis. The colour scale indicates the proportion of substitutions in each sample that are attributed to each signature. (F-J) Comparison of the relative proportion of mutations attributed to each signature between progressive (right-hand side) and regressive (left-hand side) CIS samples. P values were calculated using likelihood ratio tests of a mixed effects model with outcome (progressive or regressive) included as a fixed effect versus a model that was identical but for the fact that outcome was not included as a fixed effect. Only signature 4 (smoking-associated) was significantly different between the two groups.

FIG. 9. Genome-wide copy number changes of carcinoma-in-situ (CIS) lesions. Visualisation of copy number changes for 39 whole-genome-sequenced CIS samples. Rows represent samples, genomic position is represented on the x-axis. Local copy number gains are illustrated in red, losses in blue. Widespread changes were observed in progressive CIS samples and a subset of regressive samples.

FIG. 10. Documentation of biopsy history and chronology of lesion appearance in three misclassified regressive cases. (A) Case 1 (PD21893a) appeared to regress from a CIS lesion (July 2012) to squamous metaplasia (SqM; November 2012). However, again, CIS was subsequently reconfirmed by biopsy (May 2013). (B) Case 2 (PD21884a) had a lobectomy for T1N0 lung squamous cell cancer (LUSC) in the left upper lobe (LUL) and was under surveillance for carcinoma-in-situ (CIS) at the resection margins. A subsequent, high-grade CIS lesion (August 2009) profiled for genome-wide DNA methylation changes was considered regressive since a follow-up biopsy on the same anatomical site demonstrated the presence of a low-grade, moderately dysplastic (MoD) lesion (November 2009). A subsequent biopsy, however, was classified as CIS (February 2011) and the lesion then remained static for 26 months but eventually progressed into invasive cancer (April 2014). (C) Case 3 (PD38326a) had an initial diagnosis of CIS (November 2015) followed by regression to normal epithelium (March 2016). CIS was subsequently identified at the same site (March 2017), with invasive cancer diagnosed on subsequent biopsy (July 2017).

FIG. 11. Genomic aberrations in pre-invasive lung carcinoma-in-situ (CIS) lesions. Comparisons of the number of substitutions (A), small insertions and deletions (B), genome rearrangements (C) and copy number changes (D), showing significantly more genomic changes in progressive than regressive lesions. Although there were more clonal substitutions in progressive than regressive lesions (E), the proportion of substitutions that were clonal and the number of clones were similar (F-G). Progressive lesions had more putative driver mutations (H). Telomere lengths (base pairs) were similar between the two groups (E). All P values were calculated using likelihood ratio tests of a mixed effects model with outcome (progressive or regressive) included as a fixed effect versus a model that was identical but for the fact that outcome was not included as a fixed effect.

FIG. 12. Subclonal mutational structure in progressive and regressive CIS lesions. Heatmap showing the proportion of overlapping mutations between samples taken from the same patient. For four patients with lesions that would ultimately progress to cancer (denoted T′), over half the mutations were shared between any two given samples, suggesting that the lesions were derived from a common ancestral clone. By contrast, for two patients with lesions that would ultimately regress (denoted a′), almost no mutations were shared, suggesting that the lesions arose independently. Samples from the same patient are shown in the same colour; PD38321a and PD38322a do belong to the same patient and were mislabelled during processing.

FIG. 13. Differential molecular changes between progressive and regressive lesions. Visualisation of differential changes across the genome. (A) shows all identified differentially methylated regions (DMRs) (hypermethylated regions in yellow, hypomethylated in blue) alongside a similar analysis comparing cancer and control samples from The Cancer Genome Atlas. It was observed that 58% of DMRs identified in the progressive vs regressive analysis are also identified in cancer vs control. (B) shows copy number changes across the genome in regressive CIS, progressive CIS and TCGA cancer samples. Congruency of copy number change was observed, suggesting similar processes in the two cohorts.

FIG. 14. Principal component analysis investigating effect of various biological, clinical and technical factors affecting correct case segregation for all differentially methylated positions (DMPs) and gene expression data. (A-F) Principal component analysis for all DMPs. (A) Smoking history (pack years). (B) Chronic obstructive pulmonary disease (COPD) status. (C) Previous lung cancer history referring to the presence of lung squamous cell cancer (LUSC) prior to identification of pre-invasive lesions. (D) Age at bronchoscopy (years); age of individual when pre-invasive lesion was first biopsied. (E) Gender. (F) Sentix ID. (G-K) Principal component analysis for all gene expression data. (G) Smoking history (pack years). (H) Chronic obstructive pulmonary disease (COPD) status. (I) Previous lung cancer history referring to the presence of lung squamous cell cancer (LUSC) prior to identification of pre-invasive lesions. (J) Age at bronchoscopy (years); age of individual when pre-invasive lesion was first biopsied. (K) Gender. P-values were calculated using multivariate ANOVA.

FIG. 15. ROC analytics of gene expression predictive model. ROC and precision-recall curves for the predictive model based on gene expression data shown in FIG. 5A-C. Curves are shown for the CIS discovery set (A-B), CIS validation set (C-D) and application to TCGA LUSC data (E-F).

FIG. 16. Predictive modelling of methylation data. In addition to the predictive modelling based on probe variation shown in FIG. 6, differentially expressed methylation probes were used to create a predictor using a Prediction Analysis for Microarrays (PAM) method. The model was trained on a training set (A-C) consisting of 26 progressive samples, 11 regressive samples and 23 control samples, shown in red, green and blue, respectively. A predictor based on 141 DMPs was created. This was applied to a validation set of 10 progressive, 7 regressive and 10 control samples (D-F), predicting outcome with AUC=0.99. (G-I) Application of the predictive model to TCGA methylation data. Samples were correctly classified into TCGA LUSC and TCGA control samples with AUC=0.99. (J-M) ROC analytics and precision-recall curves for Methylation Heterogeneity Index (MHI) model presented in FIG. 5. Curves apply to cancer vs control (J-K) and progressive vs regressive (L-M), respectively. (N) Histogram of AUC values using MHI model with random samples of 2000 probes, applied to progressive vs regressive data. This demonstrates that a similar AUC is achieved with a random sample of probes as when using the entire array.

FIG. 17. Predictive modelling of copy number alteration (CNA) data. Using an analogous method to gene expression and methylation copy number data derived from methylation arrays was used to predict lesion outcome. Probe-level copy number changes were aggregated over cytogenetic bands; these data were used as input to Prediction Analysis of Microarrays (PAM). (A-C) Probability plot based on a 154 cytogenetic band signature for correct class prediction (red circles indicate progressive lesions, green circles indicate regressive lesions). The area under the curve for the 154-cytogenetic band signature is 0.86. (D-F) Application of the predictive model to previously published data (van Boerdonk et al.) replicates those result, classifying all regressive and 9/12 progressive samples correctly. This dataset included pre-invasive samples of various histological grades, rather than only CIS. (G-I) Application of the predictive model to TCGA copy number data. Samples were correctly classified into TCGA LUSC and TCGA control samples with an AUC of 0.98.

FIG. 18. wGII score correlates with mean CIN gene expression. To confirm an association between CIN gene expression and copy number change, Weighted Genome Integrity Index (wGII) was correlated with mean CIN gene expression for the 11 CIS samples where gene expression and whole-genome sequencing data was available. Pearson correlation coefficient r2=0.473.

### DETAILED DESCRIPTION OF THE INVENTION

The early detection and treatment of cancers, pre-invasive lesions and pre-cancerous cell populations, in particular by non-invasive methods remains a major unmet need. The present inventors have extensively profiled the DNA methylation patterns associated with progressive pre-invasive lesions i.e. a type of pre-cancerous cell populations that develop into cancer as compared to regressive pre-invasive lesions i.e. a type of pre-cancerous cell populations that do not develop into cancer. Based on this work, the present inventors have developed predictive methods based on a methylation heterogeneity index (MHI), which can be used to discriminate between progressive and regressive pre-invasive lesions as well as non-cancerous and cancer samples with a high degree of accuracy.

The methods and MHI provided by the present invention can be used to identify an individual having a cancer, a pre-invasive lesion that will progress to a cancer, or a pre-cancerous cell population that will progress to a cancer. Such an individual will benefit from therapeutic treatment of the cancer or preventative and/or therapeutic treatment of the progressive pre-invasive lesion or pre-cancerous cell population. Alternatively, the methods and MHI provided by the present invention can be used to identify an individual as not having a cancer, a pre-invasive lesion that will progress to a cancer, or a pre-cancerous cell population that will progress to a cancer. Such an individual would not benefit from therapeutic treatment and/or preventative treatment and therefore the potentially harmful side-effects of e.g. chemotherapy, radiotherapy and/or immunotherapy can be avoided. Accordingly, the methods of the present invention are useful for early diagnosis/prognosis of individuals suspected of having a cancer or the identification of a cancer in asymptotic individuals (e.g. as part of a routine screen). In this way, a preventative and/or therapeutic regime may be more effectively tailored to the individual.

### Definitions

The term “comprises” (comprise, comprising) should be understood to have its normal meaning in the art, i.e. that the stated feature or group of features is included, but that the term does not exclude any other stated feature or group of features from also being present. For example, a method comprising steps (a), (b) and (c) includes steps (a), (b) and (c) but may also include other steps.

The term “consists of” should also be understood to have its normal meaning in the art, i.e. that the stated feature or group of features is included, to the exclusion of further features. For example a method consisting of steps (a), (b) and (c) includes steps (a), (b) and (c) and no other steps.

For every embodiment in which “comprises” or “comprising” is used, the present invention provides a further embodiment in which “consists of” or “consisting of” is used. Thus, every disclosure of “comprises” should be considered to be a disclosure of “consists of”.

### Individual

In any of the methods of the invention disclosed herein, the term “individual” may be a human. The most preferred individual to which the methods of the invention are applicable are humans.

In any of the methods of the invention disclosed herein, the individual may be a non-human animal. For example, methods of the invention disclosed herein may be applied to non-human animals to determine the efficacy of new therapeutics, new therapeutic strategies, new modes of administration of pre-existing therapeutic strategies, or surgical methods. Thus, in any of the methods of the invention disclosed herein the individual may be a rodent, such as a rat or a mouse. In any of the methods of the invention disclosed herein, the individual may be a non-human mammal, such as a primates, cats or pigs.

In any of the methods of the invention described herein, the individual can be one who:
(a) is not suspected of having a pre-invasive lesion, a pre-cancerous cell population, or a cancer;
(b) is suspected of having a pre-invasive lesion or a pre-cancerous cell population but not suspected of having a cancer;
(c) has a pre-invasive lesion or a pre-cancerous cell population but not suspected of having a cancer;
(d) has a pre-invasive lesion or a pre-cancerous cell population and suspected of having a cancer;
(e) is suspected of having a pre-invasive lesion or a pre-cancerous cell population;
(f) has a pre-invasive lesion or a pre-cancerous cell population;
(g) is suspected of having a cancer; or
(h) has a cancer.

The individual may be suspected of having a a pre-invasive lesion, a pre-cancerous cell population, or a cancer on the basis of a clinical presentation, a diagnostic test and/or family history. The individual may have previously had a pre-invasive lesion, a pre-cancerous cell population, and/or a cancer. The individual may be in remission from a pre-invasive lesion, a pre-cancerous cell population, and/or a cancer e.g. an invasive cancer. The individual may be, or have been, a smoker. The individual may be a non-smoker. The individual may be male. The individual may be female. The individual may be an infant. The individual may be an adult. The individual may be elderly. The individual may currently be undergoing treatment for a pre-invasive lesion, a pre-cancerous cell population, and/or a cancer. The individual may be on a treatment holiday.

### Sample

In some of the methods of the invention described herein a DNA sample which has been taken from the individual is provided. The DNA sample may have been taken from a tissue, a bodily fluid and/or a circulating material previously obtained from the individual. The tissue, the bodily fluid or the circulating material may be suspected of harbouring a cancer, a pre-invasive lesion, or a pre-cancerous cell population. The tissue may have been obtained from a biopsy. The tissue may be a fresh-frozen (FF) tissue sample. The tissue may be a formalin-fixed paraffin-embedded (FFPE) tissue sample.

The bodily fluid or the circulating material from which the DNA sample has been previously obtained may be selected from the group consisting of urine, lymph, blood, a blood fraction, plasma, serum, a blood spot, lung mucus, saliva, sputum, phlegm, and combinations thereof.

The tissue, a bodily fluid and/or a circulating material previously obtained from the individual may be from the central nervous system, the eye, the ear, nose or throat, the skin, the lung, the bone, an endocrine tissue, breast tissue, the digestive system, the reproductive system, the liver, or the kidney.

The tissue, a bodily fluid and/or a circulating material and the DNA sample be processed in any way that the person performing a method of the invention described herein deems appropriate, such that a MHI may be determined for the DNA sample using a method of the present invention described herein.

### Statistical Parameters for Predictive Molecular Signature

Sensitivity and specificity metrics for the methods of the present invention for identifying a cancer, a pre-invasive lesion that will progress to a cancer, or a pre-cancerous cell population that will progress to a cancer may be defined using standard receiver operating characteristic (ROC) statistical analysis. In ROC analysis, 100% sensitivity corresponds to a finding of no false negatives, and 100% specificity corresponds to a finding of no false positives.

As used herein the term “sensitivity” (also referred to as the true positive rate) refers to a measure of the proportion of actual positives that are correctly identified as such. In other words, the sensitivity of a diagnostic test may be expressed as the number of true positives i.e. individuals correctly identified as having a disease as a proportion of all the individuals having the disease in the test population (i.e. the sum of true positive and false negative outcomes). Thus, a high sensitivity diagnostic test is desirable as it rarely misidentifies individuals having the disease. This means that a negative result obtained by a highly sensitive test has a high likelihood of ruling out the disease.

As used herein, the term “specificity” (also referred to as the true negative rate) refers to a measure of the proportion of actual negatives that are correctly identified as such. In other words, the specificity of a diagnostic test may be expressed as the number of true negatives (i.e. healthy individuals correctly identified as not having a disease) as a proportion of all the healthy individuals in the test population (i.e. the sum of true negative and false positive outcomes). Thus, a high specificity diagnostic test is desirable as it rarely misidentifies healthy individuals. This means that a positive result obtained by a highly specific test has a high likelihood of ruling in the disease.

As used herein, a “Receiver Operating Characteristic (ROC) curve” refers to a plot of true positive rate (sensitivity) against the false positive rate (1−specificity) for all possible cut-off values. These terms are well known in the art and to the skilled person.

The specificity and/or sensitivity of a method may be determined by perform said method on a validation set of samples. For samples in the validation set it is known which samples are positive samples e.g. samples derived from pre-invasive lesions or pre-cancerous cell populations known to have progressed to a cancer or cancer samples. It is also know which samples of the validation set are negative samples e.g. samples derived from pre-invasive lesions or pre-cancerous cell populations which did not progress to a cancer or healthy non-cancer samples. The extent to which the method correctly identifies the known positive samples (i.e. the sensitivity/true positive rate of the method) and/or the known negative samples (i.e. the specificity/true negative rate of the method) can thus be determined.

A further metric which can be employed to classify the accuracy of the methods of the present invention is ROC AUC. In ROC analysis, the area under the curve of a ROC plot (AUC) is a metric for binary classification. In a random binary classifier the number of true positives and false positives will be approximately equal. In this situation the AUC score for the ROC plot will be 0.5. In a perfect binary classifier the number of true positives will be 100% and the number of false positives will be 0%. In this situation the AUC score for the ROC plot will be 1, which is therefore the highest AUC score a predictive classifier can achieve.

### Assessment of Differentially Methylated Position (DMP) Methylation Status (β)

In the methods of the present invention described herein an assay to determine the methylation status (β) value for at least 100 different differentially methylated positions (DMPs) in the DNA sample is deter mined. DMPs may also be referred to in the art as methylation variable positions (MVPs).

Methylation status (β) is a well know term in the art and the skilled person is readily able to determine the β value of given DMP. A single DMP on a single DNA molecule will either be methylated (M) or unmethylated (U). Thus, the β value of a single DMP in a single DNA is binary, i.e. M=1 or U=0. However, for a population comprising a plurality of DNA molecules, there will be multiple copies of the same DMP. Thus, there may be variation in methylation status between different copies of the same DMP. Thus, for a sample comprising a population of DNA molecules the β value is measure of the average methylation status across the entire population can have thus any value between 1 and 0.

Where a β values are determined on the basis of fluorescent signals associated with methylated or unmethylated DMPs. For each sample, the β value may be calculated as the ratio of the fluorescent signal intensity of the methylated (M) and unmethylated (U) DMPs. For example, according to the following formula:

$$β = \frac{intensity of signal from M DMPs}{instesity of signal from [ U DMPs + M DMPs ] + 100}$$

Methylation of DNA is a recognised form of epigenetic modification which has the capability of altering the expression of genes and other elements such as microRNAs. In cancer development and progression, methylation may have the effect of e.g. silencing tumor suppressor genes and/or increasing the expression of oncogenes. Other forms of dysregulation may occur as a result of methylation. Methylation of DNA occurs at discrete loci which are predominately dinucleotide consisting of a CpG motif, but may also occur at CHH motifs (where H is A, C, or T). During methylation, a methyl group is added to the fifth carbon of cytosine bases to create methylcytosine.

Methylation can occur throughout the genome and is not limited to regions with respect to an expressed sequence such as a gene. Methylation typically, but not always, occurs in a promoter or other regulatory region of an expressed sequence.

A DMP as defined herein is any dinucleotide locus which may show a variation in its methylation status between phenotypes, e.g. between a progressive pre-invasive lung lesion and a regressive pre-invasive lung lesion. A DMP is preferably a CpG or a CHH dinucleotide motif. A DMP as defined herein is not limited to the position of the locus with respect to a corresponding expressed sequence.

Typically, an assessment of DNA methylation status involves analysing the presence or absence of methyl groups in DNA, for example methyl groups on the 5^{th} position of one or more cytosine nucleotides. Preferably, the methylation status of one or more cytosine nucleotides present as a CpG dinucleotide (where C stands for Cytosine, G for Guanine and p for the phosphate group attached to the backbone between the two) is assessed.

A variety of techniques are available for determining the methylation status (i.e. determine the β value) of a DMP, as will be outlined briefly below. The methods described herein encompass any suitable technique for the determination of DMP methylation status.

Methyl groups are lost from a starting DNA molecule during conventional in vitro handling steps such as PCR. To avoid this, techniques for the detection of methyl groups commonly involve the preliminary treatment of DNA prior to subsequent processing, in a way that preserves the methylation status information of the original DNA molecule. Such preliminary techniques involve three main categories of processing, i.e. bisulphite modification, restriction enzyme digestion and affinity-based analysis. Products of these techniques can then be coupled with sequencing or array-based platforms for subsequent identification or qualitative assessment of DMP methylation status.

Techniques involving bisulphite modification of DNA have become the most common methods for detection and assessment of methylation status of CpG dinucleotide. Treatment of DNA with bisulphite, e.g. sodium bisulphite, converts cytosine bases to uracil bases, but has no effect on 5-methylcytosines. Thus, the presence of a cytosine in bisulphite-treated DNA is indicative of the presence of a cytosine base which was previously methylated in the starting DNA molecule. Such cytosine bases can be detected by a variety of techniques. For example, primers specific for unmethylated versus methylated DNA can be generated and used for PCR-based identification of methylated CpG dinucleotides. A separation/capture step may be performed, e.g. using binding molecules such as complementary oligonucleotide sequences. Standard and next-generation DNA sequencing protocols can also be used.

In other approaches, methylation-sensitive enzymes can be employed which digest or cut only in the presence of methylated DNA. Analysis of resulting fragments is commonly carried out using microarrays.

Affinity-based techniques exploit binding interactions to capture fragments of methylated DNA for the purposes of enrichment. Binding molecules such as anti-5-methylcytosine antibodies are commonly employed prior to subsequent processing steps such as PCR and sequencing.

Olkhov-Mitsel and Bapat (2012) [46] provide a comprehensive review of techniques available for the identification and assessment of DMP-based biomarkers involving methylcytosine.

For the purposes of assessing the methylation status of the DMP-based biomarkers characterised and described herein, any suitable method can be employed.

Preferred methods involve bisulphite treatment of DNA, including amplification of the identified DMP loci for methylation specific PCR and/or sequencing and/or assessment of the methylation status of target loci using methylation-discriminatory microarrays.

Amplification of DMP loci can be achieved by a variety of approaches. Preferably, DMP loci are amplified using PCR. DMPs may also be amplified by other techniques such as multiplex ligation-dependent probe amplification (MLPA). A variety of PCR-based approaches may be used. For example, methylation-specific primers may be hybridized to DNA containing the DMP sequence of interest. Such primers may be designed to anneal to a sequence derived from either a methylated or non-methylated DMP locus. Following annealing, a PCR reaction is performed and the presence of a subsequent PCR product indicates the presence of an annealed DMP of identifiable sequence. In such methods, DNA is bisulphite converted prior to amplification. Such techniques are commonly referred to as methylation specific PCR (MSP) [47].

In other techniques, PCR primers may anneal to the DMP sequence of interest independently of the methylation status, and further processing steps may be used to determine the status of the DMP. Assays are designed so that the DMP site(s) are located between primer annealing sites. This method scheme is used in techniques such as bisulphite genomic sequencing [48], COBRA [49], Ms-SNuPE [50]. In such methods, DNA can be bisulphite converted before or after amplification.

Preferably, small-scale PCR approaches are used. Such approaches commonly involve mass partitioning of samples (e.g. digital PCR). These techniques offer robust accuracy and sensitivity in the context of a highly miniaturised system (pico-liter sized droplets), ideal for the subsequent handling of small quantities of DNA obtainable from the potentially small volume of cellular material present in biological samples, particularly urine samples. A variety of such small-scale PCR techniques are widely available. For example, microdroplet-based PCR instruments are available from a variety of suppliers, including RainDance Technologies, Inc. (Billerica, Mass.; http://raindancetech.com/) and Bio-Rad, Inc. (http://www.bio-rad.com/). Microarray platforms may also be used to carry out small-scale PCR. Such platforms may include microfluidic network-based arrays e.g. available from Fluidigm Corp. (www.fluidigm.com).

Following amplification of DMP loci, amplified PCR products may be coupled to subsequent analytical platforms in order to determine the methylation status of the DMPs of interest. For example, the PCR products may be directly sequenced to determine the presence or absence of a methylcytosine at the target DMP or analysed by array-based techniques.

Any suitable sequencing techniques may be employed to determine the sequence of target DNA. In the methods of the present invention the use of high-throughput, so-called “second generation”, “third generation” and “next generation” techniques to sequence bisulphite-treated DNA are preferred.

In second generation techniques, large numbers of DNA molecules are sequenced in parallel. Typically, tens of thousands of molecules are anchored to a given location at high density and sequences are determined in a process dependent upon DNA synthesis. Reactions generally consist of successive reagent delivery and washing steps, e.g. to allow the incorporation of reversible labelled terminator bases, and scanning steps to determine the order of base incorporation. Array-based systems of this type are available commercially e.g. from Illumina, Inc. (San Diego, Calif.; http://www.illumina.com/).

Third generation techniques are typically defined by the absence of a requirement to halt the sequencing process between detection steps and can therefore be viewed as real-time systems. For example, the base-specific release of hydrogen ions, which occurs during the incorporation process, can be detected in the context of microwell systems (e.g. see the Ion Torrent system available from Life Technologies; http://www.lifetechnologies.com/). Similarly, in pyrosequencing the base-specific release of pyrophosphate (PPi) is detected and analysed. In nanopore technologies, DNA molecules are passed through or positioned next to nanopores, and the identities of individual bases are determined following movement of the DNA molecule relative to the nanopore. Systems of this type are available commercially e.g. from Oxford Nanopore (https://www.nanoporetech.com/). In an alternative method, a DNA polymerase enzyme is confined in a “zero-mode waveguide” and the identity of incorporated bases are determined with florescence detection of gamma-labeled phosphonucleotides (see e.g. Pacific Biosciences; http://www.pacificbiosciences.com/).

In other methods in accordance with the invention sequencing steps may be omitted. For example, amplified PCR products may be applied directly to hybridization arrays based on the principle of the annealing of two complementary nucleic acid strands to form a double-stranded molecule. Hybridization arrays may be designed to include probes which are able to hybridize to amplification products of a DMP and allow discrimination between methylated and non-methylated loci. For example, probes may be designed which are able to selectively hybridize to an DMP locus containing thymine, indicating the generation of uracil following bisulphite conversion of an unmethylated cytosine in the starting template DNA. Conversely, probes may be designed which are able to selectively hybridize to a DMP locus containing cytosine, indicating the absence of uracil conversion following bisulphite treatment. This corresponds with a methylated DMP locus in the starting template DNA.

Following the application of a suitable detection system to the array, computer-based analytical techniques can be used to determine the methylation status of an DMP. Detection systems may include, e.g. the addition of fluorescent molecules following a methylation status-specific probe extension reaction. Such techniques allow DMP status determination without the specific need for the sequencing of DMP amplification products. Such array-based discriminatory probes may be termed methylation-specific probes.

Any suitable methylation-discriminatory microarrays may be employed to assess the methylation status of the DMPs described herein. A preferred methylation-discriminatory microarray system is provided by Illumina, Inc. (San Diego, Calif.; http://www.illumina.com/).

In particular, the Infinium HumanMethylation450 BeadChip array system may be used to assess the methylation status of DMPs as described herein. Such a system exploits the chemical modifications made to DNA following bisulphite treatment of the starting DNA molecule. Briefly, the array comprises Type I beads to which are coupled oligonucleotide probes specific for DNA sequences corresponding to the unmethylated form of a DMP, as well as separate Type I beads to which are coupled oligonucleotide probes specific for DNA sequences corresponding to the methylated form of a DMP.

The Infinium HumanMethylation450 BeadChip array system also comprises Type II beads to which are coupled two different types of oligonucleotide probe: a first probe specific for DNA sequences corresponding to the unmethylated form of a DMP and a second probe specific for DNA sequences corresponding to the methylated form of the same DMP.

Candidate DNA molecules are applied to the array and selectively hybridize, under appropriate conditions, to the oligonucleotide probe corresponding to the relevant epigenetic form. Thus, a DNA molecule derived from a DMP which was methylated in the corresponding genomic DNA will selectively attach to methylation-specific oligonucleotide probes, but will fail to attach to the non-methylation-specific oligonucleotide probe. Single-base extension of only the hybridized probes incorporates a labeled ddNTP, which is subsequently stained with a fluorescence reagent and imaged. The methylation status of the DMP may be determined by calculating the ratio of the fluorescent signal derived from the methylated and unmethylated sites.

The Illumina Infinium HumanMethylation450 BeadChip array system can be used to interrogate those same DMPs in the methods described herein. Alternative or customised arrays could, however, be employed to interrogate the DMPs defined herein, provided that they comprise means for interrogating all DMPs for a given method, as defined herein.

Techniques involving combinations of the above-described methods may also be used. For example, DNA containing DMP sequences of interest may be hybridized to microarrays and then subjected to DNA sequencing to determine the status of the DMP as described above.

In the methods described above, sequences corresponding to DMP loci may also be subjected to an enrichment process. DNA containing DMP sequences of interest may be captured by binding molecules such as oligonucleotide probes complementary to the DMP target sequence of interest. Sequences corresponding to DMP loci may be captured before or after bisulphite conversion or before or after amplification. Probes may be designed to be complementary to bisulphite converted DNA. Captured DNA may then be subjected to further processing steps to determine the status of the DMP, such as DNA sequencing steps.

Capture/separation steps may be custom designed. Alternatively a variety of such techniques are available commercially, e.g. the SureSelect target enrichment system available from Agilent Technologies (http://www.agilent.com/home). In this system biotinylated “bait” or “probe” sequences (e.g. RNA) complementary to the DNA containing DMP sequences of interest are hybridized to sample nucleic acids. Streptavidin-coated magnetic beads are then used to capture sequences of interest hybridized to bait sequences. Unbound fractions are discarded. Bait sequences are then removed (e.g. by digestion of RNA) thus providing an enriched pool of DMP target sequences separated from non-DMP sequences. In a preferred method of the invention, template DNA is subjected to bisulphite conversion and target loci are then amplified by small-scale PCR such as microdroplet PCR using primers which are independent of the methylation status of the DMP. Following amplification, samples are subjected to a capture step to enrich for PCR products containing the target DMP, e.g. captured and purified using magnetic beads, as described above. Following capture, a standard PCR reaction is carried out to incorporate DNA sequencing barcodes into DMP-containing amplicons. PCR products are again purified and then subjected to DNA sequencing and analysis to determine the presence or absence of a methylcytosine at the target genomic DMP [32].

The DMP loci defined herein are identified e.g. by Illumina® identifiers (IlmnID), which are also referred to as DMP identifiers (DMP ID). These DMP loci identifiers refer to individual DMP sites used in the commercially available Illumina® Infinium Human Methylation450 BeadChip kit. The identity of each DMP site represented by each DMP loci identifier is publicly available from the Illumina, Inc. website under reference to the DMP sites used in the Infinium Human Methylation450 BeadChip kit.

Further information regarding DMP loci identification used in Illumina, Inc products is found in the technical note entitled “Technical Note: Epigenetics. CpG Loci Identification. A guide to Illumina's method for unambiguous CpG loci identification and tracking for the Golden Gate® and Infinium® Assay for Methylation” published in 2010 and found at:

http://www.illumina.com/documents/products/technotes/technote_cpg_loci_identification.pdf.

Further information regarding the Illumina® Infinium Human Methylation450 BeadChip system can be found at:

http://www.illumina.com/content/dam/illumina-marketing/documents/products/datasheets/datasheet_humanmehylation450.pdf;

and at:

http://www.illumina.com/content/dam/illumina-marketing/documents/products/technotes/technote hm450 _data_analysis_optimization.pdf.

To complement evolving public databases to provide accurate DMP/CpG loci identifiers and strand orientation, Illumina® has developed a method to consistently designate DMP/CpG loci based on the actual or contextual sequence of each individual DMP/CpG locus. To unambiguously refer to DMP/CpG loci in any species, Illumina® has developed a consistent and deterministic DMP loci database to ensure uniformity in the reporting of methylation data. The Illumina® method takes advantage of sequences flanking a DMP locus to generate a unique DMP locus cluster ID. This number is based on sequence information only and is unaffected by genome version. Illumina's standardized nomenclature also parallels the TOP/BOT strand nomenclature (which indicates the strand orientation) commonly used for single nucleotide polymorphism (SNP) designation.

Illumina® Identifiers for the Infinium Human Methylation450 BeadChip system are also available from public repositories such as Gene Expression Omnibus (GEO) (http://www.ncbi.nlm.nih.gov/geo/). For example, at https://www.ncbi.nlm.nih.gov/geo/query/acc.cgi?acc=GPL13534.

The present invention provides a method of identifying whether or not an individual has a cancer, a pre-invasive lesion that will progress to a cancer, or a pre-cancerous cell population that will progress to a cancer, the method comprising:

(a) providing a DNA sample which has been taken from the individual;

(b) performing an assay to determine the methylation status (β) value for at least 100 different differentially methylated positions (DMPs) in the DNA sample;

(b) determining a methylation heterogeneity index (MHI) for the DNA sample, wherein the MHI is defined as the proportion of the assayed DMPs which have a β value which is intermediate between 1 and 0; and

(c) identifying whether or not the individual has a cancer, a pre-invasive lesion that will progress to a cancer, or a pre-cancerous cell population that will progress to a cancer, based on the MHI.

Step (b) may comprise performing an assay to determine the methylation status (β) value for about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, about 2000, about 2050, about 2100, about 2150, about 2200, about 2250, about 2300, about 2350, about 2400, about 2450, about 2500, about 2550, about 2600, about 2650, about 2700, about 2750, about 2800, about 2850, about 2900, about 2950, about 3000, about 3050, about 3100, about 3150, about 3200, about 3250, about 3300, about 3350, about 3400, about 3450, about 3500, about 3550, about 3600, about 3650, about 3700, about 3750, about 3800, about 3850, about 3900, about 3950, about 4000, about 4050, about 4100, about 4150, about 4200, about 4250, about 4300, about 4350, about 4400, about 4450, about 4500, about 4550, about 4600, about 4650, about 4700, about 4750, about 4800, about 4850, about 4900, about 4950, or about 5000 different DMPs in the DNA sample. Preferably, step (b) may comprise performing an assay to determine the methylation status (β) value for about 1000, about 1050, about 1100, about 1150, about 1200, about 1250, about 1300, about 1350, about 1400, about 1450, about 1500, about 1550, about 1600, about 1650, about 1700, about 1750, about 1800, about 1850, about 1900, about 1950, about 2000, about 2050, about 2100, about 2150, about 2200, about 2250, about 2300, about 2350, about 2400, about 2450, about 2500, about 2550, about 2600, about 2650, about 2700, about 2750, about 2800, about 2850, about 2900, about 2950, or about 3000 different DMPs in the DNA sample. Preferably step (b) may comprise performing an assay to determine the methylation status (β) value for about 1500, about 1550, about 1600, about 1650, about 1700, about 1750, about 1800, about 1850, about 1900, about 1950, about 2000, about 2050, about 2100, about 2150, about 2200, about 2250, about 2300, about 2350, about 2400, about 2450, or about 2500 different DMPs in the DNA sample. Preferably, preferably step (b) may comprise performing an assay to determine the methylation status (β) value for about 2000 DMPs.

Step (b) may comprise performing an assay to determine the methylation status (β) value for at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1100, at least 1150, at least 1200, at least 1250, at least 1300, at least 1350, at least 1400, at least 1450, at least 1500, at least 1550, at least 1600, at least 1650, at least 1700, at least 1750, at least 1800, at least 1850, at least 1900, at least 1950, at least 2000, at least 2050, at least 2100, at least 2150, at least 2200, at least 2250, at least 2300, at least 2350, at least 2400, at least 2450, at least 2500, at least 2550, at least 2600, at least 2650, at least 2700, at least 2750, at least 2800, at least 2850, at least 2900, at least 2950, at least 3000, at least 3050, at least 3100, at least 3150, at least 3200, at least 3250, at least 3300, at least 3350, at least 3400, at least 3450, at least 3500, at least 3550, at least 3600, at least 3650, at least 3700, at least 3750, at least 3800, at least 3850, at least 3900, at least 3950, at least 4000, at least 4050, at least 4100, at least 4150, at least 4200, at least 4250, at least 4300, at least 4350, at least 4400, at least 4450, at least 4500, at least 4550, at least 4600, at least 4650, at least 4700, at least 4750, at least 4800, at least 4850, at least 4900, at least 4950, or at least 5000 different DMPs in the DNA sample. Preferably, step (b) may comprise performing an assay to determine the methylation status (β) value for at least 1000, at least 1050, at least 1100, at least 1150, at least 1200, at least 1250, at least 1300, at least 1350, at least 1400, at least 1450, at least 1500, at least 1550, at least 1600, at least 1650, at least 1700, at least 1750, at least 1800, at least 1850, at least 1900, at least 1950, at least 2000, at least 2050, at least 2100, at least 2150, at least 2200, at least 2250, at least 2300, at least 2350, at least 2400, at least 2450, at least 2500, at least 2550, at least 2600, at least 2650, at least 2700, at least 2750, at least 2800, at least 2850, at least 2900, at least 2950, or at least 3000 different DMPs in the DNA sample.

Step (b) may comprise performing an assay to determine the methylation status (β) value for from about 1000 to about 3000, from about 1050 to about 2950, from about 1100 to about 2900, from about 1150 to about 2850, from about 1200 to about 2800, from about 1250 to about 2750, from about 1300 to about 2700, from about 1350 to about 2650, from about 1400 to about 2600, from about 1450 to about 2550, from about 1500 to about 2500, from about 1550 to about 2450, from about 1600 to about 2400, from about 1650 to about 2350, from about 1700 to about 2300, from about 1750 to about 2250, from about 1800 to about 2200, from about 1850 to about 2150, from about 1900 to about 2100, from about 1950 to about 2050 different DMPs in the DNA sample. Preferably, step (b) may comprise performing an assay to determine the methylation status (β) value for about 1500 to about 2500, from about 1550 to about 2450, from about 1600 to about 2400, from about 1650 to about 2350, from about 1700 to about 2300, from about 1750 to about 2250, from about 1800 to about 2200, from about 1850 to about 2150, from about 1900 to about 2100, from about 1950 to about 2050 different DMPs in the DNA sample.

The at least 100 different DMPs assayed in step (b) of the methods of the present invention disclosed herein may be selected from any known DMP loci. For example, the at least 100 different DMPs may be selected from any of the DMPs which can be assayed using the Illumina Infinium Human Methylation450 BeadChip system. As discussed above, the DMPs which can be assayed using the Illumina Infinium Human Methylation450 BeadChip system may be identified by their unique Illumina IDs (DMP IDs). A database of the DMPs which can be assayed using Infinium Human Methylation450 BeadChip system are also available from public repositories such as Gene Expression Omnibus (GEO) (http://www.ncbi.nlm.nih.gov/geo/). Thus, the at least 100 DMPs may be selected from those identified in the database accessible at https://www.ncbi.nlm.nih.gov/geo/query/acc.cgi?acc=GPL13534.

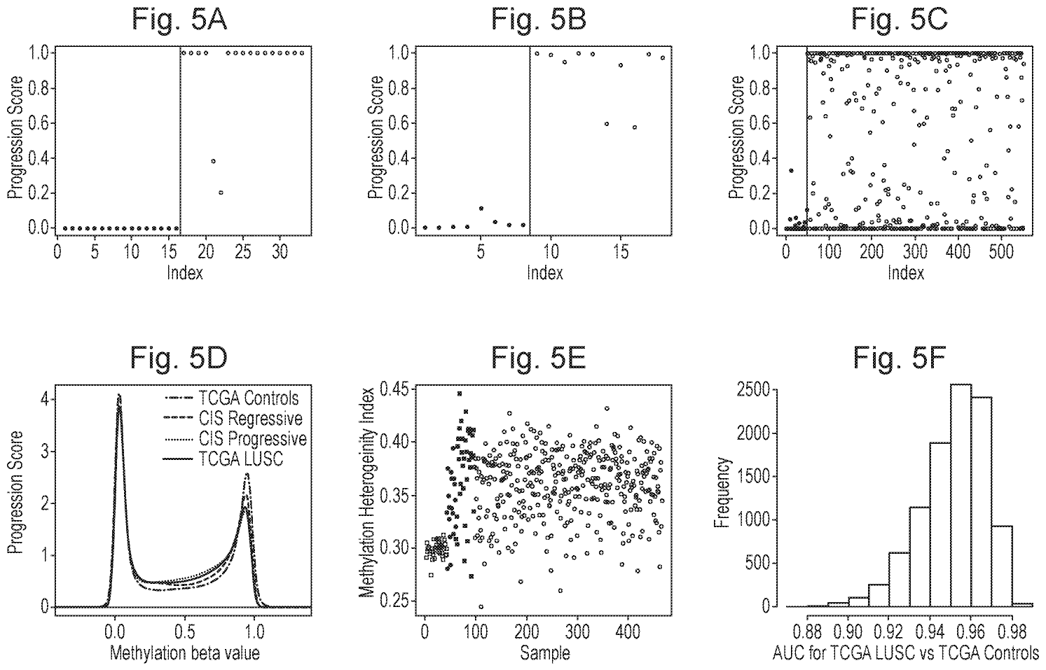

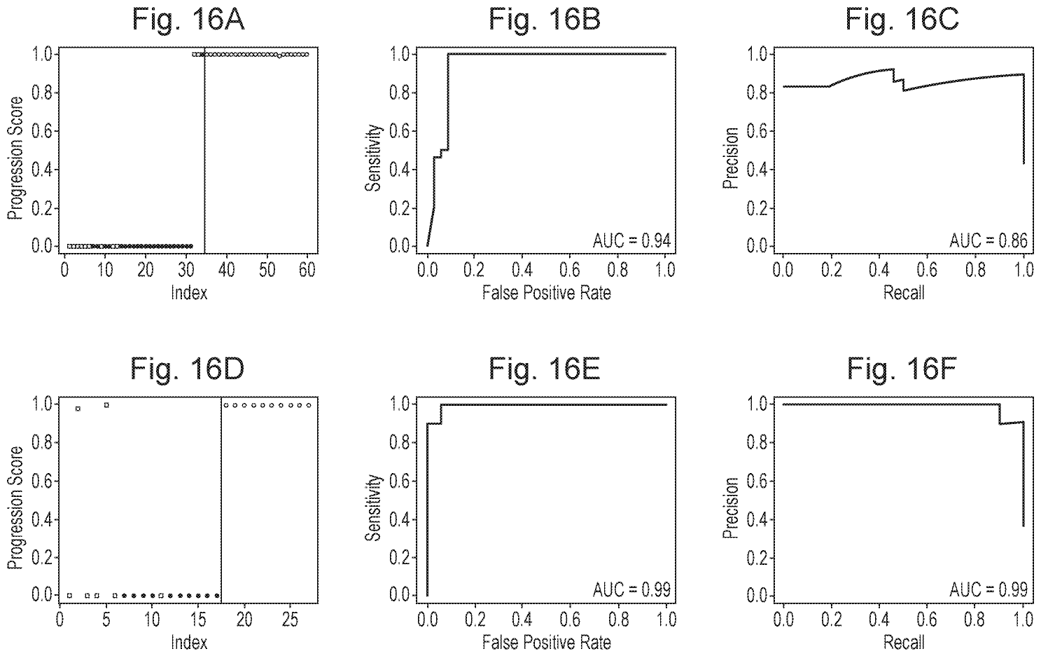

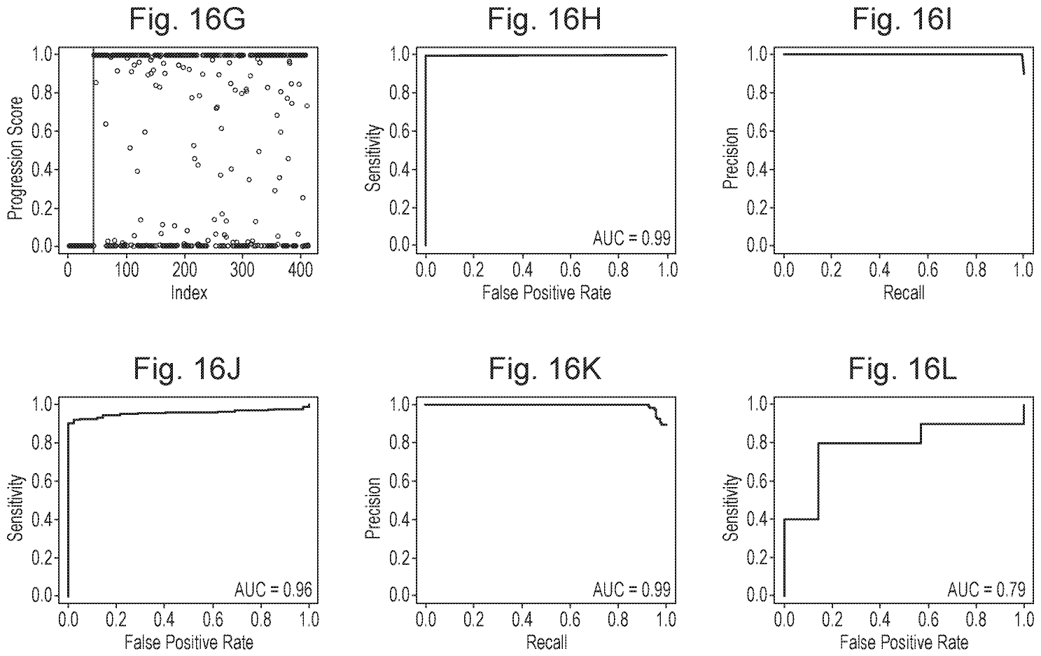

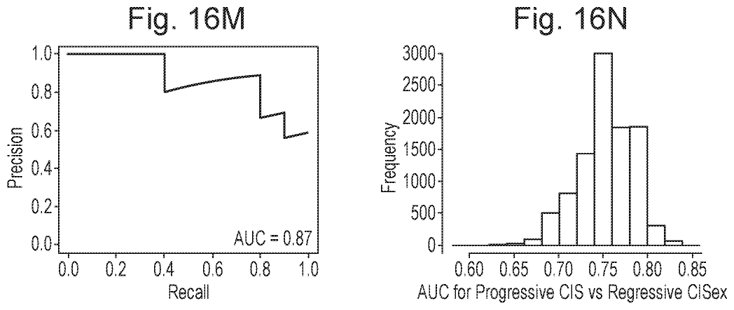

The present inventors have also developed predictive methods based on an increase in genome-wide methylation heterogeneity rather than increased methylation heterogeneity specific to particular functional pathway. In particular it was found that methods in which MHI is deter mined based on DMPs randomly selected from across the genome were able to accurately classify cancer samples versus control samples and progressive versus regressive pre-invasive cancer samples (see Example 6, FIGS. 5F and 16N). Thus, the DMPs assayed in step (b) may be substantially randomly distributed across the genome.

The DMPs assayed in step (b) may be selected from any of the DMPs identified in Tables 1-10.

Thus, in some example methods according to the present invention, the DMPs assayed in step (b) may comprise:

(i) at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1050, at least about 1100, at least about 1150, at least about 1200, at least about 1250, at least about 1300, at least about 1350, at least about 1400, at least about 1450, at least about 1500, at least about 1550, at least about 1600, at least about 1650, at least about 1700, at least about 1750, at least about 1800, at least about 1850, at least about 1900, at least about 1950, or about 2000 DMPs selected from those identified in Table 1, optionally wherein the threshold value is about 0.35, and optionally wherein the method is:
(a) a method of identifying whether or not an individual has a cancer and achieves an ROC AUC of at least about 0.95 and/or a sensitivity of at least about 95% and/or a specificity of at least about 55%; or
(b) a method of identifying whether or not an individual has a pre-invasive lesion that will progress to a cancer and achieves an ROC AUC of at least about 0.8 and/or a sensitivity of at least about 80% and/or a specificity of at least about 70%;

(ii) at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1050, at least about 1100, at least about 1150, at least about 1200, at least about 1250, at least about 1300, at least about 1350, at least about 1400, at least about 1450, at least about 1500, at least about 1550, at least about 1600, at least about 1650, at least about 1700, at least about 1750, at least about 1800, at least about 1850, at least about 1900, at least about 1950, or about 2000 DMPs selected from those identified in Table 2, optionally wherein the threshold value is about 0.37, and optionally wherein the method is:
(a) a method of identifying whether or not an individual has a cancer and achieves an ROC AUC of at least about 0.95 and/or a sensitivity of at least about 95% and/or a specificity of at least about 45%; or
(b) a method of identifying whether or not an individual has a pre-invasive lesion that will progress to a cancer and achieves an ROC AUC of at least about 0.75 and/or a sensitivity of at least about 90% and/or a specificity of at least about 65%;

(iii) at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1050, at least about 1100, at least about 1150, at least about 1200, at least about 1250, at least about 1300, at least about 1350, at least about 1400, at least about 1450, at least about 1500, at least about 1550, at least about 1600, at least about 1650, at least about 1700, at least about 1750, at least about 1800, at least about 1850, at least about 1900, at least about 1950, or about 2000 DMPs selected from those identified in Table 3, optionally wherein the threshold value is about 0.37, and optionally wherein the method is:
(a) a method of identifying whether or not an individual has a cancer and achieves an ROC AUC of at least about 0.95 and/or a sensitivity of at least about 95% and/or a specificity of at least about 45%; or
(b) a method of identifying whether or not an individual has a pre-invasive lesion that will progress to a cancer and achieves an ROC AUC of at least about 0.75 and/or a sensitivity of at least about 80% and/or a specificity of at least about 65%;

(iv) at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1050, at least about 1100, at least about 1150, at least about 1200, at least about 1250, at least about 1300, at least about 1350, at least about 1400, at least about 1450, at least about 1500, at least about 1550, at least about 1600, at least about 1650, at least about 1700, at least about 1750, at least about 1800, at least about 1850, at least about 1900, at least about 1950, or about 2000 DMPs selected from those identified in Table 4, optionally wherein the threshold value is about 0.36, and optionally wherein the method is:
(a) a method of identifying whether or not an individual has a cancer and achieves an ROC AUC of at least about 0.95 and/or a sensitivity of at least about 95% and/or a specificity of at least about 45%; or
(b) a method of identifying whether or not an individual has a pre-invasive lesion that will progress to a cancer and achieves an ROC AUC of at least about 0.75 and/or a sensitivity of at least about 85% and/or a specificity of at least about 65%;

(v) at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1050, at least about 1100, at least about 1150, at least about 1200, at least about 1250, at least about 1300, at least about 1350, at least about 1400, at least about 1450, at least about 1500, at least about 1550, at least about 1600, at least about 1650, at least about 1700, at least about 1750, at least about 1800, at least about 1850, at least about 1900, at least about 1950, or about 2000 DMPs selected from those identified in Table 5, optionally wherein the threshold value is about 0.36, and optionally wherein the method is:
(a) a method of identifying whether or not an individual has a cancer and achieves an ROC AUC of at least about 0.95 and/or a sensitivity of at least about 95% and/or a specificity of at least about 40%; or
(b) a method of identifying whether or not an individual has a pre-invasive lesion that will progress to a cancer and achieves an ROC AUC of at least about 0.8 and/or a sensitivity of at least about 85% and/or a specificity of at least about 65%;

(vi) at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1050, at least about 1100, at least about 1150, at least about 1200, at least about 1250, at least about 1300, at least about 1350, at least about 1400, at least about 1450, at least about 1500, at least about 1550, at least about 1600, at least about 1650, at least about 1700, at least about 1750, at least about 1800, at least about 1850, at least about 1900, at least about 1950, or about 2000 DMPs selected from those identified in Table 6, optionally wherein the threshold value is about 0.36, and optionally wherein the method is:
(a) a method of identifying whether or not an individual has a cancer and achieves an ROC AUC of at least about 0.95 and/or a sensitivity of at least about 95% and/or a specificity of at least about 50%; or
(b) a method of identifying whether or not an individual has a pre-invasive lesion that will progress to a cancer and achieves an ROC AUC of at least about 0.8 and/or a sensitivity of at least about 85% and/or a specificity of at least about 75%;

(vii) at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1050, at least about 1100, at least about 1150, at least about 1200, at least about 1250, at least about 1300, at least about 1350, at least about 1400, at least about 1450, at least about 1500, at least about 1550, at least about 1600, at least about 1650, at least about 1700, at least about 1750, at least about 1800, at least about 1850, at least about 1900, at least about 1950, or about 2000 DMPs selected from those identified in Table 7, optionally wherein the threshold value is about 0.34, and optionally wherein the method is:
(a) a method of identifying whether or not an individual has a cancer and achieves an ROC AUC of at least about 0.95 and/or a sensitivity of at least about 95% and/or a specificity of at least about 65%; or
(b) a method of identifying whether or not an individual has a pre-invasive lesion that will progress to a cancer and achieves an ROC AUC of at least about 0.75 and/or a sensitivity of at least about 60% and/or a specificity of at least about 75%;

(viii) at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1050, at least about 1100, at least about 1150, at least about 1200, at least about 1250, at least about 1300, at least about 1350, at least about 1400, at least about 1450, at least about 1500, at least about 1550, at least about 1600, at least about 1650, at least about 1700, at least about 1750, at least about 1800, at least about 1850, at least about 1900, at least about 1950, or about 2000 DMPs selected from those identified in Table 8, optionally wherein the threshold value is about 0.35, and optionally wherein the method is:
(a) a method of identifying whether or not an individual has a cancer and achieves an ROC AUC of at least about 0.95 and/or a sensitivity of at least about 95% and/or a specificity of at least about 55%; or
(b) a method of identifying whether or not an individual has a pre-invasive lesion that will progress to a cancer and achieves an ROC AUC of at least about 0.75 and/or a sensitivity of at least about 75% and/or a specificity of at least about 70%;

(ix) at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1050, at least about 1100, at least about 1150, at least about 1200, at least about 1250, at least about 1300, at least about 1350, at least about 1400, at least about 1450, at least about 1500, at least about 1550, at least about 1600, at least about 1650, at least about 1700, at least about 1750, at least about 1800, at least about 1850, at least about 1900, at least about 1950, or about 2000 DMPs selected from those identified in Table 9, optionally wherein the threshold value is about 0.37, and optionally wherein the method is:
(a) a method of identifying whether or not an individual has a cancer and achieves an ROC AUC of at least about 0.95 and/or a sensitivity of at least about 95% and/or a specificity of at least about 50%; or
(b) a method of identifying whether or not an individual has a pre-invasive lesion that will progress to a cancer and achieves an ROC AUC of at least about 0.75 and/or a sensitivity of at least about 80% and/or a specificity of at least about 65%; or

(x) at least about 100, at least about 150, at least about 200, at least about 250, at least about 300, at least about 350, at least about 400, at least about 450, at least about 500, at least about 550, at least about 600, at least about 650, at least about 700, at least about 750, at least about 800, at least about 850, at least about 900, at least about 950, at least about 1000, at least about 1050, at least about 1100, at least about 1150, at least about 1200, at least about 1250, at least about 1300, at least about 1350, at least about 1400, at least about 1450, at least about 1500, at least about 1550, at least about 1600, at least about 1650, at least about 1700, at least about 1750, at least about 1800, at least about 1850, at least about 1900, at least about 1950, or about 2000 DMPs selected from those identified in Table 10, optionally wherein the threshold value is about 0.38, and optionally wherein the method is:
(a) a method of identifying whether or not an individual has a cancer and achieves an ROC AUC of at least about 0.95 and/or a sensitivity of at least about 95% and/or a specificity of at least about 45%; or
(b) a method of identifying whether or not an individual has a pre-invasive lesion that will progress to a cancer and achieves an ROC AUC of at least about 0.75 and/or a sensitivity of at least about 90% and/or a specificity of at least about 65%.

### Methylation Heterogeneity Index

The methods of the present invention disclosed herein comprise the step of determining a methylation heterogeneity index (MHI) for the DNA sample, wherein the MHI is defined as the proportion of the assayed DMPs which have a β value which is intermediate between 1 and 0. As used herein the term “intermediate” can refer to any p value which is not equal to 1 or 0. As used herein the term “intermediate” can refer to any value which is not equal to about 1 or about 0. An intermediate β value may be defined with reference to a lower bound termed tₗₒ and an upper bound termed tₕᵢ where tₗₒ≠0 and tₕᵢ≠1. Thus, an intermediate β value may be defined as tₗₒ<β<tₕᵢ

In any of the embodiments of the present invention described herein, tₗₒ may be about 0.01, about 0.02, about 0.03, about 0.04, about 0.05, about 0.06, about 0.07, about 0.08, about 0.09, about 0.1, about 0.11, about 0.12, about 0.13, about 0.14, about 0.15, about 0.16, about 0.17, about 0.18, about 0.19, about 0.2, about 0.21, about 0.22, about 0.23, about 0.24, about 0.25, about 0.26, about 0.27, about 0.28, about 0.29, about 0.3, about 0.31, about 0.32, about 0.33, about 0.34, about 0.35, about 0.36, about 0.37, about 0.38, about 0.39, about 0.4, about 0.41, about 0.42, about 0.43, about 0.44, about 0.45, about 0.46, about 0.47, about 0.48, about 0.49, or about 0.5. Preferably, tₗₒ is about 0.2, about 0.21, about 0.22, about 0.23, about 0.24, about 0.25, about 0.26, about 0.27, about 0.28, about 0.29, or about 0.3. More preferably, tₗₒ is about 0.26.

In any of the embodiments of the present invention described herein, tₗₒ may be from about 0.01 to about 0.5, from about 0.02 to about 0.49, from about 0.03 to about 0.48, from about 0.04 to about 0.47, from about 0.05 to about 0.46, from about 0.06 to about 0.45, from about 0.07 to about 0.44, from about 0.08 to about 0.43, from about 0.09 to about 0.42, from about 0.1 to about 0.41, from about 0.11 to about 0.4, from about 0.12 to about 0.39, from about 0.13 to about 0.38, from about 0.14 to about 0.37, from about 0.15 to about 0.36, from about 0.16 to about 0.35, from about 0.14 to about 0.34, from about 0.18 to about 0.33, from about 0.19 to about 0.32, from about 0.2 to about 0.31, from about 0.21 to about 0.30, from about 0.22 to about 0.29, from about 0.23 to about 0.28, from about 0.24 to about 0.27, or from about 0.25 to about 0.27.

In any of the embodiments of the present invention described herein, tₕᵢ may be about 0.5, about 0.51, about 0.52, about 0.53, about 0.54, about 0.55, about 0.56, about 0.57, about 0.58, about 0.59, about 0.6, about 0.61, about 0.62, about 0.63, about 0.64, about 0.65, about 0.66, about 0.67, about 0.68, about 0.69, about 0.7, about 0.71, about 0.72, about 0.73, about 0.74, about 0.75, about 0.76, about 0.77, about 0.78, about 0.79, about 0.8, about 0.81, about 0.82, about 0.83, about 0.84, about 0.85, about 0.86, about 0.87, about 0.88, about 0.89, about 0.9, about 0.91, about 0.92, about 0.93, about 0.94, about 0.95, about 0.96, about 0.97, about 0.98, or about 0.99. Preferably, tₕᵢ is about 0.8, about 0.81, about 0.82, about 0.83, about 0.84, about 0.85, about 0.86, about 0.87, about 0.88, about 0.89, about 0.9, about 0.91, about 0.92, about 0.93, about 0.94, or about 0.95. More preferably, tₕᵢ is about 0.88.

In any of the embodiments of the present invention described herein, tₕᵢ may be from about 0.5 to about 0.99, from about 0.51 to about 0.98, from about 0.52 to about 0.97, from about 0.53 to about 0.96, from about 0.54 to about 0.95, from about 0.55 to about 0.94, from about 0.56 to about 0.93, from about 0.57 to about 0.92, from about to about 0.91, from about 0.58 to about 0.9, from about 0.59 to about 0.89, from about 0.6 to about 0.89, from about 0.61 to about 0.89, from about 0.62 to about 0.89, from about 0.63 to about 0.89, from about 0.64 to about 0.89, from about 0.65 to about 0.89, from about 0.67 to about 0.89, from about 0.68 to about 0.89, from about 0.69 to about 0.89, from about 0.7 to about 0.89, from about 0.71 to about 0.89, from about 0.72 to about 0.89, from about 0.74 to about 0.89, from about 0.76 to about 0.89, from about 0.78 to about 0.89, from about 0.79 to about 0.89, from about 0.8 to about 0.89, from about 0.81 to about 0.89, from about 0.82 to about 0.89, from about 0.83 to about 0.89, from about 0.84 to about 0.89, from about 0.85 to about 0.89, from about 0.86 to about 0.89, or from about 0.87 to about 0.89.

In any of the embodiments of the present invention described herein, tₗₒ and tₕᵢ may have the following pairs of values:

| | tₗₒ | tₕᵢ |
|---|---|---|
| 1 | from about 0.01 to about 0.5 | from about 0.5 to about 0.99 |
| 2 | from about 0.02 to about 0.49 | from about 0.51 to about 0.98 |
| 3 | from about 0.03 to about 0.48 | from about 0.52 to about 0.97 |
| 4 | from about 0.04 to about 0.47 | from about 0.53 to about 0.96 |
| 5 | from about 0.05 to about 0.46 | from about 0.54 to about 0.95 |
| 6 | from about 0.06 to about 0.45 | from about 0.55 to about 0.94 |
| 7 | from about 0.07 to about 0.44 | from about 0.56 to about 0.93 |
| 8 | from about 0.08 to about 0.43 | from about 0.57 to about 0.92 |
| 9 | from about 0.09 to about 0.42 | from about 0.58 to about 0.91 |
| 10 | from about 0.1 to about 0.41 | from about 0.59 to about 0.9 |
| 11 | from about 0.11 to about 0.4 | from about 0.6 to about 0.89 |
| 12 | from about 0.12 to about 0.39 | from about 0.61 to about 0.89 |
| 13 | from about 0.13 to about 0.38 | from about 0.62 to about 0.89 |
| 14 | from about 0.14 to about 0.37 | from about 0.63 to about 0.89 |
| 15 | from about 0.15 to about 0.36 | from about 0.64 to about 0.89 |
| 16 | from about 0.16 to about 0.35 | from about 0.65 to about 0.89 |
| 17 | from about 0.17 to about 0.34 | from about 0.66 to about 0.89 |
| 18 | from about 0.18 to about 0.33 | from about 0.67 to about 0.89 |
| 19 | from about 0.19 to about 0.32 | from about 0.68 to about 0.89 |
| 20 | from about 0.2 to about 0.31 | from about 0.69 to about 0.89 |
| 21 | from about 0.21 to about 0.3 | from about 0.7 to about 0.89 |
| 22 | from about 0.22 to about 0.29 | from about 0.71 to about 0.89 |
| 23 | from about 0.23 to about 0.28 | from about 0.72 to about 0.89 |
| 24 | from about 0.24 to about 0.27 | from about 0.73 to about 0.89 |
| 25 | from about 0.25 to about 0.27 | from about 0.74 to about 0.89 |
| 26 | from about 0.25 to about 0.27 | from about 0.75 to about 0.89 |
| 27 | from about 0.25 to about 0.27 | from about 0.76 to about 0.89 |
| 28 | from about 0.25 to about 0.27 | from about 0.77 to about 0.89 |
| 29 | from about 0.25 to about 0.27 | from about 0.78 to about 0.89 |
| 30 | from about 0.25 to about 0.27 | from about 0.79 to about 0.89 |
| 31 | from about 0.25 to about 0.27 | from about 0.8 to about 0.89 |
| 32 | from about 0.25 to about 0.27 | from about 0.81 to about 0.89 |
| 33 | from about 0.25 to about 0.27 | from about 0.82 to about 0.89 |
| 34 | from about 0.25 to about 0.27 | from about 0.83 to about 0.89 |
| 35 | from about 0.25 to about 0.27 | from about 0.84 to about 0.89 |
| 36 | from about 0.25 to about 0.27 | from about 0.85 to about 0.89 |
| 37 | from about 0.25 to about 0.27 | from about 0.86 to about 0.89 |
| 38 | from about 0.25 to about 0.27 | from about 0.87 to about 0.89 |
| 39 | about 0.26 | about 0.88 |

In the context of the present invention, the MHI is defined as the proportion of the assayed DMPs which have a β value which is intermediate between 1 and 0. Thus, the MHI can have a value between 0 and 1 (or between 0% and 100%). An MHI value of 0 indicates there is no methylation heterogeneity across the assayed DMPs i.e. all of the assayed DMPs have a non-intermediate β value e.g. each of the assayed DMPs have a β value that is less than tₗₒ or a β value that is greater than tₕᵢ. An MHI value of 1 indicates there is methylation heterogeneity for each of the assayed DMPs i.e. all of the assayed DMPs have an intermediate β value e.g. an intermediate β value defined as tₗₒ<<tₕᵢ.

The present inventors have developed predictive methods for identifying whether or not an individual has a cancer, a pre-invasive lesion that will progress to a cancer, or a pre-cancerous cell population that will progress to a cancer based on MHI. Thus, an individual may be identified as having a cancer, a pre-invasive lesion that will progress to a cancer, or a pre-cancerous cell population that will progress to a cancer if the MHI determined for a DNA sample taken from the individual is higher than the MHI that would be expected for an equivalent DNA sample taken from a healthy individual, e.g. an individual not having, a pre-invasive lesion that will progress to a cancer, or a pre-cancerous cell population that will progress to a cancer. The individual may be identified as having a cancer, a pre-invasive lesion that will progress to a cancer, or a pre-cancerous cell population that will progress to a cancer if the MHI determined for a DNA sample taken from the individual is high.

The individual may be identified as having a cancer, a pre-invasive lesion that will progress to a cancer, or a pre-cancerous cell population that will progress to a cancer based on a comparison of the MHI determined for a DNA sample taken from the individual with a “threshold value”. As used herein a “threshold value” is a numerical value between 0 and 1 to which the MHI determined for a DNA sample can be compared in order to make a binary classification for a given sample.

The individual may be identified as having a cancer, a pre-invasive lesion that will progress to a cancer, or a pre-cancerous cell population that will progress to a cancer if the MHI determined for a DNA sample taken from the individual is greater than a threshold value. The individual may be identified as not having a cancer, a pre-invasive lesion that will progress to a cancer, or a pre-cancerous cell population that will progress to a cancer if the MHI determined for a DNA sample taken from the individual is less than a threshold value.

For example, where a threshold value of X is applied, samples having an MHI less than X (MHI<X) will be classified as non-cancerous or regressive i.e. the individual from which the sample was obtained will be identified as not having a cancer, a pre-invasive lesion that will progress to a cancer, or a pre-cancerous cell population that will progress to a cancer. Conversely, samples having an MHI greater than X (X<MHI) will be classified as cancerous or progressive i.e. the individual from which the sample was obtained will be identified as having a cancer, a pre-invasive lesion that will progress to a cancer, or a pre-cancerous cell population that will progress to a cancer.

As shown in FIG. 5E (see also Example 6), an example method according to the present invention generated MHI scores of less than or equal to about 0.3 for a majority of non-cancerous TCGA control samples (see Figure E—black circles; sample numbers ˜1-30) whereas a majority of TCGA LUSC samples had MHI scores of from about 0.33 to about 0.4 (see FIG. 5E—orange circles; sample numbers ˜100+). Accordingly, an appropriate threshold value based on this analysis would be about 0.32. Thus, in this situation, where a sample is determined to have an MHI of less than 0.32 the individual from which the sample was taken will be identified as not having a cancer. Alternatively, in this example, where a sample is determined to have an MHI of greater than 0.32, the individual from which the sample was taken will be identified as having a cancer.

This example method also generated MHI scores of less than or equal to about 0.37 for a majority of regressive CIS samples (see FIG. 5E—green circles; sample numbers ˜31-37) whereas a majority of progressive samples had MHI scores of from about 0.37 or greater (see FIG. 5E—red circles; sample numbers ˜38-100). Thus, in this situation, where a sample is determined to have an MHI of less than 0.37 the individual from which the sample was taken will be identified as not having a pre-invasive lesion that will progress to a cancer, or a pre-cancerous cell population that will progress to a cancer. Alternatively, in this example, where a sample is determined to have an MHI of greater than 0.37, the individual from which the sample was taken will be identified as having a pre-invasive lesion that will progress to a cancer, or a pre-cancerous cell population that will progress to a cancer.

Thus, the threshold value may be about 0.02, about 0.04, about 0.06, about 0.08, about 0.1, about 0.12, about 0.14, about 0.16, about 0.18, about 0.2, about 0.22, about 0.24, about 0.26, about 0.28, about 0.3, about 0.32, about 0.34, about 0.36, about 0.38, about 0.4, about 0.42, about 0.44, about 0.46, about 0.48, about 0.5, about 0.52, about 0.54, about 0.56, about 0.58, about 0.6, about 0.62, about 0.64, about 0.66, about 0.68, about 0.7, about 0.72, about 0.74, about 0.76, about 0.78, about 0.8, about 0.82, about 0.84, about 0.86, about 0.88, about 0.9, about 0.92, about 0.94, about 0.96, or about 0.98. Preferably, the threshold value is about 0.2, about 0.22, about 0.24, about 0.26, about 0.28, about 0.3, about 0.32, about 0.34, about 0.36, about 0.38, about 0.4, about 0.42, about 0.44, about 0.46, about 0.48, or about 0.5. Preferably, the threshold value is about 0.26, about 0.27, about 0.28, about 0.29, about 0.3, about 0.31, about 0.32, about 0.33, about 0.34, about 0.35, about 0.36, about 0.37, about 0.38, about 0.39, about 0.4, about 0.41, or about 0.42.

In other words, in some embodiments of the methods of the present invention the individual is identified as having a cancer, a pre-invasive lesion that will progress to a cancer, or a pre-cancerous cell population that will progress to a cancer if the MHI determined for the DNA sample is greater than about 0.02, about 0.04, about 0.06, about 0.08, about 0.1, about 0.12, about 0.14, about 0.16, about 0.18, about 0.2, about 0.22, about 0.24, about 0.26, about 0.28, about 0.3, about 0.32, about 0.34, about 0.36, about 0.38, about 0.4, about 0.42, about 0.44, about 0.46, about 0.48, about 0.5, about 0.52, about 0.54, about 0.56, about 0.58, about 0.6, about 0.62, about 0.64, about 0.66, about 0.68, about 0.7, about 0.72, about 0.74, about 0.76, about 0.78, about 0.8, about 0.82, about 0.84, about 0.86, about 0.88, about 0.9, about 0.92, about 0.94, about 0.96, or about 0.98, preferably greater than about 0.2, about 0.22, about 0.24, about 0.26, about 0.28, about 0.3, about 0.32, about 0.34, about 0.36, about 0.38, about 0.4, about 0.42, about 0.44, about 0.46, about 0.48, or about 0.5, more preferably greater than about 0.26, about 0.27, about 0.28, about 0.29, about 0.3, about 0.31, about 0.32, about 0.33, about 0.34, about 0.35, about 0.36, about 0.37, about 0.38, about 0.39, about 0.4, about 0.41, or about 0.42; and/or

the individual is identified as not having a cancer, a pre-invasive lesion that will progress to a cancer, or a pre-cancerous cell population that will progress to a cancer if the MHI determined for the DNA sample is less than about 0.02, about 0.04, about 0.06, about 0.08, about 0.1, about 0.12, about 0.14, about 0.16, about 0.18, about 0.2, about 0.22, about 0.24, about 0.26, about 0.28, about 0.3, about 0.32, about 0.34, about 0.36, about 0.38, about 0.4, about 0.42, about 0.44, about 0.46, about 0.48, about 0.5, about 0.52, about 0.54, about 0.56, about 0.58, about 0.6, about 0.62, about 0.64, about 0.66, about 0.68, about 0.7, about 0.72, about 0.74, about 0.76, about 0.78, about 0.8, about 0.82, about 0.84, about 0.86, about 0.88, about 0.9, about 0.92, about 0.94, about 0.96, or about 0.98, preferably less than about 0.2, about 0.22, about 0.24, about 0.26, about 0.28, about 0.3, about 0.32, about 0.34, about 0.36, about 0.38, about 0.4, about 0.42, about 0.44, about 0.46, about 0.48, or about 0.5, more preferably less than about 0.26, about 0.27, about 0.28, about 0.29, about 0.3, about 0.31, about 0.32, about 0.33, about 0.34, about 0.35, about 0.36, about 0.37, about 0.38, about 0.39, about 0.4, about 0.41, or about 0.42.

The threshold value may be from about 0.02 to about 0.98, from about 0.04 to about 0.96, from about 0.06 to about 0.94, from about 0.08 to about 0.92, from about 0.1 to about 0.9, from about 0.12 to about 0.88, from about 0.14 to about 0.86, from about 0.16 to about 0.84, from about 0.18 to about 0.82, from about 0.2 to about 0.8, from about 0.22 to about 0.78, from about 0.24 to about 0.76, from about 0.26 to about 0.74, from about 0.28 to about 0.72, from about 0.28 to about 0.7, from about 0.26 to about 0.34, from about 0.26 to about 0.32, from about 0.28 to about 0.34, from about 0.28 to about 0.36, from about 0.28 to about 0.38, from about 0.28 to about 0.4, from about 0.28 to about 0.42, from about 0.28 to about 0.44, from about 0.28 to about 0.46, from about 0.28 to about 0.48, from about 0.28 to about 0.5, from about 0.28 to about 0.52, from about 0.28 to about 0.54, from about 0.28 to about 0.56, from about 0.28 to about 0.58, from about 0.28 to about 0.6, from about 0.28 to about 0.62, from about 0.28 to about 0.64, from about 0.28 to about 0.66, or from about 0.28 to about 0.68.

In other words, in some embodiments of the methods of the present invention the individual is identified as having a cancer, a pre-invasive lesion that will progress to a cancer, or a pre-cancerous cell population that will progress to a cancer if the MHI determined for the DNA sample is greater than from about 0.02 to about 0.98, from about 0.04 to about 0.96, from about 0.06 to about 0.94, from about 0.08 to about 0.92, from about 0.1 to about 0.9, from about 0.12 to about 0.88, from about 0.14 to about 0.86, from about 0.16 to about 0.84, from about 0.18 to about 0.82, from about 0.2 to about 0.8, from about 0.22 to about 0.78, from about 0.24 to about 0.76, from about 0.26 to about 0.74, from about 0.28 to about 0.72, from about 0.28 to about 0.7, from about 0.26 to about 0.34, from about 0.26 to about 0.32, from about 0.28 to about 0.34, from about 0.28 to about 0.36, from about 0.28 to about 0.38, from about 0.28 to about 0.4, from about 0.28 to about 0.42, from about 0.28 to about 0.44, from about 0.28 to about 0.46, from about 0.28 to about 0.48, from about 0.28 to about 0.5, from about 0.28 to about 0.52, from about 0.28 to about 0.54, from about 0.28 to about 0.56, from about 0.28 to about 0.58, from about 0.28 to about 0.6, from about 0.28 to about 0.62, from about 0.28 to about 0.64, from about 0.28 to about 0.66, or from about 0.28 to about 0.68; and/or

the individual is identified as not having a cancer, a pre-invasive lesion that will progress to a cancer, or a pre-cancerous cell population that will progress to a cancer if the MHI determined for the DNA sample is less than from about 0.02 to about 0.98, from about 0.04 to about 0.96, from about 0.06 to about 0.94, from about 0.08 to about 0.92, from about 0.1 to about 0.9, from about 0.12 to about 0.88, from about 0.14 to about 0.86, from about 0.16 to about 0.84, from about 0.18 to about 0.82, from about 0.2 to about 0.8, from about 0.22 to about 0.78, from about 0.24 to about 0.76, from about 0.26 to about 0.74, from about 0.28 to about 0.72, from about 0.28 to about 0.7, from about 0.26 to about 0.34, from about 0.26 to about 0.32, from about 0.28 to about 0.34, from about 0.28 to about 0.36, from about 0.28 to about 0.38, from about 0.28 to about 0.4, from about 0.28 to about 0.42, from about 0.28 to about 0.44, from about 0.28 to about 0.46, from about 0.28 to about 0.48, from about 0.28 to about 0.5, from about 0.28 to about 0.52, from about 0.28 to about 0.54, from about 0.28 to about 0.56, from about 0.28 to about 0.58, from about 0.28 to about 0.6, from about 0.28 to about 0.62, from about 0.28 to about 0.64, from about 0.28 to about 0.66, or from about 0.28 to about 0.68.

For any particular combination of MHI parameters (e.g. tₗₒ and tₕᵢ values) and threshold value applied to a differential methylation data set, the skilled person will be able to determine ROC AUC, sensitivity and/or specificity metrics as discussed above. These metrics may be used to evaluate the usefulness of a particular method for a particular application. For example, a method that achieves a high sensitivity (few false negatives) at the expense of specificity (greater number of false positives) may be desirable if the method is to be used as a screening assay.

For example, the skilled person would be able to select MHI parameters and a threshold value that provide a method achieving a desired ROC AUC, sensitivity and/or specificity.

Thus, the methods of the present invention disclosed herein may achieve:

(i) an ROC AUC of at least about 0.5, about 0.51, about 0.52, about 0.53, about 0.54, about 0.55, about 0.56, about 0.57, about 0.58, about 0.59, about 0.6, about 0.61, about 0.62, about 0.63, about 0.64, about 0.65, about 0.66, about 0.67, about 0.68, about 0.69, about 0.7, about 0.71, about 0.72, about 0.73, about 0.74, about 0.75, about 0.76, about 0.77, about 0.78, about 0.79, about 0.8, about 0.81, about 0.82, about 0.83, about 0.84, about 0.85, about 0.86, about 0.87, about 0.88, about 0.89, about 0.9, about 0.91, about 0.92, about 0.93, about 0.94, about 0.95, about 0.96, about 0.97, about 0.98, or about 0.99;

(ii) a specificity of at least about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%; and/or

(iii) a sensitivity of at least about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%.

Where the method of the present invention is a method for identifying whether or not an individual has a cancer the method may achieve an ROC AUC of at least about about 0.9, about 0.91, about 0.92, about 0.93, about 0.94, about 0.95, or about 0.96.

Where the method of the present invention is a method for identifying whether or not an individual has a lung cancer the method may achieves an ROC AUC of at least about about 0.9, about 0.91, about 0.92, about 0.93, about 0.94, about 0.95, or about 0.96.

Where the method of the present invention is a method for identifying whether or not an individual has a lung squamous cell carcinoma (LUSC), the method may achieves an ROC AUC of at least about about 0.9, about 0.91, about 0.92, about 0.93, about 0.94, about 0.95, or about 0.96.

Where the method of the present invention is a method for identifying whether or not an individual has a pre-invasive lesion that will progress to a cancer, the method may achieve an ROC AUC of at least about 0.66, about 0.67, about 0.68, about 0.69, about 0.7, about 0.71, about 0.72, about 0.73, or about 0.74.

Where the method of the present invention is a method for identifying whether or not an individual has a pre-invasive lung lesion that will progress to a cancer, the method may achieve an ROC AUC of at least about 0.66, about 0.67, about 0.68, about 0.69, about 0.7, about 0.71, about 0.72, about 0.73, or about 0.74.

Where the method of the present invention is a method for identifying whether or not an individual has a lung carcinoma in situ (CIS) that will progress to a cancer, the method may achieve an ROC AUC of at least about 0.66, about 0.67, about 0.68, about 0.69, about 0.7, about 0.71, about 0.72, about 0.73, or about 0.74.

The present invention also provides a methylation heterogeneity index (MHI) as defined herein for identifying in an individual a cancer, a pre-invasive lesion that will progress to a cancer, or a pre-cancerous cell population that will progress to a cancer.

The present invention further provides the use of a methylation heterogeneity index (MHI) as defined herein for identifying in an individual a cancer, a pre-invasive lesion that will progress to a cancer, or a pre-cancerous cell population that will progress to a cancer.

### Methods of Treatment

The present invention further provides a method of treating and/or preventing a cancer and/or treating a pre-invasive lesion that will progress to a cancer or a pre-cancerous cell population that will progress to a cancer in an individual, the method comprising:

identifying a cancer, a pre-invasive lesion that will progress to a cancer, or a pre-cancerous cell population that will progress to a cancer in the individual by performing a method of the present invention described herein; and

administering a cancer therapy to the individual.

For example, the present invention provides a method of treating and/or preventing a cancer and/or treating a pre-invasive lesion that will progress to a cancer or a pre-cancerous cell population that will progress to a cancer in an individual, the method comprising:

identifying a cancer, a pre-invasive lesion that will progress to a cancer, or a pre-cancerous cell population that will progress to a cancer in the individual by performing an identification method; and

administering a cancer therapy to the individual;

wherein the identification method is a method of identifying whether or not an individual has a cancer, a pre-invasive lesion that will progress to a cancer, or a pre-cancerous cell population that will progress to a cancer, the method comprising:

(a) providing a DNA sample which has been taken from the individual;

(b) performing an assay to determine the methylation status (β) value for at least 100 different differentially methylated positions (DMPs) in the DNA sample;

(b) determining a methylation heterogeneity index (MHI) for the DNA sample, wherein the MHI is defined as the proportion of the assayed DMPs which have a β value which is intermediate between 1 and 0; and

(c) identifying whether or not the individual has a cancer, a pre-invasive lesion that will progress to a cancer, or a pre-cancerous cell population that will progress to a cancer, based on the MI-II.

The cancer therapy may comprise any known treatment or procedure known in the art. The cancer therapy may comprise surgical intervention

Thus, the methods of the present invention encompass administration a cancer therapy to an individual following the identification of a cancer, a pre-invasive lesion that will progress to a cancer, or a pre-cancerous cell population that will progress to a cancer, wherein the cancer therapy comprises of one or more surgical procedures, one or more chemotherapeutic agents, one or more immunotherapeutic agents, one or more radiotherapeutic agents, one or more hormonal therapeutic agents or any combination thereof.

Surgical procedures may comprise the resection/removal of a tissue, lesion (such as pre-invasive lesion, a tumour and/or a cancerous or pre-cancerous population.

Surgical procedures may comprise the removal or one or more lung lobe (lobectomy), removal of two lung lobes (bilobectomy) removal of a lung, a lung transplant, a lymphadenectomy, a wedge resection, a segmentectomy, and a sleeve resection.

Chemotherapeutic agents include the following. Alkylating agents, which include the nitrogen mustards, nitrosoureas, tetrazines, aziridines, cisplatin and platinum based derivatives, as well as the non-classical alkylating agents. Antimetabolites, which include the anti-folates, fluoropyrimidines, deoxynucleoside analogues and thiopurines. Microtubule disrupting agents, which include the vinca alkaloids and taxanes, as well as dolastatin 10 and derivatives thereof. Topoisomerase inhibitors, which include camptothecin, irinotecan and topotecan. Topoisomerase II poisons, which include etoposide, doxorubicin, mitoxantrone and teniposide. Topoisomerase II catalytic inhibitors, which include novobiocin, merbarone, and aclarubicin. Cytotoxic antibiotics, which include anthracyclines, actinomycin, bleomycin, plicamycin, and mitomycin.

Immunotherapeutics include monoclonal antibodies, antibody-drug conjugates, immune checkpoint inhibitors. For example, the lung cancer therapy may be selected from monoclonal antibodies directed against the VEGF/VEGFR pathway such as bevacizumab (Avastin), mononclonal antibodies directed against the EGFR pathway, such as Necitumumab (Portrazza), monoclonal antibodies that inhibit the PD-1. PD-L1 pathway, such as Atezolizumab (Tecentriq), Durvalumab (Imfinzi) Nivolumab (Opdivo) and Pembrolizumab (Keytruda).

Combination therapies include carboplatin-taxol and gemcitabine-cisplastin.

The cancer therapy may be selected from Abraxane (Paclitaxel Albumin-stabilized Nanoparticle Formulation), Afatinib Dimaleate, Afinitor (Everolimus), Alecensa (Alectinib), Alectinib, Alimta (Pemetrexed Disodium), Alunbrig (Brigatinib), Atezolizumab, Avastin (Bevacizumab), Bevacizumab, Brigatinib, Carboplatin, Ceritinib, Crizotinib, Cyramza (Ramucirumab), Dabrafenib, Dacomitinib, Docetaxel, Durvalumab, Erlotinib Hydrochloride, Everolimus, Gefitinib, Gilotrif (Afatinib Dimaleate), Gemcitabine Hydrochloride, Gemzar (Gemcitabine Hydrochloride), Imfinzi (Durvalumab), Iressa (Gefitinib), Keytruda (Pembrolizumab), Mechlorethamine Hydrochloride, Mekinist (Trametinib), Methotrexate, Mustargen (Mechlorethamine Hydrochloride), Navelbine (Vinorelbine Tartrate), Necitumumab, Nivolumab, Opdivo (Nivolumab), Osimertinib, Paclitaxel, Paclitaxel Albumin-stabilized Nanoparticle Formulation, Paraplat (Carboplatin), Paraplatin (Carboplatin), Pembrolizumab, Pemetrexed Disodium, Portrazza (Necitumumab), Ramucirumab, Tafinlar (Dabrafenib), Tagrisso (Osimertinib), Tarceva (Erlotinib Hydrochloride), Taxol (Paclitaxel), Taxotere (Docetaxel), Tecentriq (Atezolizumab), Trametinib, Trexall (Methotrexate), Vizimpro (Dacomitinib), Vinorelbine Tartrate, Xalkori (Crizotinib), Zykadia (Ceritinib), and combinations thereof.

The cancer therapy may comprise proton beam therapy. The cancer therapy may comprise photodynamic therapy.

The cancer therapy may be administered to an individual already having a cancer, in an amount sufficient to cure, alleviate or partially arrest the cancer or one or more of its symptoms. Such therapeutic treatment may result in a decrease in severity of disease symptoms, or an increase in frequency or duration of symptom-free periods. An amount adequate to accomplish this is defined as “therapeutically effective amount”. Effective amounts for a given purpose will depend on the severity of the disease as well as the weight and general state of the individual.

The cancer therapy may also be administered to an individual who has not yet developed a cancer but who is identified as having a pre-invasive lesion that will progress to a cancer or a pre-cancerous cell population that will progress to a cancer. Such preventative treatment may prevent the progression of the pre-invasive lesion to a cancer or prevent the progression of the pre-cancerous cell population to a cancer, delay the progression of the pre-invasive lesion to a cancer or delay the progression of the pre-cancerous cell population to a cancer, and/or lessen the severity or extent a cancer derived from the identified pre-invasive lesion or the pre-cancerous cell population.

### Cancer

In any of the methods, MHIs or uses thereof provided by the present invention, the pre-invasive lesion may be a solid lesion or the cancer may be a solid tumour. In any of the methods, MHIs or uses thereof provided by the present invention, the pre invasive lesion or pre-cancerous cell population may be present in the central nervous system, the eye, the ear, nose or throat, the skin, the lung, the bone, an endocrine tissue, breast tissue, the digestive system, the reproductive system, the liver, or the kidney.

The pre-invasive may be normal epithelium, tissue hyperplasia, dysplasia, or lung carcinoma in situ (CIS).

The term “cancer” as used herein refers to any of the diseases characterized by the presence of cancerous tissue in a subject. As used herein, “cancerous tissue” refers to a tissue that comprises malignant neoplastic cells, exhibits an abnormal growth of cells and/or hyperproliferative cells. Cancerous tissue can be a primary malignant tumor, arising from a tissue or organ of origin, or it can be a metastatic malignant tumor, growing in a body tissue which was not the source of the original tumor.

As used herein, the term “tumour” can include a solid tumor or a cancer of hematopoietic origin. In some embodiments the tumor may be characterized by its ability to invade surrounding tissues, to metastasize to other parts of the body, and/or by its angiogenic activity. Exemplary tumors result from hepatocellular carcinoma, gastric cancer, renal cancer, prostate cancer, adrenal cancer, pancreatic cancer, breast cancer, bladder cancer, salivary gland cancer, ovarian cancer, uterine body cancer, and lung cancer.

As used herein, the term “invasive” refers to the process by which a cell, a group of cells, or a malignancy spreads from a site to adjacent sites.

The term “metastatic”, as used herein, refers to the process by which a cell, a group of cells, or a malignancy spreads from a site to sites not adjacent to the first site.

The cancer may be a cancer of the central nervous system, the eye, the ear, nose or throat, the skin, the lung, the bone, an endocrine tissue, breast tissue, the digestive system, the reproductive system, the liver, or the kidney. The cancer may be selected from the group consisting of: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyoscarcoma, and osteosarcoma; and other tumors including melanoma, xenoderma pigmentosum, keratoactanthoma, seminoma, thyroid follicular cancer, and teratocarcinoma.

The cancer may be a lung cancer. The lung cancer may be a lung squamous cell carcinoma (LUSC).

The following Examples are provided to illustrate the invention but not to limit the invention.

### EXAMPLES

### Example 1: General Study Design

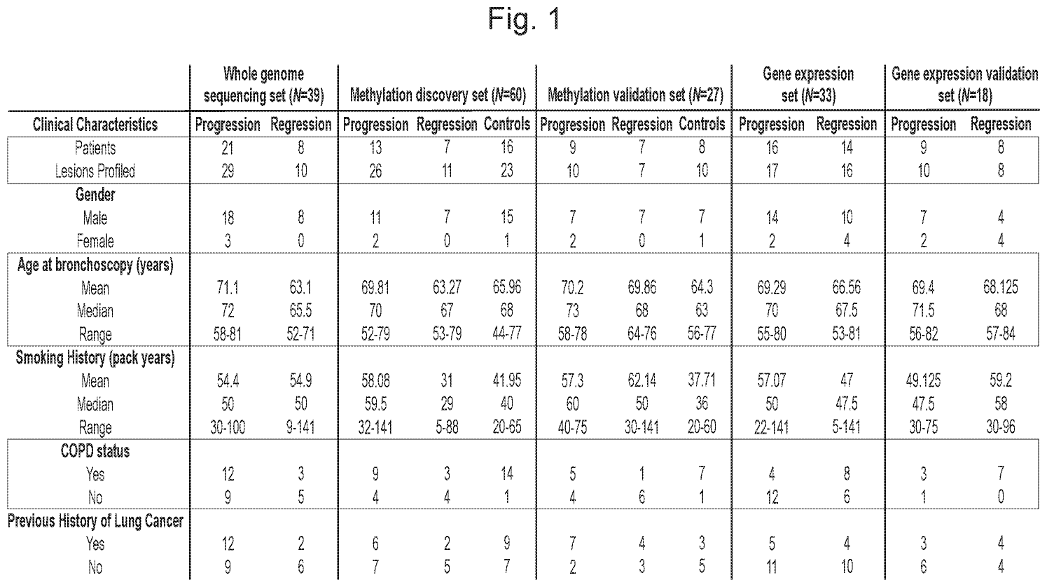

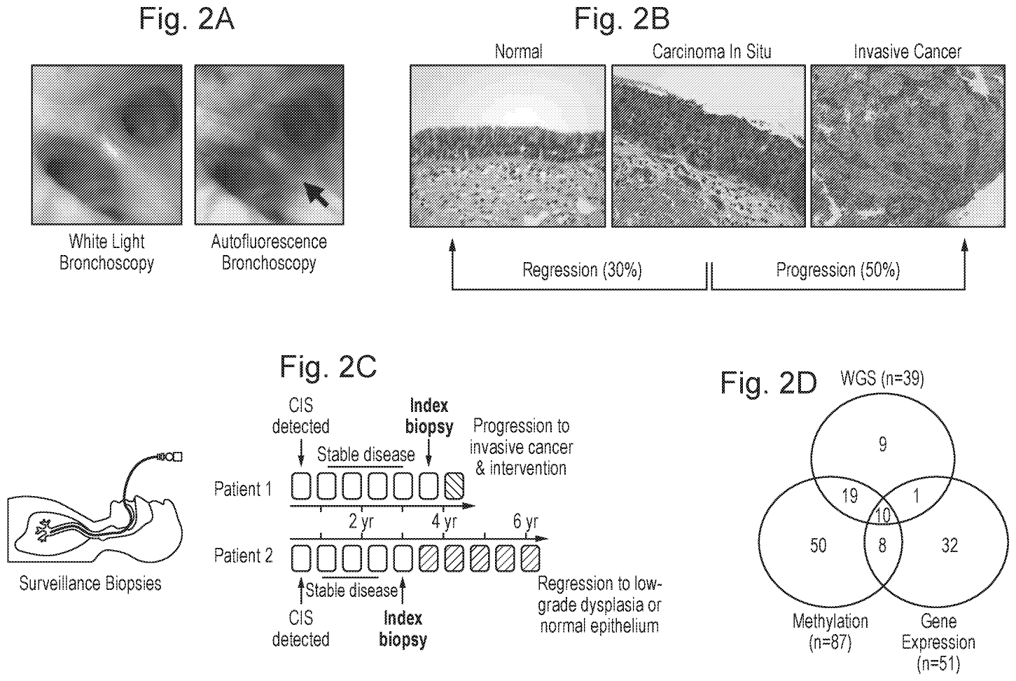

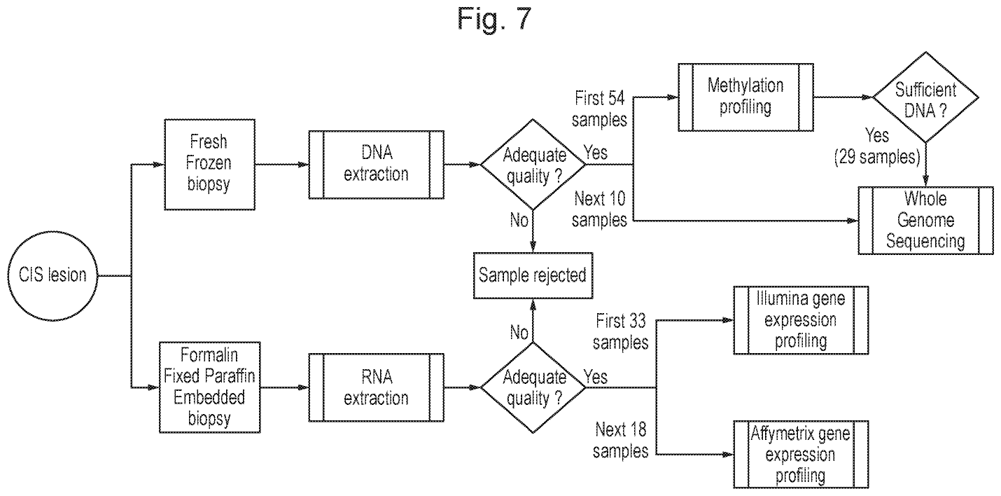

It was reasoned that information on the future clinical trajectory of a pre-invasive lung lesion might be encoded in the genetic and epigenetic profile present at diagnosis. A prospective cohort study of patients with pre-invasive squamous airway lesions was therefore undertaken. Patients were managed conservatively, undergoing surveillance AFB with biopsy and CT scanning every 4 and 12 months, respectively, with definitive cancer treatment only performed at the earliest pathological evidence of progression to invasive tumours (FIG. 2A, B) [7]. When a CIS lesion either progressed to invasive cancer or regressed to normal epithelium/low-grade disease, molecular profiling was performed on the preceding CIS biopsy from the same lesion—the ‘index biopsy’ (FIG. 2C). Index biopsies all demonstrated histologically and morphologically indistinguishable CIS and were classified as either ‘progressive’ or ‘regressive’. All such index CIS biopsies were subjected to a predetermined combination of transcriptomic, epigenetic and finally genomic profiling depending on DNA/RNA availability (FIG. 1; FIG. 2D; FIG. 7).

### Example 2: Patient Characteristics

Patients with pre-invasive lung cancer lesions were recruited through University College London Hospitals (UCLH) Early Lung Cancer Surveillance Programme (ELCSP). Full details of the surveillance protocol including eligibility criteria for patient inclusion have been previously described [7]. Briefly, the programme has recruited 140 patients to date with pre-invasive lung cancer lesions of varying histological grades. 129 index CIS biopsies were obtained from 85 patients and subjected to molecular analysis. Dependent on stored tissue quantity, in total, 51 samples from 42 patients underwent gene expression profiling; 87 samples from 47 patients underwent methylation profiling; and 39 samples from 29 patients underwent whole genome sequencing. Methylation and gene expression datasets were divided into independent discovery and validation groups.

Clinical characteristics within each analysis group are shown in FIG. 1. In comparing progressive and regressive samples, it was found that progressive samples were associated with a higher pack-year smoking history in the methylation discovery group only (p<0.01) and with increased age in the WGS group (p=0.01). No clinical differences were consistently observed across the different analysis groups.

### Example 3: Characterisation of CIS Genomic Profiles

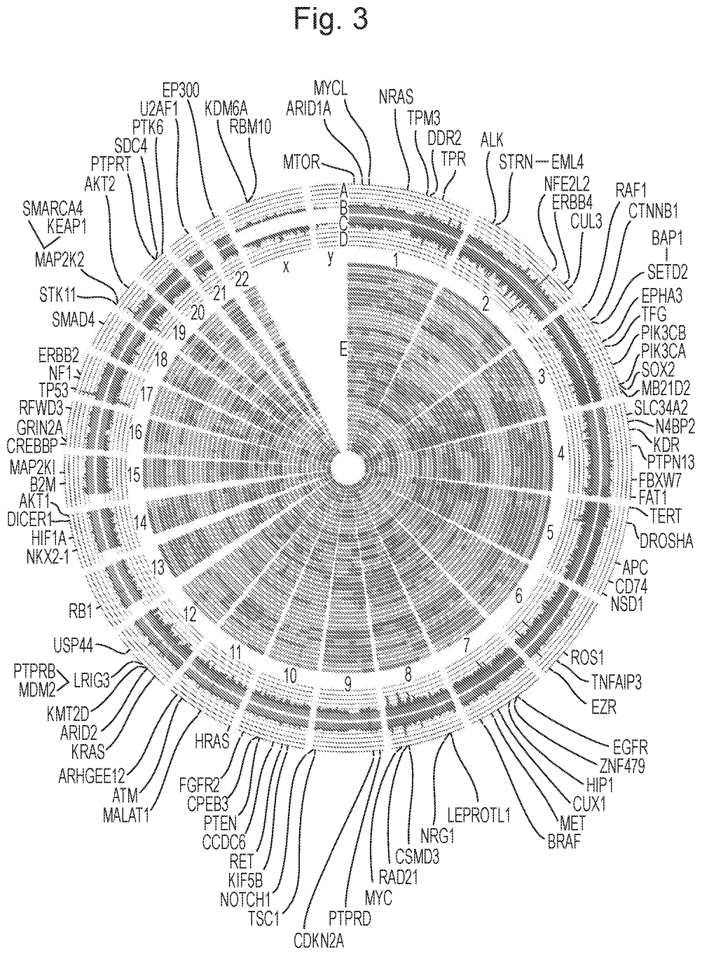

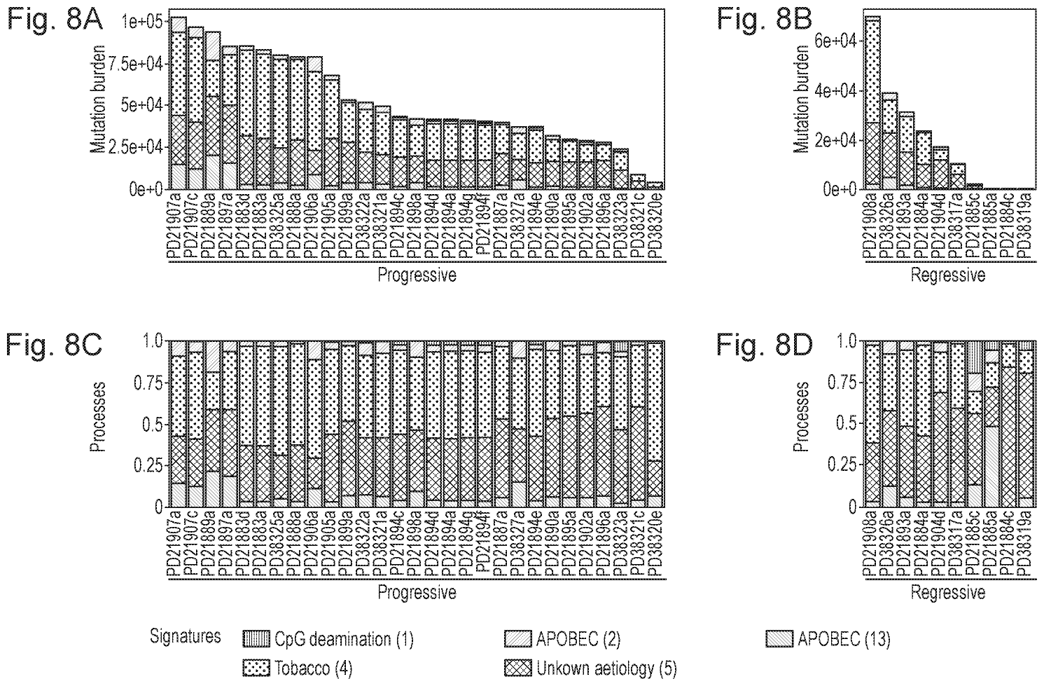

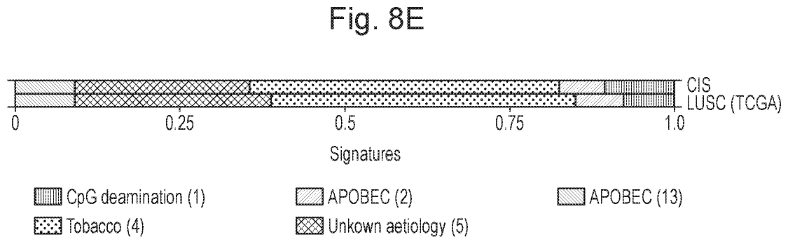

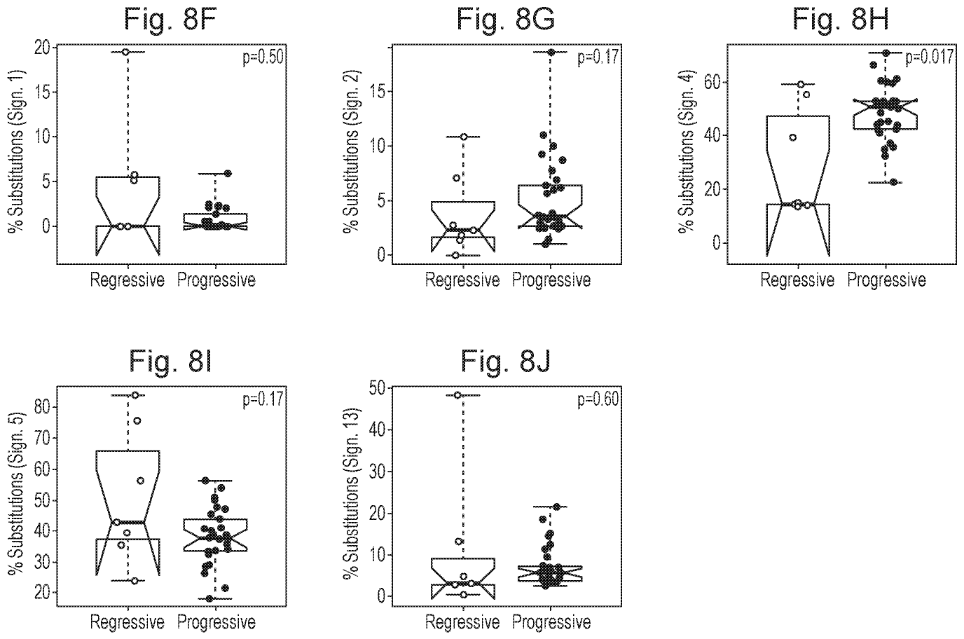

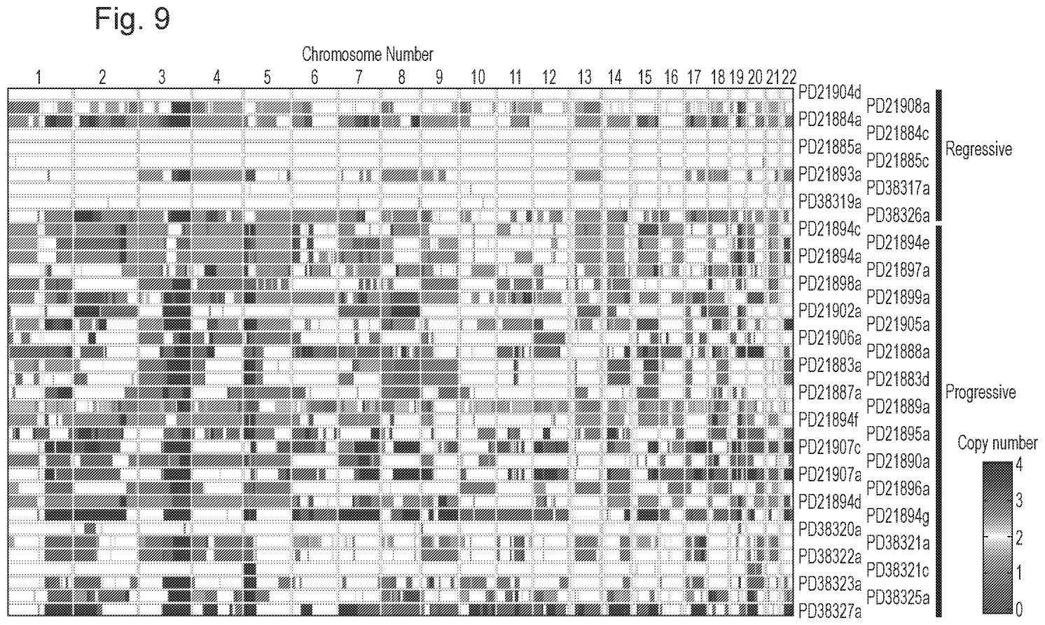

The 39 CIS lesions are the first pre-invasive LUSC lesions to be who le-genome sequenced, so the burden and spectrum of mutations in CIS was compared with publicly available LUSC exome sequencing data from The Cancer Genome Atlas (TCGA). Due to differences between whole-genome and exome sequencing, only broad comparisons were made. A similar mutation burden and copy number profile between CIS samples and TCGA LUSC tumours was observed (FIG. 3). There is congruency of type and prevalence of potential driver mutations, broadly defined as any mutation in a gene previously implicated as a driver of lung cancer, between CIS and LUSC samples [8]. Frequent alterations in TP53, CDKN2A, SOX2 and AKT2, and less frequent alterations in FAT1, KMT2D, KEAP1, EGFR and NOTCH1 in CIS lesions were observed (FIG. 3). CIS mutational signatures [9],[10] showed a strong tobacco-associated signal and were similar to those found in LUSC (FIG. 8).

Marked aneuploidy was observed in CIS lesions, with somatic copy number alterations (CNAs) present across the genome (FIG. 3; FIG. 7). The most frequent changes were associated with gain and amplification of multiple locations on distal 3q: this is known to be the most common genomic aberration in LUSC11. Other recognised copy number associations identified in our data include gain/amplification in 5p, 8q and 19q and regions of loss/deletion in 3p, 4q, 5q, 8p, 9p and 13q.12-18.

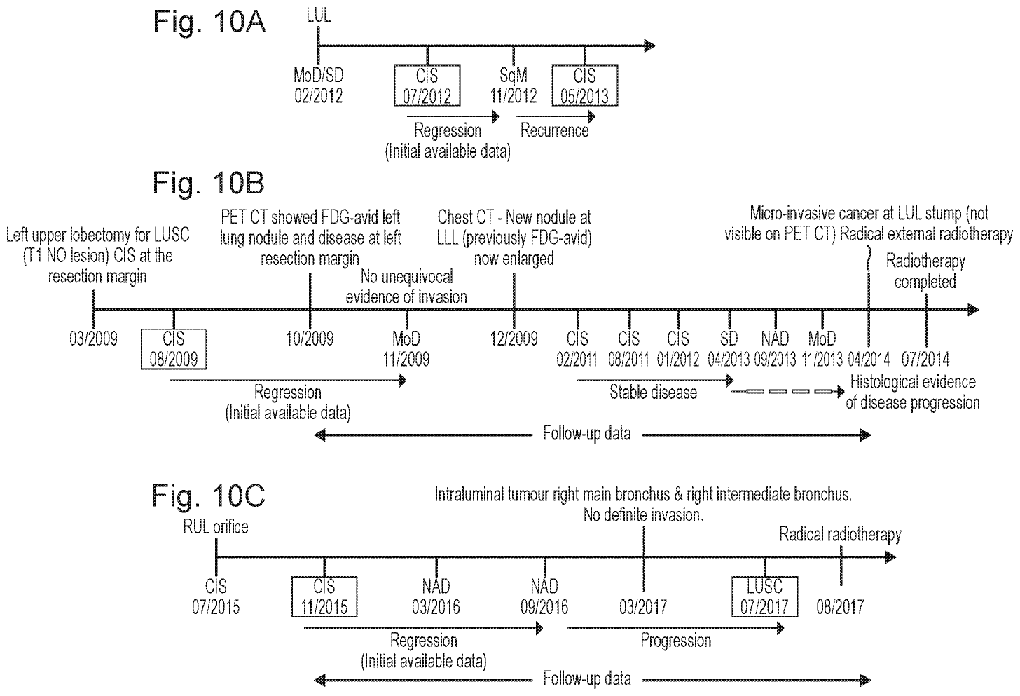

Whilst most CIS samples had the genomic appearance of neoplasms, six lesions were observed which showed markedly lower mutational load and fewer copy number alterations than the others (FIG. 7; PD21884c, PD21885a, PD21885c, PD21904d, PD38317a, PD38319a). These samples had very few genomic changes, despite being CIS histologically. All of these six samples regressed to normal epithelium or low-grade dysplasia on subsequent biopsy. Four further samples met this end-point for regression, despite widespread mutational and copy number changes. However, with longer follow up one of these cases developed CIS recurrence (FIG. 10a; PD21893a), and two developed invasive cancer on further surveillance (FIG. 10B, C; PD21884a, PD38326a). Only one sample, PD21908a, showed sustained clinical regression after 9 years of follow up despite widespread molecular changes.

All but one progressive sample and all highly mutated regressive samples showed amplification in a small region of distal 3q (chr3:172516434-178440382). This region contains known driver genes (SOX2, PIK3CA), genes associated with chromosomal instability (ACTL6A) and methyltransferases (ECE2). Progressive sample PD38320a had little change outside this region and did not harbour a TP53 mutation, suggesting that this amplification may be a crucial early event in LUSC tumorigenesis.

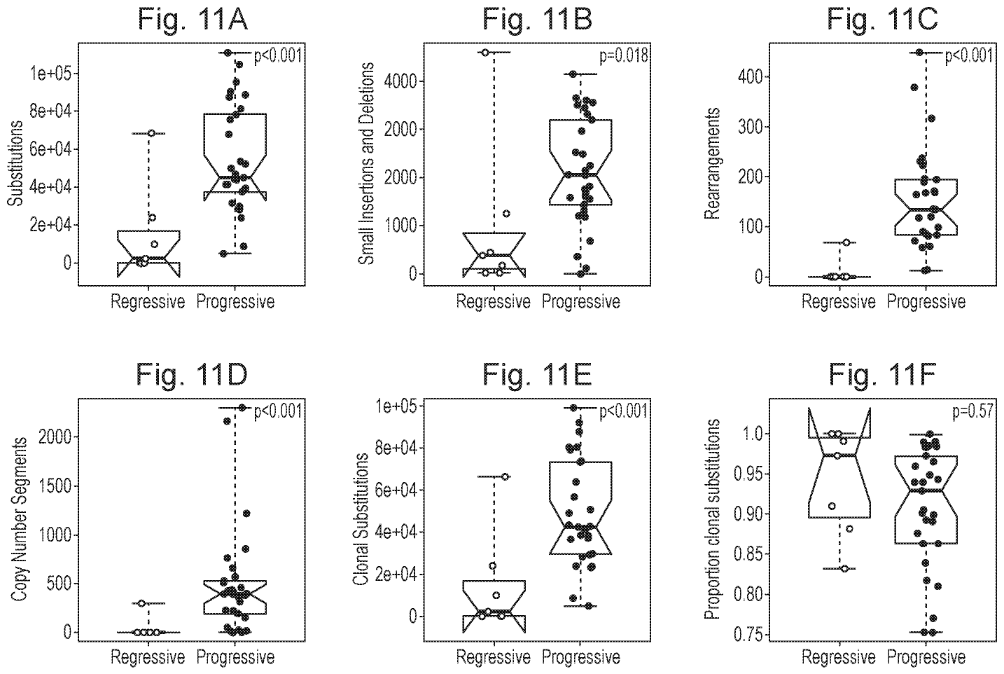

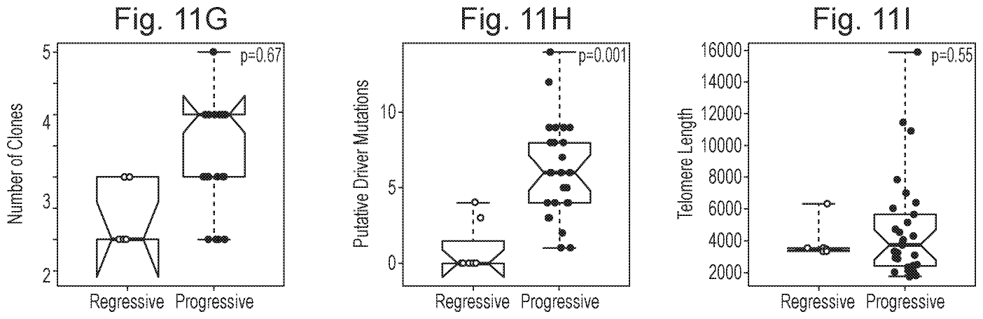

Genomic features between the 29 progressive and 10 regressive lesions were compared. The three samples which showed evidence of progression after meeting the end-point for regression were excluded from this analysis. Comparisons of mutation burden between progressive and regressive lesions were performed by mixed effects modelling, allowing us to account for samples that come from the same patient. Even after correcting for patient age, smoking history and sample purity, progressive lesions had more somatically acquired mutations than those from regressive lesions, across base substitutions (p<0.001), indels (p=0.018), structural variants (p<0.001) and copy number changes (p<0.001) (FIG. 11A-D). When the analysis was restricted only to substitutions that were fully clonal in each lesion, there were still substantially more substitutions in progressive than regressive lesions (p<0.001) (FIG. 11E), suggesting that the increase in mutation burden is not due to recent subclonal diversification in progressive lesions. All the mutational processes (or signatures [9], [10]) identified in the CIS lesions contribute to the excess of mutations in progressive compared to regressive samples; however, only tobacco-associated signature 4 showed proportionally more mutations (p=0.017) (FIG. 8F-J). Progressive lesions contained more putative driver mutations than regressive lesions (p=0.001) (FIG. 11H). Importantly, no single cancer mutation perfectly discriminated between progressive and regressive lesions.

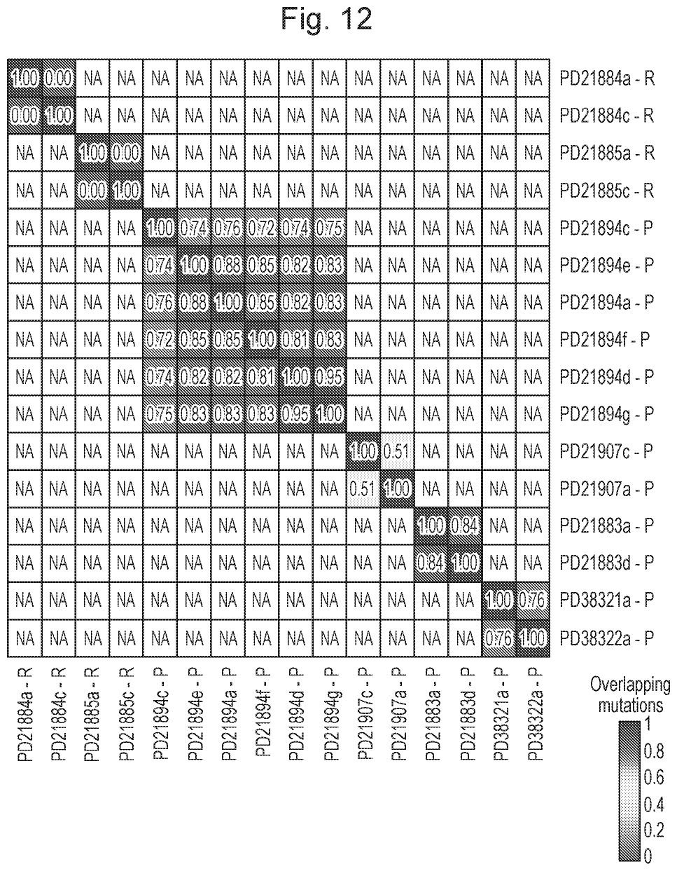

Within the biopsied lesions, clonal architecture was similar between progressive and regressive lesions (FIG. 11E-G). For four patients in whom we sequenced multiple progressive lesions, the lesions shared many somatic mutations despite their different locality in the bronchial tree, indicating their probable derivation from a common ancestral clone. By contrast, multiple regressive lesions from two further patients did not share common mutations and so are likely to have arisen independently (FIG. 12). There were no differences in telomere lengths between progressive and regressive lesions (p=0.59) (FIG. 11I).

### Example 4: CIS Transcriptomic and Epigenetic Profiles

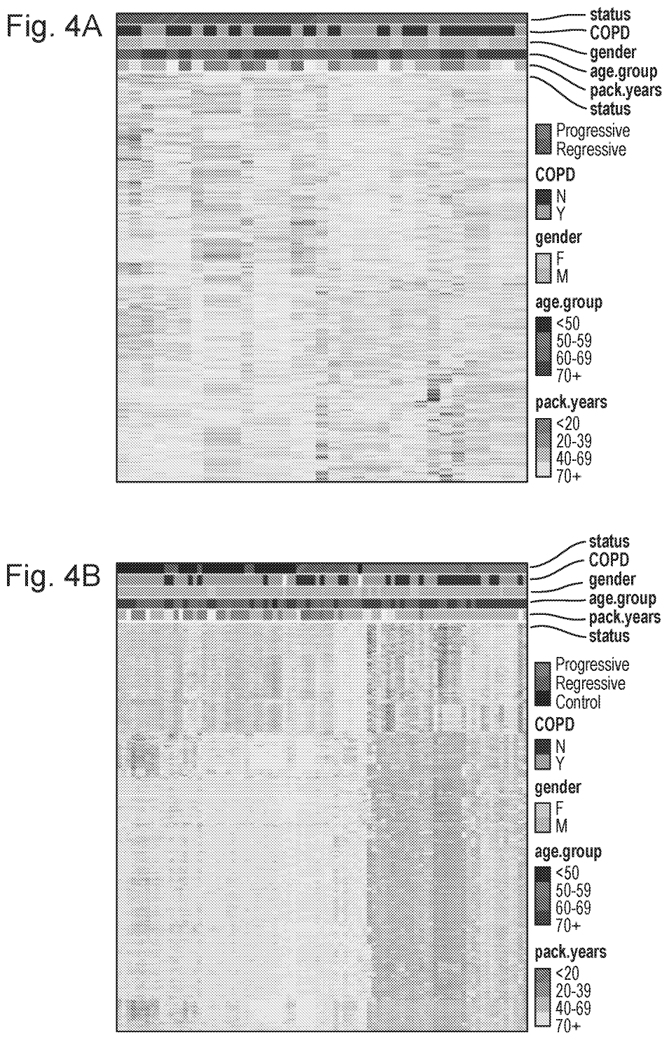

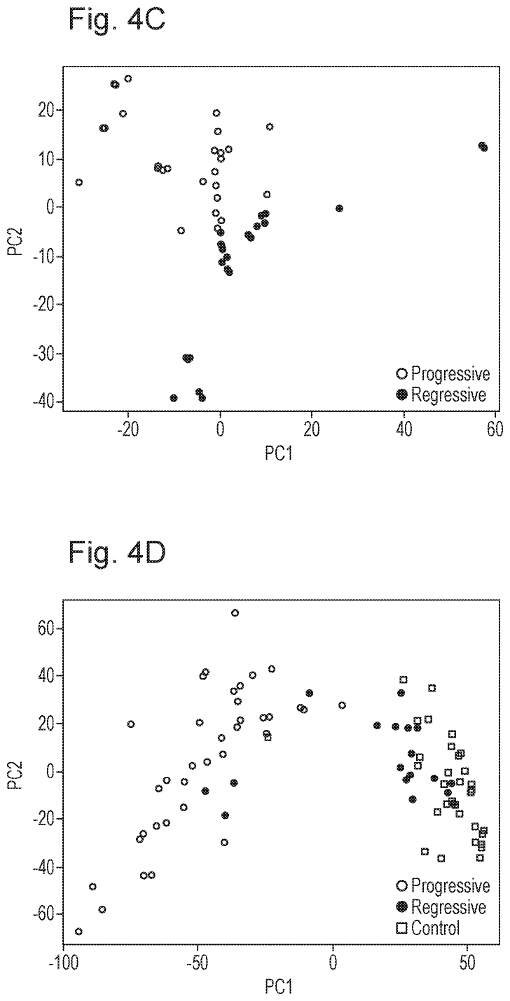

Gene expression microarrays were performed on a discovery set of 17 progressive and 16 regressive CIS lesions. Identified were 1335 genes with significant expression changes (FDR <0.01); 657 genes were up-regulated and 678 down-regulated in progressive CIS lesions (FIG. 4A).

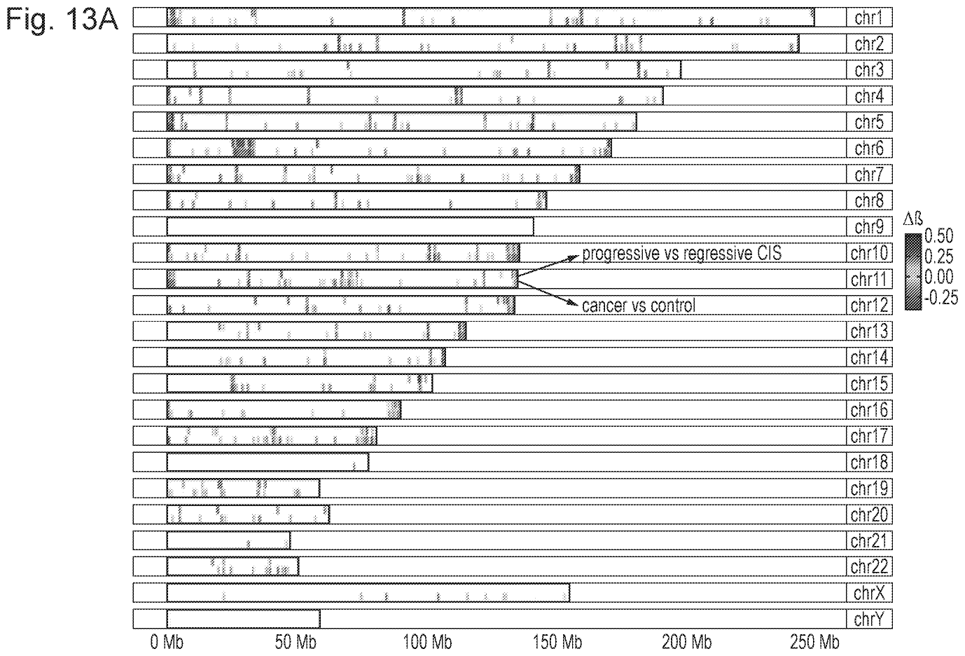

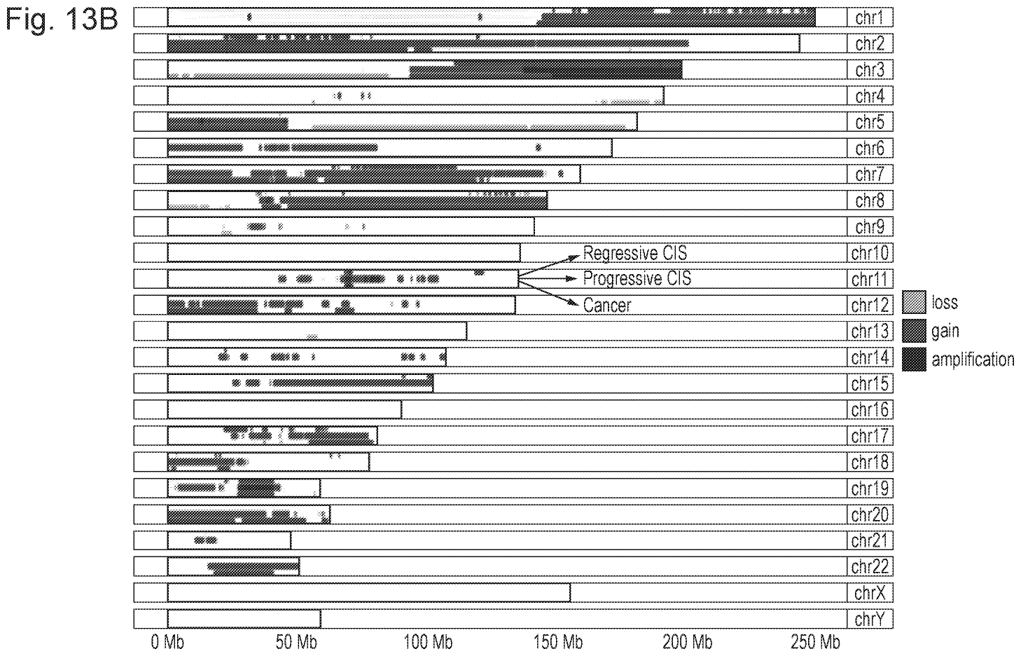

Differential analysis of methylation profiles was performed on a discovery set of 26 progressive, 11 regressive and 23 control samples. Widespread methylation changes were observed with 12,064 differentially methylated positions (DMPs), associated with 2,695 genes, at which methylation was significantly different between progressive and regressive samples (FDR <0.01; |Δβ|>0.3). 6,314 DMPs were hypermethylated and 5,750 hypomethylated in progressive CIS (FIG. 4B). 260 differentially methylated regions (DMRs) were identified, of which 151 (58%) overlap with DMRs between TCGA cancer and control data (FIG. 13). Finally, identified were 36,620 differentially variable positions (DVPs) for which probe variance was markedly different between progressive and regressive groups.

Of the 1335 genes identified, TPM3, PTPRB, SLC34A2, KEAP1, NKX2-1, SMAD4 and SMARCA4 have previously been implicated as potential lung cancer drivers. Regarding methylation, the potential driver genes NKX2-1, TERT, DDR2, LRIG3, CUX1, EPHA3, CSMD3, MET, ZNF479, GRIN2A, PTPRD, NOTCH1, CD74, NSD1 and CDKN2A contain at least one significant DMP. Several genes which are significant in our gene expression analysis are also identified in our methylation data, including multiple genes in the homeobox family (HOXC8, HOXC9, HOXC10, HOXD10, HOXA11AS), previously implicated as an early epigenetic event in multiple cancers [19]. NKX2-1 (TTF-1) is the only putative driver gene to be identified in both gene expression and methylation analyses, and is also a member of the homeobox family. It is hypermethylated and underexpressed in progressive samples compared to regressive. This gene is widely used in diagnosis of lung adenocarcinoma and both underexpression and hypermethylation have been implicated in the development of this disease [20], [21]. NKX2-1 loss has been shown to drive squamous cancer formation in combination with SOX2 overexpression [22]; focal gains in the 3q region containing SOX2 are commonly observed in progressive CIS (FIG. 10).

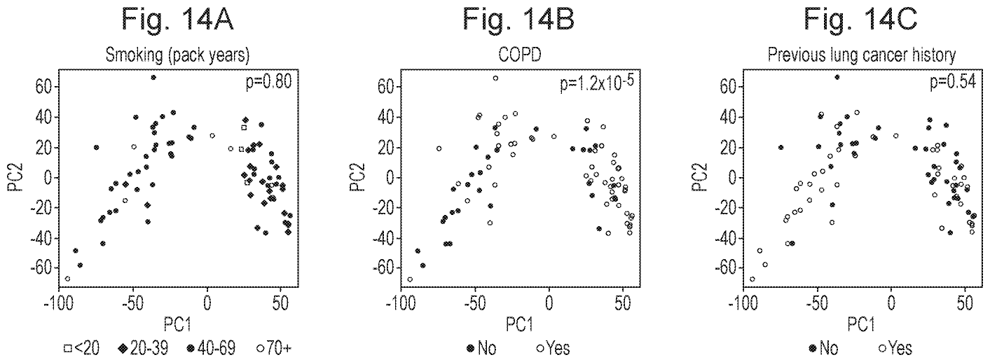

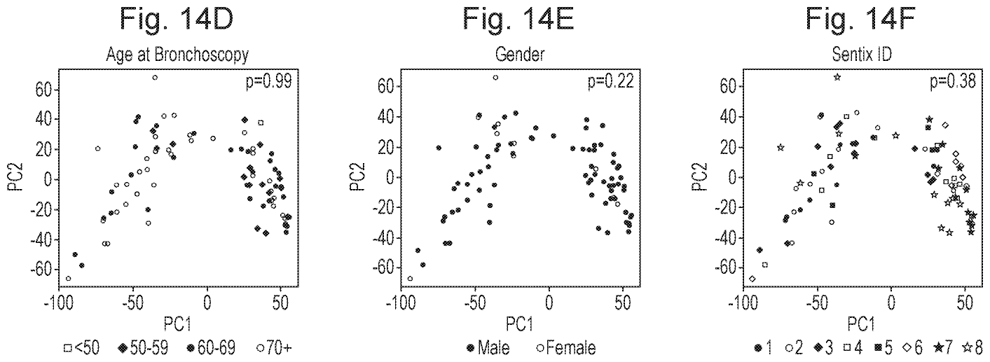

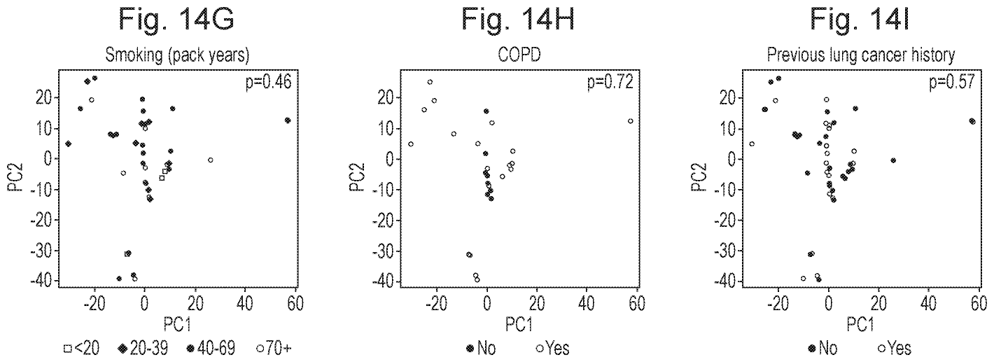

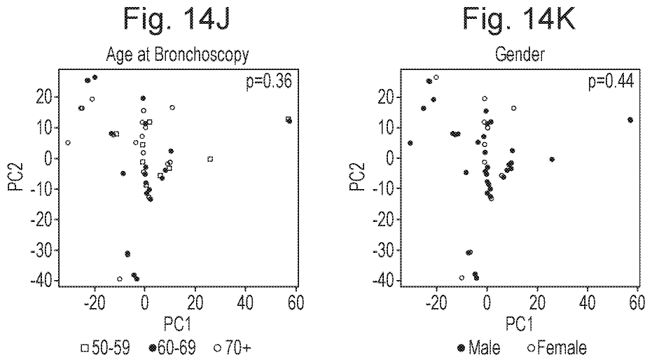

Principal component analysis of all gene expression and methylation data showed a clear distinction between the progressive and regressive subgroups (p=0.0017 and p=6.8×10-25, respectively) (FIG. 4C,D). In the methylation dataset, the regressive lesions closely clustered with the control normal epithelial cells. A history of chronic obstructive pulmonary disease (COPD) had an effect on case segregation (p=1.2×10-5) but all other clinical and technical variables analysed, including smoking status and history of lung cancer, had no effect (FIG. 14). This was also the case for PCA analysis of the gene expression data (FIG. 14G-K).

For methylation, one control and four regressive cases clustered with the progressive cases (FIG. 4D). Three of the four miss-classified regressive cases were subjected to whole-genome sequencing and were found to have more copy number alterations than other regressive samples (PD21884a, PD21893a, PD21908a). Two of these correspond to the samples discussed above, which showed signs of progression after meeting the clinical end point of regression (FIG. 10). For the control bronchial epithelium sample that was classified with the progressive lesions, CIS was detected in a biopsy specimen 12 months later from the same site. Thus, although these cases were formally treated as miss-classifications, it is likely that the molecular data underpinning the apparent errors indicate a cellular phenotype that is not consistent with a straightforward regressive lesion.

### Example 5: Molecular Signatures Predict CIS Outcome

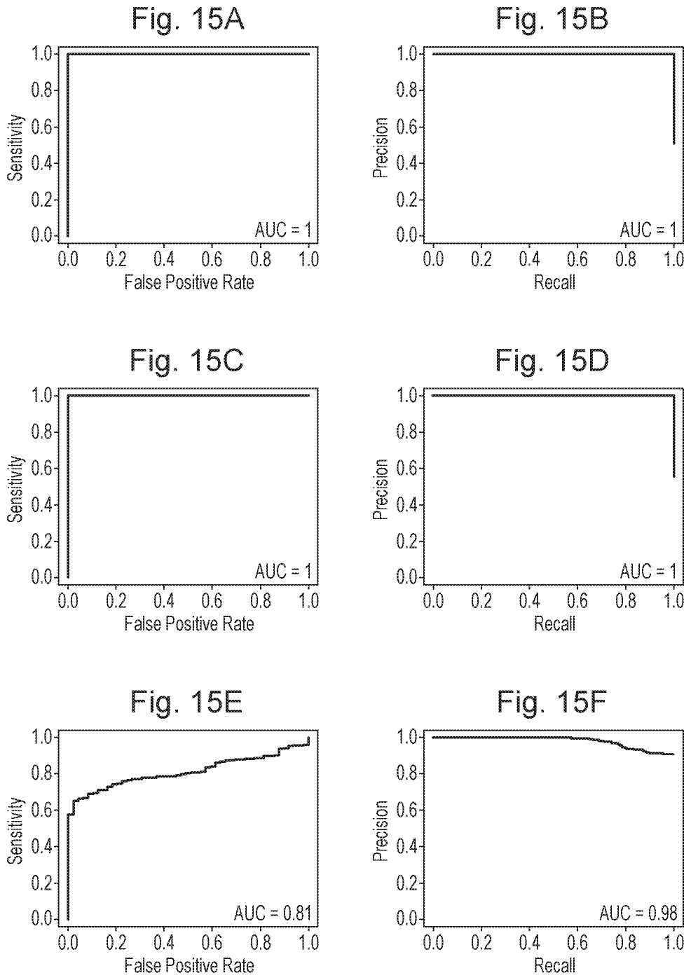

The ability to predict if a pre-invasive lesion will progress to cancer has important clinical implications. For gene expression, the above pre-defined discovery set to define our classifier were used (n=33; 17 progressive, 16 regressive; 10-fold cross-validation applied). This was applied to a separate validation set (n=18; 10 progressive, 8 regressive). All samples in the validation set were classified correctly. When applied to external data from TCGA (n=551: 502 LUSC, 49 control), the 291-gene model was able to classify LUSC vs control samples with AUC=0.81 (FIG. 5A-C; FIG. 15).

An analogous analysis was performed for methylation using a discovery set of 60 samples and a validation set of 27 samples. This classified validation samples with AUC=0.99 and classified external TCGA samples (n=412: 370 LUSC, 42 controls) into LUSC vs controls with AUC=0.99, based on a 141-DMP classifier (FIG. 16A-I).

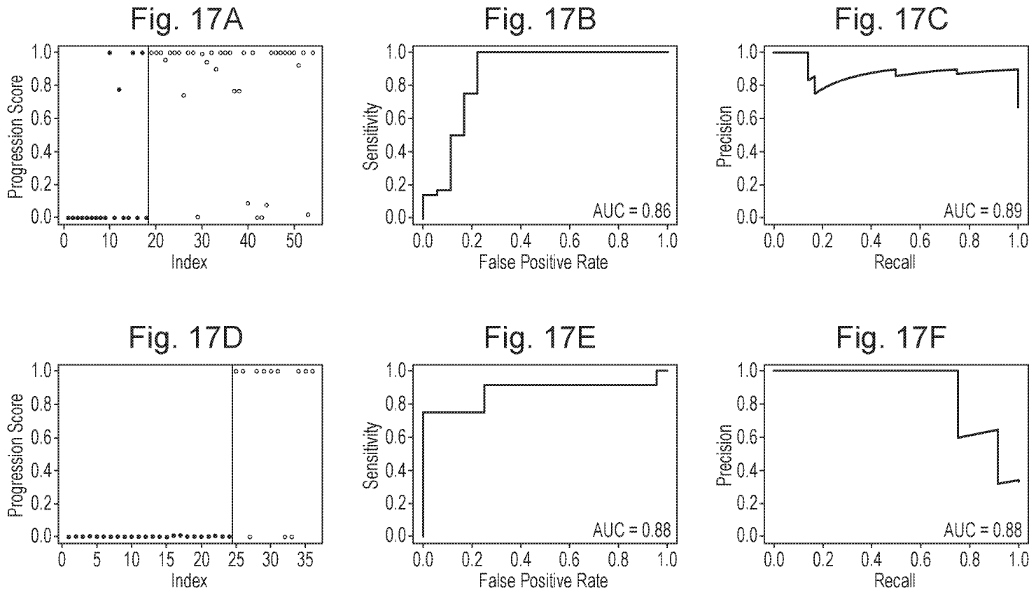

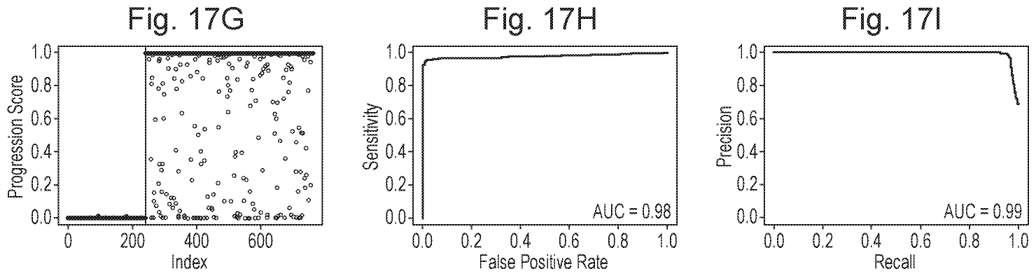

To build a predictive classifier based on copy number, copy number derived from methylation data was used to increase sample size and classified 46 of 54 samples correctly (FIG. 17A-C). The 154 predictive cytogenetic bands that we identified overlap with, but are not limited to, a model previously proposed by van Boerdonk et al. The model presented herein replicated their results, classifying 24/24 regressive samples and 9/12 progressive samples correctly [23] (FIG. 17D-F). When applied to external data from TCGA (n=763: 524 LUSC, 239 control), the model was able to classify LUSC vs control samples with AUC=0.98 (FIG. 17G-I).

Further analyses was performed using only one sample per patient to demonstrate that our results are not dependent on multiple sampling. The first available sample for each patient was selected, with CIS samples prioritised over control samples for methylation data. Results are similar to our analysis above, validating our initial results.

Although it cannot be fully excluded that lesions meeting the end point for regression will progress in future, most patients in this cohort now have several years of follow up. Of 35 regressive lesions undergoing molecular profiling, mean follow up was 67 months (median 57 months, range 11-150 months).

### Example 6: Methylation Heterogeneity Index (MHI) is Predictive of CIS Outcomes and Presence of LUSC

An increased number of methylation probes with intermediate methylation was observed for TCGA LUSC cancer samples as compared to TCGA control samples (FIG. 5D), reflecting methylation heterogeneity in the cancer samples. We therefore developed a methylation heterogeneity index (MHI), defined as the number of probes per sample with tₗₒ<β<tₕᵢ. Optimisation based on the discovery set of 26 progressive and 11 regressive samples defined values of tₗₒ=0.26 and tₕᵢ=0.88. Control samples were not used in this analysis. This model classified progressive vs regressive CIS samples in our validation set with AUC=0.74 and TCGA LUSC vs TCGA control samples with AUC=0.96 (FIG. 5E; FIG. 16J-N). Multivariate logistic regression in our CIS cohort demonstrated that this index was a predictor of progression status (p=0.017); previous history of lung cancer was also significantly associated (p=0.02), whereas smoking status, COPD status, age and gender were not.

Given the widespread nature of methylation changes, it was hypothesised that this increase in heterogeneity may be a genome-wide process rather than specific to functional pathways. To test this theory, the predictive value of MHI calculated from a sample of 2,000 probes, randomly selected from across the genome, was assessed. Tables 1-10 provide representative example sets of 2000 randomly selected DMPs and the ROC metrics obtained when each set of DMPs was used to classify TCGA LUSC samples versus TCGA controls samples (“CvC”) or progressive versus regressive CIS samples (“PvR”). Running 10,000 simulations with each using a different random sample of 2,000 probes gave a mean AUC for TCGA LUSC vs TCGA control of 0.95 (95% CI 0.92-0.98) (FIG. 5F), and for progressive vs regressive CIS of 0.75 (95% CI 0.69-0.82) (FIG. 16N). These results are similar to those obtained using the entire set of 450,000 probes, suggesting that methylation heterogeneity is a genome-wide process.

### Example 7: CIN is an Early Marker of Progression to Cancer

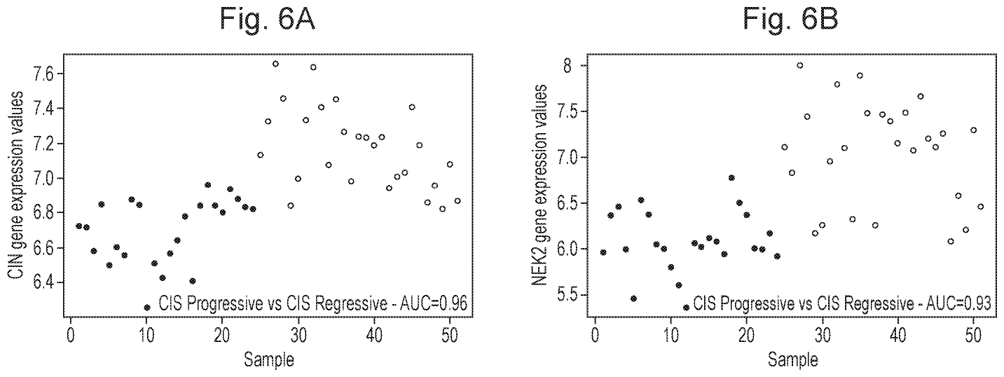

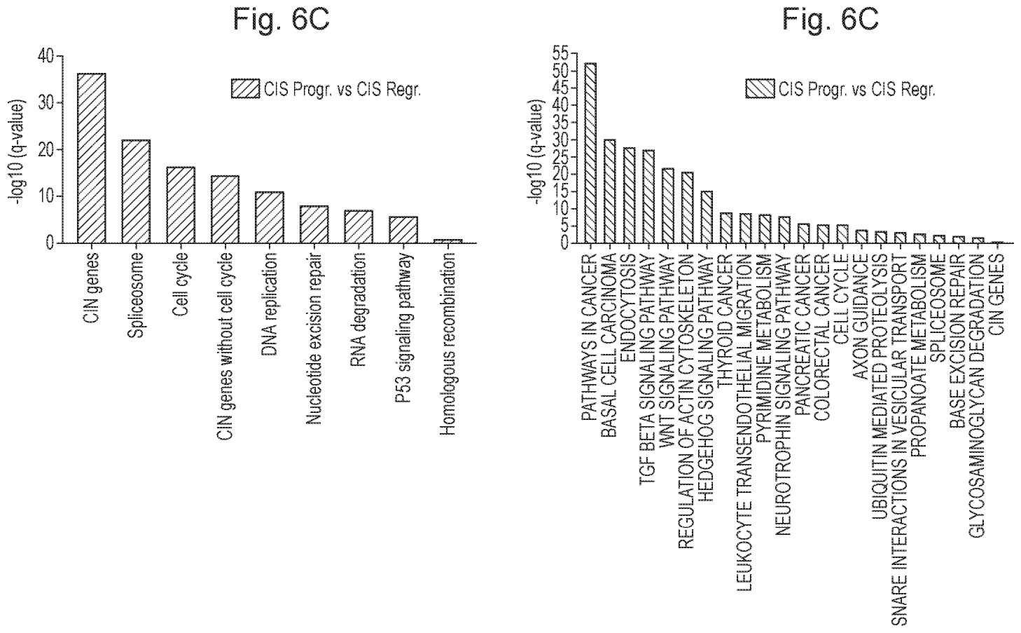

To investigate possible drivers of tumorigenic progression, a differential analysis of gene expression data between the progressive and regressive groups was performed. 5 of the top 100 genes identified have been previously associated with chromosomal instability (CIN) [24], as defined by the previously published CIN70 signature [25] (ACTL6A, ELAVL1, MAD2L1, NEK2, OIP5). All five are up-regulated in progressive compared with regressive samples. CIN-related genes can predict progression (FIG. 6A); NEK2 expression alone predicts progression with AUC=0.93 (FIG. 6B).

Pathway analysis was performed using the gage Bioconductor package [26] to compare the differentially expressed genes to KEGG gene sets. The CIN70 gene set was the most significant gene set identified (adjusted p value 8.9×10-32; up-regulated in progressive group), suggesting a role in early tumorigenesis. Cell cycle and DNA repair pathways were also implicated (FIG. 6C). Results were similar when cell-cycle associated genes were removed from the CIN70 signature, suggesting that this is a genuine CIN signal rather than a marker of proliferation.

Performing similar differential analysis of differentially methylated probes found widespread changes. The top probes identified were associated with cancer-associated cell signalling pathways, including TGF-beta, WNT and Hedgehog, as well as cell cycle and CIN-associated genes (FIG. 6D).

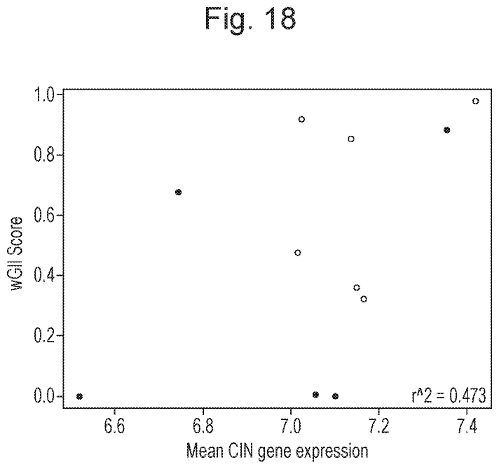

This CIN signal is consistent with the observed pattern of widespread copy number change (FIG. 3). Overall copy number variation for a sample, as measured by Weighted Genome Integrity Index (wGII) [27], correlates with mean CIN-associated gene expression of that sample (Pearson r2=0.473) (FIG. 18). It was also observed that a correlation between local copy number of a gene and expression of that gene, consistent with previous results [28], [29].

### Example 8: Materials and Methods

### Ethical Approval

All tissue and bronchial brushing samples were obtained under written informed patient consent and were fully anonymised. Study approval was provided by the UCL/UCLH Local Ethics Committee (REC references 06/Q0505/12 and 01/0148).

### Data Availability

Whole-genome sequencing data have been deposited at the European Genome Phenome Archive (https://www.ebi.ac.uk/ega/at the EBI) with accession number EGAD00001003883. All gene expression and methylation microarray data reported in this study have been deposited in the National Center for Biotechnology Information Gene Expression Omnibus (GEO, http://www.ncbi.nlm.nih.gov/geo/) public repository, and they are accessible through GEO accession number GSE108124.

### Code Availability

All code used in our analysis will be made available at http://github.com/ucl-respiratory/preinvasive on publication. All software dependencies, full version information, and parameters used in our analysis can be found here.

Unless otherwise specified, all analyses were performed in an R statistical environment (v3.5.0; www.r-project.org/) using Bioconductor1 version 3.7.

### Biological Samples

All patients with pre-invasive lung cancer lesions were recruited through University College London Hospitals (UCLH) Early Lung Cancer Surveillance Programme (ELCSP). Full details of the surveillance protocol including eligibility criteria for patient inclusion have been previously described [2]. Briefly, the programme has recruited 140 patients to date with pre-invasive lung cancer lesions of varying histological grades. Patients undergo autofluorescence bronchoscopy (AFB) and CT/PET scans every four to six months during which multiple biopsy specimens are collected. This longitudinal sequential AFB procedure provides biopsies of the same lesion sampled repeatedly over time, allowing us to monitor whether the individual lesions have progressed, regressed or remained static [2].

For a given CIS lesion under surveillance, when a biopsy from the same site showed evidence of progression to invasive cancer or regression to normal epithelium or low-grade dysplasia, we define the preceding CIS biopsy as the ‘index’ lesion. An index lesion was defined as progressive if the subsequent biopsy at the same site showed invasive cancer, or as regressive if the subsequent biopsy showed normal epithelium or low-grade disease (metaplasia, mild or moderate dysplasia). Lesions which do not satisfy one of these end-points were excluded from this study. Patients with multiple fresh-frozen (FF) and formalin-fixed, paraffin-embedded (FFPE) tissue biopsies were identified for DNA methylation and gene expression analysis, respectively. Laser-capture micro-dissection (LCM) was used to selectively isolate CIS cells for molecular analysis, reducing the extent of contamination by stromal cells.

The following protocol was used to determine which profiling methods were applied to a given CIS lesion during our initial data collection phase:
If FFPE samples were available, gene expression profiling was performed. For the first 33 samples (17 progressive and 16 regressive), gene expression profiles were generated using Illumina microarrays. Our predictive models are trained on this discovery set. Subsequently, a further set of 10 progressive and 8 regressive samples from 18 patients were profiled using a different microarray platform (Affymetrix) to validate our findings on an independent platform.
If FF samples were available, DNA from these samples was first used for methylation profiling. Samples with sufficient DNA after DNA profiling were additionally subjected to whole-genome sequencing. After acquisition of sufficient samples for our methylation dataset (54 samples; 36 progressive, 18 regressive), only 29 samples had sufficient DNA for WGS, therefore we prioritised WGS over methylation for the subsequent 10 samples.

### Tissue Processing and Laser-Capture Micro-Dissection

FF or FFPE tissue sections (7-10 μM thickness) were mounted on a MembraneSlide 1.0 PEN. Prior to cryosectioning, the slides were heat-treated for 4 h at 180° C. in a drying cabinet to inactivate nucleases. To overcome the membrane's hydrophobic nature and to allow better section adherence, the slides were then UV-treated for 30 min at 254 nm. Prior to laser-capture micro-dissection (LCM), the slides containing the FF tissue sections for DNA extraction were washed in serial ethanol dilutions (50, 75, 100%) to remove the freezing medium (OCT) and to avoid any interference with the laser's efficiency. For RNA extraction, FFPE sections were dewaxed using the Arcturus® Paradise® PLUS Reagent System (Applied Biosystems, Foster City, Calif., USA). For each case, epithelial areas of pre-invasive disease were identified by haematoxylin and eosin staining of the corresponding cryosection (˜7 μM thick). The presence of epithelial areas of interest was confirmed by histological assessment of each case by two histopathologists. LCM to isolate the tissue area/cells of interest was performed with the PALM Microbeam™ system (Carl Zeiss MicroImaging, Munich, Germany) on unstained sections. The micro-dissected material was catapulted into a 500 μl AdhesiveCap that allows capture of the isolated tissue without applying any liquid into the cap prior to LCM, thus minimizing the risk of nuclease activity. The captured cells were stored at −80° C. until DNA extraction or processed immediately for RNA.

### DNA Extraction

DNA from the micro-dissected tissue and bronchial brushing samples was extracted using QIAGEN's QIAmp DNA Mini and Micro kits, respectively (Crawley, UK). Soluble carrier RNA was used to increase tissue DNA yield. Concentration was measured using the Qubit® dsDNA High-Sensitivity assay and Qubit® 2.0 Fluorometer (Life Technologies, Paisley, UK). Nucleic acid quality and purity was estimated based on the A260/280 absorbance ratio readings using the NanoDrop-8000 UV-spectrophotometer (Thermo Scientific, Hertfordshire, UK). Only samples with an A260/280 ratio of 1.7-1.9 were included in the study.

### RNA Extraction

RNA was extracted using the High Pure FFPE RNA Kit (Roche Applied Science, West Sussex, UK) according to manufacturer's protocol. Quantification was carried out using the Quant-iT RNA assay kit and the Qubit® 2.0 fluorometer (Life Technologies, Paisley, UK). RNA integrity was analysed using a BioAnalyzer 2100 (Agilent, Stockport, UK).

### Bisulfate Conversion

For each sample undergoing methylation profiling, 200 ng of DNA were bisulfite converted using the EZ DNA methylation kit (Zymo Research Corp., Orange, Calif., USA) according to the manufacturer's modified protocol for Illumina's Infinium 450K assay. This protocol incorporates a cyclic denaturation step to improve the conversion efficiency[3]. The 10 μl final conversion reaction was concentrated down to 4 μl with a vacufuge plus vacuum concentrator (Eppendorf AG, Hamburg, Germany) and sent to UCL's Genomics Core Facility for hybridization on the 450K BeadArray according to Illumina's Infinium HD protocol (Illumina Inc., San Diego, Calif., USA) as previously described.4

### Infinium HumanMethylation450K Raw Data Extraction and Pre-Processing

Illumina's iScan fluorescent system was used to scan and image the arrays. DNA methylation data were extracted as raw intensity signals without any prior background subtraction or data normalization and were stored as IDAT files.

CpG-specific methylation levels (β-values; continuous value ranging from 0 to 1) for each sample were calculated as the ratio of the fluorescent signal intensity of the methylated (M) and unmethylated (U) alleles according to the following formula:

$$β = \frac{intensity of methy1ated allele ( M )}{intensity of [ unmethylated ( U ) + methylated ( M ) allele ] + 100}$$

All subsequent raw β-value pre-processing, normalisation and down-stream analysis was performed using the Chip Analysis Methylation Pipeline (ChAMP) Bioconductor package with default settings [5].

Analysis of differentially variable positions (DVP) was performed using iEVORA6. Beta values from ChAMP were used as input to iEVORA following normalization and batch correction.

### Genome-Wide Gene Expression Array

The extracted FFPE RNA used to generate the gene expression profiles on the discovery set was sent to UCL's Genomics Core Facility for hybridisation on the Human Whole-Genome DASL (cDNA-mediated Annealing, Selection, extension and Ligation) beadarrays according to Illumina's protocol (Illumina Inc., San Diego, Calif., USA).

The extracted FFPE RNA used to generate the gene expression profiles on the validation set was sent to UK Bioinformatics Limited for hybridisation on the Clariom™ D Transcriptome Human Pico Assay 2.0 according to Affymetrix's protocol (Thermo Fisher Scientific Waltham, Mass., USA).

### Principal Component Analysis (PCA)

In order to identify any potential factors of variability affecting sample/group segregation, we applied principal component analysis on all probes passing filters defined above (implemented in the prcomp method of the R stats package). Technical and biological variation was investigated for batch arrays, smoking (pack-years), age at initial diagnosis, gender and previous lung cancer history. The ability of these features to predict the first principal component was quantified using ANOVA analysis, implemented in the R aov method. p-values quoted are derived from this method.

### Gene Expression Analysis

Raw gene expression data were expressed as log 2 ratios of fluorescence intensities of the experimental samples. Quantile normalization was applied to Illumina data, using proprietory Illumina software. For Affymetrix data, RMA normalization was applied as defined in the affy Bioconductor package. For analyses utilizing both data sets, only genes represented on both arrays were included and ComBat7 was used to adjust for batch effects.

Differential expression analysis was performed using the limma8 Bioconductor package. Raw p-values were adjusted by the Benjamini-Hochberg procedure to give a FDR [9]. A significance threshold of FDR <0.01 was used to select differentially expressed genes. Cluster analysis and visualization was performed using the pheatmap [10] Bioconductor package.

### Real Time PCR Validation

For microarray validation, total RNA from the 33 pre-invasive LUSC lesions undergoing Illumina gene expression profiling was reverse transcribed using qScript™ cDNA Super-Mix (Quanta Biosciences, Lutterworth, UK) according to the manufacturer's protocol. Real-time quantitative PCR was carried out in eight genes using the SYBR-green master mix (Applied BioSystems, Bleiswijk, Netherlands) in an Eppendorf real-time PCR Machine (Eppendorf, Stevenage, UK). Findings were validated using quantitative PCR (qPCR) for four up-regulated (GAGES, GPNMB, MMP12 and STC2) and four down-regulated (SPDEF, LMO7, OBSCN and MT1E) genes. Gene-specific primers were designed inside or nearby the microarray sequence targeted, using Primer Express Software (PE Applied Biosystems, Bleiswijk, Netherlands). Relative gene expression was quantified using the threshold cycle (Ct) method and normalized to the amount of CTBL and CEP250, which met the criteria of less variation between samples and compatible expression level with the studied genes. Each sample was tested in triplicate and a sample without template was included in each run as a negative control. Correlations between microarrays and real time PCR data were measured using the Pearson coefficient. From microarray and real time PCR data, we calculated the progressive/regressive ratio for each gene expression. All eight genes tested were significant in our differential microarray analysis with FDR <0.05. A high degree of correlation (r=0.982) was observed between qPCR and array data.

### Predictive Modelling

For methylation, gene expression and copy number data we applied Prediction Analysis of Microarrays (PAM)[11] to predict whether a sample was progressive or regressive based on its molecular profile. The Bioconductor pamr package was used. In all presented analyses we select a threshold which minimizes the number of data inputs required whilst maintaining the minimum possible number of classification errors.

PAM calculates the probability of each sample being progressive. We describe this value as a ‘Progression Score’. ROC analytics were performed on these progression scores to determine their value as a diagnostic test, using the pROC12 and PRROC13 Bioconductor packages.

For methylation and gene expression data a predictive model was trained on the training set and subsequently applied to an independent validation set. Regressive and control samples were grouped together for the methylation data analysis. ROC analytics were performed only on the validation set. Internal cross-validation was used for methylation-derived copy number data due to smaller sample size (control samples are used as a baseline to calculate copy number, therefore are excluded from predictive analysis).

When multiple lesions from one patient were included in an analysis, these were treated as independent events as they were always taken from different sites in the lung. The outcome of a lesion (whether it progressed or regressed) was determined on a per-lesion basis; the lesion was assigned to the progressive group only if cancer developed at the same site in the lung, and to the regressive group only if normal or low-grade dysplasia was obtained from the same site in the lung.

In some cases different technologies were used, for example our gene expression discovery set used Illumina microarrays whereas our validation set used Affymetrix. In such instances, both data sets were reduced to the subset of genes covered by probes in both platforms prior to creating a predictive model. The ComBat method from the sva Bioconductor package was used to correct for batch effects between the different platforms. In the case of RNAseq data, we used the voom transformation defined in the limma Bioconductor package to derive data comparable to expression data prior to batch correction with ComBat.

A second predictive model based on methylation probe variation was also developed. For a given sample we defined Methylation Heterogeneity Index (MHI) by counting all probes with beta values between 0.26 and 0.88. These thresholds were optimized by calculating MHI for a range of different threshold values, and choosing those with the highest AUC for progressive vs regressive in our discovery cohort. We used ROC analytics to assess this model as a predictor of TCGA cancer vs control samples, and of progressive vs regressive samples in our validation cohort. We demonstrate in the main text that applying this method to a random sample of 2,000 probes performs similarly to using the entire array. We ran simulations using different sample sizes and found that performance with n=2000 was similar to that of the entire array. To investigate potential confounding variables we use binomial logistic regression, implemented in the R glm method, to assess whether outcome (progression/regression) could be predicted by MHI, smoking status, COPD, previous history of lung cancer, age or gender. Control samples derived from brushings were excluded from these analyses.

### Copy Number Variation Analysis

For samples with whole-genome sequencing available we used ASCAT14 to derive local copy number estimates as described below. To increase our sample size for comparative analyses, Copy number variation (CNV) data were obtained from non-normalised methylated and unmethylated signal intensities of probes in the 450K array as previously described [15] using the ChAMP Bioconductor package with default settings. Copy number (CN) profiles for progressive and regressive cases were obtained using the control cases for baseline normalisation. A previously defined threshold of ±0.3 was used for the identification of single CNV. Probes associated with highly polymorphic regions (e.g. major histocompatibility complex) were removed from the analysis. The analysis generated group CN frequency plots and CN profiles for each sample. For samples with both methylation and sequencing data available we observed good correlation between copy numbers derived from the two different methods.

For comparison with previous results, the ChAMP pipeline was then modified to return CNV values per-probe. Probe locations were matched to cytogenetic bands using the Ensembl GRCh37 assembly, obtained from http://grch37.rest.ensembl.org/info/assembly/homo_sapiens?content-type=application/json&bands=1, such that copy number variation could be assessed by cytogenetic band. The mean CNV value for each of 778 cytogenetic bands was calculated for each of our 54 samples. Limma analysis was used to identify bands that differed significantly between progressive and regressive samples with BH-adjusted p-value <0.05. Predictive modelling was performed using PAM to find bands predictive of progression, using the same method as for gene expression data. Due to the low number of regressive samples, an internal cross-validation method was used rather than separate discovery and validation sets.

Following identification of predictive cytogenetic bands, PAM modelling was repeated with the dataset limited to only those bands identified by van Boerdonk et al: 3q26.2-29, 3p26.3-p11.1 and 6p25.3-p24.3.16,17. This model was also accurate.

Finally, we applied our model to the validation data set of 24 regressive and 12 progressive samples used by van Boerdonk et al (GEO accession number GSE45287). These data were measured using a different microarray platform (arrayCGH). We assigned each probe to a cytogenetic band, and took the mean values to create a matrix of expression values by band. Our model was applied to the subset of chromosomal bands present in both data sets (760 of 778 bands). ComBat was used for batch correction between the two platforms. Our model correctly predicted 24/24 regressive samples and 9/12 progressive samples, replicating the results of van Boerdonk et al.

### External Validation Using TCGA

Lung cancer methylation datasets publically available through The Cancer Genome Atlas (TCGA) were downloaded using GenomicDataCommons download tools [18]. We obtained the normalized β-values of 370 LUSC samples and 42 normal controls. ComBat was used to correct for batch effects between our data and TCGA data. These data were used as an external validation set to test our predictive models, and as input for our differential analysis of progression drivers from control through CIS to cancer.

Gene-expression microarray data sets comparable to our data were not publically available. RNAseq data was available from TCGA for 502 LUSC samples and 49 control samples. We applied a voom transformation [19] to these data, which uses normalized log-counts-per-million as an approximation for expression values, and hence allows comparison of RNAseq data with our gene expression pipeline. ComBat was used to correct for batch effects. The predictive model generated using PAM on our gene expression microarray data was applied to voom-transformed RNAseq data from TCGA and shown to be predictive (FIG. 5C). We therefore demonstrate the applicability of our model to this fully independent data set. These data were again used as input to our differential analysis of progression drivers.

### Pathway Analysis

For gene expression data, the GAGE Bioconductor package [20] was used with KEGG gene sets [21]-[23] to identify pathways associated with genes differentially expressed in our analysis of progression to cancer (BH-adjusted p-value <0.01). In addition to these pathways we use the CIN70 signature defined by Carter et al. [24] to assess for a chromosomal instability signal. We also use a subset of the CIN70 genes with cell-cycle associated genes [25] removed to ensure that our signal is genuinely CIN-related, rather than a measure of proliferation.

Methylation data was analysed in the same way, using beta values as input to GAGE. In cases where there are multiple methylation probes for a single gene we use the mean beta value over that gene as input to pathway analysis. We acknowledge that using mean signal may be insensitive to single-probe methylation changes, however given the scale of changes observed we believe it will identify areas of large methylation change.

### Genomic Sequencing

We created genome-wide shotgun libraries (insert size 331-367 bp) from native DNA using the Agilent Technologies Custom SureSelect Library Prep Kit library (cat no. 930075). 150 bp paired-end sequence data were generated using the Illumina HiSeq X Ten system.

Sequenced data were realigned to the human genome (NCBI build 37) using BWA-MEM. Unmapped reads and PCR duplicates were removed. A minimum sequencing depth of 40× was required.

### Somatic Mutation Calling and Annotation

Single base somatic substitutions were identified by our in-house algorithm Cancer Variants through Expectation Maximisation (CaVEMan: https://github.com/cancerit/CaVEMan) [26]. This algorithm compares the sequence data from each tumour sample to its matched normal and calculates a mutation probability at each locus. This calculation incorporates information from aberrant cell fraction and copy number estimates from the Allele-Specific Copy number Analysis of Tumours (ASCAT) algorithm (https://www.crick.ac.uk/peter-van-loo/software/ASCAT) [14],[27]. Additional post-processing as described previously [28] was implemented. Any putative driver mutations were visually inspected with Jbrowse [29]. For every substitution that passed all filters in at least one sample, we counted the number of wild-type and mutant reads at the same position in all other samples from the same patient to see if that mutation was also present in related samples but had not been called.

### Somatic Small Insertions and Deletions

These were identified using our in-house algorithm Pindel [30], [31]. As with substitutions, all putative driver mutations were visualised with Jbrowse.

### Somatic Structural Variant Detection

Abnormally paired read pairs were grouped using an in-house tool, “Brass” [32]. Read groups overlapping genomic repeats, reads from the matched normal, or from a panel of unmatched normals were ignored. Read pair clusters were then filtered by read remapping. Read pair clusters with >50% of the reads mapping to microbial sequences were removed. Finally, candidate SV breakpoints were matched to copy number breakpoints as defined by ASCAT within 10 kb. Candidate SVs that were not associated with copy number segmentation breakpoints and with a copy number change of at least 0.3 were removed. All putative driver rearrangements were visually inspected using IGV [33], [34].

### Somatic Copy Number Events, Ploidy, and Stromal Contamination

Copy number changes were derived from whole-genome sequencing data using the ASCAT algorithm. This algorithm compares the relative representation of heterozygous SNPs and the total read depth at these positions to estimate the aberrant cell fraction and ploidy for each sample, and then to determine allele-specific copy number.

### Weighted Genome Integrity Index

To estimate the overall chromosomal instability of a sample, we use the Weighted Genome Integrity Index (wGII) score [35]. This is calculated by measuring the percentage of the genome which is abnormal, corrected such that each chromosome is equally weighted.

### Mutation Annotation

Lung cancer driver genes were selected from the COSMIC Cancer Gene Census (CGC) v85 (cancer.sanger.ac.uk) [36]. CGC data was downloaded on 20 Jun. 2018. Genes annotated in the CGC as potential drivers in lung cancer or NSCLC were included. Those specific to adenocarcinoma were excluded as our samples are precursors to squamous cancers. Genes identified in two large studies of squamous cell cancer, and some additional genes based on expert curation of the literature (ARID1A, AKT2, FAT1, PTPRB) were included if they were present in the CGC—even if they were not annotated explicitly as implicated in lung cancer. Both Tier 1 and Tier 2 genes were included. A total of 96 genes were selected as putative lung squamous cell carcinoma drivers.

Mutations affecting these putative driver genes were annotated as driver mutations if they passed the following filters:
The mutation type (e.g. missense, frameshift, amplification) must have been validated in the CGC for the affected gene.
For genes annotated as tumour suppressors, mutations determined to have High or Moderate impact using Ensembl's Variant Effect Predictor [37] were classed as driver mutations.
For genes annotated as oncogenes, we checked the specific mutation against COSMIC mutation data for lung carcinomas. If the specific mutation occurred 3 or more times in this dataset it was classed as a driver mutation.
For genes annotated as fusion proteins, translocations with a translocation partner gene matching validated translocation partner genes in the CGC were classed as driver events.
Copy number amplifications and deletions were all classed as driver events if amplifications/deletions in the affected gene have been previously validated in the CGC. We included homozygous deletions of tumour suppressor genes and amplifications to more than double the sample ploidy for oncogenes.

Driver mutation discovery was also attempted using dndscv [38]. This was underpowered, however, and only yielded TP53 and CDKN2A as genes under positive selection. This package was also used to estimate the global dNdS for both progressive and regressive lesions.

### Subclonality Analysis

The number of subclones contributing to a sample and their relative contribution was estimated by using a modified version of the sciClone Bioconductor package [39]. sciClone uses a Bayesian method to allocate mutations to clusters based on their variant allele frequency (VAF). By default, sciClone only considers regions that are copy number neutral and LOH-free. Given the significant aneuploidy in our data set we overcame this limitation by clustering on cancer cell fraction (CCF) rather than VAF. Briefly, cancer cell fraction represents the fraction of cancer cells in which a given mutation is present, therefore clonal mutations will have CCF=1. Following the method of McGranahan et al. [40], we estimated the CCF for each mutation with a 95% confidence interval. Mutations for which 1 lay within this confidence interval were labelled as ‘clonal’, other mutations as ‘subclonal’.

CCF values for each mutation were then used as input to sciClone in place of VAF values to quantify clusters present (divided by 2 such that clonal mutations have a value of 0.5). As CCF corrects for local copy number, all regions were assumed to have copy number of 2, allowing sciClone to group mutations based only on their CCF estimates. A minimum tumour sequencing depth of 10 was required for each mutation.

Where more than one sample from a given patient was available, both one dimensional and multi-dimensional clustering were performed. Results from one dimensional clustering were used in the comparison of numbers of clones and proportion of clonal mutations between progressive and regressive lesions, in order to provide as fair a comparison as possible.

### Extraction of Mutational Signatures

To obtain an approximate estimate of the contribution of different known mutational signatures to each sample, we used the MutationalPatterns Bioconductor package41. As a reference set of mutational signatures, we used a table with the relative frequency of each of the 96 trinucleotide substitutions across 30 known mutation signatures, [42], [43] available through the COSMIC website (http://cancer.sanger.ac.uk/cosmic/signatures).

After a first run which indicated the most likely contribution of each signature, it seemed that the majority of substitutions were contributed by signatures 1, 2, 4, 5, and 13, which have been described to be the strongest signatures in lung squamous cell cancer [44]. Some contribution was identified from signatures 16, 8, 18 and 3 in our initial analysis; however, in this context it is likely that these represent overfitting given that signature 16 is similar to signature 5, and signatures 8, 18 and 3 are similar to signature 4. We therefore ran the algorithm a second time, this time only using a 5×96 matrix of mutational signatures 1, 2, 4, 5 and 13. All mutations were thus forced to belong to one of these five mutational signatures.

For a comparison of the clonal vs subclonal mutational processes in each sample, substitutions were annotated as clonal or subclonal based on CCF as described above. These were then run through the MutationalPatterns package.

Comparison of Mutational Burden and Signatures with Other Cancer Types

Signatures of mutations in our CIS dataset were compared with mutational signatures found in lung squamous cell cancer. Raw whole-exome sequencing data for this cancer type was downloaded from TCGA, and run through our substitution-calling algorithm CAVEMaN as described above. We then looked at the total number of substitutions called, and estimated the contribution of each mutational signature using the methods described above. Only coding regions of the CIS whole-genome sequencing data were compared to these exomes.

### Estimation of Telomere Lengths

Telomere lengths were estimated using telomerecat [45], and were compared in progressive and regressive groups. Telomerecat is a de novo method for the estimation of telomere length (TL) from whole-genome sequencing samples. The algorithm works by comparing the ratio of full telomere reads to reads on the boundary between telomere and subtelomere. This ratio is transformed to a measure of length by taking into account the fragment length distribution. Telomerecat also corrects for error in sequencing reads by modelling the observed distribution of phred scores associated with mismatches in the telomere sequence. Samples were analysed in two groups corresponding to two separate sequencing batches, as per the telomerecat documentation.

All publications mentioned in the above specification are herein incorporated by reference. Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in genetics, epigenetics, molecular biology, cell biology, oncology or related fields are intended to be within the scope of the following claims.

### Tables

TABLE 1

Metrics:

| Number | ROC AUC | ROC | Threshold | Sensitivity | Specificity | Sensitivity | Specificity |
|---|---|---|---|---|---|---|---|
| of DMPs | (CvC¹) | (PvR²) | value | (CvC) | (CvC) | (PvR) | (PvR) |
| 2000 | 0.987870013 | 0.808641975 | 0.35 | 1 | 0.562162162 | 0.833333333 | 0.722222222 |

DMP IDs:

| | | | | | |
|---|---|---|---|---|---|
| cg13676449, | cg03790740, | cg04445871, | cg06864391, | cg18107232, | cg18346228, |
| cg21586613, | cg18496262, | cg26611623, | cg10210789, | cg20306234, | cg24952123, |
| cg01388144, | cg18236734, | cg17286012, | cg02100602, | cg10137597, | cg18204930, |
| cg07025312, | cg04227077, | cg07500501, | cg20862447, | cg08284713, | cg21904978, |
| cg23535768, | cg09436767, | cg07536161, | cg03000485, | cg25040562, | cg11459133, |
| cg13464240, | cg01745448, | cg27659035, | cg14211350, | cg00286067, | cg06065657, |
| ch.2.168147954R, | cg24530250, | cg19619807, | cg04831708, | cg01154527, | cg25000158, |
| cg18219997, | cg13042250, | cg09244100, | cg08350734, | cg13445126, | cg08231333, |
| cg13634242, | cg12377745, | cg18161670, | cg23262134, | cg06766367, | cg06202492, |
| cg26577017, | cg26556719, | cg04801704, | cg05450336, | cg17214502, | cg04086977, |
| cg20848619, | cg12872398, | cg22662036, | cg18404811, | cg10152523, | cg09962458, |
| cg21383495, | cg04305912, | cg13865352, | cg25309929, | cg01664065, | cg08929303, |
| cg18614735, | cg21020944, | cg01310330, | cg11359984, | cg08290287, | cg24782087, |
| cg00759741, | cg26721694, | cg16607815, | cg07117674, | cg16248182, | cg14157435, |
| cg18923740, | cg04200627, | cg21288315, | cg08927728, | cg23190366, | cg02461341, |
| cg16940012, | cg14330189, | cg08276984, | cg08552043, | cg23753807, | cg22484397, |
| cg26308818, | cg06598544, | cg01740282, | cg25114142, | cg19907511, | cg08150843, |
| cg18767057, | cg02285812, | cg02149136, | cg26272585, | cg13166748, | cg27476456, |
| cg24657463, | cg06820621, | cg27112339, | cg05412664, | cg17778955, | cg01383203, |
| cg18595780, | cg05238976, | cg21199045, | cg16159175, | cg22090592, | cg08182072, |
| cg13685437, | cg27537918, | cg18417373, | cg23458558, | cg01225317, | cg00776208, |
| cg24184689, | cg06982272, | cg11784830, | cg21649919, | cg10888281, | cg02848331, |
| cg02927074, | cg10144964, | cg13728106, | cg21085001, | cg21176127, | cg20891565, |
| cg03795608, | cg21833837, | cg01286631, | cg00045061, | cg06622108, | cg24485038, |
| cg08545169, | cg16503489, | cg26764761, | cg06262842, | cg07529534, | cg14613508, |
| cg02660119, | cg01778939, | cg18945035, | cg20369775, | cg13140267, | cg06387245, |
| cg16755475, | cg10089028, | cg08308360, | cg18138036, | cg18845838, | cg13678584, |
| cg04954111, | cg07123730, | cg20646491, | cg26556918, | cg06192592, | cg01498609, |
| cg25067686, | cg26969840, | cg14457918, | cg11367401, | cg01835282, | cg12810402, |
| cg25932807, | cg16521555, | cg03283421, | cg03286987, | cg08126211, | cg19861632, |
| cg07952040, | cg16727231, | cg16796458, | cg05112986, | cg13692543, | cg04298626, |
| cg06822067, | cg07562483, | cg16373202, | cg17553885, | cg11726474, | cg10612096, |
| cg05917225, | cg20825416, | cg21624883, | cg22052672, | cg05207184, | cg01569082, |
| cg02216247, | cg12630336, | cg02685990, | cg10196001, | cg27103296, | cg09002774, |
| cg14343017, | cg09431544, | cg08722720, | cg27205791, | cg05900127, | cg06767389, |
| cg00283576, | cg09528988, | cg08514594, | cg11309696, | cg21576886, | cg15677916, |
| cg02129266, | cg07311505, | cg05535877, | cg04931582, | cg12635191, | cg25891355, |
| cg16762794, | cg06932455, | cg12735489, | cg06892726, | cg19964491, | cg01102379, |
| cg25697490, | cg18494192, | cg12060877, | cg12271079, | cg21072692, | cg20260721, |
| cg23747904, | cg16235809, | cg03459952, | cg18713806, | cg19304935, | cg03687379, |
| cg15617609, | cg09458931, | cg02152119, | cg02370923, | cg12798040, | cg16080654, |
| cg05294300, | cg19486955, | cg05165087, | cg26539631, | cg24286765, | cg22905097, |
| cg25937064, | cg26031875, | cg17394996, | cg04657461, | cg14834257, | cg23075303, |
| cg19879471, | cg18799284, | cg26947626, | cg27140220, | cg03710176, | cg16887183, |
| cg04399461, | cg02076624, | cg10113820, | cg05956477, | cg00226608, | cg12423338, |
| cg27225658, | cg01497613, | cg07968590, | cg11559446, | cg05919314, | cg26632873, |
| cg09576762, | cg04630748, | cg06916740, | cg14018959, | cg12699768, | cg23319285, |
| cg00499047, | cg03035826, | cg05062612, | cg01543197, | cg09901574, | cg25175654, |
| cg05337637, | cg22695151, | cg12448724, | cg04213489, | cg08898442, | cg06062821, |
| cg05006567, | cg15642534, | cg12365681, | cg00935307, | cg08119607, | cg14101485, |
| cg02630122, | cg14777634, | cg09618102, | cg04240267, | cg11291313, | cg05233877, |
| cg18632602, | cg25588222, | cg21901300, | cg06763078, | cg23848889, | cg26872028, |
| cg27155954, | cg02227503, | cg11884230, | cg09790580, | cg14169740, | cg01369908, |
| cg02100848, | cg25107268, | cg23812119, | cg00852549, | cg05844242, | cg04428139, |
| cg15105011, | ch.19.1427912R, | cg14330675, | cg11877726, | cg06366810, | cg22876086, |
| cg21116657, | cg14154547, | cg13170728, | cg20254353, | cg19262005, | cg16429819, |
| cg05345116, | cg13360200, | cg19954314, | cg18501264, | cg08307766, | cg01128044, |
| cg01543173, | cg14637870, | cg23946462, | cg27154163, | cg23689722, | cg10028227, |
| cg03961401, | cg17752270, | cg25780671, | cg06573047, | cg06660160, | cg26900686, |
| cg02796970, | cg20577205, | cg19896142, | cg04751533, | cg25747670, | cg25700339, |
| cg14333542, | cg16794682, | cg19945897, | cg14726403, | cg01301276, | cg06329197, |
| cg03381304, | cg00440043, | cg20925841, | cg06363559, | cg12589188, | cg14682345, |
| cg23850750, | cg08477332, | cg10960055, | cg15923864, | cg09872841, | cg02790177, |
| cg21663122, | cg08443305, | cg23498628, | cg10181822, | cg04471454, | cg21226933, |
| cg26847193, | cg21644669, | cg03722909, | cg13995916, | cg06672120, | cg12440062, |
| cg08550421, | cg26293201, | cg07008213, | cg15799309, | cg26547741, | cg19177125, |
| cg13728131, | cg13877005, | cg27313642, | cg18561261, | cg26124569, | cg05595088, |
| cg03807298, | cg03438644, | cg18156135, | cg22672431, | cg10382148, | cg27143605, |
| cg04557018, | cg01392338, | cg17831277, | cg05253326, | cg11856697, | cg16376612, |
| cg05800641, | cg03953626, | cg00585901, | cg09844317, | cg25978138, | cg12996305, |
| cg18635670, | cg23426054, | cg01158679, | cg18280492, | cg18537894, | cg12604950, |
| cg22300727, | cg12521055, | cg17575811, | cg09457490, | cg12388064, | cg23639120, |
| cg27237420, | cg15014010, | cg07012128, | cg17639265, | cg27556492, | cg20429911, |
| cg04957209, | cg08678898, | cg19139589, | cg13552712, | cg09380415, | cg10227919, |
| cg09286079, | cg17281600, | cg07769790, | cg01799521, | cg12688521, | cg27526149, |
| cg04533487, | cg04658038, | cg17446719, | cg16756389, | cg12595736, | cg18323210, |
| cg23127998, | cg25556690, | cg09363735, | cg07292311, | cg04282694, | cg08072204, |
| cg24961958, | cg26338030, | cg24354818, | cg20570797, | cg03389538, | cg03331300, |
| cg24505395, | cg05497360, | cg02005664, | cg17352468, | cg10738026, | cg22534336, |
| cg02688118, | cg25168288, | cg17244673, | cg04395153, | cg07142893, | cg07330634, |
| cg20707126, | cg10888878, | cg20690483, | cg15849403, | cg23253309, | cg04505897, |
| cg27659677, | cg18098769, | cg05771377, | cg03132551, | cg10211725, | cg10717691, |
| cg19811425, | cg12354382, | cg18533282, | cg13222752, | cg26227101, | cg01923103, |
| cg01628954, | cg01125531, | cg14071853, | cg10769658, | cg04380332, | cg15393221, |
| cg23269922, | cg00434991, | cg18676313, | cg12836313, | cg05222119, | cg10021735, |
| cg22605924, | cg25597177, | cg08590069, | cg00479912, | cg23206160, | cg01212326, |
| cg02969716, | cg15799226, | cg02686091, | cg04643397, | cg08936487, | cg02228815, |
| cg02723142, | cg14166212, | cg04581377, | cg04631458, | cg13302166, | cg23178714, |
| cg03756514, | cg23334298, | cg08243626, | cg23361969, | cg21520613, | cg03256597, |
| cg10810183, | cg02543101, | cg20405384, | cg08266923, | cg12660777, | cg20631820, |
| cg14195377, | cg25963309, | cg11523020, | cg19609334, | cg14286732, | cg23985374, |
| cg15960297, | cg22808086, | cg23967544, | cg03217115, | cg04392234, | cg19470379, |
| cg02689735, | cg27542828, | cg07228402, | cg00468400, | cg16756620, | cg27434638, |
| cg16443977, | cg16287791, | cg07488381, | ch.6.158597687F, | cg01094108, | cg20384545, |
| cg06697057, | cg10799055, | cg05980785, | cg00146805, | cg15396434, | cg14221460, |
| cg26251865, | cg13547237, | cg09344943, | cg11321030, | cg11352847, | cg02521641, |
| cg02208776, | cg14718065, | cg14054343, | cg03256963, | cg08548066, | cg05878101, |
| cg21026095, | cg23808931, | cg24906525, | cg14647877, | cg12661256, | cg21901847, |
| cg07955088, | cg23516463, | cg06975834, | cg19814981, | cg02424007, | cg23027521, |
| cg00055555, | cg15704988, | cg03320827, | cg05144889, | cg08220243, | cg13944093, |
| cg23667362, | cg16995768, | cg14546505, | cg12690857, | cg14014627, | cg22204915, |
| cg13786209, | cg05764403, | cg03223580, | cg00820513, | cg04279026, | cg21855205, |
| cg07181000, | cg14951136, | cg22254072, | cg25045098, | cg20471413, | cg19889017, |
| cg15878555, | cg04520242, | cg05168764, | cg14636077, | cg12457740, | cg25816013, |
| cg20959274, | cg15448829, | cg17839959, | cg22030419, | cg22792569, | cg02506353, |
| cg15278798, | cg06563300, | cg16154524, | cg01627212, | cg14455590, | cg04522596, |
| cg24073089, | cg15226766, | cg26458816, | cg24874977, | cg24383507, | cg02580917, |
| cg21638323, | cg23966157, | cg05139152, | cg12859923, | ch.2.4964537R, | cg12395216, |
| cg10069238, | cg01206730, | cg11508406, | cg19251352, | cg24663970, | cg04835297, |
| cg14970046, | cg03724260, | cg02956254, | cg06872673, | cg00029053, | cg22918914, |
| cg08919443, | cg04412950, | cg20301962, | cg10424264, | cg03730474, | cg18808929, |
| cg09932405, | cg13911959, | cg15336091, | cg20574949, | cg14065841, | cg22856539, |
| cg18417901, | cg17573603, | cg00232805, | cg16253634, | cg06226724, | cg07064331, |
| cg07384136, | cg00321288, | cg13683516, | cg13531387, | cg22593554, | cg00689225, |
| cg26591066, | cg08620751, | cg16746221, | cg23213230, | cg08464009, | cg14548542, |
| cg21879791, | cg10230355, | cg26183708, | cg06817516, | cg14740771, | cg27608224, |
| cg15832180, | cg10803321, | cg14769991, | cg23933399, | cg06777522, | cg09966770, |
| cg06897860, | cg20473723, | cg00735239, | cg21773142, | cg17136810, | ch.15.979660F, |
| cg26767922, | cg09078081, | cg05647567, | cg04290510, | cg24952380, | cg18562759, |
| cg03711622, | cg00096440, | cg01140140, | cg26335760, | cg06087029, | cg08480774, |
| cg07732888, | cg13685139, | cg13273672, | cg16087023, | cg14849798, | cg11235291, |
| cg04324727, | cg21213853, | cg14164956, | cg00239353, | cg05989862, | cg12534466, |
| cg15241390, | cg02100984, | cg20994254, | cg03224126, | cg16709232, | cg14039054, |
| cg12639429, | cg09387732, | cg14248360, | cg04432617, | cg11926776, | cg25985659, |
| cg06668155, | cg03819320, | cg04112102, | cg13049247, | cg14905731, | cg09090941, |
| cg03099431, | cg14054013, | cg13280536, | cg09554596, | cg16023600, | cg04370829, |
| cg05777976, | cg20628205, | cg09578614, | cg24938166, | cg08586228, | cg21401219, |
| cg25204281, | cg23452443, | cg13305444, | cg19344103, | cg02942845, | cg20337753, |
| cg14858551, | cg01048080, | cg21369231, | cg24899571, | cg16805994, | cg01676467, |
| cg18462226, | cg10320446, | cg03506525, | cg04107773, | cg18125510, | cg08213047, |
| cg00265212, | cg18090197, | cg14283647, | cg23615779, | cg12431188, | cg11167537, |
| cg02641420, | cg11799819, | cg11582014, | cg23843100, | cg14155567, | cg02387639, |
| cg14469693, | cg22462714, | cg07082159, | cg27302675, | cg08122156, | cg21257635, |
| cg25426723, | cg10985970, | cg17939935, | cg25216403, | cg07568784, | cg04832534, |
| cg15805921, | cg04303382, | cg23323388, | cg24857238, | cg26128378, | cg08740240, |
| cg15120477, | cg07863439, | cg13878641, | cg17589325, | cg18900271, | cg16541998, |
| cg26131879, | cg08307850, | cg26413162, | cg07922924, | cg03252700, | cg05869080, |
| cg08128889, | cg08098980, | cg09359349, | cg17513542, | cg03596178, | cg04896437, |
| cg24808754, | cg07870603, | cg08367638, | cg24617370, | cg03391019, | cg07037019, |
| cg11705439, | cg01896341, | cg08144586, | cg14312391, | cg26141385, | cg23820560, |
| cg04996689, | cg23847282, | cg12551739, | cg08909961, | cg24141078, | cg08404009, |
| cg13885561, | cg04600297, | cg25000600, | cg23891596, | cg22464570, | cg18411500, |
| cg06444195, | cg13707690, | cg26183127, | cg11369862, | cg07421969, | cg14857764, |
| cg22898783, | cg07908805, | cg25221207, | cg20146241, | cg13518625, | cg12837642, |
| cg21840153, | cg03078690, | cg10359230, | cg18126791, | cg12270950, | cg08889797, |
| cg14553504, | cg27377517, | cg09932694, | cg14456103, | cg08569180, | cg08464824, |
| cg06941836, | cg02470904, | cg08967837, | cg04857403, | cg06432061, | cg08477779, |
| cg25492383, | cg14659678, | cg09125300, | cg16419019, | cg10996201, | cg11838071, |
| cg00336706, | cg18794839, | cg10753966, | cg05832853, | cg14422446, | cg14841879, |
| cg26552572, | cg08672296, | cg18533201, | cg10347335, | cg23060646, | cg21323630, |
| cg15914863, | cg22341848, | cg18477315, | cg22976218, | cg06713373, | cg09074625, |
| cg07011512, | cg20012308, | cg00577417, | cg27174441, | cg18693395, | cg05378013, |
| cg12884762, | cg24465384, | cg10444953, | cg01505767, | cg02048922, | ch.20.750973F, |
| cg02510299, | cg10937302, | cg02583576, | cg20642630, | cg09430946, | cg19385933, |
| cg12019650, | cg00323501, | cg06355423, | cg11788486, | cg04618230, | cg25111284, |
| cg00108617, | cg13997901, | cg05460776, | cg26501369, | cg25116237, | cg07325459, |
| cg26874542, | cg12550387, | cg19817795, | cg09473847, | cg02379533, | cg00217080, |
| cg02986791, | cg14065857, | cg01562349, | cg19720377, | cg04011671, | cg16294838, |
| cg13331136, | cg15900248, | cg01774471, | cg02224164, | cg16306083, | cg26960333, |
| cg24520479, | cg03814872, | cg20761840, | cg24799451, | cg05254747, | cg13630560, |
| cg25044817, | cg16094925, | cg10950028, | cg17625506, | cg06492264, | cg10503610, |
| cg03295928, | cg27639142, | cg12965095, | cg15078838, | cg15911153, | cg09461648, |
| cg16599703, | cg05565277, | cg12615334, | cg14582691, | cg06378244, | cg11827910, |
| cg24938498, | cg23804481, | cg04444399, | cg16568681, | cg09588653, | cg14408969, |
| cg02482346, | cg07548383, | cg06704093, | cg02069674, | cg26217185, | cg10039504, |
| cg06034771, | cg18842805, | cg13446235, | cg11398299, | cg09654157, | cg03266200, |
| cg20660197, | cg06835156, | cg25407085, | cg17438737, | cg03878802, | cg01523799, |
| cg04503318, | cg14406401, | cg10318528, | cg24646951, | cg12301744 | |

DMP IDs:

| | | | | | |
|---|---|---|---|---|---|
| cg07343511, | cg10767425, | cg05743982, | cg02544453, | cg24427089, | cg17642353, |
| cg26267614, | cg23678165, | cg10756125, | cg11404502, | cg16601415, | cg24102792, |
| cg01684361, | cg16064261, | cg27376072, | cg10484734, | cg01143426, | cg08832227, |
| cg02934462, | cg25187254, | cg20322977, | cg02474628, | cg07379167, | cg00017221, |
| cg01189607, | cg15615135, | cg08168175, | cg19285847, | cg09680131, | cg15122841, |
| cg10973236, | cg07086674, | cg00816880, | cg02013274, | cg17752083, | cg06101576, |
| cg26575633, | cg18879590, | cg18617091, | cg18932699, | cg13731311, | |
| cg13671831, | cg23568552, | cg08732684, | cg14697246, | cg25978090, | cg19014419, |
| cg26665391, | cg08703191, | cg19219655, | cg05934012, | cg02477175, | cg22940988, |
| cg15704595, | cg00972950, | cg15319522, | cg00911289, | cg16033151, | cg20347101, |
| cg22679728, | cg17537007, | cg17703048, | cg20016767, | cg15637420, | cg19515484, |
| cg21901718, | cg21990700, | cg08712866, | cg21161651, | cg26748598, | cg02675414, |
| cg24120800, | cg19056664, | cg14646111, | cg12609140, | cg11026987, | cg03765041, |
| cg14602658, | cg14614403, | cg11423130, | cg10633619, | cg16744250, | cg17098965, |
| cg16563151, | cg19949931, | cg09145851, | cg23795217, | cg09821185, | cg26568855, |
| cg16655905, | cg13803196, | cg07455051, | cg22378252, | cg16339264, | cg07009895, |
| cg21753618, | cg21130861, | cg24980638, | cg09059267, | cg21709909, | cg06793562, |
| cg01202312, | cg25335544, | cg01389917, | cg09785846, | cg02462912, | cg24660836, |
| cg21980394, | cg24696067, | cg08750504, | cg07544187, | cg02681369, | cg19046793, |
| cg11149194, | cg26878165, | cg26452915, | cg24879882, | cg08195412, | cg27057022, |
| cg25613262, | cg10443987, | cg13554714, | cg26901352, | cg26892702, | cg25975382, |
| cg24980458, | cg19126300, | cg07106394, | cg18484507, | cg05761734, | cg26222132, |
| cg19376973, | cg26178217, | cg09765014, | cg17969853, | cg26467809, | cg14413117, |
| cg14856082, | cg07729842, | cg10782668, | cg25910432, | cg21103269, | cg17267064, |
| cg25475538, | cg03720950, | cg04653522, | cg08472727, | cg02327997, | cg20030820, |
| cg19947104, | cg21574675, | cg11808658, | cg18204589, | cg05157961, | cg08118747, |
| cg07719965, | cg16719077, | cg08450106, | cg05358025, | cg15692052, | cg20942097, |
| cg17025142, | cg03822003, | cg06736160, | cg26836955, | cg00933443, | cg02478721, |
| cg26494225, | cg21530026, | cg03231024, | cg18714469, | cg21902014, | cg25350306, |
| cg04806237, | cg08917121, | cg06816517, | cg15439745, | cg24935220, | cg10026613, |
| cg24899692, | cg03140521, | cg14138116, | cg10590628, | cg16184736, | cg00019877, |
| cg19837214, | cg17777731, | cg13813391, | cg04881751, | cg19287817, | cg01776825, |
| cg06382132, | cg02284889, | cg13252206, | cg14362370, | cg00183606, | cg15129401, |
| cg00891242, | cg10353539, | cg00184203, | cg18347762, | cg01373691, | cg24714011, |
| cg14334488, | cg14568196, | cg18761696, | cg20749730, | cg21640261, | cg08039142, |
| cg27598340, | cg04673430, | cg08129312, | cg00119079, | cg26525313, | cg21378238, |
| cg15741721, | cg00355633, | cg14842376, | cg24266450, | cg20402885, | cg26391891, |
| cg07316961, | cg22486834, | cg06177967, | cg14654495, | cg01747926, | cg04010586, |
| cg09197492, | cg10432662, | cg23296791, | cg04534198, | cg24830738, | cg09731288, |
| cg09745087, | cg16315668, | cg14563363, | cg27491025, | cg05310990, | cg16988989, |
| cg06959626, | cg18408487, | cg17278595, | cg17060249, | cg13009662, | cg10773064, |
| cg12155547, | cg15692283, | cg02494150, | cg05003455, | cg21543987, | cg18712755, |
| cg19412217, | cg03098589, | cg00502972, | cg05485206, | cg26209169, | cg04822518, |
| cg11633916, | cg09727050, | cg03332188, | cg02927836, | cg03611852, | cg15504677, |
| cg16148593, | cg02321871, | cg12649175, | cg10473842, | cg10347032, | cg13056927, |
| cg14564826, | cg10958840, | cg06637032, | cg18570553, | cg26539232, | cg09825670, |
| cg03681926, | cg15286206, | cg07103722, | cg02483101, | cg16564132, | cg07353895, |
| cg24063856, | cg15457598, | cg08837308, | cg05461818, | cg08830520, | cg21270074, |
| cg04484579, | cg20417424, | cg02281970, | cg20667211, | cg12176856, | cg20216950, |
| cg07834314, | cg20276630, | cg14200305, | cg15589354, | cg07833019, | cg15424120, |
| cg01181009, | cg03493668, | cg17821649, | cg20264534, | cg17798633, | cg09625946, |
| cg08914623, | cg25034856, | cg03847768, | cg10409299, | cg23008047, | cg05013783, |
| cg22854448, | cg01518631, | cg20596162, | cg00015024, | cg18194957, | cg08759130, |
| cg00032227, | cg16260572, | cg00497630, | cg06115741, | cg07052513, | cg09367696, |
| cg00083206, | cg11754676, | cg25124739, | cg15602359, | cg05827468, | |
| cg23330212, | ch.6.98280058F, | cg14299508, | cg17811845, | cg12750745, | |
| cg00682090, | cg06418721, | cg05775627, | cg09600509, | cg14299177, | cg25417551, |
| cg24697648, | cg12651014, | cg12825836, | cg18358020, | cg08250444, | cg16657149, |
| cg16400320, | cg07605135, | cg09076926, | cg05939647, | cg17498313, | cg09372664, |
| cg10821127, | cg10926047, | cg00175153, | cg24282422, | cg26658897, | cg11254532, |
| cg09616640, | cg04992704, | cg26720913, | cg16557178, | cg02028489, | cg00407329, |
| cg21560413, | cg12258811, | cg00022594, | cg21920507, | cg23027509, | cg15857739, |
| cg08363278, | cg16276199, | cg22579218, | cg21203925, | cg07516958, | cg00470766, |
| cg14971781, | cg27313566, | cg17769836, | cg26925669, | cg26166818, | cg20618622, |
| cg14389169, | cg10011199, | cg12513284, | cg19162970, | cg00829063, | cg25472804, |
| cg1 4065590, | cg10094078, | cg10871876, | cg26195178, | cg01499197, | cg10147875, |
| cg04332534, | cg10629165, | cg07312178, | cg00283046, | cg02530437, | cg00844308, |
| cg24949747, | cg23637607, | cg10068989, | cg26081466, | cg08525922, | cg04753583, |
| cg17131553, | cg25114611, | cg21221767, | cg09936561, | cg15032166, | cg24230340, |
| cg16793043, | cg24138433, | cg00462700, | cg09172752, | cg25334393, | cg19461344, |
| cg12296472, | cg18135796, | cg19787727, | cg21389776, | cg11408477, | cg16681239, |
| cg11774921, | cg12452675, | cg04781796, | cg26464252, | cg08174482, | cg24641522, |
| cg04663931, | cg15822087, | cg05037556, | cg11011736, | cg22037779, | cg09130549, |
| cg13356253, | cg25735398, | cg16204009, | cg23211158, | cg07623995, | cg06042259, |
| cg06184870, | cg09721595, | cg11654769, | cg26935333, | cg00603274, | cg01595325, |
| cg07367113, | cg06939920, | cg00134041, | cg07177174, | cg05488439, | cg16649298, |
| cg00375589, | cg03903398, | cg04703867, | cg03820282, | cg18692276, | cg17650523, |
| cg18060030, | cg14647640, | cg12840024, | cg27607340, | cg13628643, | cg04790147, |
| cg06489295, | cg01640958, | cg02028562, | ch.15.77890571R, | cg12005429, | |
| cg10484211, | cg22547978, | cg21192621, | cg13171016, | cg13582371, | cg03790207, |
| cg10151367, | cg00383149, | cg01096487, | cg14917874, | cg02099572, | cg07197480, |
| cg24439315, | cg16493813, | cg16926844, | cg12275348, | cg03680348, | cg14634687, |
| cg00011717, | cg23454082, | cg24198941, | cg03383239, | cg22455614, | cg19779057, |
| cg06714180, | cg02925948, | cg02983495, | cg10082313, | cg20490001, | cg17959970, |
| cg18693004, | cg06010667, | cg06864443, | cg17903067, | cg05361415, | cg02973270, |
| cg23699046, | cg09674502, | cg20419410, | cg16798694, | cg00420922, | cg13082157, |
| cg27652681, | cg02107844, | cg07348535, | cg02310229, | cg06898293, | cg10881306, |
| cg00037940, | cg05307819, | cg21827955, | cg12286910, | cg17493159, | cg01367627, |
| cg05693489, | cg13614753, | cg07998374, | cg20784259, | cg26933107, | cg11119419, |
| cg02651638, | cg00503447, | cg12165685, | cg00152041, | cg18128550, | cg02282892, |
| cg15188268, | cg23300882, | cg05644498, | cg09086087, | cg10664162, | cg14028708, |
| cg05991534, | cg02430497, | cg19359550, | cg23844154, | cg06391199, | cg25913147, |
| cg01623879, | cg08966978, | cg13429194, | cg10714639, | cg06138710, | cg12172046, |
| cg06520672, | cg03072035, | cg11504805, | cg24342283, | cg02390267, | |
| cg01088093, | cg27261397, | cg21328651, | cg25024354, | cg25697800, | cg04393170, |
| cg12258454, | cg03146981, | cg10215570, | cg01503378, | cg09067715, | cg01341969, |
| cg10665071, | cg24553815, | cg14924052, | cg13607218, | cg14183712, | cg15801964, |
| cg20863756, | cg14161821, | cg04240980, | cg14599755, | cg25755575, | cg21174746, |
| cg03414898, | cg25113123, | cg27033705, | cg13227481, | cg04344119, | cg12062853, |
| cg19695166, | cg15850846, | cg12274663, | cg26217572, | cg09777883, | cg09037025, |
| cg02697527, | cg24754199, | cg.15.86030110R, | cg01144768, | cg22513396, | |
| cg19831706, | cg08810582, | cg17848348, | cg13476800, | cg06260527, | cg15626112, |
| cg04879451, | cg19241089, | cg09241022, | cg23375968, | cg02916283, | cg00338002, |
| cg10161194, | cg04554240, | cg00640479, | cg25903072, | cg17346857, | cg13888886, |
| cg05532204, | cg22321036, | cg04438595, | cg25674709, | cg04622200, | cg18754857, |
| cg27199599, | cg22593953, | cg00292971, | cg25157615, | cg09802818, | cg24389359, |
| cg26963428, | cg11650704, | cg01244015, | cg08436643, | cg00786618, | cg03369671, |
| cg23521294, | cg02257312, | cg26219843, | cg16245339, | cg02689448, | |
| cg26680793, | cg08524483, | cg05353133, | cg23489273, | cg06216293, | cg26807961, |
| cg19864705, | cg04958191, | cg06950479, | cg02949969, | cg13510114, | cg22060694, |
| cg18712973, | cg19676328, | cg18633693, | cg20048599, | cg05644480, | cg19563130, |
| cg27171580, | cg06599949, | cg16754929, | cg22556768, | cg22911462, | cg11547828, |
| cg07477815, | cg15641872, | cg10557351, | cg00445232, | cg15036475, | cg13808278, |
| cg12888358, | cg13792444, | cg11282556, | cg08991599, | cg14366939, | cg03740752, |
| cg18387099, | cg22264317, | cg08664849, | cg24493649, | cg00909993, | cg26060536, |
| cg16519772, | cg11984608, | cg19699291, | cg04718185, | cg14201956, | cg06830702, |
| cg07141702, | cg25503540, | cg09690040, | cg14334911, | cg15259233, | cg09997676, |
| cg06912515, | cg27259408, | cg24387285, | cg08817171, | cg21388793, | |
| cg13850887, | cg12958046, | cg19964273, | cg10267162, | cg13970437, | cg10605137, |
| cg23306295, | cg01656955, | cg17631894, | cg21142349, | cg03323895, | cg04910587, |
| cg13900100, | cg05155595, | cg26925343, | cg05408649, | cg06926349, | cg20180061, |
| cg16743924, | cg07182756, | cg01559912, | cg05785945, | cg03345120, | cg04321580, |
| cg12676444, | cg24902920, | cg00970313, | cg00653085, | cg03464655, | cg17491300, |
| cg02273724, | cg02388500, | cg15392919, | cg11569979, | cg26504835, | cg03221715, |
| cg06573573, | cg07344955, | cg12970730, | cg14444287, | cg21482265, | cg21381949, |
| cg18693656, | cg14621103, | cg03564931, | cg07030727, | cg05687174, | cg24200059, |
| cg14941149, | cg10090418, | cg22747516, | cg22584618, | cg26797372, | cg14702589, |
| cg10881110, | cg16681104, | cg26174880, | cg01644518, | cg02701615, | cg03580800, |
| cg07506599, | cg08756008, | cg21072934, | cg15274001, | cg15097028, | cg17215449, |
| cg04135543, | cg19040026, | cg26468336, | cg22488717, | cg11834473, | cg22424615, |
| cg22108175, | cg11865513, | cg13567725, | cg25341937, | cg10911971, | cg01562528, |
| cg00367047, | cg27189087, | cg21004654, | cg02113521, | cg07594031, | cg04936446, |
| cg14697642, | cg23249336, | cg20320158, | cg07179924, | cg02762440, | cg18236863, |
| cg11670519, | cg06955716, | cg04791678, | cg21477317, | cg22072935, | cg24623608, |
| cg02019920, | cg18576957, | cg20442586, | cg22473097, | cg22545840, | cg22250546, |
| cg25283175, | cg07533529, | cg03599357, | cg11802899, | cg01982455, | cg15456925, |
| cg02635829, | cg03957884, | cg22683154, | cg24949251, | cg15484161, | cg03508504, |
| cg18765439, | cg25953688, | cg04167480, | cg00083262, | cg04617570, | cg10132010, |
| cg25378920, | cg15082292, | cg20697010, | cg12096988, | cg07732336, | cg11165752, |
| cg16801720, | cg05077865, | cg01926614, | cg14280576, | cg17225591, | cg19125081, |
| cg20347647, | cg18868357, | cg12642705, | cg02635932, | cg09962925, | cg04849769, |
| cg08744475, | cg02083938, | cg18451817, | cg06538709, | cg12678462, | cg08241321, |
| cg10125317, | cg00336669, | cg15095622, | cg07852756, | cg18852038, | cg22872033, |
| cg23739036, | cg00570896, | cg22931642, | cg18057548, | cg07827420, | cg08305778, |
| cg17176676, | cg01333333, | cg01906944, | cg23980859, | cg09696411, | cg17600661, |
| cg19946699, | cg21578987, | cg13672501, | cg26855812, | cg03669292, | cg02260839, |
| cg03019460, | cg10046671, | cg10365900, | cg01390062, | cg20898273, | cg03084824, |
| cg27307183, | cg04998674, | cg11685898, | cg24018765, | cg25784220, | cg03668687, |
| cg19834134, | cg02246609, | cg00935782, | cg02865068, | cg03445127, | cg22141111, |
| cg02056135, | cg12729307, | cg00308767, | cg04297819, | cg17340779, | cg25008504, |
| cg09640202, | cg01275038, | cg03752935, | cg02131230, | cg10130537, | cg04036049, |
| cg21807313, | cg27596226, | cg19164917, | cg01859717, | cg03716405, | cg12448860, |
| cg23607077, | cg01220257, | cg14247588, | cg10139443, | cg22443241, | |
| cg23012683, | cg08399733, | cg05053108, | cg16560077, | cg17372269, | cg00281725, |
| cg11913694, | cg26720767, | cg01796493, | cg04790936, | cg11164618, | cg13751188, |
| cg05380145, | cg27247782, | cg16614613, | cg16799087, | cg21127109, | cg15020645, |
| cg15684210, | cg06855336, | cg11531239, | cg06815737, | cg09870910, | cg09540629, |
| cg07805642, | cg00712075, | cg13604154, | cg05129802, | cg21672889, | cg24003850, |
| cg17407629, | cg12641275, | cg06381959, | cg10016690, | cg10447982, | cg00360600, |
| cg06233202, | cg13593597, | cg25400229, | cg08951958, | cg09171127, | cg17663577, |
| cg03743981, | cg24335456, | cg16468773, | cg01387072, | cg17540671, | cg05714748, |
| cg19875976, | cg18219713, | cg25086828, | cg10522062, | cg07277041, | cg10061129, |
| cg19801921, | cg14583103, | cg05450804, | cg25092473, | cg06649691, | cg18789887, |
| cg23965022, | cg24932585, | cg09431318, | cg24399337, | cg13993218, | cg00397059, |
| cg25334860, | cg18935449, | cg11827082, | cg12477716, | cg03332810, | cg24848081, |
| cg10085057, | cg08468187, | cg13447818, | cg03018478, | cg09251600, | cg20185017, |
| cg17644557, | cg18941831, | cg14604444, | cg21335714, | cg00154920, | cg17387577, |

¹CvC—cancer vs. control samples (TCGA dataset)
²PvR—progressive vs. regressive CIS dataset

TABLE 2
Metrics:

| Number | ROC AUC | ROC | Threshold | Sensitivity | Specificity | Sensitivity | Specificity |
|---|---|---|---|---|---|---|---|
| of DMPs | (CvC¹) | (PvR²) | value | (CvC) | (CvC) | (PvR) | (PvR) |
| 2000 | 0.98458816 | 0.787808642 | 0.37 | 1 | 0.451351351 | 0.944444444 | 0.666666667 |

DMP IDs:

| | | | | | |
|---|---|---|---|---|---|
| cg13662093, | cg22630231, | cg22883887, | cg18450270, | cg17688733, | cg13781744, |
| cg09578155, | cg26903090, | cg11112226, | cg26341831, | cg13200847, | cg16002322, |
| cg04066453, | cg20048529, | cg26525008, | cg19299265, | cg07446753, | cg25515063, |
| cg03351724, | cg25815219, | cg27340057, | cg21672550, | cg08179530, | cg10734432, |
| cg05143078, | cg01896926, | cg16801797, | cg03902201, | cg03520983, | cg25021359, |
| cg15649474, | cg05944840, | cg06051213, | cg23701703, | cg24569045, | cg14723664, |
| cg07596524, | cg10780525, | cg03472655, | cg08528474, | cg23973175, | cg20044305, |
| cg06077079, | cg14045283, | cg26847866, | cg17308545, | cg15543875, | cg25364273, |
| cg14658630, | cg00547077, | cg14832358, | cg20511832, | cg24734792, | cg05066387, |
| cg03242877, | cg08365301, | cg05447556, | cg04067612, | cg14390179, | cg11356451, |
| cg07172676, | cg06898145, | cg22807877, | cg17447867, | cg10464454, | cg01208814, |
| cg03838635, | cg22210463, | cg01663953, | cg09916572, | cg21939836, | cg16910042, |
| cg06627151, | cg26344798, | cg14999396, | cg17084007, | cg24630764, | cg24219589, |
| cg15745205, | cg26487031, | cg13775832, | cg18257996, | cg09163117, | cg11212864, |
| cg13367612, | cg16603578, | cg06835074, | cg05718034, | cg27212099, | cg25754248, |
| cg14533390, | cg07894501, | cg12965553, | cg08543327, | cg27649348, | cg02388849, |
| cg06962748, | cg19262631, | cg18643445, | cg12004430, | cg00911813, | cg17718321, |
| cg15049933, | cg19610905, | cg07214490, | cg14297518, | cg06534586, | cg17033287, |
| cg24512644, | cg00604736, | cg01101742, | cg19412007, | cg10981736, | cg26091679, |
| cg21096907, | cg02900995, | cg24296484, | cg16309926, | cg10812016, | cg26246928, |
| cg09651496, | cg08811210, | cg24528350, | cg05468351, | cg20704677, | cg02385095, |
| cg05119141, | cg07358855, | cg21351852, | cg01353073, | cg01324802, | cg26297005, |
| cg08277486, | cg07387931, | cg21858614, | cg08540953, | cg05392527, | cg20283477, |
| cg18966152, | cg12669088, | cg13800569, | cg10712561, | cg10642188, | cg00049382, |
| cg01785060, | cg06193383, | cg09762897, | cg02580923, | cg07011304, | cg06195280, |
| cg13088253, | cg24941342, | cg09728393, | cg03655771, | cg05394210, | cg07594472, |
| cg16910805, | cg08281469, | cg11207300, | cg20147389, | cg26048923, | cg05300029, |
| cg23113715, | cg10640764, | cg23781334, | cg22670503, | cg01496491, | cg17675882, |
| cg07236157, | cg14031065, | cg26870584, | cg03897136, | cg15686782, | cg22043118, |
| cg23186643, | cg04283751, | cg14553765, | cg22481960, | cg24576270, | cg03871124, |
| cg20807545, | cg25308079, | cg17657618, | cg12530017, | cg10541813, | cg22655521, |
| cg03469862, | cg19449969, | cg01558195, | cg05807885, | cg01710791, | cg14651742, |
| cg04276484, | cg25016308, | cg10099518, | cg00308439, | cg18042593, | cg26599274, |
| cg13855472, | cg18902579, | cg17849156, | cg05199950, | cg06434344, | cg22718636, |
| cg12157045, | cg10220992, | cg10299061, | cg13687784, | cg17840047, | cg16202736, |
| cg18701983, | cg14535717, | cg16497583, | ch.15.31224719R, | cg26310483, | cg08701998, |
| cg13384453, | cg05373339, | cg02266725, | ch.8.49587141F, | cg05532239, | cg11369906, |
| cg18096962, | cg10918016, | cg16328610, | cg26027052, | cg26878836, | cg04628197, |
| cg01144921, | cg21639236, | cg11078084, | cg17678877, | cg03801830, | cg01614101, |
| cg22282265, | cg12403253, | cg09804833, | cg01517273, | cg02719956, | cg25320099, |
| cg27620457, | cg26678383, | cg03481903, | cg07225458, | cg01638792, | cg04572969, |
| cg16519321, | cg05209525, | cg27411220, | cg25423111, | cg27523547, | cg17440077, |
| cg01944444, | cg13497074, | cg00307383, | cg21926626, | cg18237607, | cg11635029, |
| cg00340658, | cg02455383, | cg02462443, | cg14163494, | cg25164589, | cg03636939, |
| cg18324542, | cg00909806, | cg05091796, | cg23655651, | cg16215361, | cg20990681, |
| cg19771920, | cg00848245, | cg08015388, | cg19738283, | cg03986829, | cg18530251, |
| cg15049101, | cg23460551, | cg25730573, | cg06379383, | cg11865208, | cg23903252, |
| cg05470166, | cg13964393, | cg11892946, | cg19651155, | cg25236230, | cg27109131, |
| cg07233135, | cg15234197, | cg03355690, | cg27011060, | cg00201196, | cg07162608, |
| cg12177551, | cg23618477, | ch.17.480931F, | cg09230774, | cg04149015, | cg07202281, |
| cg00377924, | cg11189434, | cg07728084, | cg22112918, | cg18253516, | cg11953250, |
| cg23927832, | cg24837397, | cg10617494, | ch.10.2809628F, | cg09484214, | cg09684160, |
| cg27084303, | cg15137445, | cg18317492, | cg18564686, | cg26112797, | cg00793432, |
| cg19811148, | cg11296363, | cg12266841, | cg14016057, | cg00495693, | cg16411204, |
| cg15350071, | cg13245817, | cg22479226, | cg20845050, | cg26106304, | cg02207944, |
| cg06745566, | cg21547982, | cg00173063, | cg26468118, | cg17093138, | cg21007509, |
| cg09203312, | cg25136294, | cg05908079, | cg21037018, | cg14522803, | cg19142181, |
| cg16626954, | cg05812171, | cg16608656, | cg04915058, | cg10572274, | cg03386823, |
| cg17112695, | cg26135441, | cg13911576, | cg03529015, | cg11267802, | cg24596472, |
| cg06172859, | cg26347089, | cg23906687, | cg22467810, | cg14556677, | cg19523819, |
| cg06908379, | cg10925860, | cg00174179, | cg22291974, | cg04574732, | cg26181334, |
| cg23714705, | cg12729822, | cg02657855, | cg12023170, | cg22249529, | cg09364338, |
| cg14440550, | cg12080909, | cg19485911, | cg23339629, | cg26500707, | cg03159329, |
| cg17600661, | cg12033014, | cg10177647, | cg09603188, | cg17338212, | cg02927682, |
| cg17240499, | cg12536891, | cg05991342, | cg24676303, | cg06410158, | cg02003612, |
| cg02498382, | cg00077450, | cg27628552, | cg16331828, | cg13360823, | cg12243597, |
| cg07899411, | cg17968946, | cg17985608, | cg01781725, | cg09704415, | cg10512203, |
| cg03458463, | cg09814448, | cg23263911, | cg14480858, | cg21139063, | cg09256716, |
| cg19142399, | cg15462501, | cg13443938, | ch.4.101815866R, | cg03699633, | cg16121929, |
| cg09901932, | ch.15.1681270R, | cg10911980, | cg08480398, | cg26694990, | cg15576869, |
| cg11058229, | cg01561839, | cg18581777, | cg15984741, | cg02441831, | cg11589966, |
| cg27149285, | cg19793499, | cg10273890, | cg03304380, | cg10423052, | cg20730432, |
| cg00512454, | cg17226446, | cg08706567, | cg09097916, | cg14173185, | cg03282105, |
| cg27246571, | cg00057231, | cg18796319, | cg08155584, | cg07116163, | cg23758252, |
| cg11413581, | cg01081584, | cg24320673, | cg04364718, | cg22046830, | cg14070275, |
| cg16262614, | cg23240663, | cg15067340, | cg15442444, | cg07509646, | cg23023263, |
| cg04332513, | cg06225767, | cg18252616, | cg16377819, | cg06817090, | cg12864853, |
| cg11781339, | cg13581847, | cg09821328, | cg20207973, | cg22131726, | cg04466253, |
| cg01566199, | cg25669504, | cg10172253, | cg10925991, | cg03468466, | cg15197202, |
| cg12419239, | cg01070760, | cg00891242, | cg14298200, | cg26372893, | cg00561739, |
| cg18585396, | cg16430887, | cg04805612, | cg06631222, | cg12250049, | cg08236479, |
| cg26888147, | cg12517843, | cg25581039, | cg10526478, | cg18010131, | cg05231018, |
| cg25702652, | cg03009463, | cg05265747, | cg23456692, | cg10957151, | cg17864091, |
| cg15783064, | cg16318947, | cg14452209, | cg00929465, | cg21592262, | cg18331396, |
| cg10978966, | cg06496222, | cg13534740, | cg13782274, | cg04946916, | cg17199481, |
| cg24060136, | cg17258607, | cg00012362, | cg05324982, | cg14059768, | cg01791523, |
| cg00471989, | cg10219957, | cg08529049, | cg17500265, | cg24543574, | cg08022436, |
| cg00820718, | cg14727777, | cg26425711, | cg13608629, | cg18338319, | cg16664496, |
| cg02856716, | cg21372914, | cg19595886, | cg25143746, | cg21437481, | cg20559561, |
| cg04380513, | cg14305028, | cg06825833, | cg04273871, | cg24778383, | cg01820106, |
| cg05959392, | cg17952075, | cg07196858, | cg25675571, | cg13240704, | cg04927502, |
| cg22075328, | cg02688422, | cg26322788, | cg18550498, | cg16627247, | cg23677990, |
| cg14257319, | cg04210056, | cg10725720, | cg06303744, | cg21151633, | cg10590909, |
| cg08700707, | cg04234156, | cg24927135, | cg17414007, | cg06618957, | cg19274270, |
| cg17185639, | cg17307348, | cg14151082, | cg27010688, | cg25547332, | cg21622025, |
| cg18432145, | cg00803827, | cg20451705, | cg06397457, | cg09757277, | cg21176026, |
| cg03282439, | cg11710211, | cg07168837, | cg00777968, | cg17552295, | cg11826475, |
| cg05473018, | cg02092906, | cg17445120, | cg10111214, | cg06366374, | cg14276730, |
| cg03521737, | cg01936977, | cg16128638, | cg01045612, | cg17782713, | cg10957733, |
| cg25629326, | cg27578963, | cg25683495, | cg07219494, | cg21298593, | cg25745651, |
| cg03325664, | cg08010619, | cg26846726, | cg02192520, | cg19214408, | cg20202505, |
| cg24759859, | cg11608005, | cg06447122, | cg24756292, | cg06051146, | cg13642914, |
| cg11814422, | cg19007537, | cg06472554, | cg12067292, | cg18557684, | cg24639504, |
| cg25421941, | cg06961233, | cg21986718, | cg17265380, | cg01343936, | cg01665432, |
| cg07938869, | cg22655975, | cg00579423, | cg00506343, | cg14195915, | cg23396177, |
| cg23056047, | cg01863964, | cg12776287, | cg03881768, | cg27453479, | cg17560340, |
| cg22730675, | cg24256745, | cg23795217, | cg13529335, | cg02450004, | cg09321082, |
| cg10754935, | cg20403938, | cg22231620, | cg08908855, | cg13495838, | cg22392501, |
| cg25935831, | cg03109316, | cg12652546, | cg04583813, | cg20200605, | cg18945467, |
| cg21942893, | cg01044331, | cg02341374, | cg20816478, | cg27380724, | cg22185043, |
| cg17344932, | cg07364009, | cg07138585, | cg11437231, | cg15212266, | cg24041637, |
| cg18462168, | cg00459997, | cg18397073, | cg18258397, | cg14591667, | cg16174348, |
| cg08081805, | cg27544046, | cg02482690, | cg11725689, | cg26740644, | cg07536072, |
| cg11444198, | cg14354631, | cg17185823, | cg26890727, | cg08802149, | cg11878872, |
| cg03228945, | cg09686236, | cg14372792, | cg10786043, | cg00681278, | cg14010829, |
| cg05031931, | cg11490961, | cg18438894, | cg26707664, | cg23967544, | cg12533010, |
| cg25733009, | cg00549463, | cg12708802, | cg26280713, | cg19643053, | cg17247293, |
| cg19497548, | cg10798048, | cg23588713, | cg13913247, | cg12279294, | cg00520612, |
| cg04900672, | cg00334399, | cg16280661, | cg02808240, | cg20301722, | cg24127414, |
| cg08828787, | cg10701286, | cg10301879, | cg16171534, | cg13425677, | cg10107725, |
| cg26525231, | cg02113604, | cg21326642, | cg26466157, | cg03331474, | cg21599100, |
| cg13938035, | cg11932091, | cg15743985, | cg02749887, | cg12023686, | cg11191210, |
| cg13696228, | cg00660167, | cg11750962, | cg20859099, | cg14087209, | cg18674446, |
| cg26238031, | cg08916508, | cg19862242, | cg19252565, | cg21566086, | cg20477084, |
| cg13408752, | cg10638657, | cg05939907, | cg11677966, | cg12057741, | cg00909232, |
| cg10159215, | cg08158976, | cg27265573, | cg10772621, | cg18083404, | cg04177091, |
| cg03116409, | cg07035659, | cg09063683, | cg08270223, | cg13898564, | cg00505997, |
| cg20904475, | cg13187967, | cg03604796, | cg12064947, | cg21147147, | cg15904821, |
| cg04760426, | cg18112681, | cg21487509, | cg10845171, | cg27051315, | cg00427296, |
| cg13918472, | cg19778944, | cg24242981, | cg18232597, | cg03719083, | cg02329576, |
| cg18784586, | cg13791827, | cg24420680, | cg02603140, | cg14470398, | cg03547366, |
| cg11797228, | cg18770442, | cg10199121, | cg16764637, | cg23926615, | cg18144065, |
| cg04311134, | cg09161138, | cg01870330, | cg18987026, | cg12396325, | cg14644209, |
| cg19942237, | cg02621020, | cg01371498, | cg05073620, | cg08042799, | cg04371528, |
| cg08035638, | cg10527285, | cg17437852, | cg01830294, | cg00236766, | cg04983349, |
| cg08388004, | cg19693446, | cg09740313, | cg00489861, | cg18411891, | cg22639293, |
| cg02569219, | cg22908423, | cg20693334, | cg24527560, | cg01452549, | cg00557308, |
| cg00420742, | cg19668728, | cg00248828, | cg16223510, | cg23668424, | cg18021368, |
| cg15377871, | cg01438403, | cg01166488, | cg19085507, | cg22143352, | cg20688928, |
| cg26055420, | cg02039171, | cg19934381, | cg07134367, | cg10740282, | cg05392844, |
| cg00746446, | cg24803290, | cg25051233, | cg21330802, | cg17493727, | cg01093938, |
| cg25540816, | cg18002602, | cg00445523, | cg23282550, | cg01738805, | cg22998610, |
| cg07560534, | cg12778430, | cg00978505, | cg26614815, | cg25103172, | cg12187567, |
| cg16291854, | cg04146405, | cg18547284, | cg05082557, | cg04245634, | cg20535425, |
| cg15195541, | cg23068913, | cg10000775, | cg12595697, | cg05106359, | cg09412808, |
| cg01886004, | cg04214629, | cg18830042, | cg11760986, | cg23047544, | cg15933527, |
| cg27023638, | cg02120774, | cg22330638, | cg19635897, | cg16792888, | cg21117476, |
| cg23485716, | cg02492791, | cg00979369, | cg12818699, | cg05764570, | cg18066403, |
| cg21871583, | cg15757195, | cg02990438, | cg22852276, | cg24448849, | cg18328477, |
| cg24941195, | cg02886027, | cg21607700, | cg13255216, | cg13656062, | cg14118546, |
| cg22201615, | cg05434655, | cg00797183, | cg08543242, | cg05270552, | cg08330972, |
| cg23864120, | cg03382346, | cg13959595, | cg05526886, | cg22093901, | cg14552839, |
| cg04329845, | cg12985418, | cg01733795, | cg18988938, | cg11507187, | cg00244882, |
| cg07501988, | cg23243012, | cg09295063, | cg10143960, | cg20414783, | cg12555033, |
| cg02730055, | cg00420354, | cg20706192, | cg19872368, | cg24244854, | cg26668626, |
| cg05191454, | cg20138861, | cg00206052, | cg24033042, | cg02515899, | cg16576213, |
| cg22049732, | cg22780731, | cg03795587, | cg24150157, | cg05181221, | cg05877661, |
| cg04419707, | cg01253856, | cg19545560, | cg23241045, | cg11866527, | cg04400533, |
| cg23151295, | cg19984911, | cg14310231, | cg16705395, | cg16601489, | cg22047013, |
| cg15084390, | cg04558115, | cg24104567, | cg26913576, | cg12947447, | cg00018128, |
| cg02173289, | cg05995135, | cg24309171, | cg01972751, | cg06502570, | cg01411982, |
| cg02138124, | cg25502179, | cg09976876, | cg08105352, | cg11315633, | cg19611418, |
| cg20119101, | cg05138298, | cg13313294, | cg17650713, | cg13831649, | cg24431318, |
| cg26421448, | cg11816081, | cg10583931, | cg22467470, | cg22045055, | cg19987800, |
| cg19635926, | cg20546518, | cg02245794, | cg00993400, | cg10507762, | cg12378753, |
| cg17566002, | cg11539173, | cg00872677, | cg14951139, | cg25600478, | cg00648452, |
| cg06926254, | cg26679912, | cg21459532, | cg23553659, | cg26117986, | cg24066843, |

DMP IDs:

| | | | | | |
|---|---|---|---|---|---|
| cg02859417, | cg04902729, | cg12559179, | cg21899596, | cg04374177, | cg23348789, |
| cg03835296, | cg13251608, | cg00308503, | cg17639493, | cg02532725, | cg26853340, |
| cg19638175, | cg18549688, | cg23881613, | cg05989740, | cg00477972, | cg08061524, |
| cg08612138, | cg03224276, | cg06055014, | cg15725639, | cg09816218, | cg15267720, |
| cg21200353, | cg24196126, | cg02648581, | cg22290045, | cg04125350, | cg17945282, |
| cg17603105, | cg18722124, | cg22947532, | cg20460852, | cg07790045, | cg01172785, |
| cg10797319, | cg20438344, | cg18075720, | cg01627483, | cg00139681, | cg23860321, |
| cg12971338, | cg26627933, | cg08853539, | cg15815318, | cg04330326, | cg08940505, |
| cg23681219, | cg23568341, | cg10721556, | cg23575754, | cg03812676, | cg17003293, |
| cg00368356, | cg16354756, | cg04766061, | cg21995039, | cg04352803, | cg15827677, |
| cg26816707, | cg11033835, | cg06518763, | cg26157270, | cg08272591, | cg05877497, |
| cg25104637, | cg18040305, | cg15558129, | cg13982366, | cg03074421, | cg26294410, |
| cg11613545, | cg12311930, | cg08840152, | cg14817370, | cg09701102, | cg05120028, |
| cg22794775, | cg13762359, | cg06088360, | cg19927508, | cg05583848, | cg02428056, |
| cg26687844, | cg03892714, | cg15633273, | cg04705952, | cg21181989, | cg27263395, |
| cg06202686, | cg10575089, | cg15611897, | cg13026370, | cg08646988, | cg16020747, |
| cg12244544, | cg06631310, | cg23911808, | cg15602074, | cg22861279, | cg23047271, |
| cg05855917, | cg07476406, | cg01894508, | cg19740859, | cg00220113, | cg13466988, |
| cg22651493, | cg11800832, | cg09667373, | cg17996619, | cg14240248, | cg21113773, |
| cg01143375, | cg11302575, | cg03448532, | cg10462597, | cg19878838, | cg26304213, |
| cg06003187, | cg00235337, | cg15424120, | cg17780413, | cg14833810, | cg27294629, |
| cg25094467, | cg13987088, | cg00544955, | cg19218509, | cg24207529, | cg01293797, |
| cg17086204, | cg15325704, | cg09725962, | cg01591025, | cg19730092, | cg04823499, |
| cg07532089, | cg05768427, | cg02898036, | cg16576106, | cg22534759, | cg16050784, |
| cg07261225, | cg04022560, | cg17046823, | cg09576649, | cg00020622, | cg02031326, |
| cg19969359, | cg06536503, | cg26180383, | cg12709880, | cg15622805, | cg16576935, |
| cg24250070, | cg17566867, | cg22514863, | cg04244183, | cg09950916, | cg06118384, |
| cg27648056, | cg15198344, | cg07781158, | cg06723337, | cg11209008, | cg05520940, |
| cg07025432, | cg18294116, | cg04214566, | cg27609721, | cg03324578, | cg05000761, |
| cg04093671, | cg27589368, | cg12080492, | cg06602847, | cg26071631, | cg06367472, |
| cg13670833, | cg09962925, | cg25407888, | cg22224628, | cg22165507, | cg00421693, |
| cg13830636, | cg25884399, | cg21877613, | cg23855986, | cg17120983, | cg04309849, |
| cg03581311, | cg10712476, | cg13576883, | cg27150870, | cg12836959, | cg04767932, |
| cg23067856, | cg25958740, | cg24509225, | cg15236035, | cg17389295, | cg00540587, |
| cg24875840, | cg14898433, | cg21396127, | cg11332388, | cg04119833, | cg02114728, |
| cg27573570, | cg05196410, | cg05393563, | cg12656632, | cg22573675, | cg23638640, |
| cg00536472, | cg22686132, | cg18764008, | cg06214606, | cg09051837, | cg08120044, |
| cg26165286, | cg13778208, | cg26363598, | cg03885270, | cg12672050, | cg16743924, |
| cg07479014, | cg23338993, | cg17464820, | cg16200569, | cg05109245, | cg24102622, |
| cg01293740, | cg08951273, | cg13159054, | cg10798171, | cg12550731, | cg07820280, |
| cg05671350, | cg22206370, | cg14818946, | cg19451466, | cg00597225, | cg06352352, |
| cg25026248, | cg10375802, | cg06686383, | cg25783099, | cg00875541, | cg25024321, |
| cg22335876, | cg06677010, | cg13379827, | cg17445830, | cg00955780, | cg07023209, |
| cg23251858, | cg05614333, | cg13457217, | cg19915020, | cg02017074, | cg01353486, |
| cg21714305, | cg22738278, | cg01385430, | cg18096620, | cg21692936, | cg00539368, |
| cg10319073, | cg16238207, | cg19491599, | cg24049990, | cg19473021, | cg23894003, |
| cg16535999, | cg26273211, | cg07166601, | cg20395892, | cg03532904, | cg18688293, |
| cg00709387, | cg00231483, | cg14583225, | cg07444288, | cg00776166, | cg05310760, |
| cg01083131, | cg07800959, | cg21063722, | cg16349612, | cg16422884, | cg07684929, |
| cg05687174, | cg22531466, | cg18283926, | cg16024836, | cg24609094, | cg15449323, |
| cg02610813, | cg15990223, | cg18038372, | cg01788994, | cg24876012, | cg05710540, |
| cg03271093, | cg08021120, | cg18950940, | cg24356321, | cg11649040, | cg08821600, |
| cg19764345, | cg14380586, | cg02988171, | cg22250498, | cg07191152, | cg24302024, |
| cg06937207, | cg19259146, | cg04983608, | cg18003112, | cg25323497, | cg14502850, |
| cg26208507, | cg14400292, | cg02937233, | cg20866932, | cg10718078, | cg23153615, |
| cg10935723, | cg26963795, | cg02487987, | cg24724937, | cg01403239, | cg01187496, |
| cg22804418, | cg10322124, | cg07877517, | cg12604058, | cg14183907, | cg27658017, |
| cg26074851, | cg11904751, | cg01630130, | cg23029526, | cg18280962, | cg19816560, |
| cg16488953, | cg07216647, | cg26228859, | cg18651470, | cg23328329, | cg27098574, |
| cg02094237, | cg25607239, | cg07051140, | cg06599514, | cg15989167, | cg18057548, |
| cg03342928, | cg15248828, | cg21054179, | cg27518892, | cg09359475, | cg18684641, |
| cg27645955, | cg26241863, | cg03225546, | cg17042996, | cg10459387, | cg12725284, |
| cg25711003, | cg24029426, | cg00340024, | cg26320601, | cg15057359, | cg10425506, |
| cg16161657, | cg09039561, | cg12307237, | cg00053086, | cg14270556, | cg25148306, |
| cg04799270, | cg24688939, | cg00658367, | cg17816357, | cg06255132, | cg10078446, |
| cg14917700, | cg13365279, | cg12179353, | cg14069287, | cg17053223, | cg24772388, |
| cg22921692, | cg01112020, | cg24768135, | cg24952328, | cg15463280, | cg01659038, |
| cg07069601, | cg16362594, | cg05983695, | cg17743732, | cg21577472, | cg10000195, |
| cg07002372, | cg15098690, | cg18628787, | cg15298722, | cg24611351, | cg09820321, |
| cg16419620, | cg23754772, | cg14335435, | cg20658167, | cg10905877, | cg25488021, |
| cg01885050, | cg01048752, | cg22191326, | cg24765188, | cg07006673, | cg02908720, |
| cg14566946, | cg06676088, | cg09907628, | cg11244340, | cg22466771, | cg16124571, |
| cg20577538, | cg15819853, | cg24279419, | cg18199666, | cg24317988, | cg13949487, |
| cg27207470, | cg06723357, | cg19985911, | cg06345736, | cg08193100, | cg15416681, |
| cg07557337, | cg11242978, | cg19821804, | cg26991025, | cg00377757, | cg03989663, |
| cg20106214, | cg01801174, | cg22870189, | cg00472281, | cg27593250, | cg26118435, |
| cg24399555, | cg06326926, | cg04875128, | cg10636896, | cg23323627, | cg08062233, |
| cg25237396, | cg16451430, | cg11504081, | cg05254132, | cg12582654, | cg26840824, |
| cg14043593, | cg03744383, | cg22500280, | cg08527328, | cg01647795, | cg26552233, |
| cg15787985, | cg08800318, | cg07779120, | cg15157791, | cg16351364, | cg14273545, |
| cg13940239, | cg18224729, | cg16496526, | cg01934041, | cg01728554, | cg03433669, |
| cg01423916, | cg12166018, | cg02408480, | cg01066494, | cg22899502, | cg16251255, |
| cg02114288, | cg03590328, | cg22492966, | cg13200125, | cg11674664, | cg12877462, |
| cg15690347, | cg10734940, | cg10226013, | cg02121996, | cg07368317, | cg01223221, |
| cg10512020, | cg05051902, | cg26385256, | cg12741441, | cg02737307, | cg00290758, |
| cg06813100, | cg01692616, | cg23323671, | cg14907788, | cg06640637, | cg15848620, |
| cg24960641, | cg11817589, | cg01618245, | cg24100636, | cg02611874, | cg16746589, |
| cg20979823, | cg19740707, | cg18496262, | cg11829253, | cg22307009, | cg20850195, |
| cg07348587, | cg03820689, | cg20300707, | cg06628473, | cg04859757, | cg07744562, |
| cg18168387, | cg24054190, | cg27062617, | cg17903544, | cg25067606, | cg23644589, |
| cg05732794, | cg18684595, | cg11821068, | cg10072850, | cg06528645, | cg06564125, |
| cg07904900, | cg24180066, | cg07551879, | cg03652676, | cg05339942, | cg01089838, |
| cg25215047, | cg05088898, | cg24324985, | cg24868015, | cg21275801, | cg20768743, |
| cg14011229, | cg22122329, | cg05691054, | cg05910443, | cg00032884, | cg09806575, |
| cg15818800, | cg00899855, | cg03518417, | cg16264966, | cg27553457, | cg10516146, |
| cg14421371, | cg08576880, | cg20902195, | cg08504583, | cg13913012, | cg14029169, |
| cg26149920, | cg24757310, | cg17555712, | cg24082799, | cg24519596, | cg08597067, |
| cg26467631, | cg27514509, | cg22792063, | cg10953487, | cg22059413, | ch.13.1744822R, |
| cg17522727, | cg23305440, | cg11938014, | cg08837215, | cg07317755, | cg09139144, |
| cg02903436, | cg06126815, | cg04316311, | cg00524443, | cg21390624, | cg08247552, |
| cg05986417, | cg01802953, | cg20849025, | cg22681074, | cg06197571, | cg21254755, |
| cg01717376, | cg08080822, | cg19343809, | cg08401938, | cg26243203, | cg03610516, |
| cg06335251, | cg09112687, | cg03215152, | cg09344631, | cg07052387, | cg16899444, |
| cg22953731, | cg07402669, | cg00862443, | cg07426960, | cg11789684, | cg26111283, |
| cg13502931, | cg23344780, | cg10683646, | cg21988233, | cg00969162, | cg07982208, |
| cg17978037, | cg27141534, | cg24476103, | cg12602711, | cg06859969, | cg11247695, |
| cg16861410, | cg11303988, | cg00865724, | cg22805632, | cg23620047, | cg09234252, |
| cg07517909, | cg08396863, | cg10030008, | cg06842518, | cg24859316, | cg22090863, |
| cg09156542, | cg07489132, | cg09129100, | cg25354918, | cg02071957, | cg06519061, |
| cg05003417, | cg07523741, | cg02212389, | cg07546684, | cg24799159, | cg18305855, |
| cg03827772, | cg25363861, | cg09413950, | cg15617706, | cg00117599, | cg14773466, |
| cg25489524, | cg15939466, | cg19448822, | cg10143349, | cg12251111, | cg12103037, |
| cg19605500, | cg02147194, | cg25707951, | cg27211781, | cg18645688, | cg02206375, |
| cg10145196, | cg06460652, | cg18397653, | cg14482474, | cg17536603, | cg02370858, |
| cg01983248, | cg20621224, | cg00500540, | cg21166347, | cg18313859, | cg17969117, |
| cg14487665, | cg22967016, | cg23362554, | cg24055029, | cg15007699, | cg04785418, |
| cg11064521, | cg24911064, | cg14286025, | cg20579926, | cg16233210, | cg13759229, |
| cg08663953, | cg07696918, | cg05999604, | cg15844005, | cg22508928, | cg22331159, |
| cg03443427, | cg07328699, | cg12690016, | cg14488081, | cg16430538, | cg01900965, |
| cg18151993, | cg01061480, | cg24612498, | cg18183774, | cg08847316, | cg19814934, |
| cg14415283, | cg09186122, | cg03895914, | cg26492480, | cg03533091, | cg19781309, |
| cg01792229, | cg06116236, | cg01544157, | cg21548414, | cg03520448, | cg13051302, |
| cg08343881, | cg07231479, | cg04193065, | cg20569141, | cg02481778, | cg22579841, |
| cg23056437, | cg06702216, | cg12917079, | cg03217587, | cg05423304, | cg09538006, |
| cg18455653, | cg03418563, | cg24467330, | cg23353952, | cg26619725, | cg19259199, |
| cg13747794, | cg13716323, | cg09944616, | cg01866162, | cg20561100, | cg20899253, |
| cg25443975, | cg12800781, | cg07387734, | cg11982718, | cg26063962, | cg27289153, |
| cg22124493, | cg16466334, | cg27035277, | cg25736617, | cg24106124, | cg03923274, |
| cg21593669, | cg00991659, | cg04254595, | cg11503966, | cg26360930, | cg03211936, |
| cg21850254, | cg22254135, | cg19469297, | cg15686385, | cg26145504, | cg23136554, |
| cg02590972, | cg00074771, | cg26193427, | cg00044299, | cg24771804, | cg20100987, |
| cg26307701, | cg05624445, | cg22347696, | cg16252868, | cg12845756, | cg00381974, |
| cg02043373, | cg06024039, | cg00495988, | cg22038409, | cg13925011, | cg10876207, |
| cg14740417, | ch.2.729349R, | cg04710830, | cg23213170, | cg08657424, | cg26677448, |
| cg15794798, | cg10021122, | cg27629782, | cg03426615, | cg17402294, | cg03579624, |
| cg26067897, | cg11613427, | cg10683054, | cg23634177, | cg05701125, | cg24407065, |
| cg04550697, | cg25025455, | cg15392111, | cg24894158, | cg03186986, | cg10039567, |
| cg16299440, | cg07925823, | cg23051887, | cg18614379, | cg01637131, | cg11827097, |
| cg22644012, | cg01426795, | cg02096793, | cg17318716, | cg10137253, | cg17357548, |
| cg26640895, | cg06942183, | cg22080086, | cg15090202, | cg18600331, | cg02769172, |
| cg21620540, | cg19194645, | cg01244134, | cg19075614, | cg13085338, | cg15918259, |
| cg09951201, | cg09789939, | cg15607538, | cg05287886, | cg19868631, | cg19725625, |
| cg06125484, | cg17569154, | cg19947233, | cg17679824, | cg05019307, | cg26954197, |
| cg03307118, | cg08619119, | cg09155346, | cg24116380, | cg18573663, | cg27420264, |
| cg02174634, | cg27021553, | cg19251291, | cg00681363, | cg01925883, | cg15926342, |
| cg01791648, | cg08326933, | cg14425888, | cg04350571, | cg19428417, | cg10438893, |
| cg13762676, | cg01347596, | cg05125843, | cg08855671, | cg05024901, | cg11479221, |
| cg06870470, | cg22877639, | cg09244748, | cg21304211, | cg02443089, | cg16528272, |
| cg05745142, | cg09793376, | cg25009629, | cg24272697, | cg15863924, | cg06925984, |
| cg25287482, | cg09384411, | cg25857090, | cg20684528, | cg07326768, | cg09578308, |
| cg11493924, | cg26267614, | cg20624538, | cg19408882, | cg12577411, | cg02035039, |
| cg14319773, | cg15378579, | cg02014809, | cg23668445, | cg10504573, | cg09633881, |
| cg02028814, | cg09622285, | cg07307484, | cg15298323, | cg00169122, | cg21282452, |
| cg02388276, | cg04196855, | cg25053907, | cg27658068, | cg01295842, | cg24853724, |
| cg04435885, | cg14519120, | cg07793808, | cg15184781, | cg02543159, | cg06188545, |
| cg10884288, | cg07125635, | cg10570405, | cg04354714, | cg06700521, | cg20728704, |
| cg18537361, | ch.19.1271107R, | cg20296141, | cg23463608, | cg27209722, | cg20449685, |
| cg26282384, | cg01634982, | cg26785234, | cg03859186, | cg04280969, | cg04706627, |
| cg07008213, | cg02963000, | cg14915854, | cg13484848, | cg13558810, | cg01142676, |
| cg16295030, | cg17512738, | cg03059807, | cg04014997, | cg00698444, | cg12881726, |
| cg25409308, | cg24984818, | cg17000128, | cg09218398, | cg17181158, | cg05716504, |
| cg03516270, | cg19674166, | cg14965968, | cg02976568, | cg14094164, | cg06613775, |
| cg09501917, | cg02585724, | cg03972012, | cg25739835, | cg25198428, | cg18055557, |
| cg00599219, | cg25923345, | cg24232092, | cg11271171, | cg01059385, | cg00704305, |
| cg16660547, | cg19333614, | cg14653814, | cg10562114, | cg11963365, | cg19699056, |
| cg20768915, | cg08913117, | cg14525390, | cg00640775, | cg24269991, | cg07423399, |
| cg05548402, | cg07564291 | | | | |

¹CvC—cancer vs. control samples (TCGA dataset)
²PvR—progressive vs. regressive CIS dataset

TABLE 3
Metrics:

| Number | ROC AUC | ROC | Threshold | Sensitivity | Specificity | Sensitivity | Specificity |
|---|---|---|---|---|---|---|---|
| of DMPs | (CvC¹) | (PvR²) | value | (CvC) | (CvC) | (PvR) | (PvR) |
| 2000 | 0.983815959 | 0.800925926 | 0.365 | 1 | 0.459459459 | 0.833333333 | 0.694444444 |

DMP IDs:

| | | | | | | |
|---|---|---|---|---|---|---|
| cg15705746, | cg20701897, | cg08610982, | cg25022612, | cg25887811, | cg18652900, | cg18861311, |
| cg00387704, | cg23559680, | cg03719625, | cg23904047, | cg27391511, | cg20741987, | cg02700346, |
| cg08440125, | cg24171047, | cg03012289, | cg24258699, | cg13819609, | cg17837191, | cg11693508, |
| cg25773695, | cg09684106, | cg24339273, | cg04138001, | cg12221574, | cg15763172, | cg22781123, |
| cg26736273, | cg17855783, | cg13074682, | cg07511080, | cg15400997, | cg03917613, | cg16904116, |
| cg22265294, | cg18944640, | cg11478320, | cg26561623, | cg06612122, | cg13828440, | cg22296620, |
| cg06913744, | cg00858400, | cg06153298, | cg22348654, | cg25905812, | cg21071540, | cg20378520, |
| cg06818777, | cg22413126, | cg02356758, | cg12737152, | cg06272648, | cg02256546, | cg23662671, |
| cg14241045, | cg08368973, | cg04982166, | cg06426011, | cg06745030, | cg12372461, | cg27494647, |
| cg07447902, | cg08145144, | cg07268337, | cg02200944, | cg12603369, | cg01914242, | cg24234899, |
| cg02525822, | cg18019509, | cg06615154, | cg09091273, | cg03471106, | cg21907663, | cg01666436, |
| cg23788856, | cg15073248, | cg06178072, | cg00302587, | cg23085167, | cg09027985, | cg04347059, |
| cg05428770, | cg13982511, | cg24462596, | cg21887597, | cg23213951, | cg02086387, | cg03931660, |
| cg14953397, | cg25487370, | cg26321476, | cg09976142, | cg08773314, | cg20066602, | cg09639735, |
| cg24129626, | cg14217684, | cg01701055, | cg21191822, | cg07807690, | cg05896371, | cg13978566, |
| cg00743094, | cg11933951, | cg24845848, | cg13140267, | cg19520201, | cg05179846, | cg02886961, |
| cg24475275, | cg21967909, | cg11293699, | cg15877314, | cg02344701, | cg06852350, | cg18421356, |
| cg03113700, | cg03252829, | cg11599539, | cg24656529, | cg21238276, | cg26672375, | cg08909961, |
| cg08440349, | cg01692757, | cg24430703, | cg20290983, | cg09135347, | cg22807707, | cg08142858, |
| cg00425827, | cg05901357, | cg09528496, | cg02907064, | cg21109075, | cg21406786, | cg04987149, |
| cg16248182, | cg17442852, | cg04168479, | cg03530573, | cg14633965, | cg07703081, | cg02847220, |
| cg10684102, | cg14329275, | cg02985381, | cg12693595, | cg01252713, | cg24961194, | cg15926585, |
| cg06607764, | cg05494008, | cg09383816, | cg00782708, | cg11539351, | cg23944440, | cg12042737, |
| cg14870461, | cg16314369, | cg06514368, | cg22372849, | cg00009970, | cg12101354, | cg07697082, |
| cg05487736, | cg27312312, | cg06836948, | cg23606023, | cg17423089, | cg00590991, | cg05658717, |
| cg01937809, | cg07581883, | cg01672894, | cg25647683, | cg21000021, | cg20685020, | cg17470184, |
| cg08426130, | cg07515865, | cg06661638, | cg00630794, | cg18426487, | cg04892570, | cg14959580, |
| cg15937174, | cg03372467, | cg18864768, | cg05656251, | cg26464892, | cg16002523, | cg05080074, |
| cg01717276, | cg03483626, | cg26659572, | cg24540120, | cg17969913, | cg12256233, | cg04341454, |
| cg20653009, | cg07123227, | cg01133401, | cg14116908, | cg02238069, | cg06908202, | cg27067654, |
| cg14289228, | cg17271677, | cg22711741, | cg06276708, | cg21300373, | cg26727666, | cg12927153, |
| cg15121288, | cg01083131, | cg03420866, | cg14187685, | cg00322667, | cg20894727, | cg10448052, |
| cg25797912, | cg19414360, | cg26021810, | cg18250135, | cg20481343, | cg03719539, | cg02277509, |
| cg07681938, | cg03837627, | cg27113374, | cg04926368, | cg22929808, | cg07116337, | cg10433052, |
| cg08624180, | cg27294324, | cg11411203, | cg02152091, | cg09753772, | cg18040813, | cg19704348, |
| cg01852715, | cg08850264, | cg09933279, | cg27407935, | cg26874534, | cg08898653, | cg20737995, |
| cg04913118, | cg14696535, | cg14603085, | cg01100703, | cg14045872, | cg11222503, | cg25310430, |
| cg11574184, | cg24133241, | cg09514588, | cg12065366, | cg15593697, | cg08279035, | cg04789362, |
| cg14685796, | cg12204166, | cg02611934, | cg00415782, | cg08419523, | cg07749454, | cg15177613, |
| cg16025094, | cg00691974, | cg04219510, | cg07362168, | cg06507377, | cg08321330, | cg24344394, |
| cg01422797, | cg13014623, | cg12760261, | cg22654142, | cg02751839, | cg11819013, | cg14908653, |
| cg03386791, | cg11245443, | cg16161425, | cg02212836, | cg18345369, | cg24472939, | cg20482112, |
| cg18882819, | cg07554154, | cg04232681, | cg13179508, | cg20225681, | cg09239106, | cg25733898, |
| cg24429708, | cg07095252, | cg18082842, | cg23666536, | cg14742802, | cg18448949, | cg16145821, |
| cg13488020, | cg03764585, | cg25287375, | cg12402108, | cg15406536, | cg01030178, | cg11343579, |
| cg01840268, | cg05513511, | cg12403329, | cg27002247, | cg01655898, | cg04323069, | cg19284277, |
| cg24010952, | cg18861713, | cg16020747, | cg10959242, | cg11453608, | cg08301941, | cg13038108, |
| cg03559815, | cg02073492, | cg22107662, | cg08784238, | cg05983405, | cg15519670, | cg20863107, |
| cg19573198, | cg00336320, | cg12059793, | cg04274844, | cg18545076, | cg21330419, | cg11019835, |
| cg24140586, | cg09186897, | cg02197542, | cg09472139, | cg07108579, | cg08313393, | cg22502492, |
| cg11432034, | cg15351214, | cg20453985, | cg14909842, | cg23978104, | cg24180975, | cg03771185, |
| cg13301676, | cg20230508, | cg04956801, | cg15281225, | cg16224854, | cg12573791, | cg00920254, |
| cg18784533, | cg11582189, | cg05882522, | cg25745713, | cg10425986, | cg10799386, | cg19809453, |
| cg00581130, | cg06200048, | cg13577361, | cg02695677, | cg06642945, | cg06181784, | cg01295022, |
| cg02222066, | cg15233114, | cg05232576, | cg24133569, | cg19171175, | cg09602803, | cg07389870, |
| cg24749475, | cg05181282, | cg03020810, | cg02403294, | cg16873684, | cg00218767, | cg23531488, |
| cg01444413, | cg23664708, | cg00114008, | cg22339091, | cg03312572, | cg16905586, | cg00791868, |
| cg21472050, | cg07012091, | cg08095532, | cg06718080, | cg11368597, | cg15646900, | cg11971077, |
| cg10154633, | cg18830952, | cg17015897, | cg07729025, | cg08521270, | cg15482928, | cg13666729, |
| cg11675148, | cg21705505, | cg12319602, | cg08958931, | cg06197571, | cg11931116, | cg14408447, |
| cg01090930, | cg05830416, | cg17083560, | cg05861639, | cg19856897, | cg27366888, | cg03294719, |
| cg19085507, | cg11282718, | cg10078639, | cg02264038, | cg04324666, | cg11339445, | cg14807682, |
| cg13560123, | cg23623659, | cg18016194, | cg18343881, | cg04706229, | cg17825384, | cg21151335, |
| cg09138430, | cg05874882, | cg23429507, | cg25736328, | cg00909242, | cg20539366, | cg03115072, |
| cg17237962, | cg03424727, | cg19412964, | cg10866062, | cg05439503, | cg05328197, | cg14161359, |
| cg13586425, | cg26301661, | cg02649987, | cg05622647, | cg23548163, | cg02628902, | cg05968145, |
| cg15519535, | cg07725970, | cg08372135, | cg08758352, | cg02528189, | cg00533649, | cg21597811, |
| cg23104539, | cg21972318, | cg26144629, | cg14350318, | cg16163116, | cg10171388, | cg00732775, |
| cg03975537, | cg07318231, | cg12995090, | cg03466331, | cg03751030, | cg23911972, | cg06626184, |
| cg11358199, | cg11040940, | cg09747670, | cg11886639, | cg24937706, | cg11663780, | cg03636964, |
| cg11658874, | cg26002474, | cg08836009, | cg12545570, | cg26925669, | cg16429975, | cg06618957, |
| cg08467687, | cg06885782, | cg19927058, | cg05849324, | cg00717693, | cg06024289, | cg17057934, |
| cg13659419, | cg06910257, | cg03727481, | cg19470159, | cg24307251, | cg10512020, | cg13475977, |
| cg08635689, | cg02577240, | cg14664030, | cg15139063, | cg11804953, | cg21501518, | cg12433831, |
| cg06406719, | cg09723161, | cg10899547, | cg09743009, | cg24692170, | cg05921579, | cg21843114, |
| cg05058313, | cg22849149, | cg25523633, | cg13291466, | cg11367354, | cg15254424, | cg22974452, |
| cg01877391, | cg17724366, | cg22241443, | cg24359109, | cg02825211, | cg12479933, | cg16055869, |
| cg14642045, | cg09337049, | cg26125625, | cg09749813, | cg14238376, | cg24293567, | cg14011734, |
| cg09901735, | cg25899154, | cg17649243, | cg24113449, | cg10929299, | cg23840821, | cg23092527, |
| cg22420249, | cg14304702, | cg23484234, | cg17775962, | cg24864413, | cg12837896, | cg00622654, |
| cg04109349, | cg22954703, | cg25725154, | cg00831633, | cg26039848, | cg18721888, | cg13065228, |
| cg00210133, | cg01079632, | cg05970768, | cg14852486, | cg21451504, | cg14324805, | cg11903188, |
| cg10436414, | cg07483007, | cg07995893, | cg18697143, | cg09588254, | cg05181941, | cg14685948, |
| cg00780845, | cg25932496, | cg10850930, | cg14453935, | cg22031783, | cg06089468, | cg16900264, |
| cg02466113, | cg01315957, | cg08007378, | cg04083553, | cg11235297, | cg21006031, | cg23530245, |
| cg04643909, | cg14029471, | cg12289476, | cg01905172, | cg03978961, | cg07697672, | cg08622729, |
| cg17286435, | cg09448707, | cg12627202, | cg00492269, | cg18178715, | cg20480723, | cg02765085, |
| cg01408558, | cg22510074, | cg06895849, | cg24877842, | cg06017847, | cg15825692, | cg26386788, |
| cg22663124, | cg26646392, | cg17802551, | cg23143553, | cg04361749, | cg03838635, | cg23613157, |
| cg13445891, | cg14794799, | cg22608996, | cg08381620, | cg21338747, | cg25736982, | cg26706909, |
| cg17579580, | cg10797387, | cg01493020, | cg13301733, | cg27325460, | cg25188407, | cg20705804, |
| cg26302548, | cg08428819, | cg23271556, | cg19134728, | cg00871371, | cg07797693, | cg22140675, |
| cg03704899, | cg09914675, | cg26648103, | cg21050076, | cg08012278, | cg03417340, | cg02591634, |
| cg13660372, | cg25618916, | cg00078270, | cg17475200, | cg09634031, | cg15976388, | cg02716776, |
| cg13226232, | cg14182690, | cg02006134, | cg12443195, | cg08549390, | cg13412461, | cg16501033, |
| cg10698984, | cg07722360, | cg01018260, | cg04287755, | cg09605893, | cg02882785, | cg14101380, |
| cg26018965, | cg06760710, | cg05005113, | cg24743310, | cg02360445, | cg03658578, | cg25412641, |
| cg17146291, | cg13308187, | cg18663341, | cg08410301, | cg13719084, | cg24669129, | cg05949800, |
| cg16695890, | cg00599402, | cg23843505, | cg17221738, | cg17582444, | cg06414854, | cg00696540, |
| cg27162464, | cg04937794, | cg02441015, | cg26718510, | cg21432302, | cg04394031, | cg19760241, |
| cg08272591, | cg19364879, | cg12878382, | cg09188099, | cg18575770, | cg03578193, | cg06197616, |
| cg02594410, | cg13423151, | cg01016637, | cg02686533, | cg05516942, | cg02107844, | cg04948483, |
| cg21483906, | cg11224334, | cg26736341, | cg04578090, | cg23311905, | cg24577452, | cg01460805, |
| cg04793826, | cg07921314, | cg11936639, | cg17467550, | cg00880452, | cg12503257, | cg09383596, |
| cg16708623, | cg09351636, | cg25973273, | cg18073115, | cg17490790, | cg27025995, | cg03928367, |
| cg11687746, | cg10624105, | cg10837783, | cg25915871, | cg08680415, | cg17351085, | cg23315637, |
| cg00753885, | cg14316440, | cg27625491, | cg22623303, | cg17355791, | cg18315835, | cg20215290, |
| cg03046445, | cg04939326, | cg20126158, | cg08440086, | cg14858504, | cg02824064, | cg07916071, |
| cg14190943, | cg16882301, | cg14150907, | cg08155908, | cg26166177, | cg25261059, | cg11736589, |
| cg05003890, | cg10922229, | cg17899761, | cg23482746, | cg08221288, | cg23933241, | cg14490686, |
| cg03293882, | cg08973927, | cg24689583, | cg12052957, | cg22647566, | cg08773078, | cg21660130, |
| cg24437943, | cg21145524, | cg23346781, | cg14425252, | cg10524152, | cg24217003, | cg26893816, |
| cg24155792, | cg05470468, | cg14653284, | cg18773383, | cg03765041, | cg15073631, | cg11461694, |
| cg15827508, | cg03068646, | cg22550549, | cg15099273, | cg11790580, | cg05036032, | cg26075639, |
| cg02820836, | cg11683125, | cg22891003, | cg19325139, | cg07636194, | cg12246416, | cg16856100, |
| cg13777150, | cg00397559, | cg04984556, | cg17818792, | cg24631877, | cg26223654, | cg18118399, |
| cg24589564, | cg22488352, | cg05255057, | cg25918166, | cg04562562, | cg02856070, | cg06700061, |
| cg03071195, | cg14591667, | cg14843216, | cg07941978, | cg05979400, | cg01671587, | cg22533025, |
| cg03679521, | cg13341536, | cg03215105, | cg03644281, | cg08557393, | cg16511994, | cg16128638, |
| cg05890616, | cg02126603, | cg07378013, | cg13195796, | cg11015497, | cg23206325, | cg16892887, |
| cg04769514, | cg16365973, | cg22317004, | cg21784383, | cg17684478, | cg04656330, | cg26938153, |
| cg12012932, | cg11945276, | cg09144608, | cg13711538, | cg16569147, | cg10134799, | cg08349602, |
| cg16803791, | cg19107655, | cg09725202, | cg13390480, | cg02255090, | cg19802165, | cg12024292, |
| cg19722781, | cg24934561, | cg00713286, | cg22541143, | cg15585264, | cg26275858, | cg06635328, |
| cg21926875, | cg21235025, | cg27493301, | cg01491071, | cg14567085, | cg19246600, | cg12719622, |
| cg08462988, | cg16026627, | cg09427030, | cg00241076, | cg17491300, | cg06809458, | cg10875257, |
| cg18270343, | cg26433034, | cg06764092, | cg13804478, | cg11690983, | cg00853296, | cg20986887, |
| cg12086462, | cg24269840, | cg11612470, | cg26155867, | cg06976395, | cg04437762, | cg03901281, |
| cg07428323, | cg12876866, | cg27193366, | cg10841775, | cg27296293, | cg17139322, | cg00556408, |
| cg08370757, | cg01728704, | cg17504145, | cg25591867, | cg03759824, | cg14980363, | cg13740709, |
| cg18047920, | cg16722931, | cg07275436, | cg24546683, | cg10632209, | cg12488585, | cg00089945, |
| cg05552908, | cg06569419, | cg07816637, | cg06720232, | cg19304935, | cg06928346, | cg08450404, |
| cg14832998, | cg22776235, | cg08090835, | cg14851383, | cg06454283, | cg03116642, | cg20133730, |
| cg17890782, | cg12013342, | cg18038372, | cg23740473, | cg12156759, | cg01418124, | cg11287055, |
| cg05852568, | cg25541621, | cg02358382, | cg13092575, | cg05413465, | cg01018492, | cg13526221, |
| cg16585972, | cg10198932, | cg16693012, | cg15358752, | cg15786705, | cg09726509, | ch.6.108097463R, |
| cg17400113, | cg18493677, | cg24607290, | cg09915983, | cg14613960, | cg25255679, | cg25611931, |
| cg23012700, | cg15646463, | cg17156100, | cg27019712, | cg05293881, | cg03530437, | cg04498014, |
| cg25021893, | cg23471482, | cg24372558, | cg25697769, | cg23537820, | cg21572688, | ch.6.1238048R, |
| cg07084358, | cg22541679, | cg19564098, | cg14259556, | cg13774184, | cg06371291, | cg04084295, |
| cg16630546, | cg14639753, | cg09273516, | cg15503722, | cg26529437, | cg24385334, | cg24511782, |
| cg04713042, | cg06007675, | cg06320418, | cg02128278, | cg27342122, | cg20676475, | cg23090583, |
| cg02035751, | cg09139144, | cg22784589, | cg19365706, | cg00162401, | cg19547370, | cg22535228, |
| cg19819595, | cg25723394, | cg18642576, | cg25235068, | cg01667892, | cg17910274, | cg13557939, |
| cg06571993, | cg18900649, | cg20310759, | cg05511613, | cg09562426, | cg12389461, | cg10234506, |
| cg10123733, | cg00193283, | cg10266386, | cg04878072, | cg24425757, | cg07810726, | cg06824467, |
| cg06100231, | cg16951108, | cg24335155, | cg19604641, | cg08189043, | cg07397372, | cg08839546, |
| cg04330806, | cg18518914, | cg25292468, | cg12980408, | cg18001780, | cg13105730, | cg14125846, |
| cg00678005, | cg04812726, | cg11485012, | cg14556787, | cg07338658, | cg18052222, | cg19206040, |
| cg19921130, | cg03328750, | cg24328944, | cg12297590, | cg24132141, | cg25037439, | cg14854517, |
| cg24348240, | cg22762745, | cg27649916, | cg24847554, | cg21463140, | cg26152188, | cg17610800, |
| cg25833031, | cg18636679, | cg02839896, | cg12365451, | cg01749076, | cg09797577, | cg04367345, |
| cg02531787, | cg20259398, | cg22490861, | cg11026604, | cg07249433, | cg18249107, | cg23193730, |
| cg11080552, | cg21621204, | cg07889936, | cg18113803, | cg13848379, | cg23138678, | cg00039463, |
| cg20933713, | cg25568297, | cg24602552, | cg01324883, | cg21960680, | cg14535531, | cg05262634, |
| cg18488623, | cg08316073, | cg25856090, | cg15602829, | cg10572274, | cg10773064, | cg24738460, |
| cg26755141, | cg22560637, | cg05340495, | cg18139178, | cg06297554, | cg13407262, | cg09519273, |
| cg04756698, | cg04325301, | cg01302562, | cg18331348, | cg23828467, | cg15438404, | cg13062396, |
| cg03312716, | cg15052335, | cg13707780, | cg27260630, | cg24334259, | cg17458653, | cg03016153, |
| cg21649660, | cg22795610, | cg11203401, | cg11361121, | cg06009620, | cg23913941, | cg24529736, |
| cg22734086, | cg13646645, | cg05952475, | cg04391048, | cg26331096, | cg01217329, | cg16650561, |
| cg01236137, | cg02253440, | cg03040282, | cg09395969, | cg03393238, | cg18997701, | cg09826784, |
| cg14979301, | cg10537699, | cg04425094, | cg02713162, | cg15982419, | cg12761101, | cg13875003, |
| cg07201717, | cg05977781, | cg04796763, | cg08392199, | cg11885471, | cg25533774, | cg03561389, |
| cg04407872, | cg17988535, | cg25565383, | cg11514356, | cg15857661, | cg25350198, | cg05332489, |
| cg19024381, | cg06595320, | cg02990814, | cg01932091, | cg08615699, | cg23916205, | cg02805690, |
| cg13447933, | cg03631294, | cg05014933, | cg00488514, | cg26162616, | cg06995285, | cg08017850, |

DMP IDs:

| | | | | |
|---|---|---|---|---|
| cg27211696, | cg13601309, | cg18777231, | cg13851904, | cg20292653, |
| cg09092052, | cg03682117, | cg00806704, | cg07156219, | cg23664192, |
| cg14601709, | cg26685195, | cg21870229, | cg02016771, | cg20316284, |
| cg10807105, | cg12310370, | cg13446110, | cg18152871, | cg25044876, |
| cg19012170, | cg00651401, | cg22621310, | cg21282663, | cg00128482, |
| cg13908074, | cg11532578, | cg02290823, | cg22329555, | cg24815999, |
| cg13632729, | cg01686177, | cg26516941, | cg15355388, | cg25966295, |
| cg24729988, | cg06164712, | cg19595912, | cg02636947, | cg02490189, |
| cg04958528, | cg20627099, | cg11749284, | cg00950385, | cg16398686, |
| cg21208943, | cg25977879, | cg15601915, | cg18292904, | cg23061027, |
| cg12181217, | cg24517325, | cg26426564, | cg26650846, | cg23526055, |
| cg22102082, | cg15287024, | cg17942436, | cg16048851, | cg26853536, |
| cg02785604, | cg18397726, | cg18649319, | cg25113008, | cg01277490, |
| cg24057718, | cg08900316, | cg25390635, | cg15536165, | cg18145080, |
| cg24866702, | cg01623627, | cg02579494, | cg02410524, | cg04791456, |
| cg01368068, | cg15858239, | cg18544809, | cg05708167, | cg22972519, |
| cg14659008, | cg21210409, | cg16583536, | cg24001710, | cg17329534, |
| cg23148467, | cg10316662, | cg13576178, | cg23652939, | cg02310562, |
| cg03524083, | cg27476279, | cg17548900, | cg12972290, | cg02153397, |
| cg09426937, | cg24199006, | cg16757423, | cg21900495, | cg09000779, |
| cg18459342, | cg21930061, | cg03623264, | cg04227238, | cg13460409, |
| cg17014647, | cg26314765, | cg06348427, | cg10684627, | cg19953799, |
| cg14142977, | cg01197871, | cg15095771, | cg04781638, | cg03442376, |
| cg18326719, | cg15522241, | cg20797270, | cg21211357, | cg17270808, |
| cg10560514, | cg16452086, | cg10958362, | cg07095599, | cg03066754, |
| cg17055959, | cg04282497, | cg03139840, | cg05791173, | cg01477361, |
| cg05980726, | cg08875705, | cg01567825, | cg23414876, | cg17003212, |
| cg23400942, | cg18101140, | cg05009601, | cg07920838, | cg11685162, |
| cg06751446, | cg03146452, | cg22728551, | cg05366812, | cg27411712, |
| cg14280533, | cg08456140, | cg00023168, | cg07716287, | cg00325866, |
| cg04250451, | cg21686944, | cg02942287, | cg14225678, | cg15717165, |
| cg24740026, | cg17439097, | cg17245223, | cg27063932, | cg18972517, |
| cg05420994, | cg05678729, | cg05694450, | cg04428805, | cg00456551, |
| cg03768171, | cg03344526, | cg14403327, | cg18842598, | cg06580891, |
| cg26477221, | cg12258307, | cg24927323, | cg16736259, | cg25171856, |
| cg26662579, | cg27568306, | cg08538869, | cg05371005, | cg11802027, |
| cg08877463, | cg10883421, | cg10243196, | cg14126608, | cg08054217, |
| cg23110891, | cg01773061, | cg22746681, | cg15505531, | cg25693302, |
| cg06196466, | cg24913355, | cg15698545, | cg17491339, | cg20502080, |
| cg03967431, | cg08202769, | cg10871876, | cg03352106, | cg17297475, |
| cg01434487, | cg26217633, | cg17682794, | cg03182044, | cg00232827, |
| cg12612104, | cg01768342, | cg03552804, | cg22539434, | cg10896623, |
| cg10971048, | cg12278754, | cg23338753, | cg04828158, | cg21519172, |
| cg05485520, | cg04574826, | cg13487575, | cg22868433, | cg11408632, |
| cg22114827, | cg16245174, | cg00121562, | cg17551002, | cg17271592, |
| cg00033666, | cg08814947, | cg13811808, | cg12435074, | cg12733430, |
| cg02565715, | cg02809401, | cg17466474, | cg13294849, | cg12486756, |
| cg01584448, | cg26819590, | cg23238147, | cg14527389, | cg26124016, |
| cg23279929, | cg27580050, | cg16258229, | cg11845159, | cg10837251, |
| cg23501836, | cg22871002, | cg24603235, | cg14685006, | cg00210210, |
| cg02572463, | cg01145544, | cg08410901, | cg01557754, | cg13631259, |
| cg08467261, | cg23890800, | cg23035555, | cg08099701, | cg12710480, |
| cg01083434, | cg26818786, | cg03554406, | cg22282877, | cg27412039, |
| cg26762173, | cg00583003, | cg00907884, | cg25611840, | cg21547118, |
| cg10338082, | cg03784054, | cg16290866, | cg11336539, | cg27380915, |
| cg00425431, | cg04591563, | cg18576588, | cg08411422, | cg02631868, |
| cg01725531, | cg11139248, | cg13996731, | cg14364447, | cg15318627, |
| cg04120546, | cg09414863, | cg06689205, | cg17716463, | cg09441223, |
| cg23090159, | cg17603689, | cg24283842, | cg02669544, | cg17066620, |
| cg24578875, | cg21455927, | cg23843620, | cg15121877, | cg06666157, |
| cg01716499, | cg00682053, | cg15650161, | cg17256260, | cg24332298, |
| cg27591375, | cg24933241, | cg10207181, | cg14910450, | cg19794674, |
| cg10010036, | cg09531376, | cg10086328, | cg09077530, | cg00356500, |
| cg08305803, | cg14167385, | cg03331400, | cg15444947, | cg16529592, |
| cg26369315, | cg22145576, | cg19879675, | cg04943065, | cg24014869, |
| cg16886828, | cg24549277, | cg08404299, | cg24733497, | cg19979646, |
| cg09635586, | cg07157834, | cg11597080, | cg20140034, | cg00862894, |
| cg20965815, | cg06709324, | cg10858828, | cg10155522, | cg09343092, |
| cg11618529, | cg08712824, | cg04222344, | cg05413277, | cg25318296, |
| cg19702771, | cg07957953, | cg26484247, | cg18704218, | cg16009120, |
| cg03393966, | cg13610138, | cg01392338, | cg20108693, | cg00161229, |
| cg07629007, | cg19614643, | cg00546248, | cg03790324, | cg08796391, |
| cg21054179, | cg23884217, | cg03118021, | cg22339356, | cg00338529, |
| cg12577199, | cg09323228, | cg12227505, | cg16201899, | cg22614759, |
| cg19226934, | cg02066441, | cg20367712, | cg13660719, | cg14893935, |
| cg13577231, | cg10730497, | cg22076160, | cg00669044, | cg02639007, |
| cg05779081, | cg14490972, | cg18904477, | cg05146223, | cg00584000, |
| cg05751979, | cg20934416, | cg04064611, | cg11286618, | cg08173404, |
| cg01512155, | cg14360865, | cg00209398, | cg09956907, | cg21205282, |
| cg07534823, | cg03176810, | cg08547601, | cg07206256, | cg19199805, |
| cg10314752, | cg21997198, | cg07167632, | cg22993707, | cg03996106, |
| cg13156014, | cg11763094, | cg11596009, | cg19024969, | cg20217872, |
| cg24076669, | cg24026690, | cg25693132, | cg12293170, | cg19416590, |
| cg03934443, | cg18740800, | cg05458429, | cg20906345, | cg02263479, |
| cg16793173, | cg22086536, | cg19741456, | cg22334962, | cg13904806, |
| cg18262937, | cg08089449, | cg25604819, | cg05556917, | cg20705565, |
| cg09909381, | cg09644451, | cg18476517, | cg00047050, | cg20625588, |
| cg23425374, | cg14834391, | cg05824174, | cg11857651, | cg12104984, |
| cg24731635, | cg04635154, | cg15684116, | cg15498349, | cg06095115, |
| cg26598649, | cg10888854, | cg16409505, | cg06561646, | cg23641964, |
| cg24188111, | cg25350011, | ch.1.2582316R, | cg04481722, | cg00606312, |
| cg18952098, | cg03070194, | cg18867659, | cg06133097, | cg27060240, |
| cg08098450, | cg23730222, | cg02820646, | cg09272217, | cg21269393, |
| cg00608605, | cg14971327, | cg04528808, | cg08545463, | cg00242951, |
| cg16203262, | cg03970319, | cg03456560, | cg16193946, | cg15997411, |
| cg04879750, | cg06036912, | cg11617975, | cg18770758, | cg02819010, |
| cg12877853, | cg16381207, | cg08855729, | cg23408285, | cg20531035, |
| cg17940673, | cg15541987, | cg27003571, | cg11438560, | cg09246103, |
| cg17212902, | cg05869173, | cg02601318, | cg03860859, | cg07356130, |
| cg14506320, | cg27295678, | cg02193951, | cg08994336, | cg10640830, |
| cg00346434, | cg12837905, | cg06804177, | cg02349264, | cg09293534, |
| cg23357526, | cg17552368, | cg18020065, | cg20014441, | cg03146637, |
| cg18798922, | cg09395540, | cg14379939, | cg18988861, | cg14424363, |
| cg19250891, | cg06625677, | cg12126027, | cg24438178, | cg05505450, |
| cg01621034, | cg14323539, | cg09049451, | cg07143957, | cg03863549, |
| cg22198397, | cg08411886, | cg17983571, | cg09418511, | cg18070458, |
| cg16932017, | cg09926486, | cg16775856, | cg04406873, | cg02327465, |
| cg26831566, | cg16253059, | cg04229103, | cg14473235, | cg07556730, |
| cg08284926, | cg20417180, | cg00105041, | cg15619546, | cg19230917, |
| cg06398114, | cg09554856, | cg17500228, | cg27009208, | cg22685009, |
| cg00452622, | cg01948223, | cg16674535, | cg24062526, | cg19304088, |
| cg15615396, | cg04971534, | cg24046689, | cg24989181, | cg23238315, |
| cg17356733, | cg18982286, | cg01281501, | cg07003900, | cg08507638, |
| cg18947720, | cg12535090, | cg15126273, | cg09937500, | cg14872736, |
| cg17028652, | cg03243497, | cg07576931, | cg15167008, | cg22061561, |
| cg03326125, | cg24579887, | cg04969808, | cg06191898, | cg04636402, |
| cg01049530, | cg25771096, | cg12389767, | cg18003214, | cg22585786, |
| cg25490475, | cg06519422, | cg16412000, | cg15712226, | cg15471946, |
| cg08476984, | cg11496559, | cg00969119, | cg23349726, | cg05590156, |
| cg11762839, | cg02182308, | cg10150813, | cg00495362, | cg10589072, |
| cg09656152, | cg26695784, | cg16100244, | cg02503395, | cg02363202, |
| cg18048405, | cg01362115, | cg12382846, | cg17078815, | cg06815976, |
| cg03172837, | cg25149218, | cg08530544, | cg22595274, | cg22493372, |
| cg26730416, | cg07805238, | cg23172664, | cg18348318, | cg07944724, |
| cg14671000, | cg10832107, | cg03480346, | cg24700702, | cg24155429, |
| cg13655928, | cg15724985, | cg13012746, | cg01317270, | cg07152925, |
| cg19597545, | cg09265940, | cg12526197, | cg12211557, | cg08148814, |
| cg05753794, | cg01649456, | cg18563413, | cg22331138, | cg26709693, |
| cg06919800, | cg26335577, | cg18634690, | cg26566283, | cg15496335, |
| cg13533935, | cg04843252, | cg11023075, | cg02757236, | cg03478444, |
| cg13494037, | cg04108334, | cg11877251, | cg14949292, | cg10648139, |
| cg03267400, | cg19276371, | cg19187616, | cg06419209, | cg20814095, |
| cg19227382, | cg02524112, | cg23225572, | cg03979378, | cg09176847, |
| cg14835575, | cg04871498, | cg19536664, | cg10035825, | cg10002178, |
| cg02951021, | cg16740092, | cg00762738, | cg11858516, | cg15928780, |
| cg05100282, | cg01472813, | cg20577572, | cg15679747, | cg02470080, |
| cg11556164, | cg09169779, | cg16123319, | cg27064287, | cg14712964, |
| cg25696949, | cg16715953, | cg22777952, | cg03128454, | cg09259115, |
| cg22378252, | cg12067928, | ch.15.403279R, | cg02339592, | cg17943175, |
| cg20044666, | cg00911488, | cg04390004, | cg06275813, | cg21961890, |
| cg07705739, | cg06685766, | cg23397427, | cg16098726, | cg20286347, |
| cg17210837, | cg27507295, | cg18107268, | cg26495758, | cg03537740, |
| cg16082606, | cg10226922, | cg21006727, | cg15923100, | cg02318426, |
| cg04994795, | cg02182587, | cg03484160, | cg04848555, | cg16209303, |
| cg00093436, | cg11682012, | cg14348182, | cg22161115, | cg14566901, |
| cg18398007, | cg11159591, | cg18444323, | cg20200035, | cg12792367, |
| cg06128028, | cg15108705, | cg06379368, | cg05717794, | cg05625955, |
| cg05529278, | cg13543375, | cg06580419, | cg05383565, | cg02881158, |
| cg00647095, | cg19250935, | cg12228123, | cg10055505, | cg06195193, |
| cg01442781, | cg09038183, | cg08308510, | cg09037532, | cg11218561, |
| cg22266072, | cg16430510, | cg19538722, | cg03816062, | cg09636245, |
| cg02188818, | cg19582557, | cg14796305, | cg11724557, | cg01628067, |
| cg06481462, | cg10261205, | cg07975813, | cg09367648, | cg16591304, |
| cg15952586, | cg23148992, | cg09774706, | cg18560328, | cg23780351, |
| cg24654176, | cg19159396, | cg14122373, | cg03943509, | cg02298881, |
| cg00144817, | cg12949975, | cg02408775, | cg07005090, | cg24046864, |
| cg17371350, | cg04215213, | cg04091325, | cg20358313, | cg10157972, |
| cg08655148, | cg19919590, | cg27614120, | cg01176047, | cg02927448, |
| cg05996795, | cg13855364, | cg14827056, | cg15540507, | cg18025976, |
| cg09195657, | cg24536827, | cg19743666, | cg16764274, | cg10811945, |
| cg02982734, | cg26824126, | cg14633135, | cg09293786, | cg15646123, |
| cg10762570, | cg09963654, | cg04043150, | cg07892597, | cg11195797, |
| cg27501522, | cg17121140, | cg02565647, | cg00372375, | cg22022568, |
| cg26023938, | cg12210283, | cg15739881, | cg09074223, | cg10719247, |
| cg14069214, | cg06586813, | cg15369054, | cg24394336, | cg18851831, |
| cg16952751, | cg05557255, | cg08275504, | cg01572696, | cg23239840, |
| cg12260192 | | | | |

¹CvC—cancer vs. control samples (TCGA dataset)
²PvR—progressive vs. regressive CIS dataset

TABLE 4
Metrics:

| Number | ROC AUC | ROC | Threshold | Sensitivity | Specificity | Sensitivity | Specificity |
|---|---|---|---|---|---|---|---|
| of DMPs | (CvC¹) | (PvR²) | value | (CvC) | (CvC) | (PvR) | (PvR) |
| 2000 | 0.983462033 | 0.797067901 | 0.36 | 1 | 0.467567568 | 0.888888889 | 0.694444444 |

DMP IDs:

| | | | | | | |
|---|---|---|---|---|---|---|
| cg00653281, | cg01477942, | cg22512438, | cg08805748, | cg13886636, | cg09569536, | cg16983730, |
| cg20099054, | cg14071249, | cg12296552, | cg13830163, | cg08498533, | cg01130974, | cg12184864, |
| cg08232654, | cg04452437, | cg06118999, | cg24278087, | cg03570976, | cg22657457, | cg09376680, |
| cg21205032, | cg09739212, | cg23679260, | cg21681549, | cg13606025, | cg23465426, | cg06844837, |
| cg27312916, | cg04237288, | cg05912079, | cg05138150, | cg24419560, | cg00707300, | cg11069071, |
| cg17851021, | cg03963049, | cg11333835, | cg16142965, | cg18394118, | cg07575518, | cg14130977, |
| cg22605799, | cg20536369, | cg18666249, | cg14507868, | cg04084172, | cg22886005, | cg06434454, |
| cg08854799, | cg19547929, | cg26137068, | cg06996147, | cg06170425, | cg09718251, | cg05322019, |
| cg01077846, | cg16408735, | cg26133372, | cg23963153, | cg24882332, | cg08147813, | cg14150071, |
| cg26139338, | cg03310779, | cg11377642, | cg08437984, | cg02389091, | cg04939555, | cg06163593, |
| cg09786098, | cg24639487, | cg19256589, | cg10992686, | cg07596883, | cg10092251, | cg27405988, |
| cg22705746, | cg08187779, | cg09835278, | cg15426626, | cg15405028, | cg07557690, | cg06550082, |
| cg00612633, | cg05852395, | cg09653641, | cg11401558, | cg21009978, | cg11219400, | cg14616667, |
| cg07204874, | cg20253172, | cg01013955, | cg25761802, | cg16505687, | cg04718185, | cg05180443, |
| cg04511125, | cg19806995, | cg03304763, | cg02157002, | cg10594414, | cg10886442, | cg23473114, |
| cg26490839, | cg12643458, | cg16825211, | cg26940676, | cg00995988, | cg03118626, | cg11185975, |
| cg15883761, | cg22866835, | cg04783338, | cg22109056, | cg10222823, | cg09262217, | cg18224993, |
| cg21654383, | cg02067350, | cg11637544, | cg11590932, | cg10501629, | cg00793562, | cg14758218, |
| cg03217800, | cg09121930, | cg00873473, | cg08289130, | cg17323956, | cg17826753, | cg02728263, |
| cg19561453, | cg13294780, | cg04890237, | cg16264642, | cg06624154, | cg13908074, | cg15756407, |
| cg12411846, | cg02237415, | cg03869448, | cg08112137, | cg09867084, | cg17644827, | cg27389262, |
| cg17008770, | cg23019936, | cg22374474, | cg25717994, | cg08211068, | cg01834225, | cg15151293, |
| cg27003594, | cg27572074, | cg07462112, | cg13636462, | cg18062810, | cg23370512, | cg26757722, |
| cg03547924, | cg12557430, | cg02537769, | cg09886557, | cg15553532, | cg00144186, | cg13557417, |
| cg15701085, | cg21563192, | cg00360769, | cg16206480, | cg09067715, | cg03466032, | cg15359249, |
| cg10173649, | cg22481950, | cg19878762, | cg14479798, | cg15554007, | cg27441384, | cg07352158, |
| cg09623400, | ch.6.1094404R, | cg23195753, | cg26797233, | ch.20.23481851F, | cg25743482, | cg04132522, |
| cg06309980, | cg05736768, | cg16145113, | cg18015844, | cg05488984, | cg00945260, | cg09404429, |
| cg10227830, | cg27265307, | cg02150674, | cg02081925, | cg24272002, | cg07835443, | cg13945148, |
| cg27485730, | cg11150520, | cg14884793, | cg18311066, | cg07983575, | cg16099804, | cg05534678, |
| cg17569124, | cg08381437, | cg05975219, | cg09471659, | cg10294200, | cg03929569, | cg23618344, |
| cg14144732, | cg04174124, | cg07661564, | cg10616306, | cg00211609, | cg03302822, | cg22237300, |
| cg05578055, | cg11166600, | cg25205727, | cg01302201, | cg13635184, | cg06164608, | cg18158859, |
| cg07752026, | cg11229513, | cg26351764, | cg24091698, | cg08191530, | cg08774694, | cg27029377, |
| cg01591487, | cg12595697, | cg21204860, | cg05998089, | cg08734053, | cg04357272, | cg08112448, |
| cg26477257, | cg11292721, | cg17445830, | cg09269652, | cg20930329, | cg21526209, | cg08719366, |
| cg03432338, | cg18903093, | cg14294629, | cg08464208, | cg00151768, | cg15443822, | cg12022895, |
| cg17903999, | cg13373085, | cg18121066, | cg12616941, | cg01631596, | cg12693099, | cg05635036, |
| cg16235748, | cg07826522, | cg13895765, | cg14717796, | cg03040622, | cg00801196, | cg04401436, |
| cg20532418, | cg00818493, | cg27591349, | cg14793835, | cg06107293, | cg14107457, | cg11062418, |
| cg06902200, | cg08213607, | cg03789420, | cg01857748, | cg26162007, | cg26786407, | cg15313740, |
| cg08271366, | cg16613990, | cg14746032, | cg03356595, | cg13461130, | cg18224492, | cg12412384, |
| cg14189988, | cg08232572, | cg26137290, | cg06000330, | cg03282408, | cg08141806, | cg09890775, |
| cg24643211, | cg07057579, | cg26367275, | cg05798339, | cg07554251, | cg08944563, | cg24343973, |
| cg14554880, | cg01951489, | cg12269535, | cg27295373, | cg01813033, | cg17856559, | cg24338843, |
| cg16766459, | cg08793689, | cg06405765, | cg00938087, | cg14021170, | cg19860353, | cg01061391, |
| cg22218709, | cg02585598, | cg14163731, | cg19319490, | cg02519286, | cg08285033, | cg02132463, |
| cg26005980, | cg25049341, | cg23163283, | cg26297299, | ch.2.2825309R, | cg01415919, | cg03180953, |
| cg20481287, | cg08685096, | cg15304497, | cg08467595, | cg11193274, | cg20675040, | cg00128951, |
| cg21110505, | cg25739316, | cg10927555, | cg11054936, | cg09761478, | cg15146834, | cg17341703, |
| cg00695112, | cg17695537, | cg26308506, | cg18669687, | cg27315170, | cg04086388, | cg00944142, |
| cg08391356, | cg19280121, | cg02807859, | cg22483286, | cg12786548, | cg09699373, | cg05171227, |
| cg13312174, | cg03454375, | cg18825594, | cg08180032, | cg07789083, | cg06696063, | cg00648274, |
| cg18873878, | cg22395192, | cg10675614, | cg01407244, | cg13000490, | cg00410534, | cg08692430, |
| cg06297863, | cg01662504, | cg13402656, | cg20002283, | cg05516295, | cg20678835, | cg00331508, |
| cg13267931, | cg08417719, | cg02017047, | cg18866586, | cg10672164, | cg05962522, | cg15697575, |
| cg24588339, | cg26576937, | cg12426467, | cg07741624, | cg25148733, | cg01268381, | cg25742035, |
| cg20662169, | cg27459437, | cg22868319, | cg10663781, | cg10601582, | cg14253096, | cg15612328, |
| cg08974450, | cg05990080, | cg25640699, | cg01909242, | cg19672271, | cg05270922, | cg11825883, |
| cg02695906, | cg24902339, | cg14403741, | cg04944682, | cg00946992, | cg14195958, | cg00494919, |
| cg26993947, | cg13015284, | cg16926284, | cg22092082, | cg10451977, | cg01204964, | cg14934533, |
| cg03177551, | cg08825075, | cg09256448, | cg27470087, | cg11072645, | cg16337763, | cg08432727, |
| cg04508233, | cg23606925, | cg06292304, | cg08267399, | cg00020474, | cg05370861, | cg08206092, |
| cg07681739, | cg14581928, | cg00320625, | cg17054360, | cg22277431, | cg06804311, | cg01743901, |
| cg15130102, | cg21958798, | cg12168219, | cg01133890, | cg17257944, | cg11247343, | cg17526300, |
| cg11290998, | cg26663609, | cg10692922, | cg22272282, | cg11565911, | cg03503871, | cg22547485, |
| cg17931890, | cg10071493, | cg20356136, | cg10775278, | cg15581269, | cg07202461, | cg19862964, |
| cg10181911, | cg13578447, | cg25923207, | cg04714110, | cg26832832, | cg13636096, | cg24030680, |
| cg01559446, | cg04103088, | cg00856157, | cg26400830, | cg26575166, | cg11789740, | cg21367866, |
| cg23563443, | cg26533107, | cg01662117, | cg12884381, | cg04786791, | cg05758434, | cg21286173, |
| cg00591791, | cg04946910, | cg27291182, | cg22198132, | cg26595643, | cg06482989, | cg18797229, |
| cg22329488, | cg01046703, | cg09480515, | cg26646411, | cg12893361, | cg24749475, | cg00028757, |
| cg19032271, | cg04609993, | cg02763234, | cg14080448, | cg21401642, | cg10153082, | cg20917484, |
| cg15090005, | cg25022341, | cg10948456, | cg20069407, | cg25785397, | cg13702678, | cg14671809, |
| cg00505381, | cg24236591, | cg00305320, | cg15648389, | cg18019017, | cg05046556, | cg24150244, |
| cg24938775, | cg13710703, | cg25175654, | cg18757974, | cg26408364, | cg13982601, | cg01722695, |
| cg00162643, | cg12071131, | cg22190705, | cg05723953, | cg01346077, | cg17826324, | cg10529796, |
| cg25017060, | cg26793846, | cg10262357, | cg11731300, | cg18404041, | cg17021436, | cg06747863, |
| cg16786297, | cg03936663, | cg05768824, | cg25942178, | cg00435576, | cg05976753, | cg19654882, |
| cg13455110, | cg13063344, | cg26944449, | cg22116290, | cg11880023, | cg21183606, | cg00657508, |
| cg26413691, | cg24277128, | cg16655385, | cg27187555, | cg16929354, | cg17933583, | ch.2.1448966F, |
| cg22656705, | cg13643287, | cg22336747, | cg22322535, | cg07549278, | cg14271505, | cg18099817, |
| cg18798446, | cg18886739, | cg14739859, | cg16986973, | cg09955859, | cg06074143, | cg02042621, |
| cg23725734, | cg13628888, | cg07562458, | cg05959111, | cg15355395, | cg23770265, | cg02382320, |
| cg25085245, | cg16478236, | cg07806394, | cg00314660, | cg00049323, | cg06897306, | cg25397562, |
| cg00952576, | cg16382382, | cg04025964, | cg23059701, | cg19725418, | cg06824583, | cg03399028, |
| cg04090147, | cg04537050, | cg15442678, | cg22123774, | cg24929264, | cg00371702, | cg07472227, |
| cg13341864, | cg05257472, | cg16984335, | cg10883542, | cg24079702, | cg02974259, | cg14254080, |
| cg00140479, | cg16719560, | cg20426994, | cg19659689, | cg25179853, | cg10504573, | cg05212260, |
| cg13256598, | cg24035898, | cg06145336, | cg20463033, | cg03724423, | cg17510645, | cg10266386, |
| cg26489439, | cg19474418, | cg18449997, | cg18393014, | cg07493232, | cg21547118, | cg15386103, |
| cg08191566, | cg19478820, | cg13457462, | cg20610891, | cg12474748, | cg10645426, | cg02602864, |
| cg12768681, | cg19472611, | cg05902438, | cg16907488, | cg08266095, | cg02860199, | cg03464514, |
| cg16340918, | cg19030875, | cg06104657, | cg04226401, | cg25684349, | cg08302365, | cg21621264, |
| cg03596877, | cg15828085, | cg09664442, | cg14655044, | cg13324919, | cg05936249, | cg05429319, |
| cg17407435, | cg18036710, | cg13949137, | cg05214130, | cg01201399, | cg10224783, | cg02025871, |
| cg20674067, | cg16981346, | cg24891702, | cg05735659, | cg00233335, | cg07745166, | cg20513448, |
| cg06107104, | cg10611115, | cg13843773, | cg16166796, | cg00292312, | cg12251571, | cg13575542, |
| cg02463791, | cg20712808, | cg17167832, | cg11678027, | cg07356415, | cg14183922, | cg20028827, |
| cg00174718, | cg02232988, | cg27247702, | cg25163297, | cg27614723, | cg04472685, | cg08205460, |
| cg27553433, | cg02852743, | cg14978826, | cg14280947, | cg16525300, | cg01392481, | cg13359415, |
| cg13983640, | cg13802151, | cg20822628, | cg02862886, | cg05983640, | cg02259743, | cg14484649, |
| cg08206623, | cg17756392, | cg03667033, | cg04484984, | cg12344605, | cg19880608, | cg22909962, |
| cg10746936, | cg10644575, | cg22062432, | cg07252851, | cg07724466, | cg06772309, | cg00681106, |
| cg21252282, | cg08812242, | cg18859033, | cg02128564, | cg04178787, | cg02584802, | cg17342141, |
| cg05867645, | cg02700626, | cg15268456, | cg07914457, | cg02267312, | cg13718539, | cg14688104, |
| cg20462795, | cg16199881, | cg11437120, | cg21103992, | cg18344930, | cg14643399, | cg27490380, |
| cg23153481, | cg14480679, | cg13910040, | cg16864658, | cg09202689, | cg14350176, | cg26455711, |
| cg11231240, | cg11797364, | cg14599823, | cg00346523, | cg00986053, | cg00191100, | cg27388462, |
| cg00052964, | cg03594972, | cg18026955, | cg04340651, | cg20404355, | cg10958964, | cg25421855, |
| cg10449839, | cg22960067, | cg09791812, | cg11861097, | cg05905168, | cg13488070, | cg10995925, |
| cg07252758, | cg19277926, | cg22540233, | cg03792768, | cg25120945, | cg19638749, | cg23790345, |
| cg18456367, | cg14793544, | cg03265268, | cg24340453, | cg11777890, | cg12340454, | cg06626184, |
| cg18802414, | cg22959932, | cg05746403, | cg15132169, | cg18752527, | cg04714201, | cg21947590, |
| cg00222175, | cg05243700, | cg01439366, | cg00572843, | cg07340574, | cg02369113, | cg12494180, |
| cg10177238, | cg18833822, | cg06475626, | cg09535963, | cg00846114, | cg18861112, | cg10837806, |
| cg06622968, | cg15991553, | cg23192255, | cg04850059, | cg27503015, | cg26719062, | cg22519189, |
| cg05928023, | cg16584092, | cg06872047, | cg04387739, | cg06221946, | cg14252602, | cg27250362, |
| cg04782470, | cg14426510, | cg12004115, | cg11267114, | cg24147849, | cg18846301, | cg04384626, |
| cg00651782, | cg02039539, | cg05020203, | cg25886345, | cg22168489, | cg06651878, | cg03584113, |
| cg15309421, | cg13627451, | cg21739009, | cg06013060, | cg19949853, | cg13662628, | cg21303803, |
| cg02931432, | cg09702334, | cg10802977, | cg06790305, | cg00977377, | cg11738446, | cg20440187, |
| cg10201671, | cg12647962, | cg03988380, | cg10797387, | cg27582017, | cg19527959, | cg04959674, |
| cg01082168, | cg16333846, | cg00820922, | cg18286127, | cg25926008, | cg17004733, | cg04209035, |
| cg23316923, | cg05052888, | cg06400428, | cg06226724, | cg18375494, | cg24655899, | cg17430370, |
| cg02916962, | cg27154651, | cg22919908, | cg18676033, | cg20140201, | cg04017533, | cg24338745, |
| cg00241941, | cg00699945, | cg16400647, | cg25241322, | cg04468281, | cg23534315, | cg25750901, |
| cg19962902, | cg11673741, | cg24348442, | cg22234061, | cg06458258, | cg13583454, | cg12601757, |
| cg01904774, | cg04026948, | cg13430439, | cg09381003, | cg25580825, | cg13003239, | cg13331550, |
| cg09829382, | cg02454028, | cg10976172, | cg08404739, | cg09280288, | cg25022355, | cg25595571, |
| cg24975662, | cg08756121, | cg20192362, | cg12518041, | cg13338734, | cg24804279, | cg05222958, |
| cg01300631, | cg21188037, | cg06734271, | cg25940827, | cg11123972, | cg17976731, | cg22367250, |
| cg24594507, | cg25036261, | cg11068238, | cg11576513, | cg15083233, | cg04682850, | cg06966887, |
| cg18469811, | cg14973797, | cg15982535, | cg00679281, | cg07979316, | cg15942382, | cg08596000, |
| cg16322208, | cg23305229, | cg10147348, | cg20377673, | cg09990790, | cg09449490, | cg25074809, |
| cg07556018, | cg06065695, | cg11041835, | cg12579676, | cg24906420, | cg25839439, | cg09954189, |
| cg00508123, | cg27378486, | cg21251385, | cg08504942, | cg08691479, | cg14125468, | cg06240275, |
| cg07250355, | cg09050372, | cg13763482, | cg01101911, | cg21015196, | cg24992817, | cg19234613, |
| cg11300777, | cg04888037, | cg02636222, | cg06157318, | cg26116551, | cg19224713, | cg08536358, |
| cg00400263, | cg16051561, | cg23886551, | cg06950023, | cg18578939, | cg18403792, | cg16446591, |
| cg01449563, | cg13922166, | cg07274618, | cg14673722, | cg08284873, | cg02106828, | cg05715003, |
| cg19803952, | cg12067736, | cg27280575, | cg02754295, | cg11715725, | cg24187253, | cg02825527, |
| cg27527108, | cg03276401, | cg01950247, | cg22505962, | cg26127114, | cg18513919, | cg01819707, |
| cg08362273, | cg10004882, | cg16583923, | cg22428623, | cg13524919, | cg25438801, | cg19399326, |
| cg20658709, | cg09005125, | cg08417229, | cg21985352, | cg01026503, | cg07002403, | cg17755082, |
| cg16102052, | cg02769522, | cg21542881, | cg15262317, | cg19653624, | cg17895511, | cg24061542, |
| cg17410974, | cg10443040, | cg07171492, | cg26766678, | cg22198946, | cg24843119, | cg01091258, |
| cg25517151, | cg22953731, | cg22757391, | cg06564811, | cg05172655, | cg13771214, | cg25511335, |
| cg13088465, | cg24166450, | cg17900495, | cg02515354, | cg19776833, | cg09654954, | cg12804677, |
| cg04315923, | cg12049949, | cg05851395, | cg07875360, | cg08743313, | cg14219854, | cg10040495, |
| cg19239230, | cg06038201, | cg19717595, | cg07108118, | cg25528916, | cg06636938, | cg18957721, |
| cg20741528, | cg08757924, | cg18274325, | cg01548743, | cg18625804, | cg00668215, | cg18331880, |
| cg11032810, | cg25625127, | cg11632592, | cg03863838, | cg17605107, | cg10615414, | cg26906021, |
| cg00525508, | cg12806763, | cg15605746, | cg01871526, | cg09205945, | cg01323840, | cg25338204, |
| cg14738670, | cg19696227, | cg13466481, | cg13463167, | cg21961970, | cg13372862, | cg16014770, |
| cg06927217, | cg04974062, | cg04536856, | cg20480070, | cg26415787, | cg23091737, | cg00927256, |
| cg16926316, | cg25109019, | cg25613180, | cg00414077, | cg10407585, | cg19539481, | cg16418598, |
| cg06151074, | cg13796518, | cg04992930, | cg20429748, | cg06507307, | cg18733255, | cg03476791, |
| cg17343451, | cg05836788, | cg05325028, | cg09413498, | cg00174713, | cg12177220, | cg24227728, |
| cg21264329, | cg15578601, | cg14124121, | cg16462006, | cg04922034, | cg02645778, | cg17585590, |
| cg10099827, | cg12492496, | cg03973420, | cg00895997, | cg21563219, | cg10662242, | cg04231677, |
| cg16430428, | cg09132058, | cg26574408, | cg15863539, | cg26870745, | cg21363811, | cg03760483, |
| cg17416793, | cg01771416, | cg18065874, | cg23246885, | cg21586203, | cg11799704, | cg27342251, |
| cg11726902, | cg24731015, | cg02162886, | cg14305711, | cg13475822, | cg26792576, | cg07894162, |
| cg04144394, | cg10207510, | cg02254885, | cg08784462, | cg08587802, | cg19510792, | cg09481972, |
| cg20458563, | cg02956372, | cg15972264, | cg04348247, | cg02959609, | cg04220893, | cg16589555, |
| cg02871995, | cg05376017, | cg04980452, | cg07250128, | cg14506751, | cg12573289, | cg09223513, |

DMP IDs:

| | | | | |
|---|---|---|---|---|
| cg12802329, | cg01952913, | cg19817544, | cg24319167, | cg23352424, |
| cg20974822, | cg08385486, | cg10824107, | cg03997502, | cg11353929, |
| cg14120667, | cg08218799, | cg07802401, | cg01458759, | cg22083053, |
| cg23225221, | cg19788317, | cg24354581, | cg00445548, | cg16214573, |
| cg12424230, | cg05888487, | cg13607699, | cg03684157, | cg19935065, |
| cg10503007, | cg13357903, | cg09852601, | cg18085405, | cg00509431, |
| cg08946995, | cg02189786, | cg18432105, | cg00454864, | cg16865965, |
| cg06832853, | cg16376379, | cg24935208, | cg01040533, | cg10886747, |
| cg02129159, | cg04971812, | cg01821426, | cg14101500, | cg03090436, |
| cg27222884, | cg17921548, | cg05483945, | cg24498500, | cg14906687, |
| cg10904061, | cg18415510, | cg24937275, | cg22870189, | cg07880384, |
| cg18709893, | cg15315015, | cg03528246, | cg03449406, | cg04892487, |
| cg19395074, | cg18688254, | cg07344528, | cg15156469, | cg22439783, |
| cg01614864, | cg01630869, | cg10130401, | cg05204798, | cg06445981, |
| cg04616793, | cg06431527, | cg01250212, | cg18306456, | cg17042098, |
| cg07130869, | cg24198944, | cg08759112, | cg17357285, | cg26530201, |
| cg26144909, | cg04962528, | cg01146614, | cg05748163, | cg00495713, |
| cg08068296, | cg15234155, | cg26871875, | cg16171484, | cg12298268, |
| cg27289137, | cg02305353, | cg05293990, | cg22043588, | cg13628577, |
| cg12459028, | cg23387692, | cg21640432, | cg08530317, | cg22154562, |
| cg03609148, | cg04146573, | cg25089357, | cg11889145, | cg16417447, |
| cg06524213, | cg11664956, | cg27262850, | cg11556559, | cg11011602, |
| cg04548096, | cg26441979, | cg14804635, | cg09253179, | cg08662052, |
| cg01456989, | cg23632029, | cg02115599, | cg08331163, | cg23979451, |
| cg02512226, | cg16202484, | cg04837923, | cg20455931, | cg07601804, |
| cg08173959, | cg05972308, | cg23984176, | cg24281422, | cg16080746, |
| cg25990848, | cg02483554, | cg03982441, | cg27462867, | cg26529376, |
| cg13444005, | cg16505927, | cg17424134, | cg10589249, | cg19947439, |
| cg16806213, | cg17932662, | cg18260397, | cg11940219, | cg09612099, |
| cg16731811, | cg19898394, | cg09992746, | cg01056242, | cg12433395, |
| cg18607468, | cg16579049, | cg26802025, | cg23993660, | cg24508208, |
| cg12993715, | cg00878630, | cg25764256, | cg07433411, | cg15607292, |
| cg19674166, | cg00824880, | cg10107836, | cg08587313, | cg01667324, |
| cg16975985, | cg00493068, | cg11474250, | cg02851047, | cg04854098, |
| cg23312431, | cg20392105, | cg01793617, | cg10854292, | cg11662638, |
| cg04341454, | cg20092892, | cg21424940, | cg20679082, | cg26830985, |
| cg23068913, | cg15137445, | cg07545636, | cg04778793, | cg18824571, |
| cg14349956, | cg05788725, | cg26524638, | cg09063661, | cg24884940, |
| cg00584026, | cg14422906, | cg21387604, | cg10646653, | cg09507215, |
| cg06784845, | cg19405965, | cg14064193, | cg04256238, | cg08034413, |
| cg25499461, | cg05151803, | cg22063966, | cg06821993, | cg22712329, |
| cg03995228, | cg13785949, | cg22387286, | cg13489040, | cg18606378, |
| cg10802379, | cg01431057, | cg07725836, | cg19157971, | cg16495743, |
| cg13824302, | cg04612488, | cg24232030, | cg20687038, | cg05607742, |
| cg14820908, | cg12594911, | cg01457413, | cg18835942, | cg14529146, |
| cg12587618, | cg27618145, | cg05941778, | cg11875676, | cg17712694, |
| cg22294755, | cg06793728, | cg10255535, | cg06812225, | cg12409821, |
| cg02629138, | cg18838436, | cg18819620, | cg06067573, | cg06937548, |
| cg11579905, | cg20135230, | cg05782288, | cg21917084, | cg00191458, |
| cg04522309, | cg21787249, | cg27627821, | cg26478215, | cg00687306, |
| cg26712188, | cg20018234, | cg03858703, | cg10700564, | cg18508148, |
| cg14634563, | cg22790758, | cg22677260, | cg09612502, | cg06444452, |
| cg23087108, | cg11094938, | cg06428425, | cg21041447, | cg07580762, |
| cg14630933, | cg13649886, | cg18274559, | cg02104049, | cg00837290, |
| cg12131007, | cg06138156, | cg11252141, | cg03106413, | cg06294637, |
| cg18515328, | cg22103909, | cg14670303, | cg14615868, | cg00233943, |
| cg27426146, | cg10361659, | cg05961792, | cg03460239, | cg14645415, |
| cg20003800, | cg13649020, | cg04708036, | cg02313013, | cg17968647, |
| cg08670136, | cg18462141, | cg10161194, | cg11416669, | cg18282791, |
| cg06224832, | cg05779088, | cg21301148, | cg00952642, | cg14610217, |
| cg03436178, | cg19355929, | cg05215272, | cg20607169, | cg08177833, |
| cg07562139, | cg14939300, | cg22734397, | cg13391638, | cg24605023, |
| cg22360318, | cg25225968, | cg25448636, | cg24305127, | cg08453750, |
| cg11526630, | cg04585227, | cg26503018, | cg00331740, | cg02363010, |
| cg10375890, | cg24174730, | cg13584718, | cg17676428, | cg18952599, |
| cg10809485, | cg20198684, | cg26384903, | cg02497868, | cg00282347, |
| cg01479645, | cg00258976, | cg11307565, | cg11155090, | cg08187425, |
| cg06057404, | cg13912011, | cg16783204, | cg13687729, | cg05973395, |
| cg10604002, | cg05583636, | cg10544564, | cg24201033, | cg17730759, |
| cg27501458, | cg21021448, | cg17628060, | cg15898112, | cg03403507, |
| cg03363904, | cg16808910, | cg00018198, | cg07455789, | cg14427972, |
| cg01804183, | cg26958654, | cg11976616, | cg26378453, | cg11007492, |
| cg13510418, | cg20154206, | cg08029329, | cg24622726, | cg02985724, |
| cg17865752, | cg16849562, | cg17876456, | cg21374864, | cg25118879, |
| cg10594818, | cg00180470, | cg13930047, | cg23762915, | cg27568306, |
| cg03290040, | cg17416146, | cg02776148, | cg01320969, | cg06872257, |
| cg11256152, | cg17626734, | cg20358437, | cg14204255, | cg18477188, |
| cg17897352, | cg06969933, | cg09385093, | cg03231329, | cg12393324, |
| cg24494556, | cg08038667, | cg07093539, | cg06223736, | cg23920441, |
| cg16488974, | cg10424330, | cg21237939, | cg20805334, | cg15371806, |
| cg19301658, | cg02710090, | cg16141378, | cg00502533, | cg04784327, |
| cg14271231, | cg26659853, | cg19603075, | cg24240466, | cg22865713, |
| cg07888957, | cg01434121, | cg00854043, | cg22134325, | cg22215508, |
| cg27066296, | cg03159004, | cg26357596, | cg25983544, | cg16747785, |
| cg22900798, | cg00326648, | cg18806193, | cg12041848, | cg13511195, |
| cg01452444, | cg12448989, | cg01338599, | cg15809578, | cg09856367, |
| cg19784873, | cg20435608, | cg07285764, | cg15118204, | cg23813556, |
| cg12524531, | cg05595345, | cg04353771, | cg03667086, | cg23087877, |
| cg12103197, | cg07677850, | cg07779444, | cg20814826, | cg23602909, |
| cg05560932, | cg03233885, | cg19858718, | cg13707768, | cg05870116, |
| cg27542828, | cg18232125, | cg22678436, | cg02542748, | cg25424029, |
| cg00432041, | cg14638988, | cg09287650, | cg12036877, | cg15349424, |
| cg04851639, | cg03170834, | cg07210996, | cg05504045, | cg27469810, |
| cg24473518, | cg06646488, | cg02202305, | cg23336452, | cg12808596, |
| cg26855148, | cg06531916, | cg09119494, | cg10182531, | cg16585985, |
| cg19978209, | cg14433217, | cg24105147, | cg08597761, | cg03521812, |
| cg15208525, | cg02910002, | cg05395476, | cg03786842, | cg14580567, |
| cg07793224, | cg25334903, | cg13036549, | cg17728125, | cg01294297, |
| cg01132100, | cg23116540, | cg11150807, | cg20448447, | cg13362627, |
| cg22118655, | cg17445840, | cg09132577, | cg24390428, | cg07864976, |
| cg01928820, | cg25828185, | cg02340063, | cg15389490, | cg16410524, |
| cg16857641, | cg15880016, | cg07273388, | cg00607609, | cg06130950, |
| cg05756257, | cg01058294, | cg10094238, | cg09060610, | cg12687069, |
| cg27055365, | cg08681117, | cg19424343, | cg26128121, | cg20727290, |
| cg04012817, | cg27287305, | cg18071071, | cg01334432, | cg14659662, |
| cg09337653, | cg00231875, | cg15034267, | cg10063565, | cg26100256, |
| cg01812927, | cg21910489, | cg12387464, | cg17335499, | cg17178761, |
| cg08955941, | cg11290860, | cg00609291, | cg01493517, | cg03315432, |
| cg09192377, | cg11043450, | cg23227503, | cg23245711, | cg25023058, |
| cg16021182, | cg07515196, | cg21539087, | cg03998871, | cg26434653, |
| cg14506657, | cg18318639, | cg24851364, | cg21035374, | cg15124201, |
| cg01721429, | cg24796852, | cg22804475, | cg03964958, | cg06118312, |
| cg09922117, | cg05361728, | cg13145462, | cg16229412, | cg25368943, |
| cg12556555, | cg00555538, | cg00235367, | cg21220965, | cg08983960, |
| cg07183320, | cg15159017, | cg14737905, | cg24255125, | cg24880024, |
| cg10452704, | cg12158926, | cg00042882, | cg09377882, | cg07534194, |
| cg07426472, | cg01688243, | cg02042823, | cg25765720, | cg21306294, |
| cg12421819, | cg03355190, | cg13959088, | cg05176051, | cg10582860, |
| cg12452788, | cg10224107, | cg11713664, | cg21827674, | cg13969273, |
| cg09846625, | cg26392989, | cg01154046, | cg00520717, | cg08017850, |
| cg26779406, | cg03563630, | cg25493335, | cg06036471, | cg06049107, |
| cg22699768, | cg17474004, | cg07510080, | cg16697977, | cg25085729, |
| cg12527995, | cg07906527, | cg04350202, | cg18194945, | cg20487287, |
| cg22016649, | cg09244071, | cg05866214, | cg22273355, | cg02742418, |
| cg05087002, | cg03041602, | cg22723098, | cg24306142, | cg04574442, |
| cg09996240, | cg02592615, | cg16478977, | cg04340481, | cg01109633, |
| cg02286506, | cg16399182, | cg22877024, | cg05715005, | cg26806527, |
| cg04972529, | cg24848467, | cg06924878, | cg23354735, | cg04795387, |
| cg04138198, | cg07031916, | cg14333338, | cg21354621, | cg11789612, |
| cg07065054, | cg25186874, | cg07843262, | cg14385337, | cg02263479, |
| cg16588163, | cg18061327, | cg14406134, | cg11245431, | cg08757037, |
| cg20968963, | cg14234680, | cg12228977, | cg27042667, | cg24670566, |
| cg16332224, | cg26212229, | cg05332306, | cg13708218, | cg17047566, |
| cg19925215, | cg13596497, | cg13420408, | cg21743182, | cg22196191, |
| cg17135392, | cg27145347, | cg00971613, | cg07141142, | cg13653024, |
| cg04836792, | cg15723874, | cg24019337, | cg00299286, | cg12860156, |
| cg15919924, | cg01444801, | cg26731119, | cg07583091, | cg07030506, |
| cg20440718, | cg08614082, | cg19663555, | cg12657025, | cg06533895, |
| cg01410279, | ch.6.159391054R, | cg03548730, | cg21821808, | cg17533884, |
| cg00599770, | cg19554522, | cg24517738, | cg19001794, | cg25132957, |
| cg13475333, | cg09404381, | cg18921306, | cg06755612, | cg02972715, |
| cg05500574, | cg12336574, | cg26556186, | cg01057573, | cg14832358, |
| cg15338159, | cg21326642, | cg21698294, | cg06866005, | cg13915277, |
| cg00094943, | cg18908568, | cg00636194, | cg24338091, | cg21920539, |
| cg16365799, | cg25949338, | cg05121480, | cg19141132, | cg00511464, |
| cg15553408, | cg06774893, | cg13843771, | cg17306884, | cg20925811, |
| cg19655195, | cg07487925, | cg18650716, | cg14101302, | cg19713959, |
| cg26265279, | cg07074575, | cg07769277, | cg13846563, | cg09094804, |
| cg10920224, | cg01839657, | cg08217411, | cg01943874, | cg18689289, |
| cg12188268, | cg26309457, | cg13316736, | cg22072063, | cg07090424, |
| cg05159799, | cg11545838, | cg27555617, | cg12205742, | cg02817007, |
| cg21269763, | cg22960962, | cg26699757, | cg17352975, | cg01017689, |
| cg15362480, | cg07841762, | cg10000775, | cg21626523, | cg21044834, |
| cg23978357, | cg05215748, | cg18369654, | cg25204440, | cg04856841, |
| cg19591595, | cg12297819, | cg04220088, | cg14002960, | cg03719475, |
| cg05721705, | cg01531949, | cg11620066, | cg06741043, | cg06081482, |
| cg25339112, | cg20347090, | cg20905680, | cg27244385, | cg14227996, |
| cg27370573, | cg00999623, | cg13392571, | cg10158715, | cg24451800, |
| cg14519664, | cg03605567, | cg25686162, | cg23654971, | cg09601629, |
| cg03155200, | cg16049691, | cg02787327, | cg04454259, | cg03353436, |
| cg09315024, | cg04825327, | cg25513379, | cg02817601, | cg08133755, |
| cg00225858, | cg17628040, | cg03986395, | cg16667631, | cg19129687, |
| cg03579058, | cg19817367, | cg09822284, | cg12211490, | cg07168837, |
| cg07269491, | cg06299996, | cg21481929, | cg07941680, | cg19835595, |
| cg13716585, | cg14558262, | cg14261840, | cg17403830, | cg01606878, |
| cg02547794, | cg24599434, | ch.10.31370070F, | cg24816464, | cg09675484, |
| cg08214329 | | | | |

¹CvC—cancer vs. control samples (TCGA dataset)
²PvR—progressive vs. regressive CIS dataset

TABLE 5
Metrics:

| Number | ROC AUC | ROC | Threshold | Sensitivity | Specificity | Sensitivity | Specificity |
|---|---|---|---|---|---|---|---|
| of DMPs | (CvC¹) | (PvR²) | value | (CvC) | (CvC) | (PvR) | (PvR) |
| 2000 | 0.983462033 | 0.80941358 | 0.36 | 1 | 0.424324324 | 0.888888889 | 0.694444444 |

DMP IDs:

| | | | | | | |
|---|---|---|---|---|---|---|
| cg25698741, | cg08301896, | cg17761815, | cg16168723, | cg14102644, | cg02566863, | cg10680235, |
| cg22255095, | cg03340244, | cg08063194, | cg14768946, | cg23956036, | cg11904056, | cg01910330, |
| cg17341174, | cg17176573, | cg17301842, | cg25602759, | cg01638213, | cg24684765, | cg08857039, |
| cg08900363, | cg24527785, | cg16508028, | cg03550727, | cg03643838, | cg24712417, | cg21796528, |
| cg06757925, | cg16624091, | cg07889420, | cg13976219, | cg01644623, | cg17015937, | cg12792509, |
| cg12206103, | cg01810471, | cg06102127, | cg26187005, | cg24407086, | cg05312779, | cg16390380, |
| cg00586732, | cg14051264, | cg10833620, | cg03939371, | cg16009558, | cg20993253, | cg24131452, |
| cg15889703, | cg27165039, | cg21507987, | cg06084952, | cg14969646, | cg26946836, | cg19026306, |
| cg04891577, | cg02644301, | cg05968988, | cg17841452, | cg13085414, | cg17012181, | cg18450931, |
| cg17904575, | cg11407514, | cg21853510, | cg20462300, | cg07723365, | cg19012958, | cg07002295, |
| cg06675273, | cg00936309, | cg24160158, | cg03055888, | cg12047375, | cg07458500, | cg12899077, |
| cg20447568, | cg04067157, | cg00721771, | cg03267697, | cg09122660, | cg06996138, | cg07967632, |
| cg06885750, | cg04929759, | cg15542798, | cg04170468, | cg25605769, | cg20147515, | cg09163478, |
| cg02524531, | cg04648382, | cg15213988, | cg03567262, | cg24504854, | cg04964031, | cg25019343, |
| cg08124791, | cg13085470, | cg02578368, | cg10995925, | cg00043005, | cg09644109, | cg17516572, |
| cg17102069, | cg01557844, | cg09810089, | cg22807241, | cg08090452, | cg17170741, | cg05844247, |
| cg26389255, | cg23731781, | cg00872471, | cg07815856, | cg12424867, | cg26413708, | cg19176998, |
| cg04894027, | cg26805129, | cg27081821, | cg17944774, | cg16275882, | cg10378364, | cg03878377, |
| cg25864024, | cg16308790, | cg19539664, | cg06300141, | cg07219542, | cg06447952, | cg09528449, |
| cg10715272, | cg04664161, | cg20573218, | cg16262614, | cg07505018, | cg03233793, | cg17417054, |
| cg24577369, | cg11371748, | cg19135761, | cg19380394, | cg09163720, | cg10326447, | cg00321007, |
| cg06950023, | cg11584101, | cg11429960, | cg03178404, | cg01447579, | cg10353108, | cg13235825, |
| cg04820254, | cg15924583, | cg07996594, | cg01835576, | cg12584520, | cg17784606, | cg25111781, |
| cg24968931, | cg06465371, | cg20598989, | cg01397661, | cg10062193, | cg16697996, | cg27543064, |
| cg24112916, | cg18909389, | cg15018122, | cg06823354, | cg22001807, | cg21475544, | cg01842720, |
| cg27541374, | cg20675389, | cg10133100, | cg10512745, | cg26909564, | cg02757696, | cg25700513, |
| cg06778747, | cg14714797, | cg00468320, | cg13777937, | cg09866104, | cg26428032, | cg03354777, |
| cg21300267, | cg14351528, | cg00003298, | cg19243691, | cg03548645, | cg03023089, | cg05621843, |
| cg13554553, | cg00099768, | cg06676563, | cg17670263, | cg24914545, | cg23711881, | cg13639881, |
| cg01563148, | cg02944934, | cg12588082, | cg09584118, | cg11682774, | cg19353425, | cg22457637, |
| cg15174783, | cg00479451, | cg14615914, | cg09170232, | cg05210826, | cg05383328, | cg04416414, |
| cg07601536, | cg04385934, | cg05264578, | cg27141850, | cg13464738, | cg20771240, | cg23750391, |
| cg27035251, | cg07912766, | cg15012214, | cg09568001, | cg20936183, | cg21060854, | cg21728844, |
| cg10773928, | cg03348901, | cg11824564, | cg10147410, | cg25627242, | cg11417496, | cg19547694, |
| cg00018606, | cg14260485, | cg16918529, | cg25212146, | cg13808641, | cg15735417, | cg18142615, |
| cg05064398, | cg01425054, | cg10598776, | cg23034818, | cg11196529, | cg02554051, | cg11277665, |
| cg09268125, | cg11200568, | cg02363655, | cg19779670, | cg14462485, | cg14737484, | cg16095615, |
| cg05526757, | cg07820783, | cg05741611, | cg10453419, | cg05942825, | cg16025468, | cg22038409, |
| cg21153697, | cg02410986, | cg06758211, | cg18648037, | cg12264551, | cg19395706, | cg16274061, |
| cg15403654, | cg22062383, | cg00846043, | cg02185971, | cg27567430, | cg05769153, | cg02321062, |
| cg06022664, | cg03186440, | cg01409873, | cg07140885, | cg25774457, | cg01567754, | cg16523463, |
| cg02053188, | cg10768548, | cg22769031, | cg17897309, | cg05562241, | cg06164961, | cg27196999, |
| cg14558568, | cg26097182, | cg03623178, | cg24294989, | cg20391652, | cg05902063, | cg18562896, |
| cg06422694, | cg12260343, | cg27131607, | cg06348826, | cg01110552, | cg22665089, | cg01281776, |
| cg25099516, | cg22695379, | cg17778120, | cg27390245, | cg04893409, | cg11654037, | cg24338843, |
| cg04069099, | cg14951488, | cg03824768, | cg12197235, | cg19495444, | cg00456557, | cg16635578, |
| cg13265869, | cg09025625, | cg10747266, | cg01496185, | cg25749432, | cg23886165, | cg18276810, |
| cg13876325, | cg24429533, | cg24578937, | cg23337116, | cg12168357, | cg01792081, | cg18039855, |
| cg07671586, | cg00886695, | cg03795316, | cg23495453, | cg05110962, | cg00607566, | cg00275557, |
| cg06512128, | cg02109381, | cg03368930, | cg25810062, | cg22989407, | cg01308419, | cg22180410, |
| cg20465661, | cg05198733, | cg19431200, | cg10138970, | cg19333614, | cg19848683, | cg09150815, |
| cg18973173, | cg05371711, | cg02343254, | cg02025157, | cg06682197, | cg20823940, | cg14740554, |
| cg06787004, | cg14663278, | cg19802542, | cg22630160, | cg02334987, | cg12921795, | cg22827685, |
| cg18060965, | cg10777887, | cg01145948, | cg07265622, | cg05488809, | cg15895213, | cg21635917, |
| cg16199381, | cg03611852, | cg24103044, | cg26884567, | cg11946666, | cg12271419, | cg10255544, |
| cg09790829, | cg07106989, | cg17140815, | cg11164142, | cg18947276, | cg11664251, | cg08671647, |
| cg21990808, | cg20708892, | cg00612625, | cg05033052, | cg05472265, | cg04749453, | cg23472261, |
| cg03658066, | cg23807071, | cg10820262, | cg07021644, | cg10181016, | cg15365032, | cg24993140, |
| cg25819602, | cg19239199, | cg23097878, | cg01616489, | cg26601317, | cg10272433, | cg08910550, |
| cg00553886, | cg26629188, | cg14199144, | cg26516701, | cg06970884, | cg17243193, | cg23158862, |
| cg15366555, | cg10535320, | cg21213332, | cg18148620, | cg24758426, | cg23671719, | cg02102832, |
| cg12996417, | cg23809645, | cg16323428, | cg23947450, | cg02452985, | cg21531126, | cg06891663, |
| cg16446408, | cg12753518, | cg14302973, | cg21767902, | cg11451477, | cg08934830, | cg12118798, |
| cg06635625, | cg15346952, | cg25288660, | cg07893916, | cg07212035, | cg00937175, | cg23258173, |
| cg00452393, | cg02801772, | cg20672711, | cg24566400, | cg18561560, | cg05575217, | cg06797280, |
| cg24761366, | cg03211828, | cg20986608, | cg01297479, | cg02584432, | cg03105244, | cg20575163, |
| cg14523881, | cg03272225, | cg11734790, | cg14803869, | cg02330214, | cg22992057, | cg19864705, |
| cg00435528, | cg22083335, | cg07341290, | cg09005762, | cg08545463, | cg08482531, | cg04914832, |
| cg11037064, | cg16389345, | cg15805053, | cg01791785, | cg17563229, | cg14845282, | cg00133811, |
| cg25122590, | cg24612198, | cg07336438, | cg17670496, | cg06813554, | cg24669528, | cg22291265, |
| cg03416562, | cg22974982, | cg24581226, | cg07112473, | cg06954346, | cg10071595, | cg11775292, |
| cg07245225, | cg20136435, | cg10496150, | cg25359645, | cg04553960, | cg11078040, | cg16902559, |
| cg16698101, | cg21344741, | cg25378939, | cg04213038, | cg26785220, | cg21874514, | cg09175575, |
| cg06008853, | cg08518715, | cg09710740, | cg18041960, | cg00909156, | cg18854413, | cg04691173, |
| cg00453202, | cg06833721, | cg23316987, | cg14939475, | cg01439876, | cg25271404, | cg01263631, |
| cg01919488, | cg22330497, | cg10454596, | cg17706606, | cg16307498, | cg21188610, | cg05267623, |
| cg13848568, | cg21235025, | cg00770008, | cg20303301, | cg23942984, | cg09694898, | cg03521124, |
| cg07144296, | cg20162206, | cg26552774, | cg14397775, | cg27248073, | cg02055351, | cg00003529, |
| cg04142692, | cg10898421, | cg23261184, | cg03577486, | cg14495458, | cg22516105, | cg09728459, |
| cg01517680, | cg15664967, | cg01557798, | cg09849600, | cg17254388, | cg00148926, | cg18554868, |
| cg17668158, | cg15739881, | cg26952662, | cg08362283, | cg18333824, | cg13527922, | cg04876072, |
| cg01129886, | cg13835436, | cg15244223, | cg12812502, | cg16487577, | cg09215582, | cg14330737, |
| cg14361627, | cg01437781, | cg26316544, | cg09948076, | cg13563094, | cg00711711, | cg23207816, |
| cg01771737, | cg09981996, | cg10905401, | cg17489908, | cg03460163, | cg19694978, | cg08508777, |
| cg21139063, | cg07259811, | cg10525820, | cg07955126, | cg14507792, | cg10682803, | cg21023447, |
| cg05107429, | cg02548912, | cg26674558, | cg12042448, | cg01561878, | cg01797899, | cg16289417, |
| cg03602777, | cg21335012, | cg07471962, | cg04573976, | cg14560022, | cg10272954, | cg21792134, |
| cg18288666, | cg18912484, | cg13431342, | cg13759632, | cg23735602, | cg17715482, | cg12419308, |
| cg11596498, | cg20956278, | cg09221382, | cg12809787, | cg05155043, | cg01559787, | cg14482998, |
| cg03346415, | cg01913568, | cg00236601, | cg05513583, | cg01110759, | cg19700260, | cg16715508, |
| cg04677295, | cg07855639, | cg16031515, | cg26932226, | cg03372779, | cg05681996, | cg15650161, |
| cg03550548, | cg18465245, | cg12923233, | cg24194053, | cg17305181, | cg06777581, | cg15114744, |
| cg00683984, | cg07829377, | cg13558971, | cg03663746, | cg04423431, | cg04215179, | cg17625891, |
| cg04654489, | cg13564459, | cg03038816, | cg09807524, | cg00566320, | cg18449050, | cg05254651, |
| cg23185829, | cg11026330, | cg00840320, | cg07956200, | cg15408737, | cg02629156, | cg27245817, |
| cg17124092, | cg00716675, | cg25979108, | cg11982471, | cg07077240, | cg13207250, | cg26777456, |
| cg20057831, | cg10128806, | cg18096962, | cg10247405, | cg19250989, | cg15741583, | cg15916004, |
| cg06992390, | cg18420512, | cg00688465, | cg16746221, | cg17271338, | cg21321359, | cg00429107, |
| cg07990939, | cg10546523, | cg10587940, | cg03604417, | cg18426166, | cg11661250, | cg21519645, |
| cg25138715, | cg26393294, | cg07062262, | cg13713450, | cg06371502, | cg15687869, | cg04270867, |
| cg27312979, | cg17088562, | cg11229872, | cg03212678, | cg26957471, | cg19316407, | cg22230581, |
| cg13844804, | cg03226993, | cg10218777, | cg12065366, | cg09951485, | cg08998375, | cg24611996, |
| cg03201274, | cg07702548, | cg23097558, | cg00111823, | cg14159004, | cg02695609, | cg10042540, |
| cg17842912, | cg13916742, | cg06761203, | cg06915524, | cg13446070, | cg03054141, | cg26430450, |
| cg23121156, | cg08265288, | cg16410464, | cg14869845, | cg01856450, | cg14080254, | cg14689122, |
| cg08948528, | cg21549161, | cg24005950, | cg16703956, | cg02537639, | cg23575107, | cg23239052, |
| cg22057720, | cg09150905, | cg00118507, | cg06893371, | cg19668990, | cg19778015, | cg21263710, |
| cg09429153, | cg25254573, | cg19936022, | cg24870050, | cg07328635, | cg03428279, | cg10790791, |
| cg18851960, | cg04906283, | cg23497678, | cg08590939, | cg12053762, | cg24383528, | cg13559409, |
| cg07316730, | cg10330187, | cg04653928, | cg12091786, | cg19935326, | cg02196734, | cg02628202, |
| cg18685243, | cg01886741, | cg00379720, | cg01447498, | cg13404472, | cg08354351, | cg03933131, |
| cg19432283, | cg07685357, | cg21010566, | cg26763618, | cg22823546, | cg02162202, | cg10259889, |
| cg09423066, | cg10453019, | cg09608765, | cg02068351, | cg27197835, | cg11639984, | cg05413700, |
| cg06697339, | cg12138286, | cg05447186, | cg01379480, | cg18372196, | cg04657470, | cg19802165, |
| cg04844543, | cg14894848, | cg11773779, | cg19325637, | cg14272860, | cg00261668, | cg04727332, |
| cg15863374, | cg26809488, | cg13370485, | cg18978493, | cg01494614, | cg24339704, | cg06862374, |
| cg01787884, | cg05402841, | cg26926612, | cg10914467, | cg27460534, | cg17340268, | cg05812599, |
| cg02684548, | cg00602326, | cg22381068, | cg14037182, | cg00956091, | cg18918390, | cg15332359, |
| cg18791923, | cg26257869, | cg06856155, | cg01146943, | cg20365074, | cg19470508, | cg19151849, |
| cg05653646, | cg08696192, | cg21128568, | cg22640209, | cg21040069, | cg09407816, | cg18427987, |
| cg16235748, | cg23370290, | cg19847130, | cg21187597, | cg25731959, | cg01781805, | cg19414780, |
| cg19578398, | cg16617510, | cg02499614, | cg03122372, | cg17439023, | cg07267067, | cg23071995, |
| cg14630099, | cg23685149, | cg18941458, | cg25280526, | cg01018477, | cg15554682, | cg00962755, |
| cg08272731, | cg01171670, | cg10380345, | cg20321153, | cg07162551, | cg00444261, | cg26344392, |
| cg04099660, | cg05406868, | cg27516940, | cg25281042, | cg04966255, | cg09388080, | cg12974138, |
| cg06086073, | cg21861233, | cg14815609, | cg21475255, | cg07682378, | cg09266149, | cg15575914, |
| cg18249580, | cg20010396, | cg03622263, | cg08047457, | cg01061890, | cg01092293, | cg12325455, |
| cg02710481, | cg11722249, | cg19238270, | cg02132702, | cg04480280, | cg16635863, | cg04216310, |
| cg13561081, | cg24803261, | cg24283027, | cg25668117, | cg14222140, | cg22237988, | cg26871386, |
| cg17510758, | cg00777310, | cg13337949, | cg07030052, | cg10672804, | cg10725861, | cg26777074, |
| cg00826767, | cg09409539, | cg08702689, | cg10755058, | cg08387463, | cg13763289, | cg00816397, |
| cg22965634, | cg18882819, | cg14951598, | cg19875969, | cg00630761, | cg21011649, | cg21859623, |
| cg25235958, | cg07835443, | cg07161166, | cg16748524, | cg07061500, | cg13785127, | cg24347098, |
| cg10197846, | cg10652956, | cg25728876, | cg20305028, | cg18548263, | cg06740598, | cg18632254, |
| cg05598539, | cg24484737, | cg18048542, | cg01441387, | cg00936733, | cg22368208, | cg26166170, |
| cg10287461, | cg23246885, | cg17758673, | cg16462648, | cg06320606, | cg23018891, | cg01848323, |
| cg18120807, | cg08656267, | cg12553843, | cg13634736, | cg21786957, | cg04729491, | cg24134292, |
| cg23247274, | cg06633543, | cg21662326, | cg16446486, | cg01261503, | cg06070508, | cg16360717, |
| cg03284670, | cg07067833, | cg04019914, | cg12550312, | cg22522688, | cg21794518, | cg16192821, |
| cg26693701, | cg00113321, | cg00346074, | cg25519723, | cg26775369, | cg00629563, | cg13782866, |
| cg25694807, | cg00839579, | cg26198700, | cg11976052, | cg10731507, | cg07355346, | cg26109510, |
| cg01382281, | cg10893014, | cg06147895, | cg07128379, | cg19618483, | cg11251827, | cg18235480, |
| cg10759927, | cg09045305, | cg09558069, | cg16521594, | cg21332334, | cg20796611, | cg05360208, |
| cg25328154, | cg08582485, | cg16225663, | cg13439914, | cg16641055, | cg26151910, | cg17177931, |
| cg01088382, | cg15128470, | cg02183170, | cg25008504, | cg22007227, | cg13774369, | cg17468663, |
| cg16739580, | cg23731836, | cg10484990, | cg04614700, | cg08393317, | cg25796445, | cg27587095, |
| cg24992577, | cg27258933, | cg27408398, | cg00972135, | cg01231779, | cg20283292, | cg11469587, |
| cg12030226, | cg05136737, | cg18166947, | cg16134323, | cg19342883, | cg08037684, | cg19574297, |
| cg22379905, | cg25423135, | cg09063201, | cg04009932, | cg13316587, | cg00840403, | cg00166372, |
| cg05709657, | cg03108928, | cg17634326, | cg10669219, | cg14155637, | cg22736037, | cg12501923, |
| cg20778294, | cg06371836, | cg26385283, | cg06861572, | cg09246203, | cg07934031, | cg13117809, |
| cg24148287, | cg14413721, | cg24036830, | cg14397941, | cg16311504, | cg13001495, | cg23107878, |
| cg03669797, | cg00286119, | cg23912186, | cg02060096, | cg00373020, | cg05260242, | cg24446178, |
| cg19042136, | cg21184951, | cg08139509, | cg07172701, | cg04771946, | cg23598378, | cg24220733, |
| cg17949127, | cg23886495, | cg22259283, | cg18391972, | cg18792170, | cg16402452, | cg15475055, |
| cg01424562, | cg13398947, | cg27518043, | cg20268683, | cg20732755, | cg14785303, | cg21181989, |
| cg03770548, | cg00055764, | cg00335892, | cg21198755, | cg01923999, | cg17662182, | cg09384937, |
| cg26221719, | cg19879906, | cg22865713, | cg17744293, | cg00802510, | cg06998422, | cg00746515, |
| cg20190559, | cg03733229, | cg13850380, | cg15269548, | cg09858237, | cg16795307, | cg05501868, |
| cg07058109, | cg08725538, | cg18220380, | cg13657092, | cg15447829, | cg20616186, | cg16865475, |
| cg02010752, | cg08589981, | cg23925190, | cg12007166, | cg26628821, | cg07451391, | cg15993027, |
| cg17084697, | cg05355067, | cg14735364, | cg16572020, | cg12749531, | cg17044768, | cg01620611, |
| cg16654143, | cg17344813, | cg06117033, | cg03760919, | cg03420791, | cg02914800, | cg19233769, |
| cg23953697, | cg26421593, | cg24144070, | cg09879122, | cg10569615, | cg02777068, | cg00277898, |
| cg04487296, | cg17125208, | cg06797835, | cg14709239, | cg09885499, | cg02691506, | cg21517055, |
| cg05697909, | cg08667148, | cg12203525, | cg14162552, | cg05249084, | cg17603988, | cg04509221, |
| cg18720580, | cg04862799, | cg07614502, | cg11233340, | cg12893030, | cg20931134, | cg05635712, |

DMP IDs:

| | | | | |
|---|---|---|---|---|
| cg10654803, | cg24152179, | cg25927642, | cg04543026, | cg14153654, |
| cg10493739, | cg20844146, | cg05750962, | cg23971517, | cg24831879, |
| cg11658054, | cg11155784, | cg24857560, | cg07354253, | cg07909749, |
| cg27303421, | cg05567709, | cg21801176, | cg05831188, | cg21448033, |
| cg12289926, | cg05787952, | cg24035112, | cg04660111, | cg26635603, |
| cg22945462, | cg08831531, | cg16072462, | cg24718971, | cg26106304, |
| cg18354986, | cg09816500, | cg20685067, | cg03946762, | cg08718293, |
| cg09736654, | cg02219949, | cg27457239, | cg21908287, | cg11195662, |
| cg25478132, | cg24213773, | cg06553422, | cg04545079, | cg24311644, |
| cg03580272, | cg14143326, | cg21845457, | cg07972159, | cg06045799, |
| cg03686522, | cg27590591, | cg18295068, | cg21907877, | cg07563050, |
| cg10239319, | cg03179043, | cg13930080, | cg02715006, | cg25590734, |
| cg25504222, | cg26334507, | cg18341054, | cg17310215, | cg13314394, |
| cg10987624, | cg11273493, | cg10946333, | cg08558733, | cg11528832, |
| cg02651871, | cg26174215, | cg03904876, | cg13692649, | cg13464448, |
| cg09594475, | cg26730347, | cg17130754, | cg25290938, | cg05784201, |
| cg02729086, | cg19950097, | cg25691239, | cg03186004, | cg00614832, |
| cg08940505, | cg15955341, | cg09858692, | cg04536922, | cg17713954, |
| cg04407147, | cg19476082, | cg26103560, | cg02984023, | cg16253537, |
| cg06700653, | cg27066989, | cg27302054, | cg01322290, | cg00399175, |
| cg05906620, | cg10873475, | cg26122063, | cg09501274, | cg12912426, |
| cg08286169, | cg07461501, | cg03585598, | cg08447479, | cg22468123, |
| cg24991933, | cg02641801, | cg00107890, | cg26670985, | cg07140158, |
| cg18391442, | cg17829914, | cg07569575, | cg00135293, | cg04010205, |
| cg08766149, | cg02162286, | cg02512286, | cg26771384, | cg12560527, |
| cg10417386, | cg11484721, | cg15396686, | cg00474713, | cg10998878, |
| cg14346035, | cg15517098, | cg04482825, | cg03948902, | cg25282715, |
| cg25493687, | cg02457207, | cg02946138, | cg23711139, | cg22258045, |
| cg05975654, | cg19382919, | cg11470063, | cg13817182, | cg03401096, |
| cg22471742, | cg11233366, | cg26307359, | cg12427469, | cg13580265, |
| cg20378837, | cg12773030, | cg11111139, | cg20792294, | cg21193977, |
| cg25952510, | cg19335617, | cg17221945, | cg24109612, | cg21535606, |
| cg17243628, | cg24030138, | cg06850558, | cg09725195, | cg17601658, |
| cg00990591, | cg17695543, | cg17110299, | cg05298628, | cg02310421, |
| cg03072483, | cg22311615, | cg11724759, | cg01834010, | cg16189671, |
| cg05387119, | cg22584089, | cg05732956, | cg04955877, | cg06413842, |
| cg23375948, | cg14581387, | cg16564525, | cg25071520, | cg26509071, |
| cg14373579, | cg07118811, | cg04801716, | cg17169289, | cg23523648, |
| cg13579607, | cg06475370, | cg24777071, | cg12614687, | cg01770019, |
| cg14723344, | cg05300946, | cg25825008, | cg05360477, | cg09531376, |
| cg08415973, | cg19603966, | cg26986989, | cg19860154, | cg02961807, |
| cg11348746, | cg01514415, | cg20639263, | cg18007641, | cg25646046, |
| cg11084470, | cg22547485, | cg02577240, | cg00031340, | cg14997715, |
| cg27337194, | cg08450280, | cg11714801, | cg13051202, | cg07224291, |
| cg04818637, | cg04923174, | cg21035905, | cg10828910, | cg15492982, |
| cg09874873, | cg03561787, | cg27461254, | cg00073010, | cg26734916, |
| cg06909288, | cg13483882, | cg27629154, | cg20710709, | cg26960896, |
| cg02667756, | cg20690667, | cg08962087, | cg10032375, | cg21878746, |
| cg11624225, | cg08362102, | cg23365739, | cg26891410, | cg18978531, |
| cg16635128, | cg18105361, | cg19658054, | cg23537820, | cg23418968, |
| cg24760647, | cg00601124, | cg10591926, | cg04744717, | cg24717029, |
| cg11193274, | cg09813817, | cg13934625, | cg07462112, | cg04408225, |
| cg24525540, | cg18255240, | cg14694896, | cg04692454, | cg00421144, |
| cg13482714, | cg01805124, | cg08655701, | cg04673937, | cg10861807, |
| cg25589945, | cg08537737, | cg21189099, | cg12944006, | cg07378067, |
| cg13454226, | cg21667215, | cg13874012, | cg20876269, | cg21827581, |
| cg26510178, | cg05989740, | cg07478208, | cg11162118, | cg21415305, |
| cg24457026, | cg00530298, | cg15036529, | cg24464525, | cg24162630, |
| cg22078859, | cg08626939, | cg00638384, | cg17102199, | cg20706468, |
| cg11625262, | cg14736911, | cg01770355, | cg04884481, | cg15345391, |
| cg16403932, | cg10333144, | cg10205753, | cg23369564, | cg08852741, |
| cg22695532, | cg04115584, | cg10160842, | cg09617135, | cg23402661, |
| cg24012887, | cg16678718, | cg25567438, | cg08390729, | cg19802368, |
| cg12339834, | cg05735075, | cg12661452, | cg04439252, | cg03520342, |
| cg02571448, | cg22117819, | cg23852646, | cg18131626, | cg19104830, |
| cg06257798, | cg04144521, | cg25994622, | cg22051925, | cg23977256, |
| cg16340023, | cg22536016, | cg07844507, | cg09786383, | cg26820922, |
| cg19954537, | cg03951219, | cg12230883, | cg07457252, | cg23282480, |
| cg21181594, | cg04641912, | cg07967851, | cg05658793, | cg11429658, |
| cg21385666, | cg23946962, | cg24253904, | cg11251499, | cg01148786, |
| cg00157656, | cg03327570, | cg03257475, | cg09436502, | cg19589427, |
| cg10901258, | cg12452849, | cg07529754, | cg16587518, | cg01802898, |
| cg12404235, | cg05733361, | cg20449614, | cg19619909, | cg03324123, |
| cg10136008, | cg19342828, | cg06740897, | cg13471599, | cg07259418, |
| cg25970447, | cg20058744, | cg08889373, | cg11312353, | cg09009644, |
| cg03452190, | cg07233838, | cg18782991, | cg15303841, | cg09253125, |
| cg16891075, | cg17591348, | cg09964873, | cg14352715, | cg20313951, |
| cg10397930, | cg13626842, | cg01825818, | cg24341944, | cg13512987, |
| cg04124789, | cg27082921, | cg23999880, | cg27099726, | cg13301676, |
| cg05132222, | cg14468741, | cg21475526, | cg12615766, | cg22347212, |
| cg05422394, | cg13369159, | cg18579761, | cg22481405, | cg11155924, |
| cg05251325, | cg00911551, | cg18997188, | cg15632901, | cg19240857, |
| cg17348730, | cg03764219, | cg15826006, | cg25772738, | cg26035118, |
| cg12986236, | cg04673590, | cg11265468, | cg02920142, | cg00565070, |
| cg24899294, | cg26127203, | cg22589339, | cg12753804, | cg05363871, |
| cg18177826, | cg24343524, | cg13781414, | cg02432487, | cg01935122, |
| cg22642498, | cg11722531, | cg13782615, | cg17111669, | cg25853960, |
| cg09383860, | cg09864712, | cg23422263, | cg15656261, | cg19906454, |
| cg07600499, | cg06346099, | cg24106894, | cg04592226, | cg26866348, |
| cg25250853, | cg27247697, | cg02630349, | cg25008346, | cg21601498, |
| cg24626003, | cg26951763, | cg21454760, | cg01673240, | cg14807090, |
| cg01242484, | cg14129735, | cg27319151, | cg06024540, | cg09565237, |
| cg03432464, | cg19930417, | cg24114936, | cg19321658, | cg25350789, |
| cg22922815, | cg13852775, | cg10397082, | cg15774391, | cg08618620, |
| cg23780110, | cg19083871, | cg16325421, | cg16782848, | cg11004323, |
| cg22256482, | cg17734135, | cg14066439, | cg11776534, | cg09561417, |
| cg18078305, | cg02775883, | cg24470119, | cg15611018, | cg24244270, |
| cg27578760, | cg24545125, | cg07953935, | cg22031466, | cg07199524, |
| cg22951582, | cg24868525, | cg26047334, | cg08637403, | cg06900311, |
| cg00197313, | cg24727480, | cg10720997, | cg12734024, | cg26051165, |
| cg10236857, | cg22692507, | cg14042352, | cg00372169, | cg22696652, |
| cg08413026, | cg19060382, | cg25641920, | cg01274715, | cg08093216, |
| cg07566286, | cg16221289, | cg16501625, | cg01766193, | cg16568681, |
| cg05161264, | cg17471928, | cg11060907, | cg10970124, | cg14914046, |
| cg06203126, | cg10131483, | cg11956813, | cg24810144, | cg02997982, |
| cg12297350, | cg03933676, | cg01971667, | cg22457860, | cg18392644, |
| cg19097494, | cg12736254, | cg01565703, | cg12829687, | cg17173498, |
| cg01782059, | cg22849665, | cg15116488, | cg27094323, | cg22424284, |
| cg02254261, | cg07637239, | cg03590216, | cg21400802, | cg08865423, |
| cg14335894, | cg15768265, | cg19772907, | cg23079522, | cg13552831, |
| cg11236966, | cg14051147, | cg18945744, | cg13249789, | cg01902605, |
| cg20019168, | cg09856153, | cg04568121, | cg24540913, | cg04138756, |
| cg14421180, | cg26948603, | cg05142765, | cg10088320, | cg14205362, |
| cg21825443, | cg13444625, | cg04942655, | cg18138842, | cg23097499, |
| cg01157046, | cg00648152, | cg14482595, | cg01360627, | cg00254486, |
| cg14464464, | cg13586696, | cg02381227, | cg13621701, | cg13666648, |
| cg24570070, | ch.21.18789295F, | cg12559474, | cg20125159, | cg02095290, |
| cg19389884, | cg02273146, | cg07865267, | cg10758227, | cg13588023, |
| cg00657810, | cg23848541, | cg11499431, | cg23194776, | cg22595235, |
| cg17116500, | cg10978503, | cg06490988, | ch.1.198498029R, | cg16773881, |
| cg13075600, | cg24631834, | cg20968010, | cg14039246, | cg12900649, |
| cg14331609, | cg05917311, | cg15652863, | cg03404211, | cg11857704, |
| cg09122035, | cg23063070, | cg18610205, | cg17468100, | cg13685151, |
| cg09432202, | cg18826598, | cg04709771, | cg23746497, | cg21929875, |
| cg01496790, | cg14321522, | cg22732615, | cg03321503, | cg20979296, |
| cg12601576, | cg21454386, | cg01773831, | cg17152757, | cg05141787, |
| cg09338113, | cg01532168, | cg22952849, | cg04778433, | cg15215830, |
| cg01849531, | cg18478175, | cg12973168, | cg12429629, | cg06648156, |
| cg09689944, | cg01277369, | cg07481886, | cg26875877, | cg06817917, |
| cg07746263, | cg24031331, | cg16636756, | cg22493397, | cg01908177, |
| cg18183214, | cg04105453, | cg08400319, | cg25419628, | cg02470625, |
| cg25433490, | cg23606925, | cg00984060, | cg15029747, | cg06561044, |
| cg00870279, | cg19164917, | cg18714679, | cg24249778, | cg06240947, |
| cg17552357, | cg16017904, | cg26936171, | cg07187503, | cg01677177, |
| cg25484127, | cg07681938, | cg03332271, | cg07568716, | cg04508340, |
| cg10325398, | cg06144990, | cg15039853, | cg08552519, | cg08223717, |
| cg21406967, | cg18689289, | cg11370532, | cg13818243, | cg22882665, |
| cg01352175, | cg27224547, | cg18787975, | cg10260935, | cg00299943, |
| cg09703963, | cg07543711, | cg13549444, | cg08602075, | cg01551238, |
| cg08793936, | cg03921149, | cg20529489, | cg12720459, | cg07749412, |
| cg16530429, | cg02947276, | cg02973171, | cg06982190, | cg19139320, |
| cg10121457, | cg08939481, | cg17190685, | cg07795402, | cg11738162, |
| cg11788367, | cg25792961, | cg08975295, | cg03035704, | cg23349726, |
| cg10473157, | cg13433994, | cg20270762, | cg26645468, | cg04118154, |
| cg25149037, | cg13360150, | cg27200895, | cg06935608, | cg02085119, |
| cg01296593, | cg09194742, | cg13951490, | cg06428091, | cg00335003, |
| cg11528517, | cg24974477, | cg00478435, | cg05778278, | cg18756536, |
| cg02390569, | cg14989602, | cg13048591, | cg14516793, | cg23951171, |
| cg16692757, | cg14188507, | cg12447832, | cg02020945, | cg00710004, |
| cg24449629, | cg14606164, | cg10283277, | cg04934807, | cg16936944, |
| cg14107978, | cg10290724, | cg00969680, | cg26096190, | cg24296786, |
| cg22048948, | cg12495975, | cg04668156, | cg24751707, | cg24110540, |
| cg20034209, | cg14073716, | cg06836380, | cg12961069, | cg04775710, |
| cg24219638, | cg08919846, | cg05308117, | cg10712263, | cg21547763, |
| cg03636234, | cg17054006, | cg08734931, | cg01046434, | cg21850069, |
| cg09275783, | cg26039962, | cg22550799, | cg26192720, | cg21558053, |
| cg08747777, | cg02689506, | cg27224971, | cg10557147, | cg23543795, |
| cg11347033, | cg06767142, | cg24645896, | cg21094949, | cg16479539, |
| cg04301760, | cg06733419, | cg12701576, | cg23336323, | cg00982799, |
| cg07003920, | cg15395971, | cg23285599, | cg12986327, | cg26935102, |
| cg18704904, | cg05126887, | cg03032816, | cg13672547, | cg01885783, |
| cg14806636, | cg18704768, | cg14430982, | cg09588815, | cg05490712, |
| cg09233429, | cg13780759, | cg09818265, | cg15387272, | cg25600410, |
| cg06615754, | cg01196842, | cg13987674, | cg03580247, | cg23944440, |
| cg08170227, | cg00078085, | cg09144302, | cg08172304, | cg06319579, |
| cg02868306, | cg10895875, | cg02500443, | cg03381359, | cg23741982, |
| cg16421619 | | | | |

¹CvC—cancer vs. control samples (TCGA dataset)
²PvR—progressive vs. regressive CIS dataset

TABLE 6
Metrics:

| Number | ROC AUC | ROC | Threshold | Sensitivity | Specificity | Sensitivity | Specificity |
|---|---|---|---|---|---|---|---|
| of DMPs | (CvC¹) | (PvR²) | value | (CvC) | (CvC) | (PvR) | (PvR) |
| 2000 | 0.983268983 | 0.823302469 | 0.355 | 1 | 0.545945946 | 0.888888889 | 0.75 |

DMP IDs:

| | | | | | | |
|---|---|---|---|---|---|---|
| cg02720207, | cg06879534, | cg13719443, | cg03012642, | cg06044899, | cg12877165, | cg04963177, |
| cg08337773, | cg09968470, | cg21881652, | cg24868830, | cg11839566, | cg07919197, | cg22922242, |
| cg17530586, | cg04649769, | cg07057191, | cg20040772, | cg20224016, | cg06814005, | cg21032203, |
| cg14633910, | cg23606162, | cg07499159, | cg09280462, | cg17927493, | cg19165390, | cg13305951, |
| cg23627040, | cg19062174, | cg14334965, | cg16184131, | cg12852134, | cg26397549, | cg13679776, |
| cg16133088, | cg04771133, | cg09109411, | cg16386641, | cg15300730, | cg14252492, | cg15565004, |
| cg11775292, | cg10197870, | cg05076253, | cg24213189, | cg22808086, | cg26647524, | cg09355771, |
| cg15773744, | cg11388866, | cg01632188, | cg01386493, | cg21313298, | cg07675334, | cg00462240, |
| cg19207017, | cg25642234, | cg23207958, | cg24639754, | cg01522975, | cg01439753, | cg00067414, |
| cg19513282, | cg08674238, | cg20454517, | cg01378151, | cg19450479, | cg01760189, | cg26221410, |
| cg17956485, | cg26832211, | cg18761696, | cg23297477, | cg27177839, | cg07726139, | cg20364632, |
| cg19134728, | cg17907608, | cg19433697, | cg20769842, | cg19247588, | cg13231680, | cg23321010, |
| cg23835677, | cg05638689, | cg11570575, | cg25307074, | cg09571404, | cg02307823, | cg25734928, |
| cg13619623, | cg12530270, | cg12314121, | cg08868202, | cg16211055, | cg11227645, | cg09939191, |
| cg16374193, | cg15354892, | cg27059127, | cg26618668, | cg13974317, | cg12426234, | cg00793774, |
| cg24652842, | cg27452922, | cg16779866, | cg23474049, | cg04958658, | cg15984460, | cg10574483, |
| cg08295543, | cg04383128, | cg05868445, | cg25998666, | cg13533335, | cg21365914, | cg24409566, |
| cg06316056, | cg03912614, | cg06938878, | cg26354128, | cg26031255, | cg19444609, | cg08589721, |
| cg14454278, | cg15998518, | cg04521626, | cg04480665, | cg05860115, | cg05124190, | cg14562327, |
| cg02850602, | cg20102673, | cg15998962, | cg01980311, | cg14374861, | cg05660795, | cg08176368, |
| cg14272438, | cg25417298, | cg23672659, | cg01887308, | cg05245170, | cg15652381, | cg14157855, |
| cg22042465, | cg03471106, | cg17120764, | cg01530687, | cg22012835, | cg08134068, | cg01758588, |
| cg23809917, | cg06230303, | cg05060843, | cg19472078, | cg17739936, | cg07631435, | cg20586654, |
| cg10904972, | cg00395632, | cg00109503, | cg09595149, | cg08928646, | cg26333594, | cg04122467, |
| cg00167684, | cg24834740, | cg03961676, | cg03894891, | cg12349422, | cg25953688, | cg14861020, |
| cg23378989, | cg27607805, | cg13057898, | cg14470978, | cg09119967, | cg19774624, | cg10068034, |
| cg10627136, | cg06765324, | cg18241308, | cg13917863, | cg05643286, | cg01436424, | cg04318602, |
| cg22429852, | cg00918522, | cg02471153, | cg00719237, | cg27160241, | cg20355694, | cg02309717, |
| cg23126152, | cg11239111, | cg25468879, | cg10307172, | cg18750756, | cg10803218, | cg23091122, |
| cg04279588, | cg25266603, | cg16686761, | cg10073571, | cg17811913, | cg01677601, | cg03652561, |
| cg17398252, | cg17133967, | cg01553231, | cg15225991, | cg24181605, | cg25031824, | cg09412707, |
| cg07562100, | cg13826890, | cg17334970, | cg08895903, | cg20751138, | cg15672329, | cg11759875, |
| cg17320454, | cg21864961, | cg06821147, | cg05627987, | cg00398764, | cg15026243, | cg21641465, |
| cg07282058, | cg00757182, | cg08226590, | cg02928885, | cg03393238, | cg05977191, | cg12000333, |
| cg08832069, | cg03028031, | cg03587117, | cg21467614, | cg02699090, | cg04304450, | cg05270291, |
| cg11185039, | cg10638657, | cg07234911, | cg24005950, | cg13415207, | cg14606067, | cg25350122, |
| cg13717434, | cg11586570, | cg09890213, | cg00532411, | cg14744743, | cg20877507, | cg11223711, |
| cg03396249, | cg18040813, | cg02712145, | cg19483007, | cg06696292, | cg22269622, | cg13152690, |
| cg19011752, | cg00574919, | cg15531369, | cg03455424, | cg19196684, | cg13592399, | cg24825210, |
| cg10247358, | cg15582719, | cg19776515, | cg12932195, | cg00244642, | cg20271620, | cg02631468, |
| cg25037595, | cg08959230, | cg14656657, | cg25199005, | cg00818828, | cg00882832, | cg02892650, |
| cg15341750, | cg00829269, | cg06056346, | cg26504835, | cg26139131, | cg10624480, | cg17184326, |
| cg26585868, | cg11424376, | cg14768335, | cg12148979, | cg04138112, | cg01733928, | cg17323243, |
| cg03834055, | cg09777506, | cg10108402, | cg05997941, | cg02376455, | cg23449295, | cg06899970, |
| cg02396224, | cg22141456, | cg24754507, | cg08632388, | cg18541417, | cg13913306, | cg17187673, |
| cg24073146, | cg19356515, | cg13724732, | cg03978579, | cg18842300, | cg21336235, | cg24809011, |
| cg18406033, | cg24029926, | cg15996406, | cg15655366, | cg14399236, | cg26445541, | cg16342149, |
| cg19777638, | cg08651763, | cg00996262, | cg04160501, | cg24376689, | cg08271996, | cg17350552, |
| cg18128147, | cg05583103, | cg01408508, | cg26451323, | cg03826463, | cg26195706, | cg02365596, |
| cg09760666, | cg14911242, | cg02930687, | cg07543967, | cg03345017, | cg07378013, | cg11779440, |
| cg01770831, | cg02936872, | cg25678745, | cg10576232, | cg25019564, | cg03606646, | cg27127142, |
| cg07370023, | cg13891702, | cg12742758, | cg01189813, | cg10802480, | cg23509098, | cg14479947, |
| cg16035419, | cg00898374, | cg09046309, | cg03385495, | cg01039401, | cg27607338, | cg10093648, |
| cg02068318, | cg06316219, | cg04271791, | cg18195416, | cg00407468, | cg23987876, | cg07642463, |
| cg19043522, | cg06926362, | cg06728974, | cg17349632, | cg09641919, | cg07510611, | cg15765502, |
| cg07017275, | cg27593250, | cg11724970, | cg19077806, | cg25503903, | cg23929381, | cg02896403, |
| cg13414542, | cg19315479, | cg06326428, | cg03623354, | cg14555543, | cg22004774, | cg13532854, |
| cg27105827, | cg02292949, | cg11979303, | cg01335372, | cg06205922, | cg09013220, | cg03957124, |
| cg00728029, | cg21060796, | cg20564913, | cg08047886, | cg13144059, | cg27052344, | cg24586121, |
| cg09297252, | cg00746386, | cg05635617, | cg20982690, | cg21081543, | cg06608166, | cg25806655, |
| cg12731773, | cg03579187, | cg06897790, | cg19320963, | cg20577572, | cg26009195, | cg22600832, |
| cg15823330, | cg18085400, | cg26259546, | ch.12.50831229F, | cg25441879, | cg19998432, | cg02565993, |
| cg19930737, | cg26778477, | cg23983726, | cg05996250, | cg24530471, | cg11382963, | cg04477202, |
| cg26533949, | cg26032101, | cg09684066, | cg24688348, | cg15408734, | cg20894495, | cg09907521, |
| cg12255698, | cg20211711, | cg05239791, | cg07283011, | cg11183632, | cg23022057, | cg06478195, |
| cg19422340, | cg03109101, | cg17002259, | cg25922105, | cg12213850, | cg08965656, | cg05695699, |
| cg06682454, | cg26888225, | cg12190521, | cg19204924, | cg16436377, | cg06830665, | cg11385003, |
| cg03133868, | cg20596240, | cg02859098, | cg10239022, | cg02067098, | cg22061523, | cg19216792, |
| cg07568313, | cg01069738, | cg17838705, | cg10555297, | cg03294328, | cg21090182, | cg06737937, |
| cg26439759, | cg15846771, | cg01172183, | cg09991425, | cg23101259, | cg08231603, | cg18867462, |
| cg03739172, | cg07594539, | cg13064555, | cg12368342, | cg14787287, | cg17045772, | cg02891785, |
| cg17438055, | cg01624081, | cg17747005, | cg04093323, | cg04929736, | cg03006477, | cg24485820, |
| cg22587703, | cg05818894, | cg11268077, | cg13398947, | cg19872068, | cg20380975, | cg09260627, |
| cg02767202, | cg18479961, | cg02275014, | cg23885932, | cg09134969, | cg02848122, | cg04014595, |
| cg03968126, | cg02245378, | cg10417567, | cg07557423, | cg13519902, | cg22507402, | cg06297005, |
| cg24337818, | cg25827351, | cg22570053, | cg09426434, | cg16518861, | cg00721170, | cg00996847, |
| cg05300440, | cg03958757, | cg14090211, | cg23371680, | cg07316743, | cg03418289, | cg12197557, |
| cg13662634, | cg03117951, | cg19906454, | cg06359394, | cg02900332, | cg15796417, | cg21426303, |
| cg16899598, | cg23905164, | cg07489502, | cg19795338, | cg03856024, | cg25479082, | cg08571049, |
| cg00959334, | cg07297322, | cg12571687, | cg12832228, | cg03543639, | cg16397620, | cg12457721, |
| cg02574033, | cg00392075, | cg15300814, | cg10936323, | cg05052194, | cg08142232, | cg07467854, |
| cg12268888, | cg27088038, | cg07664029, | cg17858618, | cg05040429, | cg21142904, | cg17323040, |
| cg16522885, | cg01847719, | cg14604871, | cg02736228, | cg16498681, | cg22768700, | cg15078061, |
| cg06178492, | cg09597767, | cg11556164, | cg22176042, | cg06721712, | cg14729455, | cg25092473, |
| cg21637884, | cg09888620, | cg14846368, | cg11220060, | cg22843765, | cg10769157, | cg05604487, |
| cg04645123, | cg01837533, | cg07231828, | cg02113214, | cg08160706, | cg07697597, | cg00217225, |
| cg17767596, | cg14099293, | cg11851915, | cg14985989, | cg19712813, | cg14187409, | cg00012203, |
| cg22744480, | cg19086110, | cg17483297, | cg12978433, | cg01236027, | cg27633877, | cg18775736, |
| cg19782746, | cg13801201, | cg08402734, | cg00660384, | cg17372223, | cg12071968, | cg10075436, |
| cg23119809, | cg09721204, | cg14317513, | cg14970987, | cg05885577, | cg06575013, | cg00146837, |
| cg06465194, | cg00566164, | cg24903668, | cg19984911, | cg26450750, | cg01892516, | cg01368068, |
| cg02460716, | cg17893857, | cg18606993, | cg04742282, | cg01317226, | cg16878641, | cg22661950, |
| cg04273431, | cg11549025, | cg19089701, | cg23685994, | cg12446082, | cg09644836, | cg09547032, |
| cg16817826, | cg08698026, | cg01815529, | cg14025606, | cg12035269, | cg05188150, | cg09774204, |
| cg12221864, | cg26062560, | cg23097296, | cg06768599, | cg05088892, | cg03936031, | cg02983911, |
| cg14282137, | cg08020177, | cg13560068, | cg16330247, | cg00930244, | cg27467076, | cg15808426, |
| cg21407906, | cg22545840, | cg01140293, | cg22489957, | cg23461741, | cg07417745, | cg05211642, |
| cg14015211, | cg04088945, | cg06239616, | cg13331446, | cg15525782, | cg26236177, | cg13656866, |
| cg27544384, | cg24563850, | cg16395892, | cg00214165, | cg11176924, | cg17468312, | cg00452958, |
| cg23038824, | cg03129904, | cg02958551, | cg12746059, | cg09030848, | cg27192635, | cg06367117, |
| cg05648324, | cg14780540, | cg00579036, | cg17772163, | cg12603560, | cg27599033, | cg06716411, |
| cg18310593, | cg17353812, | cg23368579, | cg04540493, | cg19380578, | cg10702145, | cg14425722, |
| cg15797314, | cg07335619, | cg11794814, | cg23555120, | cg03655371, | cg17342834, | cg07249274, |
| cg00872256, | cg24204532, | cg24516106, | cg18810347, | cg03320372, | cg00716309, | cg27182527, |
| cg05982757, | cg09239144, | cg09671955, | cg17423032, | cg19619721, | cg20916175, | cg14242091, |
| cg01336629, | cg11952622, | cg20603964, | cg12422026, | cg12468647, | cg16956132, | cg20033557, |
| cg06838581, | cg04730029, | cg06749592, | cg04667180, | cg26329274, | cg05899727, | cg00999988, |
| cg04011984, | cg05235151, | cg09118552, | cg27085107, | cg24939194, | cg06746426, | cg05883195, |
| cg04710115, | cg05193832, | cg01144965, | cg21940308, | cg21561807, | cg11425656, | cg20625967, |
| cg17011981, | cg19613205, | cg01374729, | cg03005685, | cg01184004, | cg14458328, | cg07554041, |
| cg16361705, | cg27452939, | cg03790236, | cg09819033, | cg17384124, | cg23495453, | cg17571933, |
| cg03658275, | cg26023902, | cg26059316, | cg10064907, | cg10155261, | cg14008106, | cg03611029, |
| cg11723713, | cg12131007, | cg22870756, | cg11234564, | cg18155632, | cg23047499, | cg04750580, |
| cg03646096, | cg24613923, | cg04573316, | cg26288502, | cg15768138, | cg13357178, | cg08655701, |
| cg04399988, | cg04617001, | cg25200151, | cg21680980, | cg00650876, | cg00407484, | cg16204135, |
| cg02899253, | cg18120808, | cg12904795, | cg01719577, | cg02742775, | cg01368217, | cg26022315, |
| cg24690231, | cg19651132, | cg02393449, | cg00767000, | cg14199148, | cg15206136, | cg24673101, |
| cg22984439, | cg23986158, | cg00876678, | cg18872418, | cg20469666, | cg02155884, | cg22048948, |
| cg08251531, | cg20232550, | cg07309904, | cg23096644, | cg06508916, | cg25158147, | cg17397592, |
| cg24931092, | cg21010973, | cg10118784, | cg19105245, | cg11141711, | cg16413842, | cg24188017, |
| cg01943478, | cg02824386, | cg11056409, | cg16842060, | cg16018754, | cg04496106, | cg04282912, |
| cg20530613, | cg00901843, | cg08135904, | cg01337515, | cg01745873, | cg17885402, | cg06446163, |
| cg21493727, | cg05903710, | cg00570069, | cg26416905, | cg13442969, | cg01159576, | cg09173265, |
| cg13327734, | cg15072038, | cg12479660, | cg07725389, | cg11201751, | cg00940248, | cg11547950, |
| cg05619320, | cg20992180, | cg04051571, | cg26018382, | cg21860968, | cg02961807, | cg08931102, |
| cg09458197, | cg19806106, | cg11150562, | cg03731171, | cg04205943, | cg03836621, | cg12678507, |
| cg17786902, | cg06347996, | cg22918914, | cg06085877, | cg13996962, | cg01857151, | cg11128457, |
| cg12615866, | cg13777294, | cg19206667, | cg06679296, | cg05044639, | cg06772736, | cg27612389, |
| cg00981837, | cg01535630, | cg05766605, | cg07202222, | cg14004197, | cg05337042, | cg00437166, |
| cg11532131, | cg08514594, | cg16930980, | cg04064998, | cg22681784, | cg23016632, | cg05083414, |
| cg10361589, | cg08462941, | cg02925831, | cg13419700, | cg10721556, | cg08146495, | cg06268921, |
| cg14864352, | cg23083824, | cg01819910, | cg02471658, | cg01056467, | cg20029393, | cg06218338, |
| cg22511413, | cg14639753, | cg08383705, | cg13499773, | cg10784030, | cg24478145, | cg13279956, |
| cg00864618, | cg01677302, | cg20702935, | cg05319389, | cg14782735, | cg00530448, | cg00068779, |
| cg25105990, | cg07251711, | cg07876340, | cg12846232, | cg15728439, | cg15126733, | cg03426840, |
| cg11517132, | cg25622317, | cg06854264, | cg03516836, | cg25706447, | cg12495853, | cg04609694, |
| cg04107773, | cg04513006, | cg07562821, | cg17066058, | cg17726692, | cg09732036, | cg11503359, |
| cg24177452, | cg05498041, | cg09296742, | cg26768605, | cg01142635, | cg16361282, | cg02961326, |
| cg11797364, | cg01798033, | cg21759268, | cg10372372, | cg00187299, | cg25237365, | cg04974323, |
| cg13445943, | cg26033466, | cg03116355, | cg16470762, | cg10120070, | cg12273325, | cg26543989, |
| cg14004678, | cg22218709, | cg00939684, | cg23889654, | cg27237541, | cg01082168, | cg06226384, |
| cg12080566, | cg21665747, | cg23570433, | cg25316969, | cg23153615, | cg04682969, | cg26395211, |
| cg24322366, | cg22734086, | cg22878622, | cg03726525, | cg27296308, | cg05577926, | cg00333824, |
| cg27340057, | cg20069765, | cg20588868, | cg13619597, | cg22869025, | cg26751316, | cg05570980, |
| cg00866814, | cg02454890, | cg03962200, | cg00201819, | cg23389215, | cg23708206, | cg10861807, |
| cg21014259, | cg06928983, | cg14127347, | cg21158087, | cg17545418, | cg24654185, | cg09978395, |
| cg19779605, | cg23018707, | cg09521871, | cg20664313, | cg00642970, | cg02314308, | cg20114103, |
| cg21488396, | cg05306024, | cg20770498, | cg17255450, | cg02520910, | cg11718113, | cg08836353, |
| cg13576994, | cg15023427, | cg02369907, | cg07315234, | cg22218806, | cg07169003, | cg18482892, |
| cg10225488, | cg00422566, | cg14011422, | cg09775121, | cg11086275, | cg03892781, | cg14472972, |
| cg02477380, | cg07077900, | cg19823649, | cg16299091, | cg23268322, | cg00325919, | cg09758462, |
| cg26698693, | cg04960243, | cg07823562, | cg14458509, | cg18085998, | cg13833923, | cg12909925, |
| cg10734734, | cg14189988, | cg03952361, | cg17990871, | cg20349695, | cg10152633, | cg15537433, |
| cg10499651, | cg08866865, | cg18044111, | cg25695424, | cg13661129, | cg00148223, | cg04755031, |
| cg19091719, | cg02693607, | cg19758448, | cg23631538, | cg17145862, | cg17809377, | cg05452899, |
| cg18441733, | cg14764956, | cg12033075, | cg27158386, | cg25551545, | cg09139409, | cg26913977, |
| cg00340382, | cg15172601, | cg16884569, | cg05187422, | cg03700143, | cg00095594, | cg06895660, |
| cg15294263, | cg21464091, | cg06154860, | cg16385583, | cg20092531, | cg17346857, | cg25628740, |
| cg13976544, | cg09889828, | cg00044372, | cg26972969, | cg22900206, | cg26297005, | cg27364766, |
| cg16114962, | cg20324928, | cg24540109, | cg26425045, | cg00561322, | cg15687138, | cg00920691, |
| cg26435172, | cg08435157, | cg02447620, | cg16474722, | cg20899382, | cg01174752, | cg01165355, |
| cg22994191, | cg00401753, | cg17525495, | cg04587084, | cg17204652, | cg14034782, | cg08642292, |
| cg00027637, | cg17244098, | cg07220007, | cg07989629, | cg04388863, | cg25546588, | cg02624855, |
| cg17066238, | cg05337637, | cg03401580, | cg17019292, | cg05565537, | cg12540815, | cg24727433, |
| cg15172007, | cg25382400, | cg15466977, | ch.10.2988224F, | cg22775123, | cg06816087, | cg06760467, |
| cg17908602, | cg04606020, | cg10455676, | cg16446486, | cg15557486, | cg04070176, | cg24635736, |
| cg14158053, | cg15158924, | cg05326036, | cg07107580, | cg23857896, | cg22449337, | cg25137687, |

DMP IDs:

| | | | | |
|---|---|---|---|---|
| cg23603891, | cg09463900, | cg07937631, | cg22065894, | cg07093389, |
| cg08885114, | cg17684296, | cg01428589, | cg20258934, | cg11742207, |
| cg14655122, | cg00703898, | cg14613195, | cg08891605, | cg04720380, |
| cg01804925, | cg07700741, | cg23151309, | cg12927915, | cg13979274, |
| cg22726349, | cg07193534, | cg04662039, | cg00249383, | cg19453136, |
| cg24794833, | cg00901683, | cg26787787, | cg08293097, | cg20800216, |
| cg10355455, | cg23953697, | cg10397765, | cg05070273, | cg16649577, |
| cg14345026, | cg02742455, | cg24201362, | cg10928257, | cg08977536, |
| cg14987534, | cg07996785, | cg27012203, | cg02855633, | cg12636814, |
| cg01812571, | cg09401340, | cg17819983, | cg20705781, | cg15277511, |
| cg26634219, | cg20765441, | cg11511562, | cg08650900, | cg09571352, |
| cg01031616, | cg03540794, | cg16501436, | cg24631735, | cg26462924, |
| cg08209365, | cg22489277, | cg11672479, | cg15763823, | cg22940546, |
| cg19782446, | cg03380518, | cg14252617, | cg03251287, | cg02247163, |
| cg12619165, | cg12257439, | cg16128096, | cg05152067, | cg20926651, |
| cg17463655, | cg00155526, | cg01413249, | cg20581154, | cg13271939, |
| cg00486122, | cg21874832, | cg13601496, | cg17019969, | cg24129390, |
| cg01699293, | cg26964651, | cg03483626, | cg21816330, | cg04467702, |
| cg21961831, | cg18021847, | cg05932360, | cg17872799, | cg17435575, |
| cg26176014, | cg01164141, | cg00107577, | cg21291771, | cg00535538, |
| cg17169998, | cg14645841, | cg26981047, | cg27166177, | cg23094728, |
| cg03454225, | cg25109721, | cg02360836, | cg07159490, | cg26720913, |
| cg03173714, | cg18532726, | cg18852633, | cg14448830, | cg24016844, |
| cg05968145, | cg18122310, | cg16550453, | cg17829314, | cg16134678, |
| cg19375627, | cg05721705, | cg20345556, | cg14287923, | cg25950235, |
| cg09974016, | cg00665343, | cg00563300, | cg10887359, | cg24183324, |
| cg00621646, | cg04225510, | cg24069188, | cg15578837, | cg07712329, |
| cg19457506, | cg24917579, | cg03826564, | cg08818525, | cg10598030, |
| cg19696123, | cg06128448, | cg25486386, | cg08214267, | cg07838272, |
| cg11779019, | cg10302026, | cg19595835, | cg09480470, | cg23885415, |
| cg07959490, | cg24235882, | cg18177616, | cg24577220, | cg11326574, |
| cg21087427, | cg01704226, | cg16651126, | cg11763094, | cg17046977, |
| cg12184660, | cg06457408, | cg27570978, | cg09073732, | cg24273394, |
| cg18802486, | cg02891801, | cg04020764, | cg04716021, | cg21900799, |
| cg03912740, | cg11418427, | cg24410945, | cg03989610, | cg13974531, |
| cg02238069, | cg13077745, | cg12682367, | cg22791936, | cg19404977, |
| cg14834267, | cg18412499, | cg04373117, | cg07279281, | cg00241664, |
| cg10341935, | cg08555325, | cg02326796, | cg08133919, | cg18052430, |
| cg18114053, | cg00325661, | cg25186965, | cg21784134, | cg10426076, |
| cg04158290, | cg16947394, | cg24472231, | cg09501274, | cg13240385, |
| cg19675223, | cg10856045, | cg25890679, | cg04424800, | cg10054150, |
| cg18493449, | cg00135852, | cg14210811, | cg06857837, | cg07665487, |
| cg14003611, | cg02650317, | cg13643774, | cg25273340, | cg16016506, |
| cg04431935, | cg26672426, | cg25125298, | cg13925692, | cg03432172, |
| cg15572235, | cg27060622, | cg04939302, | cg26823212, | cg02593579, |
| cg18335607, | cg18056973, | cg24280925, | cg24768974, | cg27251473, |
| cg19880852, | cg05840118, | cg26159205, | cg07089235, | cg26922678, |
| cg05432213, | cg18447772, | cg25492971, | cg26398791, | cg25013052, |
| cg05955287, | cg18066598, | cg06526179, | cg09518906, | cg10507961, |
| cg02598872, | cg00318322, | cg02617837, | cg14765818, | cg01043019, |
| cg05953911, | cg13521002, | cg05164321, | cg11094694, | cg24776910, |
| cg12043807, | cg04395970, | cg08316699, | cg17392730, | cg07880384, |
| cg16176048, | cg26767632, | cg16604086, | cg10096050, | cg11787828, |
| cg17504135, | cg00641009, | cg23141183, | cg20567785, | cg10739528, |
| cg03388575, | cg09954549, | cg23684682, | cg08970651, | cg02160627, |
| cg23360087, | cg06024099, | cg15175446, | cg20775959, | cg00285620, |
| cg27637086, | cg13918631, | cg07930469, | cg22595209, | cg20009594, |
| cg27434351, | cg27390220, | cg25420477, | cg18060431, | cg07421969, |
| cg23815491, | cg12057242, | cg01614726, | cg02810134, | cg05026186, |
| cg15246825, | cg06675144, | cg01306510, | cg23990535, | cg10172783, |
| cg23109867, | cg20372956, | cg09017117, | cg13337731, | cg20487152, |
| cg04586748, | cg12396999, | cg27294164, | cg11248668, | cg18206768, |
| cg14790739, | cg17347335, | cg14117180, | cg20639802, | cg25107282, |
| cg24036039, | cg02556071, | cg12750902, | cg02389084, | cg13823003, |
| cg20713810, | cg13021333, | cg12036633, | cg24278841, | cg23933044, |
| cg24158448, | cg26345203, | cg13275176, | cg15150017, | cg08478250, |
| cg01392180, | cg23496331, | cg15570464, | cg03135515, | cg26332191, |
| cg26862691, | cg21582956, | cg27434584, | cg01026796, | cg10476473, |
| cg22864266, | cg03525023, | cg03988936, | cg24803912, | cg01961387, |
| cg26130645, | cg23352712, | cg01093363, | cg11082907, | cg12857541, |
| cg05104283, | cg20783806, | cg04361015, | cg08758174, | cg02705374, |
| cg15951061, | cg15442105, | cg23314055, | cg09970194, | cg23927014, |
| cg02480675, | cg15423222, | cg20211422, | cg15234263, | cg02565647, |
| cg24434118, | cg11283100, | cg04576203, | cg12803811, | cg00122347, |
| cg26607641, | cg11951143, | cg23848568, | cg12974660, | cg27199872, |
| cg09315024, | cg05418487, | cg07369956, | cg15028282, | cg10597661, |
| cg20899383, | cg17434643, | cg21075829, | cg10839684, | cg06300469, |
| cg21701531, | cg26776175, | cg08770523, | cg01170247, | cg19606103, |
| cg17075096, | cg14307157, | cg03446863, | cg11025960, | cg25668426, |
| cg05439814, | cg26561187, | cg05397812, | cg05762616, | cg03077712, |
| cg25312372, | cg09390186, | cg01020694, | cg12964047, | cg04757389, |
| cg25827102, | cg06221087, | cg14494812, | cg10647925, | cg09875624, |
| cg24319547, | cg00211436, | cg20241254, | cg15334006, | cg25921518, |
| cg09921682, | cg18788932, | cg01578764, | cg05140231, | cg15543167, |
| cg17081914, | cg09432902, | cg17250160, | cg20982735, | cg05126147, |
| cg00036110, | cg01134183, | cg07273051, | cg06039161, | cg06528231, |
| cg11561737, | cg26785157, | cg01312316, | cg24549609, | cg22540514, |
| cg02769794, | cg03824562, | cg16404371, | cg26658039, | cg20701901, |
| cg06292304, | cg06268510, | cg16858463, | cg18919192, | cg16194715, |
| cg15619732, | cg24919790, | cg21560774, | cg26161780, | cg03754128, |
| cg17794299, | cg16682663, | cg10090985, | cg07101781, | cg26748578, |
| cg14952449, | cg20890156, | cg07651687, | cg24990327, | cg19247726, |
| cg12436019, | cg03156769, | cg19878849, | cg05014405, | cg02027717, |
| cg09579989, | cg06225581, | cg07350703, | cg10888111, | cg16458868, |
| cg24125287, | cg13811936, | cg23739862, | cg02368508, | cg14880184, |
| cg23097139, | cg00292432, | cg03918221, | cg00965023, | cg06502868, |
| cg25622896, | cg22974837, | cg02783179, | cg18782991, | cg14929208, |
| cg25430506, | cg23018092, | cg10330371, | cg03326125, | cg03013172, |
| cg17168630, | cg00034755, | cg00777546, | cg07002372, | cg11818615, |
| cg11626195, | cg17373343, | cg03002232, | cg19100034, | cg24728281, |
| cg15135286, | cg16427488, | cg26834169, | cg19262573, | cg08696192, |
| cg05894734, | cg14992154, | cg05769505, | cg05336395, | cg07202320, |
| cg06881239, | cg24760467, | cg10894626, | cg18133905, | cg20063728, |
| cg26593829, | cg24535978, | cg09327610, | cg17628642, | cg11788450, |
| cg13560841, | cg21129181, | cg10742917, | cg13730618, | cg22132876, |
| cg15797971, | cg08612468, | cg22227345, | cg19404582, | cg15377988, |
| cg26395694, | cg12069692, | cg17356718, | ch.3.42394281R, | cg03215105, |
| cg01720795, | cg21324211, | cg14039939, | cg00454290, | cg25204543, |
| cg16368763, | cg13068095, | cg25028293, | cg19768311, | cg14863019, |
| cg12393589, | cg17834136, | cg24825027, | cg23513175, | cg17595386, |
| cg06140152, | cg07616471, | cg00988050, | cg20917241, | cg11436222, |
| cg02628202, | cg14993964, | cg08986340, | cg16233920, | cg05483406, |
| cg11952315, | cg08330349, | cg06017355, | cg05749493, | cg10977770, |
| cg12966367, | cg01447112, | cg14224762, | cg27002826, | cg22809780, |
| cg18428961, | cg03729553, | cg01408845, | cg20893203, | cg16823009, |
| cg00620746, | cg26499055, | cg27209578, | cg02555074, | cg26256263, |
| cg08054032, | cg15289316, | cg03578689, | cg04250867, | cg04035559, |
| cg01276938, | cg03361575, | cg02008285, | cg02821464, | cg02480835, |
| cg12432552, | cg23451311, | cg17137218, | cg09888012, | cg24337010, |
| ch.1.28801127F, | cg02657832, | cg03539041, | cg26146562, | cg06198351, |
| cg26468430, | cg20075315, | cg03752741, | cg20585916, | cg04592413, |
| cg16452840, | cg16250355, | cg03478969, | cg23994748, | ch.4.183293081R, |
| cg04674508, | cg15632901, | cg06923531, | cg00706302, | cg25729826, |
| cg12421513, | cg24822602, | cg19296501, | cg03712221, | cg18191873, |
| cg15642292, | cg07999953, | cg05546763, | cg15114105, | cg02935052, |
| cg14411221, | cg17322632, | cg13758724, | cg06969287, | cg19195419, |
| cg10613925, | cg07236911, | cg11155082, | cg00445126, | cg05934682, |
| cg20316048, | cg06421633, | cg04140633, | cg24638819, | cg08221272, |
| cg15513936, | cg00263856, | cg05480453, | cg09036127, | cg09348948, |
| cg23207527, | cg18305652, | cg08810813, | cg21436544, | cg15384640, |
| cg04143348, | cg03426762, | cg23798120, | cg11241756, | cg03310838, |
| cg04615859, | cg26802026, | cg02625968, | cg26664797, | cg12802093, |
| cg13858909, | cg02786378, | cg10442932, | cg01628528, | cg04402966, |
| cg17224837, | cg14081015, | cg18189607, | cg15777523, | cg06888415, |
| cg13404038, | cg20504993, | cg15595502, | cg12564567, | cg15269027, |
| cg16922400, | cg15570148, | cg13082157, | cg00690746, | cg10349065, |
| cg22993667, | cg06106428, | cg12023686, | cg03431137, | cg27503015, |
| cg25305879, | cg08028037, | cg00571033, | cg03191116, | cg10729426, |
| cg00150500, | cg02930132, | cg16842717, | cg20365867, | cg11106262, |
| cg10023915, | cg06535156, | cg11465466, | cg01558960, | cg18562578, |
| cg15042747, | cg20650802, | cg24101283, | cg18988889, | cg13468764, |
| cg11155735, | cg16498741, | cg01174096, | cg23054394, | cg19815813, |
| cg24277791, | cg00547768, | cg10529662, | cg22142329, | cg19714279, |
| cg24390965, | cg09585247, | cg05122644, | cg06478094, | cg05134945, |
| cg01873311, | cg05607401, | cg21752211, | cg20735682, | cg19748509, |
| cg13273540, | cg01341643, | cg00577588, | cg10157234, | cg26826223, |
| cg01767544, | cg11195707, | cg25448856, | cg01434694, | cg15159111, |
| cg02117903, | cg00929411, | cg06518271, | cg08240383, | cg06027949, |
| cg13364146, | cg11662638, | cg11046502, | cg03167883, | cg08725389, |
| cg00769810, | cg09597585, | cg17247026, | cg01892727, | cg11795276, |
| cg08474786, | cg10299383, | cg22356381, | cg14135522, | cg21819468, |
| cg00635528, | cg16104446, | cg00929878, | cg15368722, | cg01750261, |
| cg03883605, | cg02710296, | cg16667279, | cg03461298, | cg07108206, |
| cg13138389, | cg18067420, | cg25563456, | cg15212418, | cg13741406, |
| cg08315277, | cg20841588, | cg27175386, | cg20363989, | cg10754315, |
| cg24802745, | cg00707300, | cg07135660, | cg23496755, | cg07762788, |
| cg05913684, | cg08448893, | cg04779428, | cg00371418, | cg26022093, |
| cg02882979, | cg08438529, | cg01720742, | cg02328578, | cg17881363, |
| cg05890898, | cg20610605, | cg25751371, | cg26305504, | cg12249227, |
| cg00178506, | cg08337166, | cg00371702, | cg09509553, | cg17837485, |
| cg24837680, | cg06791592, | cg21558752, | cg13874070, | cg21484512, |
| cg14103106, | cg11195797, | cg13236854, | cg06933031, | cg26125787, |
| cg27582994, | cg12347740, | cg17900356, | cg03659354, | cg26525147, |
| cg04247218, | cg08695357, | cg18952654, | cg19851574, | cg06400745, |
| cg17257720, | cg25729060, | cg23029409, | cg01323954, | cg16114441, |
| cg00066817, | cg26465251, | cg02955050, | cg19086309, | cg04094433, |
| cg24469729 | | | | |

¹CvC—cancer vs. control samples (TCGA dataset)
²PvR—progressive vs. regressive CIS dataset

TABLE 7
Metrics:

| Number | ROC AUC | ROC | Threshold | Sensitivity | Specificity | Sensitivity | Specificity |
|---|---|---|---|---|---|---|---|
| of DMPs | (CvC¹) | (PvR²) | value | (CvC) | (CvC) | (PvR) | (PvR) |
| 2000 | 0.983075933 | 0.773919753 | 0.34 | 1 | 0.683783784 | 0.611111111 | 0.777777778 |

DMP IDs:

| | | | | | | |
|---|---|---|---|---|---|---|
| cg01415527, | cg19890264, | cg02810375, | cg10991303, | cg25405821, | cg18280057, | cg22035259, |
| cg14392755, | cg13341460, | cg24700760, | cg03983713, | cg24938743, | cg00207939, | cg10410852, |
| cg14324370, | cg11145162, | cg08855022, | cg20822052, | cg15946224, | cg22518745, | cg10838683, |
| cg03897125, | cg09927637, | cg06016466, | cg04089240, | cg10596108, | cg25842896, | cg03830282, |
| cg23040992, | cg08893692, | cg09391966, | cg21044022, | cg03809526, | cg20792436, | cg00804934, |
| cg14530233, | cg08193333, | cg18633432, | cg25602765, | cg14668821, | cg15652863, | cg26225694, |
| cg03811478, | cg10948795, | cg26877565, | cg23412850, | cg22849543, | cg20090143, | ch.9.178852F, |
| cg14511745, | cg13817952, | cg13349606, | cg16545749, | cg19853517, | cg15189070, | cg10516886, |
| cg20555564, | cg25145360, | cg15114510, | cg02224994, | cg03932048, | cg01171858, | cg19096358, |
| cg19437341, | cg12466324, | cg15257836, | cg12287945, | cg04877966, | cg07107453, | cg15985905, |
| cg12153044, | cg24814257, | cg22379212, | cg15050398, | cg19494004, | cg05688618, | cg04255770, |
| cg18736063, | cg01274715, | cg02637101, | cg25760950, | cg22524735, | cg09234582, | cg17602823, |
| cg20848130, | cg03579738, | cg16811695, | cg02691360, | cg03902565, | cg15249411, | cg03520938, |
| cg18447587, | cg00357988, | cg06499647, | cg13093774, | cg20548564, | cg22680459, | cg27514986, |
| cg05844625, | cg26374823, | cg06110816, | cg12664243, | cg24680852, | cg01761409, | cg09702315, |
| cg18978493, | cg22648015, | cg22515937, | cg19798368, | cg08285446, | cg16157288, | cg01367627, |
| cg07013680, | cg12618270, | cg26462931, | cg27315555, | cg21901307, | cg14899145, | cg04555771, |
| cg19940537, | cg01667319, | cg10712091, | cg18884237, | cg02984790, | cg07002498, | cg08285289, |
| cg00816515, | cg20276503, | cg00933538, | cg03970588, | cg15038672, | cg13443661, | cg12121318, |
| cg16280705, | cg03076790, | cg16566627, | cg21821659, | cg27272293, | cg08240778, | cg06477797, |
| cg01204634, | cg12034158, | cg01168289, | cg17515456, | cg20897830, | cg18500727, | cg19035993, |
| cg23423766, | cg06986705, | cg26679589, | cg17932055, | cg10522447, | cg23460578, | cg06711380, |
| cg14968482, | cg09806629, | cg09469223, | cg27298170, | cg24579589, | cg10830518, | cg23425079, |
| cg13971283, | cg16889990, | cg02238549, | cg12353452, | cg16280922, | cg26940751, | cg11099041, |
| cg04834097, | cg16337087, | cg14513316, | cg06437464, | cg19753867, | cg26971042, | cg00472801, |
| cg02880508, | cg17906873, | cg07873449, | cg26807731, | cg23158051, | cg02151625, | cg12131419, |
| cg22813798, | cg24257490, | cg12052203, | cg05676251, | cg04042861, | cg15393004, | cg16533767, |
| cg08041408, | cg14530476, | cg26313233, | cg04781626, | cg07608565, | cg11235297, | cg19754554, |
| cg14568286, | cg09638944, | cg12349571, | cg23075144, | cg12580583, | cg13409645, | cg19276540, |
| cg05300440, | cg04581571, | cg03192163, | cg07895227, | cg27422807, | cg18552620, | cg17410431, |
| cg27241134, | cg18157915, | cg03622689, | cg05159438, | cg06295032, | cg14382070, | cg19433487, |
| cg18717355, | cg09742643, | cg06777853, | cg06937164, | cg02717150, | cg07397983, | cg13996453, |
| cg14203292, | cg05407489, | cg14820908, | cg22305258, | cg04778271, | cg02550003, | cg19218722, |
| cg12391352, | cg17589590, | cg06242242, | cg02691035, | cg05210904, | cg08903441, | cg14470978, |
| cg11662340, | ch.10.21102959F, | cg20322837, | cg07909359, | cg19862860, | cg03342640, | cg19926776, |
| cg21364231, | cg10717189, | cg11304383, | cg14119001, | cg10133953, | cg14537430, | cg09869729, |
| cg16049890, | cg24220046, | cg20214477, | cg02081051, | cg09817427, | cg26465743, | cg22547404, |
| cg05673882, | cg13624150, | cg09712606, | cg14349231, | cg07249209, | cg06349981, | cg16049367, |
| cg13503583, | cg17078815, | cg17832029, | cg11524422, | cg10642423, | cg12165772, | cg15808795, |
| cg15732221, | cg05858860, | cg27483342, | cg01964170, | cg23067535, | cg13995193, | cg24272216, |
| cg08078213, | cg23384027, | cg04704856, | cg11271171, | cg17084697, | cg18342939, | cg26269882, |
| cg09731306, | cg07672051, | cg07108351, | cg22623273, | cg08206517, | cg07041470, | cg11414046, |
| cg18164305, | cg17974378, | cg21726618, | cg08729810, | cg12940522, | cg00898480, | cg26863881, |
| cg22868624, | cg19111338, | cg10729062, | cg23004527, | cg02059813, | cg07989501, | cg25481680, |
| cg05441372, | cg08838806, | cg11172286, | cg09795127, | cg08250506, | cg13369325, | cg05517218, |
| cg15573359, | cg24077770, | cg20126647, | cg00633552, | cg25418748, | cg26291195, | cg20410537, |
| cg12212591, | cg27582986, | cg22740866, | cg07936950, | cg14556677, | cg13685951, | cg07173239, |
| cg24037425, | cg19837174, | cg26115667, | cg17900697, | cg15901804, | cg22369037, | cg24319076, |
| cg17603909, | cg20440414, | cg26175448, | cg03634479, | cg21974580, | cg05938623, | cg11600697, |
| cg04826040, | cg10334121, | cg01011748, | cg18749629, | cg25358081, | cg05646686, | cg10615842, |
| cg09022584, | cg00828762, | cg06931267, | cg01893030, | cg19225068, | cg06366789, | cg23680067, |
| cg25744613, | cg19890879, | cg13824319, | cg09032544, | cg09889920, | cg01628253, | cg04753829, |
| cg24704287, | cg08754456, | cg05985303, | cg18516317, | cg03756088, | cg12220034, | cg19494100, |
| cg02386159, | cg05120331, | cg16693369, | cg26643856, | cg06319762, | cg25315312, | cg23936023, |
| cg20816077, | cg13700148, | cg05085132, | cg00398268, | cg02823457, | cg25791279, | cg17188169, |
| cg27109971, | cg18041277, | cg11120736, | cg08322978, | cg22680252, | cg21607076, | cg14405526, |
| cg20469733, | cg16348668, | cg18437602, | cg17859110, | cg16864359, | cg06318062, | cg22347696, |
| cg07222250, | cg06463010, | cg04744409, | cg01068808, | cg08316234, | cg22619412, | cg24697184, |
| cg18508148, | cg17962527, | cg21210370, | cg27147718, | cg22399111, | cg05032550, | cg23669159, |
| cg13031623, | cg11433659, | cg23612557, | cg03041650, | cg21845957, | cg08918224, | cg19867649, |
| cg00764668, | cg09671650, | cg16906456, | cg05460486, | cg14580081, | cg18900838, | cg18652285, |
| cg20758492, | cg08779649, | cg24955955, | cg19350682, | cg16636944, | cg04789056, | cg00871381, |
| cg26281789, | cg05513069, | cg14666655, | cg07166679, | cg13632981, | cg08838779, | cg19165274, |
| cg01521397, | cg09962377, | cg14611684, | cg12809234, | cg07983394, | cg06020529, | cg05676541, |
| cg24984834, | cg08385097, | cg05556219, | cg12007828, | cg14407666, | cg02956254, | cg23208850, |
| cg01481879, | cg11314569, | cg08606748, | cg25781121, | cg17520942, | cg13061637, | cg22314787, |
| cg11215621, | cg20997159, | cg22672431, | cg22304169, | cg00533934, | cg09774787, | cg18278265, |
| cg15270950, | cg10668555, | cg01253826, | cg21644489, | cg19749688, | cg11290720, | cg02137495, |
| cg16799087, | cg22789254, | cg02538553, | cg08378129, | cg10537421, | cg24537481, | cg20568038, |
| cg06922352, | cg07252778, | cg09438612, | cg17106222, | cg09704223, | cg13871900, | cg01518225, |
| cg02350211, | cg17338254, | cg00243281, | cg04320928, | cg17545146, | cg00340161, | cg11410023, |
| cg19845873, | cg15246232, | cg03895857, | cg23312964, | cg03876150, | cg01855803, | cg09537448, |
| cg24857399, | cg13472369, | cg07985366, | cg16218204, | cg19427600, | cg02835214, | cg18589102, |
| cg00377358, | cg10708675, | cg16480209, | cg05149498, | cg18821144, | cg03653399, | cg04468741, |
| cg27239828, | cg05395403, | cg07633835, | cg11241627, | cg00318111, | cg10217202, | cg14223671, |
| ch.10.133109453R, | cg26148020, | cg20455436, | cg27107485, | cg06822952, | cg06724305, | cg19313402, |
| cg08136806, | cg11843188, | cg26134090, | cg25710630, | cg13114315, | cg06643271, | cg12153139, |
| cg16976370, | cg12171509, | cg08908089, | cg13439595, | cg12504957, | cg22573650, | cg25967957, |
| cg22454227, | cg23234832, | cg00050294, | cg20712426, | cg19266329, | cg12849359, | cg23760945, |
| cg12641569, | cg18633178, | cg27628849, | cg17790333, | cg00240195, | cg20685898, | cg14513448, |
| cg24209528, | cg11335119, | cg08730743, | cg03496220, | cg11697870, | cg10402121, | cg10081998, |
| cg22753962, | cg17272843, | cg13977320, | cg24430207, | cg06218066, | cg01333884, | cg06010020, |
| cg14988722, | cg18629334, | cg24803906, | cg02856132, | cg01418351, | cg14623518, | cg08847856, |
| cg00876127, | cg08187089, | cg21221899, | cg08805208, | cg18857159, | cg23580410, | cg22573675, |
| cg03113475, | cg25701364, | cg06548410, | cg02423080, | cg06448249, | cg25189007, | cg03983058, |
| cg08827358, | cg06671706, | cg06375525, | cg17213060, | cg00650856, | cg00326842, | cg04321823, |
| cg18705773, | cg09957386, | cg24374857, | cg15602135, | cg01203365, | cg23637124, | cg15882999, |
| cg09166091, | cg15718289, | cg15539073, | cg20561261, | cg12251075, | cg21898073, | cg15104044, |
| cg22046187, | cg12562967, | cg06802812, | cg04582254, | cg23024775, | cg23727813, | cg21634951, |
| cg01492656, | cg05516842, | cg27141788, | cg20201639, | cg12769767, | cg10901633, | cg10010997, |
| cg06142043, | cg23277715, | cg07048951, | cg23050442, | cg21160877, | cg02865068, | cg13813366, |
| cg15135803, | cg17811857, | cg16682989, | cg22073794, | cg01519017, | cg02283151, | cg21457110, |
| cg25766774, | cg06960203, | cg03491821, | cg22366214, | cg12844895, | cg02383368, | cg09160912, |
| cg22668249, | cg26147935, | cg07650252, | cg15283154, | cg19716462, | cg00003287, | cg01028844, |
| cg08179907, | cg26351966, | cg16143796, | cg02959725, | cg06945667, | cg01599099, | cg00044170, |
| cg13235817, | cg27410679, | cg02611892, | cg00040861, | cg17130812, | cg18832897, | cg25924472, |
| cg06208989, | cg24867180, | cg25390440, | cg11302515, | cg13981310, | cg14343927, | cg06046431, |
| cg11115622, | cg19024700, | cg11301233, | cg10017105, | cg06395414, | cg09913544, | cg05090319, |
| cg24659437, | cg10240853, | cg09826188, | cg01629329, | cg24705708, | cg09369818, | cg23689712, |
| cg14135174, | cg04119909, | cg17446216, | cg05502701, | cg00601916, | cg18270113, | cg21213306, |
| cg18820914, | cg13793510, | cg03628682, | cg02845365, | cg00162104, | cg07793789, | cg24424146, |
| cg26404748, | cg11282564, | cg16404074, | cg14821192, | cg20193363, | cg06996129, | cg07687899, |
| cg07202222, | cg03956243, | cg21703606, | cg06426831, | cg00960050, | cg09863630, | cg07823293, |
| cg06421257, | cg15330508, | cg06504655, | cg21119962, | cg19405836, | cg04933168, | cg03143849, |
| cg19291606, | cg21627466, | cg00593462, | cg09189118, | cg26186546, | cg02042156, | cg23209537, |
| cg24317190, | cg23818827, | cg06560282, | cg00484711, | cg04484075, | cg07944494, | cg04251571, |
| cg14317513, | cg11789795, | cg01826367, | cg19890834, | cg22729681, | cg02487453, | cg00161124, |
| cg03355327, | cg21220927, | cg12591952, | cg25618424, | cg07739686, | cg15445958, | cg08098950, |
| cg26577973, | cg05743713, | cg00807877, | cg16638748, | cg16701890, | cg16627395, | cg07545140, |
| cg03788215, | cg01495663, | cg12221565, | cg12801030, | cg06723644, | cg16451355, | cg12915834, |
| cg17820828, | cg10260999, | cg09848445, | cg17300353, | cg00912926, | cg14939765, | cg17207000, |
| cg03168447, | cg08958931, | cg04370315, | cg27441689, | cg25986496, | cg00702577, | cg21466150, |
| cg05412139, | cg23953176, | cg07119001, | cg07546943, | cg12940430, | cg01994994, | cg08721931, |
| cg19973659, | cg26841017, | cg25310559, | cg04024095, | cg02503117, | cg19479330, | cg08351336, |
| cg08107960, | cg19991079, | cg01794405, | cg00260883, | cg07557639, | cg15008984, | cg11408622, |
| cg01065422, | cg10696876, | cg22170962, | cg26960317, | cg01787944, | cg24504423, | cg09351258, |
| cg09908110, | cg15726378, | cg25837738, | cg19962902, | cg25346915, | cg20682563, | cg09519400, |
| cg01234610, | cg24525967, | cg10456710, | cg16552454, | cg10768391, | cg24041541, | cg19108289, |
| cg09767504, | cg10523966, | cg18979819, | cg15374898, | cg01608493, | cg15755548, | cg14211837, |
| cg11935041, | cg17825100, | cg04091078, | cg20731469, | cg12311651, | cg15098085, | cg21700214, |
| cg00926680, | cg08090128, | cg21815258, | cg00876267, | cg17783401, | cg01028364, | cg12744758, |
| cg01273336, | cg00364644, | ch.16.868576F, | cg07291958, | cg05296818, | cg11518032, | cg23614229, |
| cg02041946, | cg24358599, | cg18730862, | cg17364044, | cg10178263, | cg01158447, | cg04440724, |
| cg05954276, | cg12099971, | cg26455175, | cg14064335, | cg27165888, | cg15696968, | cg00230924, |
| cg15678744, | cg10258271, | cg01769273, | cg22030839, | cg03570006, | cg02783314, | cg14537748, |
| cg26282792, | cg13492803, | cg16986846, | cg01187997, | cg16414821, | cg24620797, | cg08162850, |
| cg17249210, | cg08384171, | cg05490924, | cg07362625, | cg01081636, | cg08162426, | cg02465741, |
| cg12523878, | cg04943661, | cg15287987, | cg23371570, | cg24801244, | cg09879253, | cg13814664, |
| cg04609350, | cg24640641, | ch.13.568598F, | cg15353603, | cg20652235, | cg12565126, | cg24469412, |
| cg21336434, | cg16336056, | cg13070632, | cg14393469, | cg04640885, | cg25071135, | cg02522173, |
| cg26738987, | cg06526189, | cg27533472, | cg13132307, | cg15062795, | cg03660451, | cg24610236, |
| cg22647713, | cg26885220, | cg23514400, | cg03278643, | cg24746444, | cg13135241, | cg19926395, |
| cg23499639, | cg15468976, | cg03671359, | cg20664702, | cg08109855, | cg04856167, | cg16017089, |
| cg07371504, | cg24266651, | cg05617678, | cg02768162, | cg14515211, | cg21526750, | cg08322467, |
| cg23292259, | cg01914181, | cg27089559, | cg11725265, | cg09251074, | cg00190319, | cg07957458, |
| cg24575067, | cg16793199, | cg18535410, | cg16633925, | cg21066063, | cg19415852, | cg02810156, |
| cg04294677, | cg23156029, | cg18033235, | cg15677364, | cg08137080, | cg26731644, | cg09797446, |
| cg06899491, | cg07572052, | cg11192059, | cg27087809, | cg14529146, | cg23185921, | cg18176482, |
| cg22240715, | cg06373505, | cg22045888, | cg07054526, | cg12405341, | cg08923271, | cg20789081, |
| ch.8.58671533F, | cg24702826, | cg23892644, | cg13247580, | cg15033140, | cg14043602, | cg24991874, |
| cg15836763, | cg25529393, | cg04307107, | cg26179948, | cg14794023, | cg05638739, | cg12838192, |
| cg20622410, | cg18621819, | cg13466546, | cg02711849, | cg08696261, | cg09923954, | cg05195085, |
| cg05367087, | cg11665652, | cg01779771, | cg02212491, | cg01926646, | cg02902882, | cg10807889, |
| cg03201604, | cg14355911, | cg19241311, | cg06018514, | cg00257202, | cg13430463, | cg05541417, |
| cg16316472, | cg01772149, | cg10987682, | cg05155775, | cg25563516, | cg16982658, | cg07609844, |
| cg02621540, | cg12015124, | cg23102388, | cg09797971, | cg19219435, | cg18725076, | cg15303382, |
| cg08129759, | cg02303933, | cg13406896, | cg06178942, | cg15121959, | cg26576041, | cg07347276, |
| cg21188772, | cg09246749, | cg03030879, | cg15444217, | cg18225582, | cg01660149, | cg04421891, |
| cg00965578, | cg26860257, | cg26134124, | cg23779626, | cg22869660, | cg15605011, | cg06962970, |
| cg13212575, | cg01577001, | cg05034656, | cg09410845, | cg12087941, | cg02444118, | cg12606409, |
| cg15940569, | cg18151858, | cg21448471, | cg13686179, | cg19427686, | cg22739477, | cg24368101, |
| cg05213387, | cg21471574, | cg11953913, | cg12067292, | cg09451073, | cg26513689, | cg22935921, |
| cg11875744, | cg14412282, | cg11612786, | cg17039428, | cg00589895, | cg13999688, | cg01744607, |
| cg05541329, | cg15583263, | cg12421513, | cg23896898, | cg27316970, | cg16156662, | cg07790079, |
| cg02466246, | cg03444587, | cg18970754, | cg04706556, | cg02277530, | cg24155668, | cg04213089, |
| cg17399234, | cg13601435, | cg19364137, | cg21379639, | cg01012572, | cg01196234, | cg01629749, |
| cg25167574, | cg13356455, | cg14879397, | cg13368756, | cg16338100, | cg08235889, | cg03533386, |
| cg14855972, | cg12639429, | cg04002144, | cg18020955, | cg13186559, | cg17128308, | cg04232325, |
| cg01929314, | cg03057855, | cg13520373, | cg09000385, | cg05586134, | cg10677697, | cg22855020, |
| cg27427357, | cg10074498, | cg21614723, | cg18877361, | cg01055714, | cg21663666, | cg10479243, |
| cg04662649, | cg06108254, | cg25371169, | cg21153342, | cg12032116, | cg18286850, | cg27177841, |
| cg27204535, | cg03388025, | cg00303183, | cg07181902, | cg16049941, | cg14207799, | cg14751199, |
| cg10239022, | cg13617591, | cg14014590, | cg05862338, | cg23097143, | cg16444942, | cg04295991, |
| cg20310501, | cg09804398, | cg01373911, | cg19258508, | cg16651583, | cg05260062, | cg21503330, |
| cg22153481, | cg08034440, | cg06687640, | cg26366091, | cg19948037, | cg25062797, | cg24406775, |
| cg27572999, | cg16187013, | cg11600807, | cg08065101, | cg27088844, | cg21926276, | cg27591016, |
| cg13611006, | cg01550307, | cg25599129, | cg03909081, | cg17475085, | cg17329202, | cg15779521, |

DMP IDs:

| | | | | |
|---|---|---|---|---|
| cg21337120, | cg00125601, | cg02374294, | cg14523204, | cg11011774, |
| cg09971646, | cg21372595, | cg07016939, | cg00832476, | cg09271671, |
| cg02730843, | cg19428939, | cg13921444, | cg09053097, | cg07503931, |
| cg20184238, | cg19112424, | cg23274951, | cg03742137, | cg04056669, |
| cg20761322, | cg12465353, | cg06629702, | cg27321311, | cg01195053, |
| cg06713267, | cg16728516, | cg25347801, | cg21350100, | cg04303139, |
| cg15781794, | cg22792938, | cg04258133, | cg21160610, | cg01721410, |
| cg03344290, | cg05582165, | cg12121193, | cg10830905, | cg21790764, |
| cg08209099, | cg12995090, | cg03156115, | cg22952828, | cg09515778, |
| cg18894200, | cg16111791, | cg08679238, | cg22225943, | cg13464351, |
| cg08943180, | cg20800606, | cg10225362, | cg03036210, | cg05314606, |
| cg02794695, | cg12068761, | cg00454592, | cg19598514, | cg10031614, |
| cg06023243, | cg10677245, | cg12533010, | cg26078244, | cg00223046, |
| cg13515998, | cg04508405, | cg02729269, | cg14590902, | cg10441365, |
| cg01474720, | cg16986499, | cg25266603, | cg05081774, | cg23857078, |
| cg15346952, | cg06998361, | cg19694491, | cg07821739, | cg26031288, |
| cg08033114, | cg03483632, | cg04685459, | cg13685680, | cg02156071, |
| cg09184832, | cg04423209, | cg12186991, | cg20737505, | cg11109374, |
| cg21788443, | cg04910877, | cg12366968, | cg24169417, | cg20042281, |
| cg07381283, | cg16669619, | cg12482809, | cg20923156, | cg10179363, |
| cg10013169, | cg16721202, | cg02931526, | cg01287132, | cg21919809, |
| cg06924081, | cg03609847, | cg12118915, | cg14238959, | cg09141965, |
| cg03552658, | cg26622174, | cg09355040, | cg09857273, | cg10537192, |
| cg02819921, | cg04374043, | cg18174834, | cg22027433, | cg26751855, |
| cg19518132, | cg22363670, | cg16058774, | cg11042320, | cg04735960, |
| cg14054343, | cg20536512, | cg00670339, | cg21571135, | cg10600568, |
| cg07284132, | cg19390821, | cg06500883, | cg02941376, | cg20145610, |
| cg27368533, | cg26570765, | cg13742499, | cg21851282, | cg24848349, |
| cg03491270, | cg24227081, | cg09103518, | cg15414833, | cg16602417, |
| cg18847009, | cg22507723, | cg26314566, | cg10300252, | cg13744910, |
| cg20152891, | cg15157024, | cg23288807, | cg10801143, | cg24942416, |
| cg00793186, | cg00614684, | cg22435300, | cg08868915, | cg04680150, |
| cg09934219, | cg06672545, | cg22005145, | cg10606240, | cg06246882, |
| cg04507958, | cg26934062, | cg08195921, | cg01870679, | cg12768145, |
| cg02534744, | cg02874371, | cg02802298, | cg17064564, | cg07429394, |
| cg13954548, | cg23129282, | cg15413793, | cg00372610, | cg26150754, |
| cg20428375, | cg16762581, | cg22798521, | cg11620074, | cg00200164, |
| cg10818319, | cg09056876, | cg09488352, | cg09403933, | cg23792314, |
| cg01699740, | cg22228557, | cg09976984, | cg11154829, | cg11932072, |
| cg16451430, | cg17987855, | cg17974145, | cg24046305, | cg26858890, |
| cg23089673, | cg25960063, | cg05519658, | cg02625902, | cg02839811, |
| cg06758539, | cg07506689, | cg01843020, | cg15751948, | cg14443953, |
| cg25152498, | cg17837894, | cg03267400, | cg06154531, | cg11911648, |
| cg05955436, | cg10097906, | cg20236877, | cg13180224, | cg15368868, |
| cg04441562, | cg23302291, | cg07651687, | cg02009088, | cg02613286, |
| cg16049975, | cg03080043, | cg23332678, | cg14684441, | cg03297845, |
| cg01064108, | cg14988347, | cg02032449, | cg24756528, | cg13128443, |
| cg16629569, | cg08668112, | cg08133641, | cg27316939, | cg15289882, |
| cg18106548, | cg01135780, | cg11062046, | cg18619804, | cg04550079, |
| cg12069446, | cg08058470, | cg06426961, | cg26410630, | cg22283488, |
| cg23389776, | cg08790440, | cg06199468, | cg08748913, | cg15906007, |
| cg03635225, | cg20807254, | cg23285320, | cg18849737, | cg10512769, |
| cg03619568, | cg21901946, | cg07882007, | cg13301931, | cg23521666, |
| cg00308659, | cg03172688, | cg07697895, | cg02225520, | cg01626895, |
| cg06131939, | cg04125246, | cg05981500, | ch.17.458890R, | cg08769966, |
| cg05352321, | cg07866179, | cg03243311, | cg00493617, | cg24654264, |
| cg18735089, | cg11468148, | cg25648211, | cg00812980, | cg18687753, |
| cg03897156, | cg04027757, | cg11395306, | cg16014000, | cg19359238, |
| cg07016060, | cg03071457, | cg25645310, | cg23736297, | cg17647004, |
| cg20292636, | cg04444006, | cg22512222, | cg06789840, | cg03006588, |
| cg07387813, | cg15790852, | cg04696171, | cg18955060, | cg03704308, |
| cg21542796, | cg27026033, | cg22992588, | cg19927510, | cg09979728, |
| cg27135989, | cg19412808, | cg02336334, | cg15099667, | cg15359142, |
| cg15796818, | cg11639168, | cg06912182, | cg12978227, | cg03329019, |
| cg07092593, | cg11864592, | cg19027545, | cg23657639, | cg24558425, |
| cg20300260, | cg11110536, | cg20704560, | cg26161878, | cg00284438, |
| cg10579128, | cg04067612, | cg00847017, | cg21503148, | cg12351554, |
| cg05571645, | cg22190774, | cg12392126, | cg16646892, | cg11431055, |
| cg13490677, | cg18089193, | cg05647602, | cg25785303, | cg16518015, |
| cg18064927, | cg21238812, | cg00744656, | cg11878872, | cg20047447, |
| cg15368905, | cg02207491, | cg00498603, | cg15328582, | cg21243846, |
| cg26644052, | cg11160673, | cg22032706, | cg05949660, | cg02487452, |
| cg19045345, | cg06910125, | cg18679544, | cg00665420, | cg15592184, |
| cg20769356, | cg18087694, | cg01277372, | cg16694311, | cg14741698, |
| cg12157387, | cg20599496, | cg10219840, | cg26865481, | cg01439568, |
| cg14523948, | cg27565067, | cg12568161, | cg26736341, | cg19500098, |
| cg27415425, | cg04349243, | cg07918776, | cg15543490, | cg26896668, |
| cg01471259, | cg19910128, | cg20734569, | cg04267467, | cg10493330, |
| cg05195235, | cg05003422, | cg00255142, | cg02849507, | cg24204940, |
| cg20887711, | cg25363080, | cg16721257, | cg00958927, | cg16708835, |
| cg02773019, | cg02111748, | cg08107075, | cg24463290, | cg15249357, |
| cg23580024, | cg25967872, | cg08407114, | cg11450810, | cg11808885, |
| cg17471062, | cg20120646, | cg16178603, | cg27525626, | cg26364903, |
| cg12766451, | cg09156115, | cg22280173, | cg23195510, | cg19621338, |
| cg15988457, | cg01811126, | cg14357055, | cg22191696, | cg21060340, |
| cg14567043, | cg08767627, | cg06987881, | cg22778120, | cg20211779, |
| cg18382305, | cg13851334, | cg06276663, | cg12722015, | cg13613280, |
| cg01962727, | cg10721030, | cg25871543, | cg24493258, | cg23201513, |
| cg18997124, | cg08876508, | cg00140910, | cg26224314, | cg10792544, |
| cg11255208, | cg09378105, | cg20933586, | cg02543202, | cg27466042, |
| cg14284257, | cg04533116, | cg01036164, | cg08649954, | cg10396673, |
| cg01700124, | cg25062184, | cg26677406, | cg21450547, | cg00355992, |
| cg20665464, | cg26332488, | cg07375912, | cg07464212, | cg23983517, |
| cg04003871, | cg23972551, | cg00309339, | cg02372201, | cg25112191, |
| cg16465128, | cg07609788, | cg05623562, | cg11656163, | cg10716120, |
| cg17974166, | cg08362592, | cg20663376, | cg21830223, | cg13593689, |
| cg01985396, | cg03022094, | cg01599167, | cg07990736, | cg08687507, |
| cg00301555, | cg15800530, | cg26990587, | cg21057228, | cg25520872, |
| cg10866623, | cg03790312, | cg18697929, | cg18559249, | cg00019678, |
| cg11224946, | cg22339902, | cg04285275, | cg02719954, | cg00894222, |
| cg10991175, | cg14109663, | cg19693177, | cg25457956, | cg15999311, |
| cg21752211, | cg21211357, | cg04496233, | cg17271338, | cg24864275, |
| cg17941579, | cg02520160, | cg14718379, | cg01526474, | cg22707016, |
| cg14098299, | cg01248010, | cg02875558, | cg27626387, | cg24464927, |
| cg00982312, | cg16393715, | cg03514843, | cg17873451, | cg23288827, |
| cg00573631, | cg13900327, | cg13397436, | cg25839482, | cg25381253, |
| cg05961935, | cg04222463, | cg03822003, | cg03208951, | cg14867604, |
| cg03078169, | cg18251612, | cg27371984, | cg22144942, | cg02741359, |
| cg04184946, | cg04663194, | cg19207803, | cg17811589, | cg03200120, |
| ch.9.122161992R, | cg12167564, | cg09769755, | cg01108230, | cg07356342, |
| cg25019126, | cg13474124, | cg14950169, | cg25089332, | cg21735676, |
| cg05540413, | cg15608397, | cg01124961, | cg19772723, | cg13070156, |
| cg14439950, | cg18412431, | cg10454258, | cg15291304, | cg06341731, |
| cg24894629, | cg06677089, | cg04746240, | cg06814287, | cg23940655, |
| cg12139329, | cg11190155, | cg19249110, | cg02832646, | cg19407943, |
| cg05732130, | cg19302295, | cg08076044, | cg06054963, | cg11511795, |
| cg23990557, | cg23612849, | cg10601761, | cg10614809, | cg05004940, |
| cg11044605, | cg27136228, | cg21574204, | cg17647537, | cg14506686, |
| cg07533488, | cg00879302, | cg09286090, | cg14126392, | cg06206357, |
| cg07372795, | cg13262687, | cg09696115, | cg24161793, | cg26914296, |
| cg23231727, | cg01338347, | cg14632281, | cg03641066, | cg14163324, |
| cg21810910, | cg17355791, | cg00537387, | cg04864441, | cg04315086, |
| cg08518892, | cg05233946, | cg08550026, | cg20063270, | cg00911981, |
| cg01272940, | cg24140586, | cg17604407, | cg06628109, | cg04079139, |
| cg05498866, | cg23897356, | cg02056809, | cg06651508, | cg02961807, |
| cg15639592, | cg23088110, | cg19049837, | cg03400264, | cg11784626, |
| cg24654525, | cg08238794, | cg08509840, | cg25161386, | cg21278181, |
| cg23280155, | cg06822816, | cg02408850, | cg23696864, | cg14638883, |
| cg17070539, | cg20540327, | cg27133684, | cg04789403, | cg00988346, |
| cg24753662, | cg04916107, | cg14187712, | cg04586579, | cg14158153, |
| cg08716655, | cg19060371, | cg11755053, | cg12528485, | cg05149284, |
| cg18002116, | cg11677222, | cg12157941, | cg17245548, | cg20444990, |
| cg16532497, | cg26930596, | cg16666329, | cg15891268, | cg01336821, |
| cg25260865, | cg11158179, | cg14243623, | cg25976804, | cg08496276, |
| cg03766703, | cg05915004, | cg02930348, | cg13562816, | cg26452584, |
| cg02165355, | cg05740567, | cg10907582, | cg18181229, | cg27603338, |
| cg00852041, | cg08643131, | cg19299094, | cg22658884, | cg17286790, |
| cg07044749, | cg17426592, | cg00145732, | cg18275620, | cg23745444, |
| cg26824326, | cg22698744, | cg09458237, | cg07257768, | cg14182461, |
| cg16027122, | cg12419685, | cg13218903, | cg13135180, | cg11051893, |
| cg17187521, | cg00635299, | cg12827637, | cg11997733, | cg25277809, |
| cg26122413, | cg26360533, | cg19990151, | cg01248866, | cg25664402, |
| cg01040654, | cg00492784, | cg01171706, | cg04314225, | cg13920312, |
| cg17029694, | cg02746869, | cg26249873, | cg02492920, | cg20416031, |
| cg21530045, | cg05390694, | cg22599229, | cg15153394, | cg03651583, |
| cg26233408, | cg09500443, | cg20607318, | cg14487914, | cg05710204, |
| cg22488970, | cg16766966, | cg05522145, | cg23693705, | cg26016210, |
| cg26365885, | cg24307601, | cg05890616, | cg22746529, | cg05344046, |
| cg25937854, | cg06684201, | cg06410746, | cg01219087, | cg20925763, |
| cg21800258, | cg25903779, | cg00621240, | cg07986199, | cg17283506, |
| cg26383838, | cg03896792, | cg23239344, | cg12591125, | cg03461967, |
| cg06174563, | cg23268197, | cg25741368, | cg20786909, | cg23613205, |
| cg04246445, | cg24510700, | cg26120842, | cg08217234, | cg22955555, |
| cg06008873, | cg10197666, | cg20697630, | cg10547205, | cg13670448, |
| cg14381712, | cg06982229, | cg24402151, | cg09740815, | cg12167518, |
| cg02795284, | cg05347845, | cg14417954, | cg12083336, | cg24001710, |
| cg01148127, | cg05081908, | cg23189951, | cg13763724, | cg16801887, |
| cg24225196, | cg14623917, | cg08317263, | cg27035734, | cg26905768, |
| cg19787841, | cg10814153, | cg23454038, | cg07352215, | cg21653105, |
| cg20950033, | cg02010852, | cg00419692, | cg16805189, | cg20457594, |
| cg08309809, | cg19211800, | cg00415978, | cg18435928, | cg14784820, |
| cg00208734, | cg15987431, | cg19622569, | cg00697057, | cg23384708, |
| cg12468708, | cg06880664, | cg11641702, | cg21584759, | cg05728019, |
| cg05873604, | cg01293971, | cg17907567, | cg17510922, | cg14319842, |
| cg12095807, | cg03354957, | cg18754370, | cg21312482, | cg13811446, |
| cg23059304, | cg16409970, | cg06558372, | cg20345978, | cg14240353, |
| cg02595280 | | | | |

¹CvC—cancer vs. control samples (TCGA dataset)
²PvR—progressive vs. regressive CIS dataset

TABLE 8
Metrics:

| Number | ROC AUC | ROC | Threshold | Sensitivity | Specificity | Sensitivity | Specificity |
|---|---|---|---|---|---|---|---|
| of DMPs | (CvC¹) | (PvR²) | value | (CvC) | (CvC) | (PvR) | (PvR) |
| 2000 | 0.982882883 | 0.795524691 | 0.35 | 1 | 0.6 | 0.777777778 | 0.75 |

DMP IDs:

| | | | | | | |
|---|---|---|---|---|---|---|
| cg22799902, | cg19568591, | cg13708218, | cg08897337, | cg00989226, | cg18800042, | cg25059541, |
| cg03826594, | cg00646200, | cg15215155, | cg26880493, | cg00333364, | cg20772593, | cg10180406, |
| cg20823406, | cg06250127, | cg12030835, | cg07015368, | cg04360780, | cg08473660, | cg15766408, |
| cg12201779, | cg24529736, | cg14877413, | cg04907738, | cg10635194, | cg14817783, | cg16377609, |
| cg09481972, | cg03401580, | cg10053879, | cg12589340, | cg19936912, | cg13962664, | cg05130355, |
| cg11260421, | cg12868312, | cg00810752, | cg17242812, | cg21524538, | cg17587456, | cg04628369, |
| cg18210732, | cg08655760, | cg24220031, | cg02876276, | cg18395354, | cg06834507, | cg00039177, |
| cg23899168, | cg13977235, | cg10887359, | cg00632957, | cg00381179, | cg18384588, | cg24531955, |
| cg27578726, | cg19229402, | cg00375691, | cg24337818, | cg05618595, | cg10169364, | cg16115689, |
| cg09377899, | cg18575770, | cg27598017, | cg16292333, | cg06414854, | cg00138407, | cg04002885, |
| cg01780613, | cg22330471, | cg04071694, | cg05970437, | cg06753985, | cg16300021, | cg18143824, |
| cg13378628, | cg03546977, | cg06203102, | cg10402715, | cg26469895, | cg00558031, | cg04052013, |
| cg19007570, | cg01062937, | cg14584031, | cg12552438, | cg19926250, | cg01178680, | cg01528492, |
| cg05790579, | cg20741134, | cg02971124, | cg01430870, | cg03602212, | cg26948895, | cg15583349, |
| cg08835301, | cg26985798, | cg20856330, | cg19589131, | cg16520737, | cg10185885, | cg05327509, |
| cg10373147, | cg03855338, | cg18099096, | cg01264705, | cg17991940, | cg17323956, | cg19611786, |
| cg04364311, | cg23415756, | cg19319774, | cg00843416, | cg12477297, | cg17017596, | cg04218209, |
| cg11344927, | cg02447229, | cg22431867, | cg12394529, | cg00096810, | cg17904786, | cg11408632, |
| cg27123665, | cg02488383, | cg07142893, | cg09423703, | cg24243375, | cg10502563, | cg04444104, |
| cg01178040, | cg05928219, | cg19180602, | cg03613077, | cg04483720, | cg14331623, | cg04747941, |
| cg18680626, | cg12665890, | cg03970501, | cg04383154, | cg12882657, | cg09655005, | cg05298696, |
| cg17019070, | cg26613648, | cg08751499, | cg14082645, | cg07763047, | cg25786651, | cg07533630, |
| cg00667760, | cg26394196, | cg19656282, | cg13075295, | cg11601375, | cg00831247, | cg18348226, |
| cg00187889, | cg00177013, | cg21244580, | cg08616182, | cg11959374, | cg06863619, | cg04828105, |
| cg07347137, | cg20572153, | cg00079598, | cg11116142, | cg17100158, | cg22295628, | cg15854333, |
| cg12577320, | cg21006526, | cg08918020, | cg07903141, | cg02611013, | cg17036562, | cg07732693, |
| cg26810723, | cg17714010, | cg17956609, | cg20821082, | cg09560911, | cg13817265, | cg00844246, |
| cg13249833, | cg04316311, | cg13770399, | cg22482381, | cg02963206, | cg21460686, | cg17006136, |
| cg25364469, | cg11886884, | cg24264578, | cg24510494, | cg25224568, | cg11520003, | cg05598246, |
| cg11078221, | cg01736164, | cg09556527, | cg11698244, | cg06528559, | cg00400832, | cg09690449, |
| cg27271532, | cg19705541, | cg02101892, | cg05763664, | cg17515456, | cg15880846, | cg22513169, |
| cg00314771, | cg02600514, | cg25657700, | cg16294168, | cg04880992, | cg06683080, | cg00027995, |
| cg02520729, | cg00538923, | cg08348949, | cg24221639, | cg09830481, | cg15706657, | cg02590486, |
| cg20706711, | cg21904937, | cg18685835, | cg14409414, | cg25474312, | cg27066296, | cg02719010, |
| cg20220639, | cg10402995, | cg13257331, | cg01508757, | cg18274984, | cg18099632, | cg19410789, |
| cg14157107, | cg11385094, | cg26738010, | cg01160440, | cg05665326, | cg04171803, | cg17714703, |
| cg09115485, | cg22291084, | cg10788321, | cg16628918, | cg14554491, | cg07742235, | cg12205230, |
| cg25757598, | cg06591973, | cg09260089, | cg16933386, | cg20200516, | cg13631916, | cg10215102, |
| cg01052699, | cg15190742, | cg15313332, | cg10073584, | cg20638896, | cg09537600, | cg18082788, |
| cg06040071, | cg26677892, | cg19516457, | cg08384171, | cg09964204, | cg26349332, | cg11117305, |
| cg08891605, | cg03394150, | cg26407161, | cg00273449, | cg26698744, | cg10759361, | cg20492680, |
| cg18808904, | cg25350057, | cg07824824, | cg22718360, | cg10762199, | cg12532343, | cg12476754, |
| cg10613701, | cg24154474, | cg04732357, | cg27072251, | cg03258011, | cg16908156, | cg14778267, |
| cg11779900, | cg05526905, | cg04571189, | cg10514237, | cg12114804, | cg02515468, | cg01170882, |
| cg26676090, | cg02337062, | cg21831927, | cg19137348, | cg21785145, | cg15527678, | cg25310559, |
| cg03296801, | cg05259545, | cg03790324, | cg16768868, | cg27656592, | cg11618704, | cg14614490, |
| cg06891548, | cg11231849, | cg09124093, | cg03232051, | cg20108119, | cg22578843, | cg01946966, |
| cg03682112, | cg12116137, | cg07308824, | cg08846783, | cg13763287, | cg17403817, | cg12686441, |
| cg00686451, | cg10197666, | cg27391470, | cg17125477, | cg16949933, | cg12907876, | cg25770702, |
| cg09024435, | cg16555537, | cg23018873, | cg14025044, | cg17531288, | cg02374984, | cg02140384, |
| cg25604247, | cg26552859, | cg18739887, | cg17344080, | cg05258027, | cg16264807, | cg12366960, |
| cg13050318, | cg09507968, | cg14777056, | cg13480658, | cg04784499, | cg13996619, | cg26992028, |
| cg09156769, | cg23385550, | cg02904442, | cg04699519, | cg22219248, | cg07609844, | cg11057497, |
| cg13313036, | cg04204967, | cg04194728, | cg02418175, | cg16877162, | cg19823329, | cg26608693, |
| cg20284937, | cg05096398, | cg23725394, | cg18825221, | cg01931792, | cg23605175, | cg27584448, |
| cg15426626, | cg06460189, | cg10722298, | cg26316082, | cg00391668, | cg04917258, | cg03681834, |
| cg03152912, | cg25230151, | cg01675238, | cg10607599, | cg27658304, | cg07113642, | cg22188603, |
| cg01635736, | cg23092635, | cg13540075, | cg02320356, | cg19856444, | cg22928329, | cg02269083, |
| cg14227086, | cg09292581, | cg04918402, | cg19591077, | cg15554941, | cg09685505, | cg22517995, |
| cg18584550, | cg02083376, | cg03584646, | cg16465939, | cg18731811, | cg26410632, | cg03458344, |
| cg17592812, | cg17777408, | cg04528352, | cg02979457, | cg05432469, | cg12966875, | cg15514283, |
| cg19326622, | cg04876069, | cg01902939, | cg13135456, | cg18907942, | cg22306928, | cg21530308, |
| cg10539861, | cg25820062, | cg00195749, | cg02696763, | cg01174771, | cg08985570, | cg16078060, |
| cg00310410, | cg06862167, | cg08972588, | cg18882060, | cg24732823, | cg01709551, | cg09264088, |
| cg06858186, | cg24727343, | cg25747030, | cg13329217, | cg21646955, | cg01264126, | cg17903544, |
| cg16969458, | cg10553890, | cg10923219, | cg20157577, | cg20379007, | cg19483330, | cg24513791, |
| cg23431851, | cg09506661, | cg12493645, | cg26147935, | cg12888861, | cg01701655, | cg11905611, |
| cg17366325, | cg06644719, | cg27615067, | cg16173736, | cg20764417, | cg04785083, | cg03430348, |
| cg08679180, | cg22764925, | cg15273575, | cg02110031, | cg00651020, | cg02063488, | cg19494010, |
| cg07545587, | cg20070536, | cg18855140, | cg26635367, | cg00087482, | cg13833527, | cg03392605, |
| cg18626427, | cg13024368, | cg24937269, | cg01751605, | cg23854567, | cg14546076, | cg10536303, |
| cg19711724, | cg24084681, | cg13404546, | cg03084704, | cg26268565, | cg08352032, | cg14730085, |
| cg04081091, | cg13605404, | cg25445977, | cg25023908, | cg21330577, | cg22753340, | cg17485681, |
| cg07528974, | cg25070253, | cg15727320, | cg02398342, | cg00684125, | cg02325880, | cg13831388, |
| cg17854981, | cg11201229, | cg07270872, | cg04036070, | cg15915361, | cg04961911, | cg23445511, |
| cg04515702, | cg11280390, | cg15168615, | cg22816621, | cg17110261, | cg12145080, | cg23077029, |
| cg10248492, | cg06947206, | cg11593949, | cg26940910, | cg07797805, | cg03883555, | cg14588738, |
| cg25819219, | cg07268726, | cg17718703, | cg20979296, | cg01623984, | cg13433612, | cg02805465, |
| cg02025435, | cg06358566, | cg03654735, | cg15180789, | cg08718230, | cg26621911, | cg12514789, |
| cg00360414, | cg24126784, | cg12616935, | cg13767625, | cg24870774, | cg26004761, | cg04574442, |
| cg27636914, | cg09136346, | cg03698393, | cg23529856, | cg11049298, | cg16051002, | cg26826109, |
| cg21919658, | cg08614082, | cg07888967, | cg10509185, | cg17830515, | cg04562562, | cg16794345, |
| cg07856155, | cg15564865, | cg08935021, | cg10235817, | cg25938042, | cg06618298, | cg13560072, |
| cg17734409, | cg11137321, | cg17003736, | cg23584871, | cg23042540, | cg09263407, | cg13262752, |
| cg16246747, | cg25691167, | cg04834436, | cg21194021, | cg25699073, | cg23566793, | cg16190388, |
| cg09877105, | cg05376469, | cg07238926, | cg23013931, | cg06697294, | cg25473115, | cg03440386, |
| cg03715846, | cg10415122, | cg25012919, | cg21499918, | cg14302308, | cg22752023, | cg05048199, |
| cg24672547, | cg05870820, | cg26892394, | cg19822764, | cg20248954, | cg22820233, | cg18476176, |
| cg17833862, | cg13219080, | cg11728867, | cg07805040, | cg20842326, | cg18396992, | cg17836177, |
| cg22403344, | cg03442951, | cg10187059, | cg26079857, | cg03125765, | cg26538736, | cg22695389, |
| cg02128278, | cg26251398, | cg15284082, | cg13319175, | cg22330021, | cg26142132, | cg06232130, |
| cg06983586, | cg02395768, | cg06952117, | cg03474731, | cg14244360, | cg27295716, | cg01975461, |
| cg13266951, | cg21297992, | cg09749638, | cg01770810, | cg04503318, | cg04252616, | cg20593868, |
| cg16601461, | cg16703390, | cg02225716, | cg25820257, | cg25352592, | cg21671806, | cg10683503, |
| cg08813062, | cg04478795, | cg14116388, | cg23526055, | cg06545361, | cg08884854, | cg13279019, |
| cg09959790, | cg00418743, | cg16329509, | cg22304255, | cg14773282, | cg11331556, | cg25038330, |
| cg19389001, | cg11728578, | cg16408843, | cg01961752, | cg18033092, | cg18124330, | cg27266060, |
| cg27518993, | cg09273910, | cg07880741, | cg00293631, | cg01397661, | cg08632659, | cg13076757, |
| cg01284193, | cg06376558, | cg06854487, | cg18545655, | cg13359948, | cg23009355, | cg09459982, |
| cg26651290, | cg06850241, | cg07348809, | cg09942424, | cg08616951, | cg03826759, | cg24845165, |
| cg18580232, | cg22032894, | cg07443717, | cg19103429, | cg14167139, | cg08222002, | cg17177755, |
| cg11813810, | cg10091265, | cg22852235, | cg09440782, | cg24406162, | cg15632825, | cg09882901, |
| cg01558401, | cg25122820, | cg02172381, | cg12495983, | cg11742688, | cg06146910, | cg01903440, |
| cg25806609, | cg25371672, | cg19025034, | cg00157199, | cg15235832, | cg19823847, | cg10633838, |
| cg11008132, | cg15001687, | cg19754520, | cg00784791, | cg11255154, | cg24311251, | cg07063853, |
| cg00505606, | cg21618751, | cg18407136, | cg07858908, | cg02961109, | cg11303842, | cg22736718, |
| cg02173004, | cg02147240, | cg18867708, | cg02585483, | cg00998135, | cg15790839, | cg11902995, |
| cg04818078, | cg23054394, | cg01973139, | cg23037411, | cg17329292, | cg17498476, | cg08553816, |
| cg01246961, | cg04006520, | cg06160397, | cg22796923, | cg03100752, | cg00133403, | cg03587235, |
| cg22558060, | cg24184792, | cg21949274, | cg04616155, | cg11203211, | cg12001304, | cg23437166, |
| cg16221875, | cg12425283, | cg10850197, | cg06146607, | cg21404878, | cg03365548, | cg14043316, |
| cg00789328, | cg25989856, | cg03515557, | cg09986680, | cg15884014, | cg19109909, | cg24505395, |
| cg24411488, | cg07007717, | cg06363918, | cg17223218, | cg16366809, | cg08535411, | cg03718677, |
| cg18711899, | cg21332304, | cg07494646, | cg08822204, | cg15687973, | cg27065003, | cg21751145, |
| cg00226085, | cg01487013, | cg24798956, | cg18086386, | cg21546601, | cg10426893, | cg23052267, |
| cg14928734, | cg22907892, | cg15131647, | cg11438065, | cg13775593, | cg20139898, | cg14039779, |
| cg04624362, | cg25840433, | cg06182018, | cg07046849, | cg01237501, | cg03363248, | cg13682692, |
| cg24828358, | cg00607627, | cg09454613, | cg03127370, | cg05521499, | cg11478534, | cg13593448, |
| cg03999872, | cg08281341, | cg19067392, | cg15386368, | cg26133266, | cg16681649, | cg23552821, |
| cg06233376, | cg01716445, | cg10107292, | cg10762466, | cg02094983, | cg00573355, | cg24170269, |
| cg22705929, | cg10231183, | cg02732358, | cg14298200, | cg17486213, | cg20608032, | cg18222235, |
| cg14897833, | cg19890431, | cg08244522, | cg23186787, | cg06012730, | cg24727024, | cg09786321, |
| cg24366680, | cg11673304, | cg11357813, | cg02826735, | cg22255483, | cg10473266, | cg17136126, |
| cg26457116, | cg03667317, | cg14058851, | cg10911287, | cg06573604, | cg01964795, | cg09174009, |
| cg21291641, | cg15824312, | cg27648946, | cg25698741, | cg08702740, | cg09772097, | cg25388823, |
| cg26070020, | cg13529217, | cg22345878, | cg06525892, | cg02628107, | cg11869184, | cg17350023, |
| cg20746422, | cg25599242, | cg20265358, | cg13063900, | cg11012980, | cg13393110, | cg00867889, |
| cg05959525, | cg03464573, | cg06174296, | cg23632528, | cg02414586, | cg12487540, | cg08860190, |
| cg25515982, | cg23468144, | cg16358099, | cg20684312, | cg13847437, | cg03885975, | cg03850694, |
| cg22992815, | cg16071091, | cg06424576, | cg23583501, | cg18597883, | cg24325215, | cg26928125, |
| cg14304761, | cg23513591, | cg14263118, | cg03239638, | cg06203471, | cg23877720, | cg14121593, |
| cg18935832, | cg12898275, | cg02834497, | cg19445044, | cg24996315, | ch.7.3356624R, | cg17886303, |
| cg16822143, | cg25199283, | cg10434075, | cg13056927, | cg02663352, | cg00830444, | cg06842781, |
| cg04657146, | cg03599855, | cg16405637, | cg19537490, | cg12141659, | cg08393305, | cg12347757, |
| cg11106287, | cg02206373, | cg25262481, | cg05383565, | cg25629790, | cg07440387, | cg06876040, |
| cg07146119, | cg09428340, | cg09765297, | cg04238983, | cg08575249, | cg03228985, | cg23722790, |
| cg24393783, | cg18570392, | cg13432682, | cg18205883, | cg17389949, | cg13365781, | cg19959647, |
| cg27219362, | cg00115567, | cg25575883, | cg01053260, | cg13362105, | cg20879606, | cg14990071, |
| cg10577216, | cg21963048, | cg11577097, | cg15631458, | cg21700723, | cg04678315, | cg27022566, |
| cg18513357, | cg05116382, | cg18317694, | cg00191858, | cg18739537, | cg13260278, | cg17838765, |
| cg09791673, | cg07805221, | cg06525280, | cg13165758, | cg02417857, | cg16118379, | cg06704455, |
| cg09202227, | cg21230538, | cg00936442, | cg17319198, | cg08676534, | cg17329304, | ch.19.58852620F, |
| cg19832529, | cg07333545, | cg04633513, | cg05898629, | cg03233656, | cg04347477, | cg06496728, |
| cg14802097, | cg19697725, | cg01603549, | cg22321318, | cg01715949, | cg18290983, | cg07484827, |
| cg20178075, | cg07700741, | cg01207931, | cg13733266, | cg07911865, | cg06692927, | cg23457901, |
| cg14118666, | cg22704775, | cg03856786, | cg14584529, | cg02575159, | cg02741808, | cg02980079, |
| cg03310242, | cg01206211, | cg12149972, | cg01284448, | cg08859411, | cg13464401, | cg03832522, |
| cg00709966, | cg19170321, | cg27146741, | cg23456689, | cg23521973, | cg00886770, | cg23712370, |
| cg15359163, | cg19410967, | cg23017002, | cg21037008, | cg19814830, | cg17386057, | cg20624830, |
| cg26132452, | cg09681360, | cg20651681, | cg19529146, | cg00727673, | cg00666685, | cg02564272, |
| cg12434735, | cg02596486, | cg00643814, | cg14225926, | cg05885647, | cg00685795, | cg09378441, |
| cg00421089, | cg23036426, | cg16299341, | cg16156182, | cg02960016, | cg17027945, | cg24377330, |
| cg07355688, | cg01244650, | cg24096323, | cg21182694, | cg17309711, | cg01109023, | cg20296493, |
| cg23029801, | cg02793099, | cg10132838, | cg17483139, | cg19764594, | cg26508844, | cg05012972, |
| cg27169073, | cg15673955, | cg26343258, | cg09439054, | cg01408881, | cg21771915, | cg06062590, |
| cg13413982, | cg13596833, | cg18891864, | cg17709703, | cg04598128, | cg27012108, | cg19543471, |
| cg23023044, | cg06664023, | cg25874782, | cg12290311, | cg07507114, | cg05439521, | cg18108022, |
| cg27059354, | cg16090330, | cg01357925, | cg11958128, | cg03579474, | cg24631877, | cg16243665, |
| cg08744475, | cg01879083, | cg18776207, | cg06467040, | cg26322816, | cg22752049, | cg21099332, |
| cg15010544, | cg09178368, | cg11663953, | cg23605797, | cg25805964, | cg20785336, | cg00032756, |
| cg07003587, | cg07469961, | cg17362900, | cg15628953, | cg05298088, | cg17786902, | cg21866596, |
| cg21864845, | cg19602370, | cg23302730, | cg11988531, | cg00964137, | cg15924577, | cg12008066, |
| cg14609729, | cg21529323, | ch.11.1222938R, | cg04830442, | cg06932521, | cg23232755, | cg12554051, |
| cg22403782, | cg05241355, | cg19890277, | cg15751117, | cg15907819, | cg00171126, | cg26872742, |
| cg08180459, | cg09346904, | cg22881834, | cg22382805, | cg06624121, | cg24450494, | cg17350448, |
| cg00099129, | cg01062519, | cg19678610, | cg01029592, | cg24326661, | cg24063856, | cg12761732, |
| cg25731428, | cg22118524, | cg09745688, | cg24973755, | cg02343197, | cg04164499, | cg22717608, |

DMP IDs:

| | | | | |
|---|---|---|---|---|
| cg06180108, | cg04800682, | cg20281912, | cg22307732, | cg27386912, |
| cg00300637, | cg00194681, | cg14633965, | cg03484267, | cg05395156, |
| cg26189067, | cg04873514, | cg26723492, | cg22780475, | cg06802374, |
| cg08902132, | cg16654603, | cg17966362, | cg08299809, | cg02116996, |
| cg10765655, | cg00681278, | cg06675705, | cg27364547, | cg20261082, |
| cg00765737, | cg14494721, | cg07171518, | cg12229172, | cg07382554, |
| cg23171972, | cg14400258, | cg04134096, | cg12624087, | cg06857323, |
| cg08690094, | cg14046988, | cg16800111, | cg17828223, | cg17929645, |
| cg07521912, | cg17302461, | cg24784697, | cg06212175, | cg19002857, |
| cg24296250, | cg06958535, | cg13378865, | cg23490773, | cg10717504, |
| cg04216466, | cg04189768, | cg21770622, | cg11092416, | cg11420883, |
| cg19163818, | cg25994470, | cg24250070, | cg15890849, | cg01755539, |
| cg19965868, | cg10245752, | cg02131230, | cg10422777, | cg06871262, |
| cg23329372, | cg02806802, | cg14484973, | cg22345576, | cg02362467, |
| cg21876918, | cg16047567, | cg24033871, | cg22032789, | cg16509569, |
| cg13590558, | cg08608215, | cg09444257, | cg14434089, | cg07534440, |
| cg23528488, | cg09239756, | cg24430528, | cg13357416, | cg03642503, |
| cg26875902, | cg22195704, | cg14065224, | cg09002358, | cg14566661, |
| cg04955877, | cg16872560, | cg25723394, | cg24590788, | cg05033333, |
| cg11724135, | cg19793718, | cg00764217, | cg24298878, | cg14399790, |
| cg13941830, | cg15276956, | cg06044900, | cg11848113, | cg13848707, |
| cg19743791, | cg00794552, | cg00712280, | cg11766283, | cg11485154, |
| cg06895640, | cg24691332, | cg11176493, | cg09917805, | cg14542968, |
| cg23139473, | cg07478501, | cg25169790, | cg17496887, | cg03462677, |
| cg11115582, | cg06724693, | cg23171099, | cg03379367, | cg00036258, |
| cg24241429, | cg18561247, | cg14095100, | cg13249096, | cg04737832, |
| cg24589712, | cg18921306, | cg14027204, | cg20979003, | cg08309135, |
| cg05028639, | cg01467209, | cg04281464, | cg19443418, | cg01724683, |
| cg25862117, | cg03029755, | cg05096209, | cg02369775, | cg06607594, |
| cg01747547, | cg22099241, | cg19193155, | cg06065164, | cg26872053, |
| cg01221623, | cg08287637, | cg26119740, | cg08504407, | cg27065415, |
| cg21108361, | cg04292159, | cg25446309, | cg05908979, | cg09361748, |
| cg03932361, | cg23344452, | cg15180637, | cg00509451, | cg16886259, |
| cg19994157, | cg25512518, | cg15079124, | cg02782292, | cg24463309, |
| cg14473597, | cg02048505, | cg06612452, | cg09339907, | cg25977879, |
| cg05117633, | cg02255950, | cg14061886, | cg07126637, | cg11371493, |
| cg14223585, | cg18016565, | cg10121434, | cg07197991, | cg08416638, |
| cg10323457, | cg09077126, | cg05146762, | cg06985415, | cg23942526, |
| cg11804692, | cg19913426, | cg01556502, | cg25391814, | cg18280125, |
| cg01845091, | cg02829706, | cg02319986, | cg05936441, | cg11317705, |
| cg00037457, | cg22320183, | cg00956010, | cg03236170, | cg14475913, |
| cg25310784, | cg11721178, | cg14033806, | cg02858512, | cg19637083, |
| cg18603446, | cg01908020, | cg16944941, | cg00929465, | cg13656078, |
| cg25121589, | cg21885650, | cg13986860, | cg27464657, | cg18565479, |
| cg03699469, | cg04592706, | cg22804222, | cg24409808, | cg00233335, |
| cg02262056, | cg06816651, | cg03332581, | cg20964024, | cg23919111, |
| cg24540109, | cg07280182, | cg07540629, | cg02995130, | cg02869963, |
| cg18988170, | cg04197371, | cg23912509, | cg22414499, | cg15014355, |
| cg04822495, | cg04652645, | cg27119004, | cg21317088, | cg10770742, |
| cg02546173, | cg14864022, | cg14534674, | cg09386807, | cg02603518, |
| cg13503583, | cg00155310, | cg05361709, | cg09674469, | cg01659459, |
| cg12718298, | cg05036212, | cg06534586, | cg05005659, | cg03201507, |
| cg00530284, | cg16314899, | cg03552359, | cg16794017, | cg15560495, |
| cg20500869, | cg11565512, | cg15247645, | cg15868235, | cg24042452, |
| cg17819963, | cg08620751, | cg14165841, | cg01905589, | cg20699780, |
| cg02144332, | cg17296220, | cg04453352, | cg13829550, | cg11969755, |
| cg00894008, | cg11722376, | cg08188471, | cg21700582, | cg04365255, |
| cg00841988, | cg03693486, | cg15946653, | cg00964063, | cg08580836, |
| cg00038239, | cg15933046, | cg00608362, | cg15904939, | cg07201215, |
| cg26986596, | cg22256309, | cg21951425, | cg21583812, | cg23543760, |
| cg19621179, | cg23228609, | cg16276856, | cg11362010, | cg24255159, |
| cg01649317, | cg27546583, | cg01882179, | cg02848093, | cg04058237, |
| cg10721090, | cg09091424, | cg14114297, | cg22860643, | cg23400446, |
| cg01554235, | cg04632069, | cg06423949, | cg14370713, | cg19700006, |
| cg23679332, | cg08446111, | cg25219188, | cg27228868, | cg10168149, |
| cg21470600, | cg21322848, | cg01182309, | cg08933467, | cg14036776, |
| cg10015051, | cg05354180, | cg17988021, | cg15922176, | cg10725623, |
| cg14577472, | cg13324568, | cg03115562, | cg05043032, | cg18935391, |
| cg06286618, | cg22501604, | cg01849198, | cg10990993, | cg06842886, |
| cg05573767, | cg07983575, | cg16013925, | cg24198584, | cg10746622, |
| cg25237692, | cg14450339, | cg01906054, | cg04432275, | cg20865740, |
| cg07662551, | cg06744119, | cg06200048, | cg09938275, | cg08806699, |
| cg06777853, | cg14314653, | cg24279419, | cg03181118, | cg01258789, |
| cg10459029, | cg15289517, | cg07573020, | cg12903374, | cg06023279, |
| cg19646445, | cg10850838, | cg17335595, | cg24807889, | cg06728232, |
| cg14573411, | cg13012494, | cg26711732, | cg08749862, | cg10779184, |
| cg21722639, | cg00859112, | cg03122583, | cg19642394, | cg06506128, |
| cg12695487, | cg00497219, | cg27057602, | cg06513974, | cg24711774, |
| cg04632724, | cg24621034, | cg08766742, | cg04428185, | cg03877802, |
| cg08686995, | cg16506815, | cg27090784, | cg26688911, | cg08971940, |
| cg22608507, | cg01868382, | cg01213651, | cg20383558, | cg17292662, |
| cg02176148, | cg03336984, | cg25471644, | cg06506966, | cg19252891, |
| cg06874403, | cg23913077, | cg13076732, | cg10740660, | cg18903583, |
| cg01679682, | cg11738446, | cg04653012, | cg18425700, | cg01475377, |
| cg13530817, | cg02853107, | cg27056501, | cg18139612, | cg13451356, |
| cg21455600, | cg06667968, | cg15558675, | cg17803054, | cg23138179, |
| cg20674725, | cg08902025, | cg02929681, | cg25788533, | cg14218629, |
| cg13210403, | cg21198712, | cg06637893, | cg19676671, | cg05114861, |
| cg03554264, | cg14521546, | cg19066391, | cg01550144, | cg08924969, |
| cg23051703, | cg08873940, | cg27579854, | cg00641792, | cg04298253, |
| cg11852404, | cg14420670, | cg24568082, | cg04611479, | cg22272545, |
| cg03997992, | cg04738827, | cg10939691, | cg05796338, | cg07183637, |
| cg12020396, | cg13270055, | cg16284178, | cg18027654, | cg27339550, |
| cg00503760, | cg14193007, | cg14751177, | cg13446420, | cg17124408, |
| cg14497781, | cg06774612, | cg26870570, | cg01717254, | cg19326153, |
| cg04024417, | cg11281224, | cg01209566, | cg19185339, | cg00987080, |
| cg04349219, | cg14032528, | cg00601486, | cg17878506, | cg21211418, |
| cg00360230, | cg00088007, | cg05953230, | cg27398640, | cg12825804, |
| cg16625683, | cg11417675, | cg22619719, | cg08046963, | cg04274978, |
| cg01633093, | cg24285919, | cg01762626, | cg14082739, | cg00359010, |
| cg11115235, | cg15535174, | cg14964274, | cg04269065, | cg26904198, |
| cg10089357, | cg01493522, | cg14354292, | cg10598428, | cg01385367, |
| cg04754615, | cg11002686, | cg18027227, | cg17319109, | cg09344028, |
| cg04649598, | cg26664844, | cg13729116, | cg23908305, | cg23296408, |
| cg19260500, | cg07190450, | cg03797305, | cg00926162, | cg14857380, |
| cg03522447, | cg00455018, | cg05050882, | cg21101447, | cg01087392, |
| cg24251890, | cg00337466, | cg11354594, | cg10035303, | cg18281955, |
| cg14597791, | cg00511475, | cg03820120, | cg12267497, | cg04056144, |
| cg21936775, | cg22261467, | cg04303024, | cg19336883, | cg23048951, |
| cg21308575, | cg01538066, | cg03085321, | cg07427438, | cg05362417, |
| cg25403488, | cg27621340, | cg01479818, | cg26054232, | cg19559587, |
| cg04854282, | cg05655987, | cg26385438, | cg18504218, | cg19189012, |
| cg23955431, | cg19973135, | cg07567308, | cg12412926, | cg03358113, |
| cg26996818, | cg18864804, | cg23588713, | cg13533935, | cg03184439, |
| cg15629072, | cg22147449, | cg04064050, | cg21273088, | cg19317871, |
| cg01296927, | cg12516669, | cg24686009, | cg14709103, | cg05179659, |
| cg11239016, | cg21496913, | cg23402821, | cg00953233, | cg17305672, |
| cg00949801, | cg01840183, | cg23520245, | cg11278506, | cg08561253, |
| cg10909324, | cg15422644, | cg12880602, | ch.8.130345735R, | cg09791883, |
| cg20867633, | cg14820908, | cg21027124, | cg05694245, | cg17863681, |
| cg20988589, | cg22530111, | cg05944174, | cg08213047, | cg02360676, |
| cg05762553, | cg06986158, | cg15618428, | cg20171711, | cg05169846, |
| cg19452873, | cg22466678, | cg08425760, | cg06523744, | cg22684151, |
| cg21594297, | cg25982969, | cg07517893, | cg27134827, | cg01513714, |
| cg07938826, | cg27219748, | cg11944518, | cg07626433, | cg08144858, |
| cg12212198, | cg21618455, | cg01941817, | cg13709211, | cg01108705, |
| cg25120705, | cg00379559, | cg08478539, | cg22660544, | cg20879272, |
| cg02954324, | cg18043120, | cg05711037, | cg09364677, | cg09728506, |
| cg23378495, | cg26854559, | cg17214295, | cg02141358, | cg00368844, |
| cg03848697, | cg07675184, | cg05704496, | cg10238302, | cg11875744, |
| cg21517683, | cg15217867, | cg14468634, | cg10943348, | cg00128425, |
| cg13491192, | cg05100279, | cg19731367, | cg26153045, | cg23224666, |
| cg04361126, | cg00288050, | cg27350063, | cg19384032, | cg26958859, |
| cg07106625, | cg15741162, | cg02629281, | cg14072140, | cg05130518, |
| cg13319698, | cg17652998, | cg06208339, | cg26384697, | cg08888487, |
| cg09319946, | cg07792738, | cg12073016, | cg18595800, | cg25203007, |
| cg25868769, | cg14366598, | cg04288999, | cg07774777, | cg19998328, |
| cg10612997, | cg22638597, | cg21036766, | cg12844324, | cg20777920, |
| cg26376835, | cg00322698, | cg19071225, | cg12629698, | cg09957391, |
| cg05931345, | cg07751222, | cg11577646, | cg16376218, | cg05182555, |
| cg25142010, | cg25284213, | cg11487037, | cg23223756, | cg25528143, |
| cg04221877, | cg20841906, | cg01352175, | cg24113782, | cg27532847, |
| cg03864000, | cg27621484, | cg02525939, | cg08087125, | cg02319990, |
| cg17787161, | cg07589077, | cg20009097, | cg01199793, | cg15904283, |
| cg25432696, | cg25366822, | cg19572637, | cg04093671, | cg08292120, |
| cg24932457, | cg17187764, | cg03260799, | cg07421682, | cg13052680, |
| cg24534872, | cg20648004, | cg20942990, | cg23449295, | cg26567788, |
| cg08714590, | cg10035294, | cg03941744, | cg14009451, | cg24985066, |
| cg22052659, | cg07302934, | cg24894584, | cg21099575, | cg23275644, |
| cg15634032, | cg07554871, | cg21475255, | cg21686812, | cg23364226, |
| cg07839313, | cg20883730, | cg15928093, | cg04672706, | cg05228404, |
| cg09159389, | cg26745394, | cg13188812, | cg21742975, | cg13277380, |
| cg05904966, | cg00761344, | cg01620309, | cg12593776, | cg20460101, |
| cg09306214, | cg13671374, | cg23524195, | cg01196592, | cg08506163, |
| cg01200482, | cg23977670, | cg04592976, | cg21914262, | cg05872702, |
| cg00767395, | cg18996590, | cg24670063, | cg22635660, | cg03496157, |
| cg13209441, | cg01028657, | cg07379581, | cg09510263, | cg05540221, |
| cg26314765, | cg23759823, | cg05243042, | cg07085476, | cg00181125, |
| cg11601336, | cg15151929, | cg21938736, | cg20431774, | cg16318240, |
| cg17781710, | cg13536757, | cg05206633, | cg13540981, | cg22940954, |
| cg22991943, | cg26841647, | cg18391058, | cg27401116, | cg26157446, |
| cg02371363, | cg11555878, | cg03930088, | cg08106730, | cg13042605, |
| cg10810305, | ch.21.818333F, | cg10173240, | cg14514987, | cg20536841, |
| cg23526824, | cg21438062, | cg21095808, | cg06189303, | cg03001867, |
| cg03132015, | cg10244666, | cg13844500, | cg15613982, | cg11417319, |
| cg00763094, | cg05017639, | cg15552238, | cg18751231, | cg19621008, |
| cg12000760 | | | | |

¹CvC—cancer vs. control samples (TCGA dataset)
²PvR—progressive vs. regressive CIS dataset

TABLE 9
Metrics:

| Number | ROC AUC | ROC | Threshold | Sensitivity | Specificity | Sensitivity | Specificity |
|---|---|---|---|---|---|---|---|
| of DMPs | (CvC¹) | (PvR²) | value | (CvC) | (CvC) | (PvR) | (PvR) |
| 2000 | 0.982882883 | 0.800925926 | 0.365 | 1 | 0.513513514 | 0.833333333 | 0.666666667 |

DMP IDs:

| | | | | | | |
|---|---|---|---|---|---|---|
| cg20413202, | cg22716151, | cg08668997, | cg02869960, | cg24193339, | cg19744691, | cg12070911, |
| cg00584713, | cg14886145, | cg00684075, | cg08790550, | cg27524972, | cg25447202, | cg14188862, |
| cg04607372, | cg09133563, | cg22714942, | cg16649755, | cg12810212, | cg21286526, | cg27017658, |
| cg19437325, | cg03897139, | cg26945715, | cg05335686, | cg24376212, | cg14199148, | cg14859724, |
| cg04366994, | cg24757553, | cg15396799, | cg08633290, | cg05937046, | cg21160429, | cg18697160, |
| cg08554603, | cg02900760, | cg03631656, | cg00501904, | ch.15.1087158F, | cg09664596, | cg26380443, |
| cg01446612, | cg17313494, | cg00219303, | cg01627483, | cg16710791, | cg19743791, | cg18235734, |
| cg14497327, | cg10149054, | cg14608758, | cg18645492, | cg18459869, | cg06836726, | cg20291162, |
| cg06482904, | cg06510234, | cg13630953, | cg22546859, | cg24719827, | cg26598152, | cg20013963, |
| cg13938218, | cg20170687, | cg04201253, | cg00877632, | cg01635742, | cg09775785, | cg21360245, |
| cg21789136, | cg16045566, | cg09395562, | cg18761058, | cg08468689, | cg04475846, | cg00554993, |
| cg16272710, | cg02217955, | cg25362120, | cg03317811, | cg05399115, | cg27563778, | cg20592017, |
| cg06982745, | cg00321082, | cg01569295, | cg01439112, | cg04784839, | cg10167060, | cg21538511, |
| cg11815480, | cg14464526, | cg13956700, | cg02705800, | cg21518865, | cg22749810, | cg26315964, |
| cg26509915, | cg07148818, | cg25738373, | cg22436229, | cg13640730, | cg25522046, | cg14328230, |
| cg16024318, | cg01729717, | cg19388446, | cg20076240, | cg09883798, | cg12854032, | cg24859484, |
| cg12966876, | cg16299665, | cg18778721, | cg00622655, | cg18755984, | cg00364747, | cg09473180, |
| cg16537756, | cg22801799, | cg15448599, | cg02595807, | cg20856497, | cg13738661, | cg04874673, |
| cg27057509, | cg21521454, | cg13737776, | cg26279668, | cg22842064, | cg27049517, | cg17918501, |
| cg16257086, | cg25841675, | cg25848652, | cg00145969, | cg00962271, | cg16679386, | cg02031326, |
| cg05631737, | cg01614643, | cg26627511, | cg05006432, | cg20558299, | cg21389884, | cg13772514, |
| cg21494953, | cg20022869, | cg14911708, | cg18543871, | cg16626420, | cg25261329, | cg02300308, |
| cg02994216, | cg19795980, | cg22342249, | cg15763984, | cg21856645, | cg04079399, | cg20597569, |
| cg06225923, | cg00126948, | cg09445705, | cg03979510, | cg20641545, | cg03288954, | cg09987740, |
| cg08684639, | cg25637972, | cg25404930, | cg25904964, | cg26884154, | cg19749688, | cg01489419, |
| cg27036638, | cg10601057, | cg04385193, | cg01785605, | cg03958112, | cg11386080, | cg06554062, |
| cg26080154, | cg16911810, | cg13907181, | cg16185419, | cg04628692, | cg00785106, | cg00964321, |
| cg07594209, | cg23925154, | cg19882119, | cg19730813, | cg15444167, | cg18658231, | cg21876918, |
| cg02529142, | cg15745973, | cg26956009, | cg27482328, | cg20178075, | cg00291661, | cg08529825, |
| cg15531536, | cg07611933, | cg04294677, | cg11815796, | cg23143093, | cg12960782, | cg02222722, |
| cg08516217, | cg03440386, | cg08070327, | cg04835284, | cg14992633, | cg21483700, | cg03851192, |
| cg16463452, | cg17755964, | cg11126196, | cg02391713, | cg14554101, | cg15910502, | cg12601843, |
| cg01554970, | cg06601203, | cg17298788, | cg08090385, | cg05951364, | cg19973659, | cg19248242, |
| cg22251172, | cg10007075, | cg17310611, | cg24037813, | cg14146407, | cg26906629, | cg11249283, |
| cg24848467, | cg02192855, | cg25052664, | cg27046641, | cg13650689, | cg01840268, | cg21348233, |
| cg17536595, | cg25621518, | cg13195569, | ch.12.2471797R, | cg11283995, | cg05419198, | cg13595495, |
| cg07095459, | cg12595281, | cg15122358, | cg25369980, | cg03137908, | cg20055230, | cg25979053, |
| cg04802696, | cg03440850, | cg16372869, | cg04769798, | cg08749862, | cg06092312, | cg02847674, |
| cg09030452, | cg25461163, | cg02126031, | cg19484381, | cg15449217, | cg21548155, | cg04360675, |
| cg17541290, | cg19938199, | cg15970666, | cg27522367, | cg09159619, | cg24459953, | cg16736018, |
| cg07983907, | cg06733155, | cg10457885, | cg06573254, | cg07354565, | cg27080194, | cg23582982, |
| cg00526336, | cg04756168, | cg05250874, | cg06615754, | cg21112576, | cg01089589, | cg22912701, |
| cg02831090, | cg01162549, | cg05197660, | cg01213651, | cg11270806, | cg17541810, | cg19548738, |
| cg15379354, | cg15472403, | cg06844496, | cg05096467, | cg20784391, | cg12354056, | cg00320288, |
| cg00640775, | cg02489228, | cg24343835, | cg26405376, | cg07724623, | cg11343017, | cg20161976, |
| cg16163382, | cg06666175, | cg03224013, | cg06554744, | cg00670742, | cg06774595, | cg09464883, |
| cg03143033, | cg22522985, | cg13280612, | cg01995811, | cg13617561, | cg10091738, | cg00044466, |
| cg13100600, | cg20539321, | cg01982240, | cg06626556, | cg02086742, | cg10946333, | cg06885583, |
| cg08099141, | cg12931642, | cg20960405, | cg04349815, | cg02965892, | cg13064457, | cg05406088, |
| cg15264811, | cg19145886, | cg00366850, | cg03168008, | cg20254763, | cg10412231, | cg20583488, |
| cg17882976, | cg11814875, | cg11232054, | cg01544157, | cg13832988, | cg06337230, | cg22831533, |
| cg02002217, | cg21019820, | cg24946544, | cg25962735, | cg27460715, | cg20803849, | cg16480368, |
| cg02124291, | cg23946462, | cg08745199, | cg18213530, | cg05697866, | cg00750074, | cg20349377, |
| cg06927165, | cg13792025, | cg21525413, | cg11744116, | cg04548463, | cg04433035, | cg03893872, |
| cg26366347, | cg00437985, | cg09749049, | cg02868521, | cg09816693, | cg09196684, | cg05210373, |
| cg19102120, | cg22904096, | cg23141333, | cg23214285, | cg14259326, | cg02246800, | cg06415087, |
| cg25492971, | cg00159243, | cg10553204, | cg16653173, | cg00734567, | cg02399048, | cg22334000, |
| cg20309339, | cg19196414, | cg07385362, | cg07700962, | cg06576940, | cg14992232, | cg05412333, |
| cg07475000, | cg12955069, | cg17491548, | cg15645029, | cg24804144, | cg17025142, | cg07498938, |
| cg15709265, | cg00959636, | cg15671688, | cg18233942, | cg20040320, | cg20728105, | cg23172884, |
| cg00709515, | cg06815194, | cg25559262, | cg23788628, | cg18562935, | cg04779631, | cg27485235, |
| cg26156687, | cg08350549, | cg03236137, | cg14354472, | cg11100117, | cg13326801, | cg10160397, |
| cg00852846, | cg20317180, | cg00983437, | cg05134054, | cg23314055, | cg11883836, | cg27629589, |
| cg20314490, | cg11511669, | cg07733129, | cg02359409, | cg22770727, | cg17957172, | cg12737497, |
| cg11177450, | cg18737971, | cg18241635, | cg03476862, | cg26654790, | cg11526560, | cg01467836, |
| cg03622689, | cg19751098, | cg08893269, | cg17500915, | cg18617770, | cg06829968, | cg04583904, |
| cg23689630, | cg26692463, | cg24640182, | cg15120085, | cg04239375, | cg00427553, | cg26693439, |
| cg00740691, | cg18470456, | cg15071133, | cg02891686, | cg10515180, | cg16753400, | cg09475815, |
| cg10604168, | cg14217534, | cg15604097, | cg00421624, | cg20528787, | cg14011070, | cg03620158, |
| cg12076543, | cg17462438, | cg03860853, | cg05156550, | cg22922403, | cg13089859, | cg02060584, |
| cg02696351, | cg16625170, | cg16863673, | cg03977782, | cg05226168, | cg09408098, | cg00468846, |
| cg08514385, | cg23217419, | cg00299105, | cg00819114, | cg06588483, | cg10161743, | cg16848116, |
| cg25757869, | cg26022064, | cg21370255, | cg01297670, | cg06233503, | cg15905979, | cg15646011, |
| cg23155785, | cg10335931, | cg27393285, | cg26790247, | cg10266211, | cg00402366, | cg00428193, |
| cg13159388, | cg14555682, | cg17465227, | cg00318084, | cg03782800, | cg02493602, | cg20557567, |
| cg09958967, | cg11558102, | cg07045873, | ch.6.83452513F, | cg05082738, | cg25032968, | cg09353354, |
| cg09529165, | cg01295994, | cg15552238, | cg03489969, | cg05544413, | cg08115957, | cg11705954, |
| cg17609948, | cg23073439, | cg21426559, | cg27087572, | cg17244673, | cg25894334, | cg21843586, |
| cg24948743, | cg13575252, | cg24131141, | cg15725228, | cg10226952, | cg03710295, | cg25125340, |
| cg08138817, | cg25721452, | cg11746924, | cg01650677, | cg13451048, | cg17778313, | cg10139015, |
| cg16946687, | cg19734870, | cg03441171, | cg14738643, | cg11546709, | cg26885268, | cg19979370, |
| cg14756353, | cg11859384, | cg00533866, | cg27210447, | cg06607764, | cg06127746, | cg22580065, |
| cg00711584, | cg20583060, | cg23900712, | cg13885451, | cg08727867, | cg05493580, | cg11895474, |
| cg04433909, | cg05173382, | cg26031678, | cg01653942, | cg03995571, | cg01743841, | cg10949322, |
| cg19275632, | cg01676680, | cg04181013, | cg22744783, | cg03033080, | cg00087153, | cg25251204, |
| cg23174322, | cg27608224, | cg03629577, | cg26881527, | cg02873997, | cg14657818, | cg13311607, |
| cg20121012, | cg02556526, | cg10204325, | cg11581903, | cg16034458, | cg26662578, | cg22232269, |
| cg15642885, | cg15496005, | cg14119706, | cg25839330, | cg16317901, | cg06781712, | cg05085336, |
| cg11653912, | cg05205239, | cg08245509, | cg13805400, | cg02134353, | cg04930982, | cg02719956, |
| cg00081084, | cg18695750, | cg09285213, | cg23996123, | cg00538458, | cg16156662, | cg17434062, |
| cg12276019, | cg17155018, | cg09315586, | cg12499211, | cg19464231, | cg04316624, | cg19407023, |
| cg06819855, | cg02715668, | cg14191955, | cg26770187, | cg16353049, | cg23489038, | cg13856867, |
| cg15385623, | cg06812309, | cg12575583, | cg02164516, | cg09859768, | cg00020390, | cg18075761, |
| cg08187425, | cg27644247, | cg03920169, | cg03972665, | cg14157435, | cg10420150, | cg03663215, |
| cg05490029, | cg12535280, | cg07421075, | cg14242680, | cg26106304, | cg17428423, | cg16154689, |
| cg00759427, | cg00217055, | cg26668906, | cg26251429, | cg04684864, | cg10082647, | cg24128630, |
| cg12620893, | cg26212352, | cg16878652, | cg01789576, | cg13478001, | cg00461882, | cg11170896, |
| cg06533700, | cg07224914, | cg25472614, | cg17412258, | cg02171771, | cg27154163. | cg15898113, |
| cg24689177, | cg00712390, | cg17600231, | cg07395648, | cg18539086, | cg15153394, | cg27560781, |
| cg14935163, | cg03394764, | cg17693669, | cg01234748, | cg00934299, | cg19066589, | cg16452678, |
| cg04015522, | cg24641302, | cg07247610, | cg05947391, | cg03240059, | cg19603596, | cg26998693, |
| cg20082357, | cg01187464, | cg00045902, | cg25919687, | cg22259536, | cg19369775, | cg02592062, |
| cg13165846, | cg13133420, | cg20080624, | cg24851586, | cg11484576, | cg26389380, | cg02503981, |
| cg18736141, | cg27147174, | cg02939105, | cg04079215, | cg09730735, | cg02862539, | cg21336456, |
| cg22094205, | cg12111697, | cg02165801, | cg27201802, | cg22068659, | cg14181947, | cg23355040, |
| cg10929210, | cg22023952, | cg17162271, | cg27607338, | cg27277366, | cg01681236, | cg13355424, |
| cg15995125, | cg27312071, | cg14661946, | cg18070630, | cg08113691, | cg09514875, | cg17206034, |
| cg23502639, | cg20387100, | cg09828721, | cg00723019, | cg24691042, | cg21392204, | cg08155625, |
| cg12884169, | cg18795569, | cg11922086, | cg00576108, | cg14245228, | cg03601754, | cg12525096, |
| cg13670898, | cg26885400, | cg11683764, | cg16201899, | cg14745270, | cg07300192, | cg17967261, |
| cg26189021, | cg16070743, | cg00987395, | cg17319486, | cg06392442, | cg08731084, | cg08269461, |
| cg07325893, | cg00525277, | cg16435782, | cg23646856, | cg18005693, | cg10779492, | cg22062239, |
| cg06567373, | cg27410383, | cg16511795, | cg05006892, | cg19564098, | cg23624008, | cg19175649, |
| cg07973140, | cg21845273, | cg16127573, | cg13287621, | cg14806252, | cg08658318, | cg10453481, |
| cg25018458, | cg09418673, | cg20020844, | cg18988327, | cg21177558, | cg07235868, | cg21790764, |
| cg06886789, | cg05967120, | cg11773346, | cg21643128, | cg02517337, | cg17972600, | cg13031171, |
| cg05926586, | cg26351951, | cg19698789, | cg09454230, | cg11128808, | cg20241801, | cg11139390, |
| cg27112381, | cg23098599, | cg14207163, | cg06076122, | cg25417675, | cg20425384, | cg19462523, |
| cg12833931, | cg26333156, | cg23856428, | cg15158031, | cg23246703, | cg12382045, | cg13868356, |
| cg22018874, | cg08937395, | cg03214931, | cg22637431, | cg23610023, | cg07197515, | cg08848774, |
| cg19344806, | cg26575173, | cg25198545, | cg09569258, | cg17087698, | cg07855525, | cg04936610, |
| cg24493811, | cg01318291, | cg20559422, | cg00936699, | cg12145289, | cg19901801, | cg18734591, |
| cg11988910, | cg24525461, | cg15053322, | cg19987425, | cg19017312, | cg04285064, | cg17997329, |
| cg24203741, | cg25036483, | cg13629999, | cg17774233, | cg00694560, | cg02685484, | cg17972213, |
| cg03293770, | cg05220751, | cg15013982, | cg24641522, | cg00476282, | cg14690510, | cg14047478, |
| cg10338848, | cg09732255, | cg11950642, | cg15461550, | cg17391087, | cg16065574, | cg00515408, |
| cg27219446, | cg19459093, | cg03581113, | cg20247380, | cg01633858, | cg07533951, | cg19648605, |
| cg07074042, | cg10092957, | cg15679651, | cg05120156, | cg05871694, | cg16145703, | cg19407960, |
| cg18896979, | cg25794571, | cg06793796, | cg02658330, | cg11213687, | cg18609120, | cg13962286, |
| cg01047779, | cg16680214, | cg05331340, | cg05881566, | cg09588074, | cg26198776, | cg05411944, |
| cg03038132, | cg27447254, | cg21994724, | cg23532985, | cg14957639, | cg25054754, | cg06364044, |
| cg01822827, | cg27524580, | cg10423170, | cg24358846, | cg03525644, | cg26299767, | cg11050603, |
| cg20838009, | cg20648385, | cg04712004, | cg25485192, | cg02629833, | cg02303933, | cg02013018, |
| cg05456921, | cg05086178, | cg11809094, | cg09465142, | cg00290994, | cg21501195, | cg13724813, |
| cg12639192, | cg06286274, | cg19647611, | ch.3.42394281R, | cg17445157, | cg10490742, | cg08214329, |
| cg18398551, | cg18244157, | cg22531183, | cg24762359, | cg21029167, | cg09617441, | cg27408637, |
| cg15717250, | cg01041222, | cg06592860, | cg06895849, | cg17023648, | cg03029752, | cg18991240, |
| cg08246494, | cg06916446, | cg15988843, | cg17499430, | cg06734730, | cg06319822, | cg09726412, |
| cg24900963, | cg12742826, | cg14467415, | cg23498628, | cg25104727, | cg07894352, | cg14697246, |
| cg07480495, | cg20748791, | cg18906079, | cg13433320, | cg22097768, | cg04259606, | cg05380808, |
| cg18479593, | cg15648729, | cg09700855, | cg01381374, | cg09309261, | ch.17.458890R, | cg05092861, |
| cg25243773, | cg02853954, | cg11705975, | cg01606867, | cg13210239, | cg04645430, | cg11550865, |
| cg25834613, | cg06923967, | cg00214165, | cg12734920, | cg17042000, | cg25490175, | cg07560662, |
| cg06869755, | cg10191283, | cg06887159, | cg24790706, | cg16633750, | cg25910506, | cg06424719, |
| cg08944683, | cg01556502, | cg03460756, | cg06443114, | cg00072957, | cg06876404, | cg20630605, |
| cg11588889, | cg05082557, | cg06842519, | cg00149455, | cg23339936, | cg04054313, | cg08224217, |
| cg14087413, | cg22626579, | cg00283472, | cg23586792, | cg25074170, | cg03766703, | cg21619639, |
| cg17021648, | cg03396347, | cg09810078, | cg25955341, | cg26162020, | cg14297518, | cg04158367, |
| cg24774154, | cg05582797, | cg04403488, | cg03877381, | cg20240462, | cg21771679, | cg19426827, |
| cg10723962, | cg07835953, | cg19061632, | cg22697684, | cg17412248, | cg05970033, | cg25152312, |
| cg10869069, | cg04898765, | cg06507579, | cg21575923, | cg09887804, | cg11731437, | cg23912072, |
| cg26870438, | cg04501624, | cg00186954, | cg04911940, | cg16648952, | cg10577511, | cg20313298, |
| cg00039480, | cg12284870, | cg16319085, | cg13223402, | cg01258039, | cg21084982, | cg17683336, |
| cg19222784, | cg08347473, | cg24406240, | cg00111532, | cg19279346, | cg09601912, | cg24397007, |
| cg00354484, | cg09088508, | cg01777643, | cg01254644, | cg15323828, | cg16838729, | cg11643245, |
| cg16558172, | cg11893955, | cg03429569, | cg06451949, | cg23665802, | cg16535510, | cg25934064, |
| cg04784546, | cg21817012, | cg12009759, | cg13990321, | cg18848688, | cg01620410, | cg02087039, |
| cg06665913, | cg10020290, | cg03931078, | cg14977279, | cg06344571, | cg16744710, | cg06111140, |
| cg01221716, | cg02141929, | cg15868421, | cg21579912, | cg21999284, | cg00071161, | cg01000236, |
| cg01157304, | cg22333259, | cg10307345, | cg10395920, | cg23180600, | cg08581953, | cg05474467, |
| cg25884488, | cg12588082, | cg00278359, | cg25325588, | cg26381352, | cg03554749, | cg15778400, |
| cg16405210, | cg17030415, | cg01693108, | cg08738443, | cg03573455, | cg16473703, | cg09909523, |
| cg27527018, | cg20448791, | cg24974217, | cg13693582, | cg22022799, | cg06926306, | cg04981556, |
| cg17591270, | cg19663246, | cg11936145, | cg26117023, | cg22287624, | cg13423383, | cg07428430, |
| cg07936323, | cg05937194, | cg03769629, | cg26039848, | cg07677930, | cg19046826, | cg07379703, |
| cg18567941, | cg06057715, | cg24516799, | cg24123106, | cg01933559, | cg25960090, | cg26651978, |
| cg09307564, | cg22948077, | cg06502071, | cg04837959, | cg00096603, | cg16333269, | cg22491279, |

DMP IDs:

| | | | | |
|---|---|---|---|---|
| cg18324518, | cg17393296, | cg08076830, | cg25345738, | cg24796842, |
| cg18024037, | cg27412284, | cg10530153, | cg05917188, | cg12411965, |
| cg05280148, | cg08814105, | cg03032025, | cg17761898, | cg08458400, |
| cg12090885, | cg13312668, | cg27455578, | cg07704627, | cg12085155, |
| cg01190276, | cg01364137, | cg13876163, | cg26098117, | cg17171325, |
| cg19253809, | cg12525474, | cg04343292, | cg09088834, | cg02622835, |
| cg14541653, | cg03311459, | cg12217954, | cg26530701, | cg03207667, |
| cg18468235, | cg27374726, | cg10473158, | cg12430467, | cg06640047, |
| cg24722577, | cg05412028, | cg07100411, | cg03948536, | cg18785274, |
| cg25635913, | cg08683938, | cg12485926, | cg20138861, | cg10201663, |
| cg00469541, | cg10230355, | cg16562913, | cg09735014, | cg06325088, |
| cg09584711, | cg15783027, | cg15505615, | cg03523799, | cg13266717, |
| cg04807152, | cg21371235, | cg17205254, | cg12081325, | cg10849498, |
| cg14432251, | cg07252731, | cg13084158, | cg14614314, | cg08550205, |
| cg09460231, | cg03918288, | cg04350121, | cg17819732, | cg05256304, |
| cg13283153, | cg15321293, | cg10006442, | cg13643138, | cg27343123, |
| cg27182529, | cg04056504, | cg21791158, | cg03316934, | cg26022315, |
| cg13848990, | cg16362201, | cg03066788, | cg13026629, | cg03929366, |
| cg04479713, | cg10628035, | cg21489303, | cg06801811, | cg07935320, |
| cg25586329, | cg04370442, | cg07505515, | cg22214565, | cg03463076, |
| cg14582957, | cg10456459, | cg08615818, | cg14941541, | cg10011856, |
| cg02449373, | cg04267691, | cg19645410, | cg20003034, | cg25695166, |
| cg18935353, | cg19856145, | cg19832347, | cg10202113, | cg23386046, |
| cg12627901, | cg10694598, | cg08626831, | cg06180356, | cg10776533, |
| cg24842334, | cg22423750, | cg16360777, | cg16434657, | cg15865026, |
| cg17173423, | cg13500576, | cg04344875, | cg00387090, | cg12027254, |
| cg04567173, | cg00992348, | cg05723527, | cg23829318, | cg27475586, |
| cg22815953, | cg16849201, | cg15496307, | cg16738646, | cg04443870, |
| cg12763978, | cg26137068, | cg16493566, | cg10208821, | cg18959161, |
| cg05794480, | cg14542334, | cg09895325, | cg01048526, | cg02358190, |
| cg09996789, | cg26926665, | cg17465752, | cg00661202, | cg05468716, |
| cg20584841, | cg06351831, | cg14750616, | cg17143192, | cg10815483, |
| cg08172065, | cg16314254, | cg15694585, | cg21030424, | cg16230307, |
| cg01829024, | cg06007607, | cg12340891, | cg00621289, | cg06616765, |
| cg25331127, | cg03170318, | cg10398590, | cg05043047, | cg27103616, |
| cg00726615, | cg14808360, | cg01835635, | cg05352321, | cg03839252, |
| cg05127440, | cg18540299, | cg25128696, | cg21209395, | cg10230427, |
| cg09998519, | cg06705767, | cg17213048, | cg06333135, | cg23019935, |
| cg13117809, | cg11783089, | cg03032816, | cg16245174, | cg09490603, |
| cg17940234, | cg06668194, | cg13003878, | cg07908999, | cg12291741, |
| cg16553757, | cg04167764, | cg14799457, | cg01357222, | cg16667845, |
| cg19975917, | cg09777637, | cg02057386, | cg06000610, | cg21088460, |
| cg14908186, | cg03397307, | cg13661129, | cg17962638, | cg07485723, |
| cg00024416, | cg15315056, | cg03534360, | cg06060191, | cg23943264, |
| cg10786876, | cg22159483, | cg01253818, | cg19448829, | cg19747659, |
| cg19586881, | cg19162158, | cg02666966, | cg20459712, | cg22143289, |
| cg17217443, | cg09370299, | cg14329365, | cg14624773, | cg12091936, |
| cg16928487, | cg17472736, | cg27120667, | cg03730622, | cg11074549, |
| cg14454907, | cg13802192, | cg16722292, | cg20484352, | cg00758420, |
| cg11898688, | cg02187231, | cg16968115, | cg09874052, | cg06670742, |
| cg08160663, | cg23977063, | cg18794577, | cg02834722, | cg06670612, |
| cg03170881, | cg03517614, | cg05544940, | cg20584011, | cg07079421, |
| cg01753188, | cg05484879, | cg04156606, | cg02226688, | cg01808050, |
| cg26226110, | cg23859703, | cg22055815, | cg15084543, | cg03401656, |
| cg12594001, | cg10942903, | cg05264446, | cg06048102, | cg06148118, |
| cg12717591, | cg23754665, | cg15448721, | cg04706540, | cg20588045, |
| cg26494782, | cg01223986, | cg12905673, | cg16364191, | cg15881727, |
| cg06185830, | cg13371870, | cg19007031, | cg10389385, | cg15161854, |
| cg20486052, | cg25052970, | cg12577321, | cg09189896, | cg02068171, |
| cg00236005, | cg04522544, | cg04737405, | cg15576058, | cg10092198, |
| cg16922012, | cg20819191, | cg26504305, | cg05572456, | cg20870668, |
| cg01831404, | cg15364548, | cg01765164, | cg19778582, | cg08709672, |
| cg18582260, | cg04236205, | cg24721350, | cg13378687, | cg19694978, |
| cg07805238, | cg16301768, | cg03990819, | cg14558812, | cg10118998, |
| cg14263118, | cg09686639, | cg13818368, | cg01564165, | cg26936429, |
| cg21799246, | cg06258207, | cg14081251, | cg25764931, | cg27228562, |
| cg21649569, | cg14897238, | cg13050802, | cg20768717, | cg21490485, |
| cg18553657, | cg24495681, | cg18388639, | cg14721506, | cg06684201, |
| cg14182513, | cg21789849, | cg10376360, | cg07520919, | cg03146079, |
| cg16496928, | cg20625393, | cg13844552, | cg04894116, | cg17911539, |
| cg04435483, | cg18839504, | cg10285337, | cg11942059, | cg13614631, |
| cg14771658, | cg24937306, | cg20323571, | cg22826818, | cg04290666, |
| cg06373470, | cg21315798, | cg17050941, | cg04515533, | cg19179687, |
| cg10932907, | cg08676812, | cg19581696, | cg26967100, | cg09902062, |
| cg12223009, | cg16371032, | cg14304336, | cg18364779, | cg08268388, |
| cg07394400, | cg18119407, | cg22052514, | cg16325394, | cg11774232, |
| cg01369378, | cg17001035, | cg14114517, | cg21532618, | cg08909592, |
| cg09627874, | cg11501438, | cg15063355, | cg02440872, | cg27531496, |
| cg07141033, | cg01143145, | cg20401393, | cg23689428, | cg12483005, |
| cg20102368, | cg10456035, | cg23024824, | cg02070289, | cg02699829, |
| cg06830319, | cg07224455, | cg02222860, | cg06374962, | cg01739446, |
| cg04889446, | cg07197290, | cg15242490, | cg20111340, | cg12678421, |
| cg04560810, | cg05529848, | cg03950655, | cg16712637, | cg04911180, |
| ch.19.1633300R, | cg09284703, | cg05812089, | cg23347182, | cg05143633, |
| ch.17.1508712R, | cg04228709, | cg01758041, | cg20232748, | cg09569432, |
| cg14318946, | cg11908131, | cg21249729, | cg00260919, | cg14228029, |
| cg00621646, | cg20203357, | cg08060515, | cg01943155, | cg07996580, |
| cg16657149, | cg11803251, | cg23567366, | cg21485906, | cg13691697, |
| cg22045977, | cg03232953, | cg13581422, | cg19113920, | cg13149523, |
| cg03537938, | cg05777319, | cg00363082, | cg26228696, | cg05518939, |
| cg03671276, | cg12785993, | cg02162534, | cg15225201, | cg14515211, |
| cg17349151, | cg04554122, | cg11891239, | cg10246581, | cg01015769, |
| cg11328885, | cg20502080, | cg21874005, | cg14697642, | cg03448619, |
| cg14554488, | cg25310676, | cg13927504, | cg15721359, | cg19857071, |
| cg05060704, | cg10542584, | cg12871835, | cg25319276, | cg23089549, |
| cg16624482, | cg16175263, | cg12013757, | cg21300719, | cg17614974, |
| cg05182249, | cg04902669, | cg01498384, | cg13082642, | cg23680451, |
| cg10433904, | cg00126959, | cg20112838, | cg27138600, | cg17755208, |
| cg13475379, | cg18704110, | cg27313403, | cg23387220, | cg08090857, |
| cg11331988, | cg25751266, | cg14585793, | cg14376424, | cg02603662, |
| cg08969094, | cg16682257, | cg20902104, | cg24071851, | cg06324635, |
| cg14345213, | cg22865215, | cg09633973, | cg16915540, | cg15197881, |
| cg26872780, | cg13966710, | cg09079593, | cg00274965, | cg16223976, |
| cg10069121, | cg21747188, | cg14435997, | cg18450555, | cg11183000, |
| cg19900515, | cg11277665, | cg00428492, | cg03148419, | cg01906733, |
| cg11006791, | cg23971440, | cg20073050, | cg18291941, | cg21361702, |
| cg05442602, | cg20936013, | cg01200289, | cg04202120, | cg08133931, |
| cg04249605, | cg19874450, | cg02709907, | cg04484994, | cg20631270, |
| cg10291995, | cg10250660, | cg21942670, | cg07513561, | cg03618918, |
| cg01301233, | cg13576178, | cg11829200, | cg24929556, | cg03520624, |
| cg23892924, | cg02770112, | cg24072599, | cg02491717, | cg06697094, |
| cg07643314, | cg05193832, | cg16549153, | cg11804789, | cg03999934, |
| cg21158087, | cg00552490, | cg14398548, | cg00231945, | cg20845544, |
| cg03064403, | cg11033938, | cg08131811, | cg11769400, | cg18899777, |
| cg20325293, | cg01422797, | cg23545299, | cg21785536, | cg00924909, |
| cg22994808, | cg19524023, | cg19829229, | cg18101784, | cg11468953, |
| cg09626299, | cg14851345, | cg05301359, | cg25983635, | cg10164300, |
| cg03207310, | cg09304270, | cg12061219, | cg06622725, | cg23745282, |
| cg27408238, | cg15187606, | cg15854296, | cg14474669, | cg20884298, |
| cg11414030, | cg02891785, | cg03877680, | cg25947555, | cg20587188, |
| cg19724668, | cg09961645, | cg25091458, | cg11370512, | cg27634258, |
| cg17182302, | cg14144564, | cg12994505, | cg22875013, | cg13683280, |
| cg14278893, | cg18081687, | cg03293697, | cg25033993, | cg12421755, |
| cg23501051, | cg18326719, | cg08586216, | cg19075111, | cg10773601, |
| cg08066755, | cg09722556, | cg26338867, | cg26231732, | cg04274288, |
| cg05928362, | cg03147452, | cg06969845, | cg05059613, | cg14102731, |
| cg11894951, | cg17846785, | cg25581330, | cg14781667, | cg03479572, |
| cg02530753, | cg24023618, | cg06860422, | cg00318347, | cg02519718, |
| cg16936953, | cg20240014, | cg02884588, | cg03481230, | cg10436014, |
| cg14033944, | cg06619906, | cg13272280, | cg07032164, | cg12776171, |
| cg09671309, | cg05725703, | cg00525823, | cg26960562, | cg19518695, |
| cg00789198, | cg07873926, | cg02554810, | cg13352750, | cg17676132, |
| cg08900821, | cg03267026, | cg26059822, | cg11219637, | cg13676616, |
| cg26504248, | cg17807806, | cg12517318, | cg14491820, | cg04984575, |
| cg20029393, | cg21043481, | cg11242791, | cg16147305, | cg26605046, |
| cg19083497, | cg05626376, | cg13747145, | cg16442852, | cg06559864, |
| cg15155199, | cg14534148, | cg08694295, | cg12370248, | cg12014145, |
| cg10942056, | cg26114100, | cg21921789, | cg01020987, | cg21837669, |
| cg26641996, | cg11991859, | cg10097906, | cg24436906, | cg19920460, |
| cg17267720, | cg24268495, | cg09241455, | cg14627760, | cg04212127, |
| cg03045323, | cg12005760, | cg19690494, | cg27553955, | cg16047567, |
| cg23301120, | cg02058715, | cg03052789, | cg09403666, | cg15412291, |
| cg12393589, | cg18391978, | cg16639922, | cg02190572, | cg06850283, |
| cg15971826, | cg05983695, | cg09132619, | cg23015361, | cg13445358, |
| cg10686758, | cg20300260, | cg11141692, | cg17181966, | cg08427573, |
| cg06757138, | cg19704073, | cg10632215, | cg01002120, | cg00580978, |
| cg15080590, | cg23930683, | cg17331566, | cg07535628, | cg18372607, |
| cg00682069, | cg02110273, | cg24251035, | cg02806032, | cg20498033, |
| cg04013393, | cg01293485, | cg09165088, | cg08334780, | cg13798575, |
| cg07318372, | cg15860287, | cg25196088, | cg26681399, | cg25885077, |
| cg04967200, | cg26286082, | cg07152091, | cg17873815, | cg08319294, |
| cg04154903, | cg10389114, | cg01578265, | cg03008693, | cg13668397, |
| cg00974095, | cg26659079, | cg12165546, | cg05421651, | cg12445565, |
| cg12998491, | cg25935985, | cg25355006, | cg20264529, | cg03549146, |
| cg15358103, | cg19089701, | cg17291136, | cg19526455, | cg23831517, |
| cg16632994, | cg00218415, | cg22126440, | cg05957190, | cg23517999, |
| cg19446838, | cg21012711, | cg10290504, | cg24877129, | cg20608032, |
| cg27662379, | cg14550265, | cg05452899, | cg21877201, | cg22439857, |
| cg15787227, | cg14451728, | cg22753376, | cg22480949, | cg20429172, |
| cg17575960, | cg00612107, | cg19290962, | cg04819655, | cg22599563, |
| cg06966887, | cg24342628, | cg24684816, | cg05343184, | cg23767433, |
| cg17900861, | cg02358477, | cg14135522, | cg25161912, | cg03975755, |
| cg16986846, | cg15139906, | cg03625287, | cg24328964, | cg05449607, |
| cg08612137, | cg09211336, | cg14240536, | cg08295543, | cg22508782, |
| cg05042938, | cg13393678, | cg16853982, | cg19089394, | cg03504865, |
| cg15166039, | cg23925301, | cg01027929, | cg09872523, | cg03951219, |
| cg21282054 | | | | |

¹CvC—cancer vs. control samples (TCGA dataset)
²PvR—progressive vs. regressive CIS dataset

TABLE 10

Metrics:

| Number | ROC AUC | ROC | Threshold | Sensitivity | Specificity | Sensitivity | Specificity |
|---|---|---|---|---|---|---|---|
| of DMPs | (CvC¹) | (PvR²) | value | (CvC) | (CvC) | (PvR) | (PvR) |
| 2000 | 0.982786358 | 0.781635802 | 0.38 | 1 | 0.481081081 | 0.944444444 | 0.694444444 |

DMP IDs:

| | | | | | | |
|---|---|---|---|---|---|---|
| cg01818539, | cg05897028, | cg12302070, | cg04086771, | cg24084128, | cg19032799, | cg00457195, |
| cg20012308, | cg06336984, | cg16488974, | cg15702701, | cg14978345, | cg13823701, | cg19945912, |
| cg14536097, | cg16682206, | cg09022552, | cg11964006, | cg04041474, | cg21094737, | cg26075931, |
| cg13179085, | cg07270851, | cg08116113, | cg21001198, | cg08912666, | cg12528349, | cg03346119, |
| cg14526204, | cg27137978, | cg16869647, | cg19643921, | cg17219656, | cg10969412, | cg07693617, |
| cg22318806, | cg24523250, | cg00442109, | cg03524533, | cg11034122, | cg10809282, | cg06721975, |
| cg13216724, | cg24567464, | cg15894948, | cg22881750, | cg00620476, | cg01781850, | cg25469212, |
| cg17504641, | cg22851295, | cg22236890, | cg14660024, | cg26394055, | cg10850823, | cg00212909, |
| cg18144593, | cg18323376, | cg14623306, | cg17656267, | cg16268380, | cg02607915, | cg01335162, |
| cg04368242, | cg04323427, | cg17881037, | cg22735222, | cg07935669, | cg23074401, | cg00801196, |
| cg11310087, | cg08045766, | cg22724500, | cg26541854, | cg22626579, | cg26581982, | cg23448486, |
| cg24027378, | cg21878746, | cg02458384, | cg09443484, | cg21562321, | cg25685524, | cg17208748, |
| cg26152754, | cg09502177, | cg05575601, | cg26851112, | cg15340874, | cg05807485, | cg03174013, |
| cg15318176, | cg24843443, | cg24994111, | cg10650290, | cg15573925, | cg17502984, | cg03626579, |
| cg21317088, | cg00329300, | cg08119412, | cg22020752, | cg07353685, | cg07289618, | cg15993652, |
| cg12551746, | cg17580261, | cg23125449, | cg18669186, | cg08245509, | cg24582500, | cg11614536, |
| cg03997398, | cg17005595, | cg14846617, | cg05508084, | cg04809622, | cg11132336, | cg17340796, |
| cg26372400, | cg19976997, | cg04794420, | cg05860220, | cg19237113, | cg23927976, | cg12983042, |
| cg08734237, | cg09959112, | cg04853978, | cg00940278, | cg20429994, | cg20709805, | cg04542080, |
| cg09651522, | cg04810755, | cg02494781, | cg22800723, | cg09115607, | cg23539381, | cg26773803, |
| cg16203594, | cg19750643, | cg24075113, | cg08951891, | cg09261289, | cg08595806, | cg10806197, |
| cg23942884, | cg09540738, | cg20090561, | cg14094767, | cg11953434, | cg01704862, | cg02359773, |
| cg02729086, | cg21998505, | cg17515456, | cg07300060, | cg20339650, | cg05819416, | cg04792644, |
| cg02710296, | cg14011140, | cg23866596, | cg12847125, | cg24949795, | cg16531924, | cg12597309, |
| cg15904623, | cg00844439, | cg12746706, | cg24821313, | cg19090574, | cg20794824, | cg25731401, |
| cg14630748, | cg06370650, | cg00995893, | cg00879843, | cg13858139, | cg20947113, | cg02491234, |
| cg10192047, | cg19062258, | cg00137629, | cg01171597, | cg02818572, | cg08266366, | cg02942591, |
| cg00877739, | cg11841562, | cg17875102, | cg22700036, | cg14345917, | cg11076814, | cg08066306, |
| cg27212541. | cg19857714, | cg26488009, | cg23825213, | cg11240431, | cg26956263, | cg07475954, |
| cg22280173, | cg22476550, | cg19856749, | cg05590267, | cg19793034, | cg11492040, | cg09963814, |
| cg10356655, | cg03712816, | cg20886165, | cg25329844, | cg09513416, | cg20014063, | cg27257926, |
| cg02571054, | cg11210004, | cg02117903, | cg12078155, | cg08401143, | cg14986870, | cg13232821, |
| cg07870854, | cg04802221, | cg15465439, | cg01063701, | cg27258025, | cg13611997, | cg24531022, |
| cg19569340, | cg06727269, | cg16723362, | cg04825775, | cg23766918, | cg06690633, | cg10496560, |
| cg09284703, | cg13081999, | cg06528584, | cg10195011, | cg10240356, | cg17462109, | cg16413619, |
| cg08625564, | cg04970150, | cg22926528, | cg27061971, | cg07542748, | cg01350169, | cg19642727, |
| cg24074495, | cg01241721, | cg01565013, | cg02749752, | cg08682475, | cg13175480, | cg23270434, |
| cg02206636, | cg20254353, | cg07273342, | cg10037053, | cg02024246, | cg06334134, | cg06574757, |
| cg01339183, | cg01060059, | cg27582790, | cg11170685, | cg26335527, | cg04185893, | cg20933586, |
| cg03669949, | cg14176575, | cg18411941, | cg17310977, | cg14811390, | cg15113090, | cg02787737, |
| cg11263852, | cg03560597, | cg22112000, | cg08038074, | cg14339116, | cg15168995, | cg23555120, |
| cg09592804, | cg17568841, | cg13757263, | cg04656009, | cg26434376, | cg11843090, | cg21219157, |
| cg19027827, | cg18967180, | cg26954138, | cg23948756, | cg17589341, | cg20872981, | cg09518851, |
| cg06677470, | cg10723962, | cg03299487, | cg05048159, | cg03663984, | cg18133934, | cg18564458, |
| cg10452531, | cg15676677, | cg14088161, | cg15693066, | cg00965391, | cg27417659, | cg21439823, |
| cg15482690, | cg08703722, | cg18479229, | cg09123158, | cg01380104, | cg24072202, | cg16137862, |
| cg00474657, | cg17555714, | cg11576486, | cg00581783, | cg24633027, | cg25885280, | cg04031424, |
| cg25862975, | cg09957391, | cg17486644, | cg16154857, | cg19093405, | cg21043746, | cg17034203, |
| cg20743280, | cg12508214, | cg04181528, | cg10504108, | cg18685773, | cg21775668, | cg08635403, |
| cg27065742, | cg08109681, | cg20235099, | cg14045725, | cg26069037, | cg24307799, | cg12575583, |
| cg13919821, | cg00405112, | cg17208278, | cg02328930, | cg01075852, | cg22874858, | cg01466121, |
| cg27021553, | cg09168548, | cg06325515, | cg00704126, | cg16859636, | cg12902062, | cg26515755, |
| cg07639650, | cg16720616, | cg14108356, | cg25738176, | cg00580652, | cg03008707, | cg03726569, |
| cg18256640, | cg23087300, | cg04234631, | cg17466748, | cg17619093, | cg15807180, | cg14366939, |
| cg26524541, | cg23067856, | cg22122329, | cg02847384, | cg03344290, | cg18247179, | cg06288071, |
| cg16381019, | cg24204951, | cg04422150, | cg07862612, | cg23667127, | cg13588355, | cg15486123, |
| cg08113002, | cg21528399, | cg00776960, | cg14970096, | cg26235861, | cg21442419, | cg05447833, |
| cg03360272, | cg01830294, | cg19498375, | cg23563438, | cg23018181, | cg03480605, | cg22948288, |
| cg10623043, | cg07768763, | cg11051022, | cg18241635, | cg08710432, | cg12468496, | cg26025333, |
| cg00328720, | cg13549904, | cg13718827, | cg26636193, | cg11942329, | cg24386905, | cg23978064, |
| cg21631754, | cg27410756, | cg02530022, | cg15257489, | cg01018726, | cg19645221, | cg00323631, |
| cg18832029, | cg19506755, | cg00707688, | cg11558474, | cg00293381, | cg14847009, | cg15452970, |
| cg09045419, | cg26904991, | cg22952917, | cg13815390, | cg05438727, | cg20774312, | cg26917540, |
| cg21324936, | cg04093381, | cg07484827, | cg02753187, | cg06362985, | cg05824482, | cg27130769, |
| cg09835740, | cg10884288, | cg05502312, | cg08041014, | cg14449803, | cg22805603, | cg16109089, |
| cg02324021, | cg25376491, | cg14955103, | cg01884057, | cg02078598, | cg15096085, | cg07301795, |
| cg26112846, | cg14934242, | cg10380221, | cg09915500, | cg21238404, | cg22907891, | cg00578414, |
| cg13900327, | cg25932761, | cg10223982, | cg03882585, | cg21591742, | cg08145177, | cg13753183, |
| cg02435495, | cg19219560, | cg10092652, | cg16886403, | cg23345004, | cg00442112, | cg19584649, |
| cg07509167, | cg18721605, | cg23272399, | cg00055726, | cg27176828, | cg03300898, | cg16027622, |
| cg03901886, | cg01914785, | cg01601628, | cg18507062, | cg12804104, | cg07617384, | cg09733103, |
| cg24956253, | cg00617064, | cg06995548, | cg11375831, | cg18499545, | cg27495643, | cg13464351, |
| cg16460466, | cg19564512, | cg03371404, | cg04484842, | cg21629591, | cg18282041, | cg12882815, |
| cg08082763, | cg16190266, | cg07379167, | cg03790325, | cg05685023, | cg04777612, | cg25407372, |
| cg18599162, | cg22331074, | cg00094255, | cg13209368, | cg25219246, | cg10494028, | cg13525397, |
| cg13858394, | cg22796923, | cg15235987, | cg22226680, | cg23863157, | cg07348311, | cg04156607, |
| cg01316819, | cg07235439, | cg02114786, | cg19315466, | cg12013817, | cg10078750, | cg03116258, |
| cg25381543, | cg02167203, | cg13272258, | cg17200737, | cg16627549, | cg15145873, | cg14183455, |
| cg27600084, | cg04780697, | cg15741354, | cg12840248, | cg00953256, | cg17826786, | cg10899006, |
| cg12639453, | cg00826626, | cg04134748, | cg01565703, | cg12567615, | cg21506441, | cg00142759, |
| cg11798873, | cg17881997, | cg10999312, | cg15589939, | cg02658681, | cg05225993, | cg17044311, |
| cg10987503, | cg13848291, | cg09126273, | cg11930665, | cg22249456, | cg10615414, | cg09160955, |
| cg24834709, | cg25940730, | cg18363918, | cg27136270, | cg19726715, | cg19430622, | cg01550215, |
| cg04468198, | cg19611002, | cg18416096, | cg09272748, | cg24571227, | cg03420068, | cg14723344, |
| cg24794669, | cg12623045, | cg18986589, | cg17884327, | cg25061763, | cg03571764, | cg17573720, |
| cg02779230, | cg16265542, | cg23818888, | cg05852523, | cg26540931, | cg18509719, | cg15827031, |
| cg27209130, | cg19498794, | cg06920390, | cg06617876, | cg13677149, | cg01021385, | cg21323630, |
| cg09633533, | cg27153059, | cg03918304, | cg03224209, | cg15056556, | cg20511489, | cg22365276, |
| cg15790839, | cg25890048, | cg03685886, | cg10634217, | cg06907405, | cg06379532, | cg05529686, |
| cg18132916, | cg25298293, | cg11063170, | cg18243017, | cg15408080, | cg10324902, | cg10057843, |
| cg12281657, | cg18865207, | cg00817731, | cg25422735, | cg19640439, | cg02957197, | cg00739593, |
| cg26010099, | cg01923724, | cg06887778, | cg01961105, | cg02583576, | cg10036003, | cg11888982, |
| cg14850730, | cg11659749, | cg06448249, | cg17767567, | cg22542595, | cg12339531, | cg06985415, |
| cg14089493, | cg09057080, | cg06221215, | cg03887906, | cg06015997, | cg16523490, | cg25770096, |
| cg00250743, | cg24541378, | cg26320696, | cg24005340, | cg12095397, | cg09998519, | cg26319363, |
| cg24219671, | cg27643205, | cg01754525, | cg24425342, | cg00388262, | cg16823009, | cg01765403, |
| cg04699214, | cg09431544, | cg00858415, | cg13345299, | cg09306188, | cg15522984, | cg12706425, |
| cg22346355, | cg13373757, | cg03107393, | cg18761696, | cg05605377, | cg02264459, | cg07920434, |
| cg19496554, | cg15429732, | cg04311134, | cg14645856, | cg16045533, | cg16718942, | cg05819296, |
| cg03332188, | cg25960785, | cg23195510, | cg21708468, | cg21389743, | cg16377368, | cg24740404, |
| cg21080473, | cg07528216, | cg10442913, | cg19193384, | cg00480356, | cg03513682, | cg17504145, |
| cg05783135, | cg11884792, | cg02930107, | cg13390388, | cg03722487, | cg22860780, | cg17842068, |
| cg06162003, | cg03773809, | cg24155961, | cg03786920, | cg07100532, | cg14619560, | cg04295543, |
| cg11746924, | cg06271986, | cg24943514, | cg27012908, | cg02473687, | cg00222995, | cg09147140, |
| cg23767433, | cg10176011, | cg11000292, | cg24009736, | cg26622232, | cg04435807, | cg10222822, |
| cg06576010, | cg02798749, | cg06217230, | cg08973382, | cg27310761, | cg24431734, | cg13821571, |
| cg26312951, | cg04739200, | cg15836710, | cg14706575, | cg26537335, | cg00593689, | cg09508770, |
| cg21932384, | cg17872799, | cg22656048, | cg11980819, | cg04023982, | cg10093254, | cg03098515, |
| cg17735372, | cg18845375, | cg16404569, | cg00894440, | cg27448574, | cg09530108, | cg06121226, |
| cg22032789, | cg00538458, | cg10122766, | cg07234508, | cg27525433, | cg08547367, | cg22964929, |
| cg23793121, | cg09798090, | cg03957898, | cg17982504, | cg00890916, | cg06761167, | cg04738447, |
| cg09607363, | cg05627103, | cg01183053, | cg16919317, | cg03339987, | cg02826525, | cg23840866, |
| cg03559454, | cg14452121, | cg11292536, | cg01508023, | cg07252731, | cg08766915, | cg12906975, |
| cg03313574, | cg24723140, | cg03928410, | cg11136111, | cg25503323, | cg25264554, | cg24207176, |
| cg12232903, | cg01635736, | cg10703481, | cg04833165, | cg16037137, | cg17416730, | cg24687805, |
| cg11031869, | cg25045785, | cg27038348, | cg23320693, | cg24701725, | cg00831931, | cg06119630, |
| cg24229963, | cg01344060, | cg18922994, | cg04531363, | cg16483725, | cg20685475, | cg05195247, |
| cg04037228, | cg08600430, | cg19015951, | cg08080475, | cg13075600, | cg01407244, | cg04774711, |
| cg10891888, | cg02404739, | cg22935821, | cg14509403, | cg08816023, | cg10993442, | cg14378039, |
| cg11069276, | cg25072359, | cg25683325, | cg18102337, | cg18105800, | cg13258831, | cg03981954, |
| cg12202913, | cg05308970, | cg07271405, | cg16091920, | cg05716567, | cg14529331, | cg07645809, |
| cg23968739, | cg01435791, | cg27126575, | cg24730705, | cg19728226, | cg20897830, | cg03605361, |
| cg19895164, | cg20141824, | cg22830590, | cg14489649, | cg25990985, | cg14667192, | cg02916102, |
| cg22381068, | cg07489029, | cg04474085, | cg04428799, | cg23090606, | cg26880735, | cg10603004, |
| cg19375644, | cg05566397, | cg02115117, | cg25411363, | cg06270147, | cg02632822, | cg02725908, |
| cg00713113, | cg11833125, | cg10311806, | cg05202822, | cg03585611, | cg00407413, | cg17379886, |
| cg09387486, | cg17463161, | cg05621259, | cg05437823, | cg23855505, | cg21614759, | cg01962509, |
| cg06023066, | cg04517079, | cg05198297, | cg02390624, | cg27597790, | cg16726706, | cg16662785, |
| cg15089892, | cg12646585, | cg21441657, | cg21220374, | cg27662454, | cg01137760, | cg09411252, |
| cg07927219, | cg11834681, | cg26700716, | cg06578825, | cg25106220, | cg00402366, | cg22116290, |
| cg17022244, | cg18892461, | cg21703538, | cg23839571, | cg08849558, | cg20219159, | cg12188928, |
| cg14371292, | cg15559398, | cg26687406, | cg10336707, | cg21576151, | cg07561552, | cg02275713, |
| cg12089360, | cg18339598, | cg19086455, | cg15996397, | cg10428345, | cg12638731, | cg24180569, |
| cg07812887, | cg18150247, | cg18489945, | cg22562040, | cg16885608, | cg12529801, | cg05602715, |
| cg23733133, | cg06198069, | cg22327527, | cg05341848, | cg13780295, | cg27430637, | cg27451531, |
| cg09642428, | cg19374779, | cg23569090, | cg27566403, | cg23201812, | cg02917040, | cg19616851, |
| cg00235367, | cg00626856, | cg02938130, | cg20785333, | cg06534061, | cg25071674, | cg08036764, |
| cg27577928, | cg11516377, | cg27635859, | cg21865128, | cg13135180, | cg24157982, | cg27619457, |
| cg10100698, | cg10650299, | cg08559317, | cg14591419, | cg09323728, | cg10978983, | cg05926722, |
| cg14794043, | cg25810484, | cg03937687, | cg07057177, | cg10565512, | cg07871590, | cg02534659, |
| cg10958126, | cg12198913, | cg11084470, | cg00203617, | cg10154122, | cg19350728, | cg16414852, |
| cg09321400, | cg18801906, | cg17718960, | cg17875845, | cg22123387, | cg13304350, | cg02194396, |
| cg02412050, | cg15264610, | cg17131178, | cg01293971, | cg27513517, | cg00789004, | cg24714606, |
| cg10025462, | cg10564626, | cg22614521, | cg16032428, | cg13754915, | cg19723459, | cg10127221, |
| cg17384436, | cg10183603, | cg00645755, | cg26015513, | cg14455590, | cg13908476, | cg06363041, |
| cg08860941, | cg05658793, | cg25730564, | cg14447193, | cg26391564, | cg23479065, | cg07900024, |
| cg12374682, | cg20980321, | cg10980436, | cg07265541, | cg20744625, | cg12062843, | cg23036947, |
| cg08300443, | cg16386523, | cg00821051, | cg23223091, | cg13996395, | cg07112173, | cg10152810, |
| cg11354594, | cg13770361, | cg05187965, | cg09169215, | cg23796525, | cg25087851, | cg10047480, |
| cg15261861, | cg04498913, | cg00686339, | cg07171893, | cg18127003, | cg24005158, | cg22321036, |
| cg24312874, | cg01941047, | cg02636872, | cg26836577, | cg08078213, | ch.19.1033996R, | cg05814351, |
| cg07138115, | cg22291711, | cg09614724, | cg05764376, | cg27100471, | cg06606318, | cg01026204, |
| cg04694744, | cg17965019, | cg09294998, | cg12381164, | cg10024909, | cg23016776, | cg04543742, |
| cg26881277, | cg20666533, | cg15391433, | cg05818351, | cg03314569, | cg03533141, | cg11765438, |
| cg19708659, | cg16058485, | cg19258425, | cg02286602, | cg12944006, | cg22425568, | cg21240492, |
| cg03455964, | cg07150445, | cg04062576, | cg12821702, | cg10981580, | cg14472551, | cg26931307, |
| cg23871507, | cg03152084, | cg19153329, | cg09339907, | cg11526630, | cg20665459, | cg26614262, |
| cg06493080, | cg03379270, | cg26329440, | cg13860849, | cg05025179, | cg19832721, | cg25878872, |
| cg00132782, | cg04851639, | cg20975708, | cg09247619, | cg18427336, | cg14164099, | cg13098071, |
| cg06680397, | cg05967120, | cg10714492, | cg05612640, | cg09172260, | cg13156207, | cg09764611, |
| cg26094983, | cg13473120, | cg10349879, | cg05421688, | cg08807341, | cg11303630, | cg02240876, |
| cg05992977, | cg16845701, | cg26781150, | cg20773023, | cg06434775, | cg12527260, | cg09248009, |
| cg10961853, | cg19780993, | cg26451355, | cg14509390, | cg02282640, | cg21838625, | cg20935553, |
| cg00397849, | cg05152568, | cg17309077, | cg02716317, | cg14475875, | cg25722373, | cg25206096, |
| cg14029580, | cg23235135, | cg06845079, | cg23695037, | cg13652372, | cg07739472, | cg25687360, |
| cg12038583, | cg10200728, | ch.9.1059422R, | cg14002752, | cg12956380, | cg05917225, | cg19209385, |
| cg13753900, | cg03856748, | cg06547541, | cg10706553, | cg09670530, | cg02632742, | cg05448271, |

DMP IDs:

| | | | | |
|---|---|---|---|---|
| cg06998361, | cg13899678, | cg15429502, | cg24874828, | cg15442811, |
| cg07905888, | cg27044148, | cg20691722, | cg27508378, | cg23060513, |
| cg25605243, | cg23369748, | cg05863637, | cg27366994, | cg16802027, |
| cg08173959, | cg13973002, | cg25064146, | cg01503329, | ch.2.62306258F, |
| cg18545076, | cg04025739, | cg19973659, | cg23475963, | cg00346247, |
| cg14424530, | cg14783987, | cg02175263, | cg14176088, | cg12935478, |
| cg03099611, | cg03408904, | cg00866215, | cg20181325, | cg04989913, |
| cg04713453, | cg23610841, | cg02097616, | cg18758482, | cg04319617, |
| cg17501982, | cg10143323, | cg06526872, | cg18296078, | cg13033972, |
| cg13463245, | cg08137352, | cg20786131, | cg23601994, | cg06436504, |
| cg14174317, | cg27320127, | cg00955035, | cg08077682, | cg26104143, |
| cg14075393, | cg13845561, | cg08926571, | cg13344344, | cg09591406, |
| cg00745735, | cg18022883, | cg09575269, | cg01764713, | cg03893692, |
| cg14275095, | cg07782479, | cg14457284, | cg22034315, | cg16222698, |
| cg16003301, | cg18609891, | cg17419660, | cg19950823, | cg06514344, |
| cg10516516, | cg16217297, | cg10689175, | cg00804078, | cg23840275, |
| cg13227697, | cg07342790, | cg06383625, | cg10589539, | cg20420559, |
| cg06075311, | cg20659025, | cg03799473, | cg01505421, | cg12951726, |
| cg06776173, | cg13665714, | cg09298466, | cg13902189, | cg24463320, |
| cg20278525, | cg15790852, | cg10950441, | cg20261917, | cg06098368, |
| cg13425294, | cg15950337, | cg07817705, | cg21428710, | cg08037401, |
| cg03508786, | cg13341380, | cg10866445, | cg09292244, | cg23405212, |
| cg22105613, | cg08725319, | cg25210134, | cg19453769, | cg06173626, |
| cg25024001, | cg08797423, | cg21960110, | cg19004134, | cg14966074, |
| cg02675859, | cg23651976, | cg01222316, | cg23533267, | cg20913750, |
| cg02201774, | cg00926215, | cg19258868, | cg24905196, | cg17970176, |
| cg08796706, | cg06582782, | cg03650674, | cg27479571, | cg23892924, |
| cg20332195, | cg22079747, | cg09850969, | cg09657673, | cg10308265, |
| cg16672637, | cg24603490, | cg12781834, | cg00577560, | cg21226059, |
| cg15952465, | cg13257263, | cg09303678, | cg15369608, | cg24217984, |
| cg03454607, | cg18254147, | cg11351709, | cg02456292, | cg01207010, |
| cg23641852, | cg22389279, | cg20947377, | cg16051002, | cg06900514, |
| cg26332253, | cg12528649, | cg26434983, | cg05654645, | cg13827212, |
| cg20665464, | cg06100552, | cg03410576, | cg11509669, | cg03864483, |
| cg17729667, | cg24451858, | cg24875857, | cg12905371, | cg17203290, |
| cg24459227, | cg23845153, | cg21130958, | cg16888565, | cg17135058, |
| cg06778159, | cg17649243, | cg18054725, | cg15526406, | cg03600886, |
| cg17508302, | cg21082921, | cg19595850, | cg17321888, | cg01833755, |
| cg21454161, | cg17284440, | cg07435282, | cg23249369, | cg06312477, |
| cg04326854, | cg16492735, | cg07956346, | cg04882870, | cg23367987, |
| cg19013339, | cg25310824, | cg15734436, | cg04896959, | cg03645984, |
| cg26417779, | cg21088460, | cg05968822, | cg19638039, | cg19847643, |
| cg07297039, | cg22969808, | cg11874976, | cg04739647, | cg11539066, |
| cg02764239, | cg21916596, | cg25590640, | cg11342234, | cg05161529, |
| cg15192750, | cg07294717, | cg10864318, | cg14096962, | cg23206103, |
| cg01811109, | cg15089864, | cg17000666, | cg04180299, | cg07171867, |
| cg17624938, | cg20641095, | cg23350385, | cg06993191, | cg09619146, |
| cg10186232, | cg01431063, | cg05310920, | cg17011709, | cg11997570, |
| cg03492327, | cg22692158, | cg05185926, | cg26581165, | cg16293249, |
| cg25716871, | cg13092433, | cg06044662, | cg10213928, | cg06352730, |
| cg23792592, | cg10589813, | cg19504950, | cg00662782, | cg00130223, |
| cg23633543, | cg06445348, | cg24697582, | cg02106116, | cg07020407, |
| cg14320938, | cg00066663, | cg06221449, | cg12754571, | cg07010222, |
| cg09315329, | cg12721730, | cg14723921, | cg13677144, | cg00160902, |
| cg01257975, | cg13826459, | cg10703692, | cg23318893, | cg18237277, |
| cg25484139, | cg09761121, | cg16438722, | cg22332276, | cg14556070, |
| cg26370066, | cg19517135, | cg09927637, | cg10524701, | cg12870705, |
| cg05765605, | cg08751876, | cg05338680, | cg03459202, | cg13261883, |
| cg09506001, | cg09303201, | cg18115040, | cg03988778, | cg26989374, |
| cg13452923, | cg11331028, | cg10842141, | cg03889876, | cg27646848, |
| cg27040423, | cg01993468, | cg18683453, | cg04689080, | cg27458060, |
| cg03837111, | cg15203301, | cg05176958, | cg18440893, | cg24284436, |
| cg15605744, | cg12462377, | cg23528982, | cg07962620, | cg22104103, |
| cg23815702, | cg01222380, | cg25034423, | cg06922352, | cg15650170, |
| cg04997017, | cg05660881, | cg02934500, | cg18084083, | cg06911282, |
| cg14191369, | cg11344668, | cg08876130, | cg04048461, | cg15085372, |
| cg27228562, | cg24298311, | cg11265488, | cg14457607, | cg04627891, |
| cg23015917, | cg27302373, | cg17128068, | cg19727059, | cg00701064, |
| cg10428805, | cg02765085, | cg19628686, | cg25720022, | cg04295117, |
| cg21771200, | cg01585293, | cg04676715, | cg24140841, | cg16336248, |
| cg11202422, | cg04095732, | cg23883514, | cg24866538, | cg14691596, |
| cg22934237, | cg13957558, | cg11301967, | cg17317439, | cg06099048, |
| cg11823624, | cg00354484, | cg04739306, | cg02324789, | cg21575951, |
| cg18213440, | cg26513391, | cg15386550, | cg25586011, | cg02582587, |
| cg08682334, | cg23397578, | cg04011030, | cg13927605, | cg10314172, |
| cg08638512, | cg06295928, | cg16505455, | cg22101703, | cg23542495, |
| cg10469659, | cg12026546, | cg01106145, | cg23079262, | cg14631703, |
| cg08453750, | cg11275685, | cg23266743, | cg24217894, | cg26676091, |
| cg10312334, | cg10468845, | cg03845214, | cg15984718, | cg17109042, |
| cg27002826, | cg01092293, | cg18161654, | cg00816770, | cg19221369, |
| cg02600551, | cg19049837, | cg18030105, | cg00251125, | cg06116271, |
| cg00600854, | cg07128111, | cg01963240, | cg04421631, | cg24819686, |
| cg18606310, | cg02676148, | cg04832325, | cg24746100, | cg03814316, |
| cg13922421, | cg24641901, | cg22891408, | cg00082874, | cg27541349, |
| cg07912251, | cg24114371, | cg05979332, | cg02165978, | cg26057765, |
| cg04254347, | cg07175848, | cg19745314, | cg00099017, | cg02652018, |
| cg22599005, | cg20702802, | cg22535442, | cg09117643, | cg08617069, |
| cg10763234, | cg05785222, | cg16670554, | cg24982402, | cg00530127, |
| cg06979110, | cg03241725, | cg13672200, | cg25475603, | cg21410961, |
| cg11421509, | cg18834029, | cg11850943, | cg21777089, | cg04458281, |
| cg15320905, | cg12443285, | cg05842120, | cg01329010, | cg20302082, |
| cg06553114, | cg27231712, | cg26138460, | cg04885072, | cg24473594, |
| cg08126211, | cg13811423, | cg17741192, | cg22976700, | cg22294773, |
| cg03738384, | cg24926589, | cg21130718, | cg26888727, | cg21963854, |
| cg00308909, | cg01943843, | cg26913927, | cg21987731, | cg01035616, |
| cg04918978, | cg09655272, | cg02109189, | cg00706833, | cg25503001, |
| cg24158045, | cg24194539, | cg22584802, | cg01712159, | cg15756653, |
| cg20007465, | cg26585416, | cg01165043, | cg17503823, | cg24675735, |
| cg07533951, | cg24118495, | cg25975823, | cg10519271, | cg05883167, |
| cg12367789, | cg03658275, | cg16279541, | cg14380111, | cg13660372, |
| cg19769182, | cg02313118, | cg08740477, | cg07216337, | cg02212280, |
| cg04169534, | cg10792987, | cg13990947, | cg25708790, | cg18399472, |
| cg17982116, | cg13734953, | cg08029329, | cg14937069, | cg00304388, |
| cg19640303, | cg26470924, | cg02705444, | cg16525575, | cg19110895, |
| cg18739166, | cg00500333, | cg10809485, | cg25685761, | cg06398114, |
| cg15936676, | cg17112155, | cg25439798, | cg13808420, | cg15994321, |
| cg26841068, | cg08815583, | cg00414561, | cg21674704, | cg03875678, |
| cg09258878, | cg07960083, | cg05433039, | cg23933345, | cg00049033, |
| cg17788149, | cg19521934, | cg27537046, | cg14716686, | cg24188437, |
| cg24875029, | cg27044841, | cg02629313, | cg00649606, | cg27472102, |
| cg10858225, | cg23197280, | cg26309854, | cg02428792, | cg01203924, |
| cg03082589, | cg04043917, | cg00753399, | cg01578632, | cg07135601, |
| cg00534492, | cg01168890, | cg06878866, | cg17355791, | cg17796897, |
| cg14638988, | cg13719843, | cg17924366, | cg27036581, | cg06974458, |
| cg14275354, | cg10997640, | cg27409991, | cg16630572, | cg19628333, |
| cg12661145, | cg10163222, | cg05386508, | cg23240355, | cg11085304, |
| cg18639049, | cg13745489, | cg00662142, | cg20697025, | cg04789747, |
| cg23533824, | cg18041435, | cg25224543, | cg03508063, | cg06434864, |
| cg12165215, | cg15907372, | cg20655068, | cg22383888, | cg07101841, |
| cg10235275, | cg24535910, | cg20100668, | cg11116429, | cg17887478, |
| cg17209284, | cg09017174, | cg17701073, | cg03553826, | cg07919443, |
| cg01959071, | cg00144832, | cg26770055, | cg00281842, | cg03349020, |
| cg23055236, | cg03464689, | cg05957603, | cg20418308, | cg17109036, |
| cg21814278, | cg21636353, | cg04989813, | cg12950645, | cg17136699, |
| cg12148714, | cg23791011, | cg20465064, | cg17067993, | cg11115267, |
| cg23732962, | cg01883427, | cg04034967, | cg09382096, | cg20703895, |
| cg16535018, | cg18098567, | cg19177884, | cg21545859, | cg21035843, |
| cg08126815, | cg25728876, | cg02046017, | cg14793764, | cg15425670, |
| cg12135887, | cg08411860, | cg05373263, | cg17445044, | cg26017700, |
| cg12195798, | cg17581870, | cg01159980, | cg14859667, | cg04012535, |
| cg15258031, | cg14500718, | cg07378821, | cg06284756, | cg23638624, |
| cg02989760, | cg04983685, | cg18193127, | cg21985862, | cg12918130, |
| cg19980718, | cg02998861, | cg23686029, | cg22059713, | cg01936347, |
| cg03624415, | cg07522194, | cg01188466, | cg17480076, | cg26390965, |
| cg08530030, | cg27441225, | cg09972364, | cg15701317, | cg24141233, |
| cg12401343, | cg10337322, | cg13531223, | cg03521077, | cg01806741, |
| cg10115182, | cg12578575, | cg19039291, | cg08092513, | cg19584530, |
| cg07047197, | cg20985479, | cg27210447, | cg00286986, | cg26872137, |
| cg08987606, | cg25902554, | cg22724741, | cg14235416, | cg13713066, |
| cg20266894, | cg04341730, | cg10712561, | cg01555791, | cg07570677, |
| cg26943001, | cg04405541, | cg26317237, | cg13732582, | cg21911500, |
| cg02904493, | cg17592667, | cg17225591, | cg17339543, | cg15522517, |
| cg25764931, | cg02515217, | cg11754318, | cg18758796, | cg03152913, |
| cg13820205, | cg00870242, | cg07412254, | cg23392162, | cg04019016, |
| cg01244514, | cg27597681, | cg18500556, | cg21838880, | cg27071234, |
| cg18265435, | cg02725362, | cg12739454, | cg11645658, | cg15558634, |
| cg23205183, | cg17175419, | cg00998132, | cg00771084, | cg13727629, |
| cg25818522, | cg14209781, | cg15869463, | cg14583999, | cg05529448, |
| cg00533183, | cg19411441, | cg05791870, | cg23302321, | cg05673966, |
| cg04779788, | cg02197677, | cg16069236, | cg10786226, | cg27594176, |
| cg05083414, | cg24685926, | cg03013999, | cg21021448, | cg04418091, |
| cg27509381, | cg15751117, | cg07893474, | cg21249274, | cg13490677, |
| cg00765312, | cg02303361, | cg03366312, | cg22162435, | cg19895474, |
| cg07433769, | cg22124859, | cg02622866, | cg20550458, | cg20870668, |
| cg19668255, | cg08141245, | cg24555089, | cg11579758, | cg02207312, |
| cg16201146, | cg03534635, | cg20878190, | cg04650641, | cg12757011, |
| cg23491236, | cg15249629, | cg08495374, | cg05877367, | cg17453456, |
| cg05964373, | cg15803018, | cg05706898, | cg24209738, | cg19356389, |
| cg17333242, | cg14753809, | cg13468961, | cg05414026, | cg05020203, |
| cg17467879, | cg18436324, | cg14022183, | cg13184823, | cg20629089, |
| cg24932585, | cg15757195, | cg09147880, | cg27665823, | cg18011946, |
| cg18592459, | cg24926465, | cg13017345, | cg20056634, | cg07096218, |
| cg07775417, | cg01588379, | cg27611274, | cg24484138, | cg11198094, |
| cg07126216, | cg23994025, | cg25218649, | cg10293297, | cg00816406, |
| cg07841477, | cg16605942, | cg15684329, | cg03779973, | cg01874697, |
| cg26085162, | cg15121420, | cg00099768, | cg22620027, | cg18168101, |
| cg13930544 | | | | |

¹CvC—cancer vs. control samples (TCGA dataset)
²PvR—progressive vs. regressive CIS dataset

### REFERENCES

1 Huber, W. et al. Orchestrating high-throughput genomic analysis with Bioconductor. Nat Methods 12, 115-121, doi:10.1038/nmeth.3252 (2015).
2 Jeremy George, P. et al. Surveillance for the detection of early lung cancer in patients with bronchial dysplasia. Thorax 62, 43-50, doi:10.1136/thx.2005.052191 (2007).
3 Bibikova, M. et al. High density DNA methylation array with single CpG site resolution. Genomics 98, 288-295, doi:10.1016/j.ygeno.2011.07.007 (2011).
4 Sandoval, J. et al. Validation of a DNA methylation microarray for 450,000 CpG sites in the human genome. Epigenetics 6, 692-702 (2011).
5 Morris, T. J. et al. ChAMP: 450 k Chip Analysis Methylation Pipeline. Bioinformatics 30, 428-430, doi:10.1093/bioinformatics/btt684 (2014).
6 Teschendorff, A. E. et al. DNA methylation outliers in not vial breast tissue identify field defects that are enriched in cancer. Nat Commun 7, 10478, doi:10.1038/ncomms10478 (2016).
7 Johnson, W. E., Li, C. & Rabinovic, A. Adjusting batch effects in microarray expression data using empirical Bayes methods. Biostatistics 8, 118-127, doi:10.1093/biostatistics/kxj037 (2007).
8 Ritchie, M. E. et al. limma powers differential expression analyses for RNA-sequencing and microarray studies. Nucleic acids research 43, e47, doi:10.1093/nar/gkv007 (2015).
9 Benjamini, Y., Drai, D., Elmer, G., Kafkafi, N. & Golani, I. Controlling the false discovery rate in behavior genetics research. Behav Brain Res 125, 279-284 (2001).
10 Kolde, R. Pheatmap: pretty heatmaps. R package version 61 (2012).
11 Tibshirani, R., Hastie, T., Narasimhan, B. & Chu, G. Diagnosis of multiple cancer types by shrunken centroids of gene expression. Proceedings of the National Academy of Sciences of the United States of America 99, 6567-6572, doi:10.1073/pnas.082099299 (2002).
12 Robin, X. et al. pROC: an open-source package for R and S+ to analyze and compare ROC curves. BMC Bioinfoimatics 12, 77, doi:10.1186/1471-2105-12-77 (2011).
13 Keilwagen, J., Grosse, I. & Grau, J. Area under precision-recall curves for weighted and unweighted data. PLoS One 9, e92209, doi:10.1371/journal.pone.0092209 (2014).
14 Raine, K. M. et al. ascatNgs: Identifying Somatically Acquired Copy-Number Alterations from Whole-Genome Sequencing Data. Current protocols in bioinformatics 56, 15 19 11-15 19 17, doi:10.1002/cpbi.17 (2016).
15 Feber, A. et al. Using high-density DNA methylation arrays to profile copy number alterations. Genome Biol 15, R30, doi:10.1186/gb-2014-15-2-r30 (2014).
16 van Boerdonk, R. A. et al. DNA copy number alterations in endobronchial squamous metaplastic lesions predict lung cancer. American journal of respiratory and critical care medicine 184, 948-956, doi:10.1164/rccm.201102-02180C (2011).
17 van Boerdonk, R. A. et al. DNA copy number aberrations in endobronchial lesions: a validated predictor for cancer. Thorax 69, 451-457, doi:10.1136/thoraxjnl-2013-203821 (2014).
18 Grossman, R. L. et al. Toward a Shared Vision for Cancer Genomic Data. N Engl J Med 375, 1109-1112, doi:10.1056/NEJMp1607591 (2016).
19 Law, C. W., Chen, Y., Shi, W. & Smyth, G. K. voom: Precision weights unlock linear model analysis tools for RNA-seq read counts. Genome biology 15, R29, doi:10.1186/gb-2014-15-2-r29 (2014).
20 Luo, W., Friedman, M. S., Shedden, K., Hankenson, K. D. & Woolf, P. J. GAGE: generally applicable gene set enrichment for pathway analysis. BMC Bioinformatics 10, 161, doi:10.1186/1471-2105-10-161 (2009).
21 Kanehisa, M. & Goto, S. KEGG: kyoto encyclopedia of genes and genomes. Nucleic acids research 28, 27-30 (2000).
22 Kanehisa, M., Sato, Y., Kawashima, M., Furumichi, M. & Tanabe, M. KEGG as a reference resource for gene and protein annotation. Nucleic acids research 44, D457-462, doi:10.1093/nar/gkv1070 (2016).
23 Kanehisa, M., Furumichi, M., Tanabe, M., Sato, Y. & Morishima, K. KEGG: new perspectives on genomes, pathways, diseases and drugs. Nucleic acids research 45, D353-D361, doi:10.1093/nar/gkw1092 (2017).
24 Carter, S. L., Eklund, A. C., Kohane, I. S., Harris, L. N. & Szallasi, Z. A signature of chromosomal instability inferred from gene expression profiles predicts clinical outcome in multiple human cancers. Nat Genet 38, 1043-1048, doi:10.1038/ng1861 (2006).
25 Whitfield, M. L. et al. Identification of genes periodically expressed in the human cell cycle and their expression in tumors. Mol Biol Cell 13, 1977-2000, doi:10.1091/mbc.02-02-0030. (2002).
26 Jones, D. et al. cgpCaVEManWrapper: Simple Execution of CaVEMan in Order to Detect Somatic Single Nucleotide Variants in NGS Data. Current protocols in bioinformatics 56, 15 10 11-15 10 18, doi:10.1002/cpbi.20 (2016).
27 Van Loo, P. et al. Allele-specific copy number analysis of tumors. Proc Natl Acad Sci USA 107, 16910-16915, doi:10.1073/pnas.1009843107 (2010).
28 Nik-Zainal, S. et al. The life history of 21 breast cancers. Cell 149, 994-1007, doi:10.1016/j.cell.2012.04.023 (2012).
29 Skinner, M. E., Uzilov, A. V., Stein, L. D., Mungall, C. J. & Holmes, I. H. JBrowse: a next-generation genome browser. Genome Res 19, 1630-1638, doi:10.1101/gr.094607.109 (2009).
30 Raine, K. M. et al. cgpPindel: Identifying Somatically Acquired Insertion and Deletion Events from Paired End Sequencing. Curr Protoc Bioinformatics 52, 15 17 11-12, doi:10.1002/0471250953.bi1507s52 (2015).
31 Ye, K., Schulz, M. H., Long, Q., Apweiler, R. & Ning, Z. Pindel: a pattern growth approach to detect break points of large deletions and medium sized insertions from paired-end short reads. Bioinformatics 25, 2865-2871, doi:10.1093/bioinformatics/btp394 (2009).
32 Papaemmanuil, E. et al. RAG-mediated recombination is the predominant driver of oncogenic rearrangement in ETV6-RUNX1 acute lymphoblastic leukemia. Nature genetics 46, 116-125, doi:10.1038/ng.2874 (2014).
33 Robinson, J. T. et al. Integrative genomics viewer. Nat Biotechnol 29, 24-26, doi:10.1038/nbt.1754 (2011).
34 Thorvaldsdottir, H., Robinson, J. T. & Mesirov, J. P. Integrative Genomics Viewer (IGV): high-performance genomics data visualization and exploration. Brief Bioinform 14, 178-192, doi:10.1093/bib/bbs017 (2013).
35 Endesfelder, D. et al. Chromosomal instability selects gene copy-number variants encoding core regulators of proliferation in ER+ breast cancer. Cancer Res 74, 4853-4863, doi:10.1158/0008-5472.CAN-13-2664 (2014).
36 Forbes, S. A. et al. COSMIC: somatic cancer genetics at high-resolution. Nucleic acids research 45, D777-D783, doi:10.1093/nar/gkw1121 (2017).
37 McLaren, W. et al. The Ensembl Variant Effect Predictor. Genome Biol 7, 122, doi:10.1186/s13059-016-0974-4 (2016).
38 Martincorena, I. et al. Universal Patterns of Selection in Cancer and Somatic Tissues. Cell 171, 1029-1041 e1021, doi:10.1016/j.cell.2017.09.042 (2017).
39 Miller, C. A. et al. SciClone: inferring clonal architecture and tracking the spatial and temporal patterns of tumor evolution. PLoS Comput Biol 10, e1003665, doi:10.1371/journal.pcbi.1003665 (2014).
40 McGranahan, N. et al. Clonal neoantigens elicit T cell immunoreactivity and sensitivity to immune checkpoint blockade. Science 351, 1463-1469, doi:10.1126/science.aaf1490 (2016).
41 Blokzijl, F., Janssen, R., van Boxtel, R. & Cuppen, E. MutationalPatterns: comprehensive genome-wide analysis of mutational processes. Genome Med 10, 33, doi:10.1186/s13073-018-0539-0 (2018).
42 Alexandrov, L. B. et al. Clock-like mutational processes in human somatic cells. Nat Genet 47, 1402-1407, doi:10.1038/ng.3441 (2015).
43 Alexandrov, L. B. et al. Signatures of mutational processes in human cancer. Nature 500, 415-421, doi:10.1038/nature12477 (2013).
44 Martincorena, I. & Campbell, P. J. Somatic mutation in cancer and not vial cells. Science 349, 1483-1489, doi:10.1126/science.aab4082 (2015).
45 Farmery, J. H. R., Smith, M. L. & Lynch, A. G. Telomerecat: A Ploidy-Agnostic Method For Estimating Telomere Length From Whole Genome Sequencing Data. bioRxiv, doi:10.1101/139972 (2017).
46 Olkhov-Mitsel, E and Bapat, B: Strategies for discovery and validation of ethylated and hydroxymethylated DNA biomarkers. Cancer Medicine 2012, 1(2): 237-260.
47 Herman, J. G., Graff, J. R., Myohanen, S., Nelkin, B. D. & Baylin, S. B.: Methylation-specific PCR: a novel PCR assay for methylation status of CpG islands. Proc. Natl Acad. Sci. USA 1996, 93: 9821-9826.
48 Frommer, M. et al.: A genomic sequencing protocol that yields a positive display of 5-methylcytosine residues in individual DNA strands. Proc. Natl Acad. Sci. USA 1992, 89: 1827-1831.
49 Xiong, Z. & Laird, P. W.: COBRA: a sensitive and quantitative DNA methylation assay. Nucleic Acids Res. 1997, 25: 2532-2534.
50 Gonzalgo, M. L. & Jones, P. A.: Rapid quantitation of methylation differences at specific sites using methylationsensitive single nucleotide primer extension (Ms-SNuPE). Nucleic Acids Res. 1997, 25: 2529-2531.
51 Paul D S, Guilhamon P, Karpathakis A, Butcher L M, Thirlwell C, Feber A, Beck S: Assessment of RainDrop BS-seq as a method for large-scale, targeted bisulfite sequencing. Epigenetics 2014, 9.

1. A method of identifying whether or not an individual has a cancer, a pre-invasive lesion that will progress to a cancer, or a pre-cancerous cell population that will progress to a cancer, the method comprising:
(a) providing a DNA sample which has been taken from the individual;
(b) performing an assay to determine the methylation status (β) value for at least 100 different differentially methylated positions (DMPs) in the DNA sample;
(b) determining a methylation heterogeneity index (MHI) for the DNA sample, wherein the MHI is defined as the proportion of the assayed DMPs which have a β value which is intermediate between 1 and 0; and
(c) identifying whether or not the individual has a cancer, a pre-invasive lesion that will progress to a cancer, or a pre-cancerous cell population that will progress to a cancer, based on the MHI.

2. The method of claim 1, wherein one or more of the at least 100 DMPs is a CpG.

3. The method of claim or 2, wherein an intermediate β value is defined as tₗₒ<β<tₕᵢ, optionally wherein tₗₒ is about 0.2, about 0.21, about 0.22, about 0.23, about 0.24, about 0.25, about 0.26, about 0.27, about 0.28, about 0.29, or about 0.3, and/or tₕᵢ is about 0.8, about 0.81, about 0.82, about 0.83, about 0.84, about 0.85, about 0.86, about 0.87, about 0.88, about 0.89, about 0.9, about 0.91, about 0.92, about 0.93, about 0.94, or about 0.95, preferably wherein tₗₒ is about 0.26 and tₕᵢ is about 0.88.

4. The method of any one of the preceding claims, wherein the individual is identified as having a cancer, a pre-invasive lesion that will progress to a cancer, or a pre-cancerous cell population that will progress to a cancer if the MHI determined for the DNA sample is greater than a threshold value, optionally wherein the threshold value is from about 0.25 to about 0.45.

5. The method of any one of the preceding claims, wherein the individual is identified as having a cancer, a pre-invasive lesion that will progress to a cancer, or a pre-cancerous cell population that will progress to a cancer if the MHI deter mined for the DNA sample is greater than from about 0.25 to about 0.45

6. The method of any one of the preceding claims, wherein in step (b) a β value is determined for at least 200, at least 300, at least 500, at least 750, at least 1000, at least 1250, at least 1500, at least 2000, at least 5000, at least 10,000, at least 50,000, at least 100,000, at least 150,000, at least 200,000, at least 250,000, at least 300,000, at least 3500,00, at least 400,000, at least 450,000, or at least 500,000 DMPs.

7. The method of any one of the preceding claims, wherein the DMPs are substantially randomly distributed throughout the genome.

8. The method of any one of the preceding claims, wherein in step (b) at least about 100,000, at least about 200,000, at least about 300,000, at least about 400,000, at least about 500,000, at least about 750,0000, at least 1×10⁶, at least 1×10⁷, at least 1×10⁸, or at least 1×10⁹ individual DNA molecules are assayed per DMP.

9. The method of any one of the preceding claims, wherein the method achieves an ROC AUC of at least about 0.5, at least about 0.51, at least about 0.52, at least about 0.53, at least about 0.54, at least about 0.55, at least about 0.56, at least about 0.57, at least about 0.58, at least about 0.59, at least about 0.6, at least about 0.61, at least about 0.62, at least about 0.63, at least about 0.64, at least about 0.65, at least about 0.66, at least about 0.67, at least about 0.68, at least about 0.69, at least about 0.7, at least about 0.71, at least about 0.72, at least about 0.73, at least about 0.74, at least about 0.75, at least about 0.76, at least about 0.77, at least about 0.78, at least about 0.79, at least about 0.8, at least about 0.81, at least about 0.82, at least about 0.83, at least about 0.84, at least about 0.85, at least about 0.86, at least about 0.87, at least about 0.88, at least about 0.89, at least about 0.9, at least about 0.91, at least about 0.92, at least about 0.93, at least about 0.94, at least about 0.95, at least about 0.96, at least about 0.97, at least about 0.98, or at least about 0.99.

10. The method of any one of the preceding claims, wherein the method achieves a specificity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%.

11. The method of any one of the preceding claims, wherein the method achieves a sensitivity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%.

12. A method according to any one of the preceding claims for identifying whether or not an individual has a cancer, which achieves an ROC AUC of at least about 0.9, at least about 0.91, at least about 0.92, at least about 0.93, at least about 0.94, at least about 0.95, or at least about 0.96, preferably wherein the method achieves an ROC AUC of about 0.95 or about 0.96, optionally wherein the cancer is a lung cancer, preferably wherein the lung cancer is lung squamous cell carcinoma (LUSC).

13. A method according to any one of claims 1-17 for identifying whether or not an individual has a pre-invasive lesion that will progress to a cancer, which achieves an ROC AUC of at least about 0.66, at least about 0.67, at least about 0.68, at least about 0.69, at least about 0.7, at least about 0.71, at least about 0.72, at least about 0.73, at least about 0.74, or at least about 0.75, preferably wherein the method achieves an ROC AUC of about 0.74 or about 0.75, optionally wherein the pre-invasive lesion is a pre-invasive lung lesion, optionally wherein the pre-invasive lung lesion is a lung carcinoma in situ (CIS).

14. The method of any one of the preceding claims, wherein an intermediate β value is defined as 0.26<β<0.88, optionally wherein in step (b) a β value is determined for at least about 1500 DMPs, preferably about 2000 DMPs, optionally wherein the threshold value is from about 0.25 to about 0.45, preferably from about 0.28 to about 0.42, preferably from about 0.3 to about 0.4, preferably from about 0.32 to about 0.38, or preferably from about 0.34 to about 0.38.

15. The method according to any one of the preceding claims, wherein step (b) comprises bisulphite conversion of the DNA.

16. The method according to any one of the preceding claims, wherein step (b) comprises:
(i) performing a sequencing step to determine the sequence of the DNA molecules, preferably wherein before sequencing an amplification step is performed, preferably wherein the amplification step is performed by PCR; and/or
(ii) (a) hybridising the DNA to an array comprising probes capable of discriminating between methylated and non-methylated forms of DNA and applying a detection system to the array to discriminate methylated and non-methylated forms of DNA, optionally wherein before hybridisation an amplification step is performed, preferably wherein the amplification step is performed by PCR; or
(b) performing an amplification step using methylation-specific primers, wherein the methylation status of the DNA is determined by the presence or absence of an amplified product.

17. The method of any one of the preceding claims, wherein the DNA sample has been taken from a tissue, a bodily fluid and/or a circulating material previously obtained from the individual.

18. The method of claim 17, wherein the tissue has been obtained from a biopsy, optionally wherein the tissue, the bodily fluid or the circulating material is suspected of harbouring a cancer, a pre-invasive lesion, or a pre-cancerous cell population.

19. The method of any one of the preceding claims, wherein the assay to determine the methylation status of the DMPs outputs a signal for methylated CpGs (M) and a signal for the unmethylated CpGs (U), optionally wherein the value is calculated as intensity of M/(intensity of U+intensity of M+100), optionally wherein the signals for M and U are fluorescent signals.

20. A method of treating and/or preventing a cancer and/or treating a pre-invasive lesion that will progress to a cancer or a pre-cancerous cell population that will progress to a cancer in an individual, the method comprising:
identifying a cancer, a pre-invasive lesion that will progress to a cancer, or a pre-cancerous cell population that will progress to a cancer in the individual by performing a method according to any one of the preceding claims; and
administering a cancer therapy to the individual, optionally wherein the therapy comprises surgical intervention.

21. A methylation heterogeneity index (MHI) as defined in any one of the preceding claims for identifying in an individual a cancer, a pre-invasive lesion that will progress to a cancer, or a pre-cancerous cell population that will progress to a cancer.

22. The MHI of claim 21, wherein the MHI is determined by performing a method according to any one of claims 1-20.

23. The method of any one of claims 1-20 or the MHI of claim 21 or 21, wherein the DNA sample is from an individual:
(a) not suspected of having a pre-invasive lesion, a pre-cancerous cell population, or a cancer;
(b) suspected of having a pre-invasive lesion or a pre-cancerous cell population but not suspected of having a cancer;
(c) having a pre-invasive lesion or a pre-cancerous cell population but not suspected of having a cancer;
(d) having a pre-invasive lesion or a pre-cancerous cell population and suspected of having a cancer;
(e) suspected of having a pre-invasive lesion or a pre-cancerous cell population;
(f) having a pre-invasive lesion or a pre-cancerous cell population;
(g) suspected of having a cancer; or
(h) having a cancer.

24. The method or MHI according to any one of the preceding claims, wherein pre-invasive lesion is a solid lesion or the cancer is a solid tumour.

25. The method or MHI according to any one of the preceding claims, wherein:
(a) the pre-invasive lesion or pre-cancerous cell population is present in the central nervous system, the eye, the ear, nose or throat, the skin, the lung, the bone, an endocrine tissue, breast tissue, the digestive system, the reproductive system, the liver, or the kidney;
(b) the cancer is a cancer of the central nervous system, the eye, the ear, nose or throat, the skin, the lung, the bone, an endocrine tissue, breast tissue, the digestive system, the reproductive system, the liver, or the kidney;
(c) the pre-invasive lesion is normal epithelium, tissue hyperplasia, dysplasia, or lung carcinoma in situ (CIS); and/or
(d) the cancer is a lung cancer, optionally wherein the lung cancer is a lung squamous cell carcinoma (LUSC).
